(19) United States
(12) Patent Application Publication
(10) Pub. No.: US 2016/0032404 A1
(43) Pub. Date: Feb. 4, 2016

(54) BIOMARKERS FOR PREDICTING RESPONSE OF DLBCL TO TREATMENT WITH A BTK INHIBITOR

(71) Applicant: Pharmacyclics LLC, Sunnyvale, CA (US)

(72) Inventors: Karl SCHWEIGHOFER, Sunnyvale, CA (US); Karl ECKERT, Sunnyvale, CA (US); Hsu-Ping KUO, Sunnyvale, CA (US); Betty CHANG, Cupertino, CA (US); Darrin BEAUPRE, Campbell, CA (US); Leo CHEUNG, Fremont, CA (US)

(21) Appl. No.: 14/815,921

(22) Filed: Jul. 31, 2015

Related U.S. Application Data
(60) Provisional application No. 62/032,430, filed on Aug. 1, 2014.
(60) Provisional application No. 62/119,668, filed on Feb. 23, 2015.
(60) Provisional application No. 62/127,484, filed on Mar. 3, 2015.

(51) Int. Cl. C12Q 1/68; G01N 33/574

(52) U.S. Cl. CPC C12Q 1/6886; G01N 33/57407; G01N 33/5748; C12Q 2600/158; C12Q 2600/106; C12Q 2600/156; G01N 2800/52; G01N 2333/82; G01N 2333/70503; G01N 2333/4706

(57) ABSTRACT

Disclosed herein, are methods, systems, compositions, arrays, and kits for using biomarkers, biomarker genes (e.g. EP300, MLL2, BCL-2, RB1, LRP1B, PIM1, TSC2, TNFRSF11A, SMAD4, PAX5, CARD11, ACTG2, LOR, GAPT, CCND2, SELL, GEN1, HDAC9, CD79B, MYD88, and ROS1) or biomarker gene expression levels for stratifying a patient having a hematological malignancy such as DLBCL for treatment, and administering a TEC inhibitor to selected patients. Also disclosed herein are methods, systems, compositions, arrays, and kits for using biomarkers, biomarker genes, or biomarker gene expression levels for monitoring a patient during treatment of a hematological malignancy such as DLBCL or FL or for optimizing a treatment regimen with a TEC inhibitor.

This application claims benefit of priority of U.S. Provisional Application No. 62/032,430 filed Aug. 1, 2014; U.S. Provisional Application No. 62/119,668 filed Feb. 23, 2015; and U.S. Provisional Application No. 62/127,484 filed Mar. 3, 2015, each of which are herein incorporated by reference.

### BACKGROUND OF THE INVENTION

Bruton's tyrosine kinase (Btk), a member of the Tec family of non-receptor tyrosine kinases, is a key signaling enzyme expressed in all hematopoietic cells types except T lymphocytes and natural killer cells. Btk plays an essential role in the B-cell signaling pathway linking cell surface B-cell receptor (BCR) stimulation to downstream intracellular responses.

Diffuse large B cell lymphoma (DLBCL) is the most prevalent type of aggressive non-Hodgkin's lymphoma (NHL) in the United States. The ABC subtype of DLBCL (ABC-DLBCL) accounts for approximately 30% total DLBCL diagnoses. While majority of the patients with DLBCL show response to the initial treatment, approximately one-third of patients have refractory disease or experience relapse after the standard therapies. B cell receptor (BCR) signaling is an important growth and survival pathway in various B cell malignancies, including DLBCL.

### SUMMARY OF THE INVENTION

Disclosed herein, in certain embodiments, is a method for selecting an individual having diffuse large B cell lymphoma (DLBCL) for treatment with ibrutinib, comprising: (a) determining the presence or absence of a modification in one or more biomarker genes selected from EP300, MLL2, BCL-2, RB1, LRP1B, PIM1, TSC2, TNFRSF11A, SMAD4, PAX5, and CARD11; and (b) administering to the individual a therapeutically effective amount of ibrutinib if there is an absence of modifications in the one or more biomarker genes selected from EP300, MLL2, BCL-2, RB1, LRP1B, PIM1, TSC2, TNFRSF11A, SMAD4, PAX5, and CARD11. Also disclosed herein, in certain embodiments, is a method of monitoring whether an individual receiving ibrutinib for treatment of diffuse large B cell lymphoma (DLBCL) has developed or is likely to develop resistance to the therapy, comprising: (a) determining the presence or absence of a modification in one or more biomarker genes selected from EP300, MLL2, BCL-2, RB1, LRP1B, PIM1, TSC2, TNFRSF11A, SMAD4, PAX5, and CARD11; and (b) characterizing the individual as resistant or is likely to become resistant to therapy with ibrutinib if the individual has modifications in the one or more biomarker genes selected from EP300, MLL2, BCL-2, RB1, LRP1B, PIM1, TSC2, TNFRSF11A, SMAD4, PAX5, and CARD11. Further disclosed herein, in some embodiments, is a method of optimizing the therapy of an individual receiving ibrutinib for treatment of diffuse large B cell lymphoma (DLBCL), comprising: (a) determining the presence or absence of a modification in one or more biomarker genes selected from EP300, MLL2, BCL-2, RB1, LRP1B, PIM1, TSC2, TNFRSF11A, SMAD4, PAX5, and CARD11; and (b) modifying, discontinuing, or continuing the treatment based on the presence or absence of modifications in the one or more biomarker genes selected from EP300, MLL2, BCL-2, RB1, LRP1B, PIM1, TSC2, TNFRSF11A, SMAD4, PAX5, and CARD11. In some embodiments, the method further comprises determining the presence or absence of a modification in two or more biomarker genes selected from EP300, MLL2, BCL-2, RB1, LRP1B, PIM1, TSC2, TNFRSF11A, SMAD4, PAX5, and CARD11. In some embodiments, the one or more biomarker genes are selected from BCL-2, RB1, LRP1B, PIM1, and TSC2. In some embodiments, the one or more biomarker genes are selected from MLL2, RB1, TSC2 and combinations thereof, and the DLBCL is ABC-DLBCL. In some embodiments, the modification is base substitution, insertion, deletion, DNA rearrangement, copy number alteration, or a combination thereof. In some embodiments, EP300, MLL2, BCL-2, RB1, LRP1B, PIM1, TSC2, TNFRSF11A, SMAD4, PAX5, and CARD11 comprise one or more modifications in each gene. In some embodiments, the modification associated with the EP300, MLL2, BCL-2, RB1, LRP1B, PIM1, TSC2, TNFRSF11A, SMAD4, PAX5, and CARD11 genes results in modifications in the EP300, MLL2, BCL2, RB1, LRP1B, PIM1, TSC2, TNFRSF11A, SMAD4, PAX5, and CARD11 proteins. In some embodiments, the modification associated with the BCL-2 gene results in modifications in the BCL-2 protein. In some embodiments, the BCL-2 protein comprises one or more modifications at positions corresponding to amino acid residues 4, 9, 33, 47, 48, 49, 60, 68, 74, 113, 114, 120, 122, 129, 131, 165, 197, 198, 200, 201, 203, and 206. In some embodiments, the modifications include A4S, Y9H, G33R, G47A, I48S, F49L, A60T, R68K, T74N, T74S, A113G, E114A, H120Y, T122S, R129H, A131V, E165D, G197R, G197S, A198V, G200S, D201N, S203N, and 206W. In some embodiments, DLBCL is activated B-cell DLBCL (ABC-DLBCL), germinal center B-cell like DLBCL (GBC-DLBCL), or unclassified DLBCL. In some embodiments, the DLBCL is a relapsed or refractory DLBCL. In some embodiments, the method further comprises testing a sample containing nucleic acid molecules encoding the biomarker genes selected from EP300, MLL2, BCL-2, RB1, LRP1B, PIM1, TSC2, TNFRSF11A, SMAD4, PAX5, and CARD11 obtained from the individual, and determining whether each of the genes selected from EP300, MLL2, BCL-2, RB1, LRP1B, PIM1, TSC2, TNFRSF11A, SMAD4, PAX5, and CARD11 contains one or more modifications. In some embodiments, the nucleic acid molecule is RNA. In some embodiments, the nucleic acid molecule is DNA. In some embodiments, the DNA is genomic DNA. In some embodiments, testing comprises amplifying the nucleic acid molecules encoding the genes selected from EP300, MLL2, BCL-2, RB1, LRP1B, PIM1, TSC2, TNFRSF11A, SMAD4, PAX5, and CARD11. In some embodiments, amplification is by isothermal amplification or polymerase chain reaction (PCR). In some embodiments, amplification is by PCR. In some embodiments, testing comprises contacting nucleic acids with sequence specific nucleic acid probes, wherein the sequence specific nucleic acid probes bind to nucleic acids encoding modified genes selected from EP300, MLL2, BCL-2, RB1, LRP1B, PIM1, TSC2, TNFRSF11A, SMAD4, PAX5, and CARD11 and do not bind to nucleic acid encoding wild-type genes selected from EP300, MLL2, BCL-2, RB1, LRP1B, PIM1, TSC2, TNFRSF11A, SMAD4, PAX5, and CARD11. In some embodiments, testing comprises PCR amplification using the sequence specific nucleic acid probes. In some embodiments, the method further comprises obtaining the sample from the individual. In some embodiments, the sample contains one or more tumor cells from the individual. In some embodiments, the sample contains circulating tumor DNA (ctDNA). In some embodiments, the sample is a tumor biopsy sample, a blood sample, a serum sample, a lymph sample or a bone marrow aspirate. In some embodiments, the sample is a sample obtained prior to the first administration of ibrutinib. In some embodiments, the sample is a sample obtained at 1 week, 2 weeks, 3 weeks, 1 month, 2 months, 3 months, 4 months, 5 months, 6 months, 7 months, 8 months, 9 months, 10 months, 11 months, 12 months, 14 months, 16 months, 18 months, 20 months, 22 months, or 24 months following the first administration of ibrutinib. In some embodiments, the sample is obtained 1, 2, 3, 4, 5, 6, 7, 8, 9, 10 times over the course of treatment with ibrutinib. In some embodiments, ibrutinib is administered once a day, two times per day, three times per day, four times per day, or five times per day. In some embodiments, ibrutinib is administered at a dosage of about 40 mg/day to about 1000 mg/day. In some embodiments, ibrutinib is administered orally. In some embodiments, the method further comprises administering an additional therapeutic agent. In some embodiments, the additional therapeutic agent is selected from among a chemotherapeutic agent or radiation therapeutic agent. In some embodiments, the chemotherapeutic agent is selected from among chlorambucil, ifosfamide, doxorubicin, mesalazine, thalidomide, lenalidomide, temsirolimus, everolimus, fludarabine, fostamatinib, paclitaxel, docetaxel, ofatumumab, rituximab, dexamethasone, prednisone, CAL-101, ibritumomab, tositumomab, bortezomib, pentostatin, endostatin, or a combination thereof. In some embodiments, ibrutinib is administered simultaneously, sequentially or intermittently with the additional therapeutic agent.

Disclosed herein, in certain embodiments, is a method for selecting an individual having diffuse large B cell lymphoma (DLBCL) for treatment with ibrutinib, comprising: (a) determining the presence or absence of a modification to an aromatic residue at amino acid position 196 in CD79B and at least one modification at amino acid positions 198 or 265 in MYD88; and (b) administering to the individual a therapeutically effective amount of ibrutinib if there is a presence of the modification to an aromatic residue in CD79B and at least one modification at amino acid positions 198 or 265 in MYD88. Also disclosed herein, in certain embodiments, is a method of monitoring whether an individual receiving ibrutinib for treatment of diffuse large B cell lymphoma (DLBCL) is responsive or is likely to respond to therapy, comprising: (a) determining the presence or absence of a modification to an aromatic residue at amino acid position 196 in CD79B and at least one modification at amino acid positions 198 or 265 in MYD88; and (b) characterizing the individual as responsive or is likely to respond to therapy with ibrutinib if the individual has the modification to an aromatic residue at amino acid position 196 in CD79B and at least one modification at amino acid positions 198 or 265 in MYD88. In some embodiments, the aromatic residue is selected from among phenylalanine or tryptophan. Further disclosed herein, in certain embodiments, is a method of optimizing the therapy of an individual receiving ibrutinib for treatment of diffuse large B cell lymphoma (DLBCL), comprising: (a) determining the presence or absence of a modification to an aromatic residue at amino acid position 196 in CD79B and at least one modification at amino acid positions 198 or 265 in MYD88; and (b) modifying, discontinuing, or continuing the treatment based on the presence or absence of the modification to an aromatic residue at amino acid position 196 in CD79B and at least one modification at amino acid positions 198 or 265 in MYD88. In some embodiments, the presence of the combination of the modifications in CD79B and MYD88 indicates the individual is responsive or is likely to be responsive to treatment with ibrutinib. In some embodiments, the aromatic residue is phenylalanine or tryptophan. In some embodiments, the modification at amino acid position 196 in CD79B is Y196F. In some embodiments, the modification at amino acid position 198 in MYD88 is S198N. In some embodiments, the modification at amino acid position 265 in MYD88 is L265P. In some embodiments, the combination of the modifications in CD79B and MYD88 is Y196F and S198N or Y196F and L265P. In some embodiments, the DLBCL is activated B-cell DLBCL (ABC-DLBCL) or unclassified DLBCL. In some embodiments, the DLBCL is a relapsed or refractory DLBCL. In some embodiments, the method further comprises testing a sample containing nucleic acid molecules encoding CD79B and MYD88 polypeptides obtained from the individual, and determining whether each of the CD79B and MYD88 polypeptides contains the modifications. In some embodiments, the nucleic acid molecule is RNA or DNA. In some embodiments, the DNA is genomic DNA. In some embodiments, testing comprises amplifying the nucleic acid molecules encoding CD79B and MYD88 polypeptides. In some embodiments, amplification is by isothermal amplification or polymerase chain reaction (PCR). In some embodiments, amplification is by PCR. In some embodiments, testing comprises contacting nucleic acids with sequence specific nucleic acid probes, wherein the sequence specific nucleic acid probes bind to nucleic acids encoding modified CD79B and MYD88 polypeptides and do not bind to nucleic acid encoding wild-type CD79B and MYD88 polypeptides. In some embodiments, testing comprises PCR amplification using the sequence specific nucleic acid probes. In some embodiments, the method further comprises obtaining the sample from the individual. In some embodiments, the sample contains one or more tumor cells from the individual. In some embodiments, the sample contains circulating tumor DNA (ctDNA). In some embodiments, the sample is a tumor biopsy sample, a blood sample, a serum sample, a lymph sample or a bone marrow aspirate. In some embodiments, the sample is a sample obtained prior to the first administration of ibrutinib. In some embodiments, the sample is a sample obtained at 1 week, 2 weeks, 3 weeks, 1 month, 2 months, 3 months, 4 months, 5 months, 6 months, 7 months, 8 months, 9 months, 10 months, 11 months, 12 months, 14 months, 16 months, 18 months, 20 months, 22 months, or 24 months following the first administration of ibrutinib. In some embodiments, the sample is obtained 1, 2, 3, 4, 5, 6, 7, 8, 9, 10 times over the course of treatment with ibrutinib. In some embodiments, ibrutinib is administered once a day, two times per day, three times per day, four times per day, or five times per day. In some embodiments, ibrutinib is administered at a dosage of about 40 mg/day to about 1000 mg/day. In some embodiments, ibrutinib is administered orally. In some embodiments, the method further comprises administering an additional therapeutic agent. In some embodiments, the additional therapeutic agent is selected from among a chemotherapeutic agent or radiation therapeutic agent. In some embodiments, the chemotherapeutic agent is selected from among chlorambucil, ifosfamide, doxorubicin, mesalazine, thalidomide, lenalidomide, temsirolimus, everolimus, fludarabine, fostamatinib, paclitaxel, docetaxel, ofatumumab, rituximab, dexamethasone, prednisone, CAL-101, ibritumomab, tositumomab, bortezomib, pentostatin, endostatin, or a combination thereof. In some embodiments, ibrutinib is administered simultaneously, sequentially or intermittently with the additional therapeutic agent.

Disclosed herein, in certain embodiments, is a method for selecting an individual having diffuse large B cell lymphoma (DLBCL) for treatment with ibrutinib, comprising: (a) determining the presence or absence of a modification at amino acid position 15 in ROS1; and (b) administering to the individual a therapeutically effective amount of ibrutinib if there is an absence of the modification at amino acid position 15 in ROS1. Also disclosed herein, in certain embodiments, is a method of monitoring whether an individual receiving ibrutinib for treatment of diffuse large B cell lymphoma (DLBCL) has developed or is likely to develop resistance to the therapy, comprising: (a) determining the presence or absence of a modification at amino acid position 15 in ROS1; and (b) characterizing the individual as resistant or is likely to become resistant to therapy with ibrutinib if the individual has the modification at amino acid position 15 in ROS1. Further disclosed herein, in certain embodiments, is a method of optimizing the therapy of an individual receiving ibrutinib for treatment of diffuse large B cell lymphoma (DLBCL), comprising: (a) determining the presence or absence of a modification at amino acid position 15 in ROS1; and (b) modifying, discontinuing, or continuing the treatment based on the presence or absence of the modification at amino acid position 15 in ROS1. In some embodiments, the modification at amino acid position 15 in ROS1 is A15G. In some embodiments, the A15G modification in ROS1 further indicates the individual has developed or likely to develop a progressive DLBCL. In some embodiments, DLBCL is activated B-cell DLBCL (ABC-DLBCL), germinal center B-cell like DLBCL (GBC-DLBCL), or unclassified DLBCL. In some embodiments, the DLBCL is a relapsed or refractory DLBCL. In some embodiments, the method further comprises testing a sample containing nucleic acid molecules encoding the ROS1 polypeptide obtained from the individual, and determining whether the ROS1 polypeptide contains the modification at amino acid position 15. In some embodiments, the nucleic acid molecule is RNA or DNA. In some embodiments, the DNA is genomic DNA. In some embodiments, testing comprises amplifying the nucleic acid molecules encoding the ROS1 polypeptide. In some embodiments, amplification is by isothermal amplification or polymerase chain reaction (PCR). In some embodiments, amplification is by PCR. In some embodiments, testing comprises contacting nucleic acids with sequence specific nucleic acid probes, wherein the sequence specific nucleic acid probes bind to nucleic acids encoding a modified ROS1 polypeptide and do not bind to nucleic acid encoding the wild-type ROS1 polypeptide. In some embodiments, testing comprises PCR amplification using the sequence specific nucleic acid probes. In some embodiments, the method further comprises obtaining the sample from the individual. In some embodiments, the sample contains one or more tumor cells from the individual. In some embodiments, the sample contains circulating tumor DNA (ctDNA). In some embodiments, the sample is a tumor biopsy sample, a blood sample, a serum sample, a lymph sample or a bone marrow aspirate. In some embodiments, the sample is a sample obtained prior to the first administration of ibrutinib. In some embodiments, the sample is a sample obtained at 1 week, 2 weeks, 3 weeks, 1 month, 2 months, 3 months, 4 months, 5 months, 6 months, 7 months, 8 months, 9 months, 10 months, 11 months, 12 months, 14 months, 16 months, 18 months, 20 months, 22 months, or 24 months following the first administration of ibrutinib. In some embodiments, the sample is obtained 1, 2, 3, 4, 5, 6, 7, 8, 9, 10 times over the course of treatment with ibrutinib. In some embodiments, ibrutinib is administered once a day, two times per day, three times per day, four times per day, or five times per day. In some embodiments, ibrutinib is administered at a dosage of about 40 mg/day to about 1000 mg/day. In some embodiments, ibrutinib is administered orally. In some embodiments, the method further comprises administering an additional therapeutic agent. In some embodiments, the additional therapeutic agent is selected from among a chemotherapeutic agent or radiation therapeutic agent. In some embodiments, the chemotherapeutic agent is selected from among chlorambucil, ifosfamide, doxorubicin, mesalazine, thalidomide, lenalidomide, temsirolimus, everolimus, fludarabine, fostamatinib, paclitaxel, docetaxel, ofatumumab, rituximab, dexamethasone, prednisone, CAL-101, ibritumomab, tositumomab, bortezomib, pentostatin, endostatin, or a combination thereof. In some embodiments, ibrutinib is administered simultaneously, sequentially or intermittently with the additional therapeutic agent.

Disclosed herein, in certain embodiments, is a method of assessing an individual having diffuse large B cell lymphoma (DLBCL) for treatment, comprising: (a) determining the expression level of at least one biomarker gene selected from ACTG2, LOR, GAPT, CCND2, SELL, GEN1, and HDAC9; and (b) administering to the individual a therapeutically effective amount of ibrutinib if there is an increase in expression level in at least one biomarker gene selected from ACTG2, LOR, GAPT, CCND2, SELL, GEN1, and HDAC9 relative to a control. Also disclosed herein, in certain embodiments, is a method of monitoring the disease progression in an individual having diffuse large B cell lymphoma (DLBCL), comprising: (a) determining the expression level of at least one biomarker gene selected from ACTG2, LOR, GAPT, CCND2, SELL, GEN1, and HDAC9; and (b) characterizing the individual as having a stable DLBCL if the individual shows an increase in expression level in at least one biomarker gene selected from ACTG2, LOR, GAPT, CCND2, SELL, GEN1, and HDAC9 relative to a control. In some embodiments, the expression level of the at least one biomarker gene selected from ACTG2, LOR, GAPT, CCND2, SELL, GEN1, and HDAC9 increase by 0.5-fold, 1-fold, 1.5-fold, 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, 10-fold, 15-fold, 20-fold, 50-fold, or more compared to the control. In some embodiments, the control is the expression levels of the ACTG2, LOR, GAPT, CCND2, SELL, GEN1, and HDAC9 genes in an individual who has a progressive DLBCL. In some embodiments, DLBCL is activated B-cell DLBCL (ABC-DLBCL). In some embodiments, the DLBCL is a relapsed or refractory DLBCL. In some embodiments, the method further comprises testing a sample containing nucleic acid molecules encoding the ACTG2, LOR, GAPT, CCND2, SELL, GEN1, and HDAC9 genes obtained from the individual, and determining the expression levels of the ACTG2, LOR, GAPT, CCND2, SELL, GEN1, and HDAC9 genes. In some embodiments, the nucleic acid molecule is RNA. In some embodiments, testing comprises detecting the nucleic acid molecules using a microarray. In some embodiments, the method further comprises amplifying the nucleic acid molecules. In some embodiments, amplification is by isothermal amplification or polymerase chain reaction (PCR). In some embodiments, amplification is by PCR. In some embodiments, the method further comprises obtaining the sample from the individual. In some embodiments, the sample contains one or more tumor cells from the individual. In some embodiments, the sample contains circulating tumor DNA (ctDNA). In some embodiments, the sample is a tumor biopsy sample, a blood sample, a serum sample, a lymph sample or a bone marrow aspirate. In some embodiments, the sample is a sample obtained prior to the first administration of ibrutinib. In some embodiments, the sample is a sample obtained at 1 week, 2 weeks, 3 weeks, 1 month, 2 months, 3 months, 4 months, 5 months, 6 months, 7 months, 8 months, 9 months, 10 months, 11 months, 12 months, 14 months, 16 months, 18 months, 20 months, 22 months, or 24 months following the first administration of ibrutinib. In some embodiments, the sample is obtained 1, 2, 3, 4, 5, 6, 7, 8, 9, 10 times over the course of treatment with ibrutinib. In some embodiments, ibrutinib is administered once a day, two times per day, three times per day, four times per day, or five times per day. In some embodiments, ibrutinib is administered at a dosage of about 40 mg/day to about 1000 mg/day. In some embodiments, ibrutinib is administered orally. In some embodiments, the method further comprises administering an additional therapeutic agent. In some embodiments, the additional therapeutic agent is selected from among a chemotherapeutic agent or radiation therapeutic agent. In some embodiments, the chemotherapeutic agent is selected from among chlorambucil, ifosfamide, doxorubicin, mesalazine, thalidomide, lenalidomide, temsirolimus, everolimus, fludarabine, fostamatinib, paclitaxel, docetaxel, ofatumumab, rituximab, dexamethasone, prednisone, CAL-101, ibritumomab, tositumomab, bortezomib, pentostatin, endostatin, or a combination thereof. In some embodiments, ibrutinib is administered simultaneously, sequentially or intermittently with the additional therapeutic agent.

Disclosed herein, in certain embodiments, is a kit for carrying out the methods disclosed herein, comprising one or more reagents for determining the presence or absence of a modification in one or more biomarker genes selected from EP300, MLL2, BCL-2, RB1, LRP1B, PIM1, TSC2, TNFRSF11A, SMAD4, PAX5, and CARD11 in the sample. In some embodiments, the kit comprises nucleic acid probes or primers that bind to the nucleic acid molecules encoding EP300, MLL2, BCL-2, RB1, LRP1B, PIM1, TSC2, TNFRSF11A, SMAD4, PAX5, or CARD11.

Disclosed herein, in certain embodiments, is a kit for carrying out the methods disclosed herein, comprising one or more reagents for determining the presence or absence of a modification to an aromatic residue at amino acid position 196 in CD79B and at least one modification at amino acid positions 198 or 265 in MYD88 in the sample. In some embodiments, the kit comprises nucleic acid probes or primers that bind to the nucleic acid molecules encoding CD79B or MYD88 polypeptides.

Disclosed herein, in certain embodiments, is a kit for carrying out the methods disclosed herein, comprising one or more reagents for determining the presence or absence of a modification at amino acid position 15 in ROS1 in the sample. In some embodiments, the kit comprises nucleic acid probes or primers that bind to the nucleic acid molecules encoding the ROS1 polypeptide.

Disclosed herein, in certain embodiments, is a kit for carrying out the methods disclosed herein, comprising one or more reagents for determining the expression level of at least one biomarker gene selected from ACTG2, LOR, GAPT, CCND2, SELL, GEN1 and HDAC9 in the sample. In some embodiments, the kit comprises nucleic acid probes or primers that bind to the nucleic acid molecules encoding ACTG2, LOR, GAPT, CCND2, SELL, GEN1 or HDAC9. In some embodiments, the kit comprises an antibody that binds to a protein encoded by ACTG2, LOR, GAPT, CCND2, SELL, GEN1 or HDAC9.

Disclosed herein, in certain embodiments, is a system of assessing an individual having diffuse large B cell lymphoma (DLBCL) for treatment comprising: (a) a digital processing device comprising an operating system configured to perform executable instructions, and an electronic memory; (b) a dataset stored in the electronic memory, wherein the dataset comprises data for one or more biomarker genes in a sample, wherein the biomarker genes are selected from the group consisting of EP300, MLL2, BCL-2, RB1, LRP1B, PIM1, TSC2, TNFRSF11A, SMAD4, PAX5, and CARD11; and (c) a computer program including instructions executable by the digital processing device to create an application comprising: (i) a first software module configured to analyze the dataset to determine the presence or absence of modifications in one or more biomarker genes; and (ii) a second software module to assign the individual as a candidate for treatment with ibrutinib if there is an absence of modifications in the one or more biomarker genes. In some embodiments, the one or more biomarker genes are selected from BCL-2, RB1, LRP1B, PIM1, and TSC2. In some embodiments, the modification is base substitution, insertion, deletion, DNA rearrangement, copy number alteration, or a combination thereof. In some embodiments, EP300, MLL2, BCL-2, RB1, LRP1B, PIM1, TSC2, TNFRSF11A, SMAD4, PAX5, and CARD11 comprise one or more modifications in each gene. In some embodiments, the modifications associated with the EP300, MLL2, BCL-2, RB1, LRP1B, PIM1, TSC2, TNFRSF11A, SMAD4, PAX5, and CARD11 genes further comprise modifications in the EP300, MLL2, BCL-2, RB1, LRP1B, PIM1, TSC2, TNFRSF11A, SMAD4, PAX5, and CARD11 proteins. In some embodiments, the modifications associated with the BCL-2 gene further comprise modifications in the BCL-2 protein. In some embodiments, the BCL-2 protein comprises modifications at positions corresponding to amino acid residues 4, 9, 33, 47, 48, 49, 60, 68, 74, 113, 114, 120, 122, 129, 131, 165, 197, 198, 200, 201, 203, and 206. In some embodiments, the modifications include A4S, Y9H, G33R, G47A, I48S, F49L, A60T, R68K, T74N, T74S, A113G, E114A, H120Y, T122S, R129H, A131V, E165D, G197R, G197S, A198V, G200S, D201N, S203N, and 206W. In some embodiments, DLBCL is activated B-cell DLBCL (ABC-DLBCL), germinal center B-cell like DLBCL (GBC-DLBCL), or unclassified DLBCL. In some embodiments, the DLBCL is a relapsed or refractory DLBCL. In some embodiments, the method further comprises a sample obtained from the individual, wherein the sample contains nucleic acid molecules encoding the biomarker genes selected from EP300, MLL2, BCL-2, RB1, LRP1B, PIM1, TSC2, TNFRSF11A, SMAD4, PAX5, and CARD11. In some embodiments, the nucleic acid molecule is RNA or DNA. In some embodiments, the DNA is genomic DNA. In some embodiments, the sample is a tumor biopsy sample, a blood sample, a serum sample, a lymph sample or a bone marrow aspirate. In some embodiments, the sample contains circulating tumor DNA (ctDNA). In some embodiments, the method further comprises an analytical device configured to provide biomarker data; wherein the analytical device is coupled to the digital processing device. In some embodiments, the analytical device performs microarray analysis. In some embodiments, the digital processing device is connected to a computer network. In some embodiments, the second software module further generates a report, wherein the second software module is executed by the digital processing device. In some embodiments, the second software module further transmits the report to an end-user, wherein the second software module is executed by the digital processing device.

Disclosed herein, in certain embodiments, is a nucleic acid hybridization array comprising nucleic acid probes for evaluating whether an individual receiving ibrutinib for treatment of diffuse large B cell lymphoma (DLBCL) has developed or is likely to develop resistance to the therapy, consisting essentially of nucleic acid probes which hybridize to biomarker genes selected from the group consisting of EP300, MLL2, BCL-2, RB1, LRP1B, PIM1, TSC2, TNFRSF11A, SMAD4, PAX5, and CARD11. In some embodiments, at least one of the nucleic acid probes hybridizes to a biomarker gene selected from BCL-2, RB1, LRP1B, PIM1, and TSC2. In some embodiments, the biomarker gene comprises one or more modifications. In some embodiments, the modification is base substitution, insertion, deletion, DNA rearrangement, copy number alteration, or a combination thereof. In some embodiments, EP300, MLL2, BCL-2, RB1, LRP1B, PIM1, TSC2, TNFRSF11A, SMAD4, PAX5, and CARD11 comprise one or more modifications in each gene. In some embodiments, the use of the array comprises of determining the presence or absence of modifications in one or more biomarker genes in a sample obtained from the individual; and characterizing the individual as resistant or is likely to become resistant to therapy with ibrutinib if the individual has modifications in the one or more biomarker genes. In some embodiments, the sample is a tumor biopsy sample, a blood sample, a serum sample, a lymph sample or a bone marrow aspirate.

Disclosed herein, in certain embodiments, is a system of assessing an individual having diffuse large B cell lymphoma (DLBCL) for treatment comprising: (a) a digital processing device comprising an operating system configured to perform executable instructions, and an electronic memory; (b) a dataset stored in the electronic memory, wherein the dataset comprises data for one or more biomarker genes in a sample, wherein the biomarker genes are selected from the group consisting of ACTG2, LOR, GAPT, CCND2, SELL, GEN1, and HDAC9; and (c) a computer program including instructions executable by the digital processing device to create an application comprising: (i) a third software module configured to analyze the dataset to determine the expression level of one or more biomarker genes; (ii) a forth software module configured to match the expression level of one or more biomarker genes to a control; and (iii) a fifth software module to assign the individual as a candidate to treatment with ibrutinib if there is an increase in expression level in the one or more biomarker genes relative to the control. In some embodiments, the expression levels of the at least one biomarker gene selected from ACTG2, LOR, GAPT, CCND2, SELL, GEN1, and HDAC9 increase by 0.5-fold, 1-fold, 1.5-fold, 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, 10-fold, 15-fold, 20-fold, 50-fold, or more compared to the control. In some embodiments, the control is the expression levels of the ACTG2, LOR, GAPT, CCND2, SELL, GEN1, and HDAC9 genes in an individual who as a progressive DLBCL. In some embodiments, DLBCL is activated B-cell DLBCL (ABC-DLBCL). In some embodiments, the DLBCL is a relapsed or refractory DLBCL. In some embodiments, the system further comprises a sample obtained from the individual, wherein the sample contains nucleic acid molecules encoding the biomarker genes selected from ACTG2, LOR, GAPT, CCND2, SELL, GEN1, and HDAC9. In some embodiments, the nucleic acid molecule is RNA or DNA. In some embodiments, the DNA is genomic DNA. In some embodiments, the sample is a tumor biopsy sample, a blood sample, a serum sample, a lymph sample or a bone marrow aspirate. In some embodiments, the sample contains circulating tumor DNA (ctDNA). In some embodiments, the system further comprises an analytical device configured to provide biomarker data; wherein the analytical device is coupled to the digital processing device. In some embodiments, the analytical device performs microarray analysis. In some embodiments, the digital processing device is connected to a computer network. In some embodiments, the fifth software module further generates a report, wherein the fifth software module is executed by the digital processing device. In some embodiments, the fifth software module further transmits the report to an end-user, wherein the fifth software module is executed by the digital processing device.

Disclosed herein, in certain embodiments, is a nucleic acid hybridization array comprising nucleic acid probes for evaluating whether an individual having diffuse large B cell lymphoma (DLBCL) has a stable DLBCL, consisting essentially of nucleic acid probes which hybridize to biomarker genes selected from the group consisting of ACTG2, LOR, GAPT, CCND2, SELL, GEN1, and HDAC9. In some embodiments, the use of the array comprises: (a) determining the expression level of the biomarker genes in a sample; (b) comparing the expression levels of the biomarker genes to a control; and (c) characterizing the individual as having a stable DLBCL if the individual shows an increase in expression level in at least one biomarker gene relative to a control. In some embodiments, the control is the expression levels of the ACTG2, LOR, GAPT, CCND2, SELL, GEN1, and HDAC9 genes in an individual who has a progressive DLBCL. In some embodiments, the sample is a tumor biopsy sample, a blood sample, a serum sample, a lymph sample or a bone marrow aspirate.

Disclosed herein, in certain embodiments, is a method of selecting an individual having a non-Hodgkin's lymphoma for treatment with ibrutinib, comprising: (a) determining the expression level of the biomarker gene BCL-2; and (b) administering to the individual a therapeutically effective amount of ibrutinib if there is no increased expression level in the biomarker gene BCL-2 relative to a control. Also disclosed herein, in certain embodiments, is a method of monitoring the disease progression in an individual having a non-Hodgkin's lymphoma, comprising: (a) determining the expression level of the biomarker gene BCL-2; and (b) characterizing the individual as developed an insensitivity to ibrutinib if the individual shows an increase in expression level in the biomarker gene BCL-2 relative to a control. In some embodiments, the expression level of the biomarker gene BCL-2 increases by 0.5-fold, 1-fold, 1.5-fold, 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, 10-fold, 15-fold, 20-fold, 50-fold, or more compared to the control. In some embodiments, the control is the expression levels of the biomarker gene BCL-2 in an individual who is not insensitive toward ibrutinib. In some embodiments, the control is the expression levels of the biomarker gene BCL-2 in an individual who has not been treated with ibrutinib. In some embodiments, the non-Hodgkin's lymphoma is Burkitt lymphoma, chronic lymphocytic leukemia (CLL), small lymphocytic lymphoma (SLL), diffuse large B-cell lymphoma (DLBCL), follicular lymphoma (FL), immunoblastic large cell lymphoma, precursor B-lymphoblastic lymphoma, mantle cell lymphoma, marginal zone B-cell lymphomas, Waldenstrom's macroglobulinemia, lymphoplasmacytic lymphoma, hairy cell leukemia, mediastinal large B-cell lymphoma, cutaneous lymphomas, mycosis fungoides, anaplastic large cell lymphoma, peripheral T-cell lymphomas, enteropathy associated T cell lymphoma (EATL), hepatosplenic gamma delta T cell lymphoma, or precursor T-lymphoblastic lymphoma. In some embodiments, the non-Hodgkin's lymphoma is Burkitt lymphoma, chronic lymphocytic leukemia (CLL), small lymphocytic lymphoma (SLL), diffuse large B-cell lymphoma (DLBCL), follicular lymphoma (FL), immunoblastic large cell lymphoma, precursor B-lymphoblastic lymphoma, mantle cell lymphoma, marginal zone B-cell lymphomas, Waldenstrom's macroglobulinemia, lymphoplasmacytic lymphoma, hairy cell leukemia, or mediastinal large B-cell lymphoma. In some embodiments, the non-Hodgkin's lymphoma is DLBCL. In some embodiments, the non-Hodgkin's lymphoma is FL. In some embodiments, the non-Hodgkin's lymphoma is a relapsed or refractory non-Hodgkin's lymphoma. In some embodiments, the relapsed or refractory non-Hodgkin's lymphoma is a relapsed or refractory DLBCL. In some embodiments, the relapsed or refractory non-Hodgkin's lymphoma is a relapsed or refractory FL. In some embodiments, the methods further comprise testing a sample containing nucleic acid molecules encoding the BCL-2 gene. In some embodiments, the nucleic acid molecule is RNA. In some embodiments, testing comprises detecting the nucleic acid molecules using a microarray. In some embodiments, the methods further comprise amplifying the nucleic acid molecules. In some embodiments, amplification is by isothermal amplification or polymerase chain reaction (PCR). In some embodiments, amplification is by PCR. In some embodiments, the method further comprises obtaining the sample from the individual. In some embodiments, the sample contains one or more tumor cells from the individual. In some embodiments, the sample contains circulating tumor DNA (ctDNA). In some embodiments, the sample is a tumor biopsy sample, a blood sample, a serum sample, a lymph sample or a bone marrow aspirate. In some embodiments, ibrutinib is administered once a day, two times per day, three times per day, four times per day, or five times per day. In some embodiments, ibrutinib is administered at a dosage of about 40 mg/day to about 1000 mg/day. In some embodiments, ibrutinib is administered orally. In some embodiments, the method further comprises administering an additional therapeutic agent. In some embodiments, the additional therapeutic agent is selected from among a chemotherapeutic agent or radiation therapeutic agent. In some embodiments, the chemotherapeutic agent is selected from among chlorambucil, ifosfamide, doxorubicin, mesalazine, thalidomide, lenalidomide, temsirolimus, everolimus, fludarabine, fostamatinib, paclitaxel, docetaxel, ofatumumab, rituximab, dexamethasone, prednisone, CAL-101, ibritumomab, tositumomab, bortezomib, pentostatin, endostatin, or a combination thereof. In some embodiments, ibrutinib is administered simultaneously, sequentially or intermittently, with the additional therapeutic agent.

Disclosed herein, in certain embodiments, is a method of monitoring the disease progression in an individual having a non-Hodgkin's lymphoma, comprising: (a) determining the mutation rate of the biomarker gene BCL-2; and (b) characterizing the individual as developed an insensitivity to ibrutinib or likely to develop an insensitivity to ibrutinib if the individual shows an increase in the mutation rate in the biomarker gene BCL-2 relative to a control. In some embodiments, the mutation rate of the biomarker gene BCL-2 increases by 0.5-fold, 1-fold, 1.5-fold, 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, 10-fold, 15-fold, 20-fold, 50-fold, or more compared to the control. In some embodiments, the control is the biomarker gene BCL-2 is a wild type BCL-2 gene. In some embodiments, the control is the biomarker gene BCL-2 from an individual who is not insensitive toward ibrutinib. In some embodiments, the control is the biomarker gene BCL-2 from an individual who has not been treated with ibrutinib. In some embodiments, the non-Hodgkin's lymphoma is Burkitt lymphoma, chronic lymphocytic leukemia (CLL), small lymphocytic lymphoma (SLL), diffuse large B-cell lymphoma (DLBCL), follicular lymphoma (FL), immunoblastic large cell lymphoma, precursor B-lymphoblastic lymphoma, mantle cell lymphoma, marginal zone B-cell lymphomas, Waldenstrom's macroglobulinemia, lymphoplasmacytic lymphoma, hairy cell leukemia, mediastinal large B-cell lymphoma, cutaneous lymphomas, mycosis fungoides, anaplastic large cell lymphoma, peripheral T-cell lymphomas, enteropathy associated T cell lymphoma (EATL), hepatosplenic gamma delta T cell lymphoma, or precursor T-lymphoblastic lymphoma. In some embodiments, the non-Hodgkin's lymphoma is Burkitt lymphoma, chronic lymphocytic leukemia (CLL), small lymphocytic lymphoma (SLL), diffuse large B-cell lymphoma (DLBCL), follicular lymphoma (FL), immunoblastic large cell lymphoma, precursor B-lymphoblastic lymphoma, mantle cell lymphoma, marginal zone B-cell lymphomas, Waldenstrom's macroglobulinemia, lymphoplasmacytic lymphoma, hairy cell leukemia, or mediastinal large B-cell lymphoma. In some embodiments, the non-Hodgkin's lymphoma is DLBCL. In some embodiments, the non-Hodgkin's lymphoma is FL. In some embodiments, the non-Hodgkin's lymphoma is a relapsed or refractory non-Hodgkin's lymphoma. In some embodiments, the relapsed or refractory non-Hodgkin's lymphoma is a relapsed or refractory DLBCL. In some embodiments, the relapsed or refractory non-Hodgkin's lymphoma is a relapsed or refractory FL. In some embodiments, the method further comprises testing a sample containing nucleic acid molecules encoding the BCL-2 gene. In some embodiments, the nucleic acid molecule is RNA. In some embodiments, testing comprises detecting the nucleic acid molecules using a microarray. In some embodiments, the method further comprises amplifying the nucleic acid molecules. In some embodiments, amplification is by isothermal amplification or polymerase chain reaction (PCR). In some embodiments, amplification is by PCR. In some embodiments, the method further comprises obtaining the sample from the individual. In some embodiments, the sample contains one or more tumor cells from the individual. In some embodiments, the sample contains circulating tumor DNA (ctDNA). In some embodiments, the sample is a tumor biopsy sample, a blood sample, a serum sample, a lymph sample or a bone marrow aspirate. In some embodiments, ibrutinib is administered once a day, two times per day, three times per day, four times per day, or five times per day. In some embodiments, ibrutinib is administered at a dosage of about 40 mg/day to about 1000 mg/day. In some embodiments, ibrutinib is administered orally. In some embodiments, the method further comprises administering an additional therapeutic agent. In some embodiments, the additional therapeutic agent is selected from among a chemotherapeutic agent or radiation therapeutic agent. In some embodiments, the chemotherapeutic agent is selected from among chlorambucil, ifosfamide, doxorubicin, mesalazine, thalidomide, lenalidomide, temsirolimus, everolimus, fludarabine, fostamatinib, paclitaxel, docetaxel, ofatumumab, rituximab, dexamethasone, prednisone, CAL-101, ibritumomab, tositumomab, bortezomib, pentostatin, endostatin, or a combination thereof. In some embodiments, ibrutinib is administered simultaneously, sequentially or intermittently, with the additional therapeutic agent.

Disclosed herein, in certain embodiments, is a method of treating a non-Hodgkin's lymphoma comprising administering to an individual in need thereof a therapeutically effective amount of a combination comprising a BTK inhibitor and a BCL-2 inhibitor. In some embodiments, the combination provides a synergistic therapeutic effect compared to administration of the BTK inhibitor and the BCL-2 inhibitor alone. In some embodiments, the non-Hodgkin's lymphoma is Burkitt lymphoma, chronic lymphocytic leukemia (CLL), small lymphocytic lymphoma (SLL), diffuse large B-cell lymphoma (DLBCL), follicular lymphoma (FL), immunoblastic large cell lymphoma, precursor B-lymphoblastic lymphoma, mantle cell lymphoma, marginal zone B-cell lymphomas, Waldenstrom's macroglobulinemia, lymphoplasmacytic lymphoma, hairy cell leukemia, mediastinal large B-cell lymphoma, cutaneous lymphomas, mycosis fungoides, anaplastic large cell lymphoma, peripheral T-cell lymphomas, enteropathy associated T cell lymphoma (EATL), hepatosplenic gamma delta T cell lymphoma, or precursor T-lymphoblastic lymphoma. In some embodiments, the non-Hodgkin's lymphoma is DLBCL. In some embodiments, the non-Hodgkin's lymphoma is FL. In some embodiments, the non-Hodgkin's lymphoma is a relapsed or refractory non-Hodgkin's lymphoma. In some embodiments, the non-Hodgkin's lymphoma is an ibrutinib resistant non-Hodgkin's lymphoma. In some embodiments, the BTK inhibitor is ibrutinib. In some embodiments, the BCL-2 inhibitor is ABT-199.

Disclosed herein, in certain embodiments, is a method of treating an ibrutinib-resistant non-Hodgkin's lymphoma comprising administering to an individual in need thereof a therapeutically effective amount of a combination comprising ibrutinib and a BCL-2 inhibitor. In some embodiments, the combination provides a synergistic therapeutic effect compared to administration of ibrutinib and the BCL-2 inhibitor alone. In some embodiments, the ibrutinib-resistant non-Hodgkin's lymphoma is Burkitt lymphoma, chronic lymphocytic leukemia (CLL), small lymphocytic lymphoma (SLL), diffuse large B-cell lymphoma (DLBCL), follicular lymphoma (FL), immunoblastic large cell lymphoma, precursor B-lymphoblastic lymphoma, mantle cell lymphoma, marginal zone B-cell lymphomas, Waldenstrom's macroglobulinemia, lymphoplasmacytic lymphoma, hairy cell leukemia, mediastinal large B-cell lymphoma, cutaneous lymphomas, mycosis fungoides, anaplastic large cell lymphoma, peripheral T-cell lymphomas, enteropathy associated T cell lymphoma (EATL), hepatosplenic gamma delta T cell lymphoma, or precursor T-lymphoblastic lymphoma. In some embodiments, the ibrutinib-resistant non-Hodgkin's lymphoma is ibrutinib-resistant DLBCL. In some embodiments, the ibrutinib-resistant non-Hodgkin's lymphoma is ibrutinib-resistant FL. In some embodiments, the ibrutinib-resistant non-Hodgkin's lymphoma is a relapsed or refractory ibrutinib-resistant non-Hodgkin's lymphoma. In some embodiments, the BCL-2 inhibitor is ABT-199.

Disclosed herein, in certain embodiments, are methods of treating a non-Hodgkin's lymphoma comprising, administering to an individual in need thereof a therapeutically effective amount of a combination comprising a BTK inhibitor, a BCL-2 inhibitor, and a PI3K inhibitor. In some embodiments, the combination provides a synergistic therapeutic effect compared to administration of the BTK inhibitor with the BCL-2 inhibitor or administration of the BTK inhibitor with the PI3K inhibitor. In some embodiments, the non-Hodgkin's lymphoma is Burkitt lymphoma, chronic lymphocytic leukemia (CLL), small lymphocytic lymphoma (SLL), diffuse large B-cell lymphoma (DLBCL), follicular lymphoma (FL), immunoblastic large cell lymphoma, precursor B-lymphoblastic lymphoma, mantle cell lymphoma, marginal zone B-cell lymphomas, Waldenstrom's macroglobulinemia, lymphoplasmacytic lymphoma, hairy cell leukemia, mediastinal large B-cell lymphoma, cutaneous lymphomas, mycosis fungoides, anaplastic large cell lymphoma, peripheral T-cell lymphomas, enteropathy associated T cell lymphoma (EATL), hepatosplenic gamma delta T cell lymphoma, or precursor T-lymphoblastic lymphoma. In some embodiments, the non-Hodgkin's lymphoma is DLBCL. In some embodiments, the DLBCL is GCB-DLBCL. In some embodiments, the non-Hodgkin's lymphoma is a relapsed or refractory non-Hodgkin's lymphoma. In some embodiments, the non-Hodgkin's lymphoma is an ibrutinib resistant non-Hodgkin's lymphoma. In some embodiments, the BTK inhibitor is ibrutinib. In some embodiments, the BCL-2 inhibitor is ABT-199. In some embodiments, the PI3K inhibitor is IPI-145. In some embodiments, the combination comprises ibrutinib, ABT-199, and IPI-145.

Disclosed herein, in certain embodiments, are methods of treating a non-Hodgkin's lymphoma comprising, administering to an individual in need thereof a therapeutically effective amount of a combination comprising a BTK inhibitor, a BCL-2 inhibitor, and a corticosteroid. In some embodiments, the combination provides a synergistic therapeutic effect compared to administration of the BTK inhibitor with the BCL-2 inhibitor or administration of the BTK inhibitor with the corticosteroid. In some embodiments, the non-Hodgkin's lymphoma is Burkitt lymphoma, chronic lymphocytic leukemia (CLL), small lymphocytic lymphoma (SLL), diffuse large B-cell lymphoma (DLBCL), follicular lymphoma (FL), immunoblastic large cell lymphoma, precursor B-lymphoblastic lymphoma, mantle cell lymphoma, marginal zone B-cell lymphomas, Waldenstrom's macroglobulinemia, lymphoplasmacytic lymphoma, hairy cell leukemia, mediastinal large B-cell lymphoma, cutaneous lymphomas, mycosis fungoides, anaplastic large cell lymphoma, peripheral T-cell lymphomas, enteropathy associated T cell lymphoma (EATL), hepatosplenic gamma delta T cell lymphoma, or precursor T-lymphoblastic lymphoma. In some embodiments, the non-Hodgkin's lymphoma is DLBCL. In some embodiments, the DLBCL is GCB-DLBCL. In some embodiments, the non-Hodgkin's lymphoma is a relapsed or refractory non-Hodgkin's lymphoma. In some embodiments, the non-Hodgkin's lymphoma is an ibrutinib resistant non-Hodgkin's lymphoma. In some embodiments, the BTK inhibitor is ibrutinib. In some embodiments, the BCL-2 inhibitor is ABT-199. In some embodiments, the corticosteroid is dexamethasone. In some embodiments, the combination comprises ibrutinib, ABT-199, and dexamethasone.

Disclosed herein, in certain embodiments, are compositions comprising a BTK inhibitor, a BCL-2 inhibitor, and a PI3K inhibitor. In some embodiments, the BTK inhibitor is ibrutinib. In some embodiments, the BCL-2 inhibitor is ABT-199. In some embodiments, the PI3K inhibitor is IPI-145. In some embodiments, the composition comprises ibrutinib, ABT-199, and IPI-145.

Disclosed herein, in certain embodiments, are compositions comprising a BTK inhibitor, a BCL-2 inhibitor, and a corticosteroid. In some embodiments, the BTK inhibitor is ibrutinib. In some embodiments, the BCL-2 inhibitor is ABT-199. In some embodiments, the corticosteroid is dexamethasone. In some embodiments, the composition comprises ibrutinib, ABT-199, and dexamethasone.

Disclosed herein, in certain embodiments, is a use of ibrutinib for treating diffuse large B cell lymphoma (DLBCL) in an individual having an absence of a modification in the one or more biomarker genes selected from EP300, MLL2, BCL-2, RB1, LRP1B, PIM1, TSC2, TNFRSF11A, SMAD4, PAX5, and CARD11. In some embodiments, the individual has an absence of a modification in two or more biomarker genes selected from EP300, MLL2, BCL-2, RB1, LRP1B, PIM1, TSC2, TNFRSF11A, SMAD4, PAX5, and CARD11. In some embodiments, the one or more biomarker genes are selected from BCL-2, RB1, LRP1B, PIM1, and TSC2. In some embodiments, the modification associated with the EP300, MLL2, BCL-2, RB1, LRP1B, PIM1, TSC2, TNFRSF11A, SMAD4, PAX5, and CARD11 genes results in a modification in the EP300, MLL2, BCL2, RB1, LRP1B, PIM1, TSC2, TNFRSF11A, SMAD4, PAX5, and CARD11 proteins. In some embodiments, the BCL-2 protein comprises one or more modifications at positions corresponding to amino acid residues 4, 9, 33, 47, 48, 49, 60, 68, 74, 113, 114, 120, 122, 129, 131, 165, 197, 198, 200, 201, 203, and 206. In some embodiments, the modifications include A4S, Y9H, G33R, G47A, I48S, F49L, A60T, R68K, T74N, T74S, A113G, E114A, H120Y, T122S, R129H, A131V, E165D, G197R, G197S, A198V, G200S, D201N, S203N, and 206W. In some embodiments, DLBCL is activated B-cell DLBCL (ABC-DLBCL), germinal center B-cell like DLBCL (GBC-DLBCL), or unclassified DLBCL. In some embodiments, the DLBCL is a relapsed or refractory DLBCL.

Disclosed herein, in certain embodiments, is a use of ibrutinib for treating diffuse large B cell lymphoma (DLBCL) in an individual having a modification to an aromatic residue in CD79B and at least one modification at amino acid positions 198 or 265 in MYD88. In some embodiments, the modification at amino acid position 196 in CD79B is Y196F. In some embodiments, the modification at amino acid position 198 in MYD88 is S198N. In some embodiments, the modification at amino acid position 265 in MYD88 is L265P. In some embodiments, the individual has a combination of the modifications in CD79B and MYD88 of Y196F and S198N or Y196F and L265P. In some embodiments, the DLBCL is activated B-cell DLBCL (ABC-DLBCL) or unclassified DLBCL. In some embodiments, the DLBCL is a relapsed or refractory DLBCL.

Disclosed herein, in certain embodiments, is a use of ibrutinib for treating diffuse large B cell lymphoma (DLBCL) in an individual having an absence of a modification at amino acid position 15 in ROS1. In some embodiments, the modification at amino acid position 15 in ROS1 is A15G. In some embodiments, the A15G modification in ROS1 further indicates the individual has developed or likely to develop a progressive DLBCL. In some embodiments, DLBCL is activated B-cell DLBCL (ABC-DLBCL), germinal center B-cell like DLBCL (GBC-DLBCL), or unclassified DLBCL. In some embodiments, the DLBCL is a relapsed or refractory DLBCL.

### BRIEF DESCRIPTION OF THE DRAWINGS

Various aspects of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates a conceptual schematic for patient selection, maintenance therapy, or optimization of therapeutic regimen based on the modifications or expression levels of the biomarkers or biomarker genes described herein.

FIG. 2 illustrates a conceptual schematic of an exemplary computer server to be used for processing a system and a method described herein.

FIG. 3 exemplifies the distribution of DLBCL patients stratified according to progressive disease (PD), stable disease (SD), partial response (PR) and complete response (CR). Tumor biopsy samples were collected, pre-dose, from 51 patients. The patients were selected from 1106 cohort 1, 1106 cohort 2, and 04753 cohort.

FIG. 4 exemplifies the mutations which did not thwart response to ibrutinib treatment, in DLBCL patients. The frequency of occurrence is shown either as the number of patients or as a percentage of patients. The numbers in parenthesis indicate the number of patients or the percentage of patients with mutations in 1106 cohort 2.

FIG. 5 exemplifies the mutations which affected response to ibrutinib treatment, in DLBCL patients. The frequency of occurrence is shown either as the number of patients or as a percentage of patients. The numbers in parenthesis indicate the number of patients or percentage of patients with mutations in 1106 cohort 2.

FIG. 6 illustrates the role of mutations in BCR signaling.

FIG. 7 illustrates relationship between BCR signaling and activation of NF-κB gene transcription via NF-κB signaling pathways. The arrows 1, 2 and 3 illustrate the three main BCR signaling arcs leading to NF-κB signaling pathways.

FIG. 8 illustrates the relation between of A15G mutation in the signal peptide region of ROS1 and post ibrutinib treatment relapsed tumor in DLBCL patient 11096-091-201. The mutational frequency of A15G in the resp-prog (refractory to drug/relapsed stage) tumor biopsy was compared to the same in pre-dose stage and prim-met (metastasis) stage tumor biopsies.

FIG. 9 illustrates the distribution of DLBCL patients, selected for gene expression profiling using the Affymetrix U133 plus 2.0 gene array chip, among response groups. PD stands for progressive disease, SD stands for stable disease, PR stands for partial response and CR stands for complete remission. Samples were collected, pre-dose, from these 67 patients. The patients were selected from 1106 cohorts 1 and 2.

FIG. 10A and FIG. 10B illustrate the removal of unwanted variation between the gene expression profile data, obtained using the Affymetrix U133 plus 2.0 gene array chip, from 1106 cohorts 1 and 2, before (FIG. 10A) and after (FIG. 10B) correction of batch effect.

FIG. 11 exemplifies the expression profile of genes which are positively (rows 1-7) or negatively (rows 8-9) correlated with progression free survival (PFS) among the ABC subtype DLBCL patients of 1106 cohorts 1 and 2.

FIGS. 12A-FIG. 12D show examples of genes with positive correlation between expression and PFS in DLBCL patients. FIG. 12A and FIG. 12B illustrate that the expression levels of CCND2 are higher in complete remission (CR) patients than in patients with progressive disease (PD); FIG. 12C and FIG. 12D illustrate that the expression levels of SELL are higher in complete remission (CR) patients than in patients with progressive disease (PD).

FIGS. 13A-FIG. 13D show examples of genes with negative correlation between expression and PFS in DLBCL patients. FIG. 13 A and FIG. 13B illustrate that the expression levels of FGR are higher in complete remission (CR) patients than in patients with progressive disease (PD); FIG. 13C and FIG. 13D illustrate that the expression levels of IGHA1 are higher in complete remission (CR) patients than in patients with progressive disease (PD).

FIG. 14A and FIG. 14B show expression levels for the analyte, OPN (FIG. 14A) in DLBCL patients selected from 1106 cohorts 1, 2 and 04753 cohort. The analyte levels were higher in PD patients (1106-PD, 04753-PD) than in the SD (1106-SD, 04753-SD) and CR patients (1106-CR, 04753-CR) (FIG. 14B).

FIG. 15A and FIG. 15B show expression levels for the analyte, MMP-7 (FIG. 15A) in DLBCL patients selected from 1106 cohorts 1, 2 and 04753 cohort. The analyte levels are higher in PD patients (1106-PD, 04753-PD) than in the SD (1106-SD, 04753-SD) and CR patients (1106-CR, 04753-CR) (FIG. 15B).

FIG. 16A and FIG. 16B show expression levels for the analyte, ALDR (FIG. 16A) in DLBCL patients selected from 1106 cohorts 1, 2 and 04753 cohort. The analyte levels were higher in PD patients (1106-PD, 04753-PD) than in the SD (1106-SD, 04753-SD) and CR patients (1106-CR, 04753-CR) (FIG. 16B).

FIG. 17A and FIG. 17B show expression levels for the analyte, HGF (FIG. 17A) in DLBCL patients selected from 1106 cohorts 1, 2 and 04753 cohort. The analyte levels were higher in PD patients (1106-PD, 04753-PD) than in the SD (1106-SD, 04753-SD) and CR patients (1106-CR, 04753-CR) (FIG. 17B).

FIGS. 18A-FIG. 18C show comparison of BCL-2 gene expressions in either ibrutinib-resistant TMD8 cells or wild-type TMD8 cells. BCL-2 gene expression is higher in ibrutinib-resistant TMD8 cells than in wild-type TMD8 cells.

FIG. 19 illustrates BCL-2 gene expressions in different subspecies of DLBCL tumor samples. Lower BCL-2 gene expression was observed in the tumor samples from patients with better responses to ibrutinib.

FIG. 20 illustrates BCL-2 mutation rates in different tumor samples. Tumor samples obtained from patients with either partial response (PR) or complete response (CR) showed lower rate of mutation than tumor samples obtained from patients with progressive disease (PD) or stable disease (SD).

FIG. 21A-FIG. 21C illustrate BCL-2 expression in DoHH2 cell-lines. FIG. 21A and FIG. 21B show the expression of BCL-2 gene in DoHH2, a non-Hodgkin's B-cell line, normalized to GAPDH and Actin, respectively. FIG. 21C shows the expressin of BCL-2 at the protein level.

FIG. 22A-FIG. 22D show the effect of the combination of ibrutinib and ABT-199 on wild type DoHH2 proliferation. FIG. 22A illustrates the synergy score heat map of ibrutinib and ABT-199. FIG. 22B shows the percentage of growth of DoHH2 wild-type cells in the presence of ABT-199 and ibruitnib. FIGS. 22C and 22D show the synergy score of the ibrutinib and ABT-199 combination.

FIG. 23A-FIG. 23D show the effect of the combination of ibrutinib and ABT-199 on ibrutinib resistant DoHH2 proliferation. FIG. 23A illustrates the synergy score heat map of ibrutinib and ABT-199. FIG. 23B shows the percentage of growth of DoHH2 ibrutinib resistant cells in the presence of ABT-199 and ibruitnib. FIGS. 23C and 23D show the synergy score of the ibrutinib and ABT-199 combination.

FIG. 24A-FIG. 24D show the effect of the combination of ibrutinib and ABT-199 on ibrutinib resistant DoHH2 proliferation. FIG. 24A illustrates the synergy score heat map of ibrutinib and ABT-199. FIG. 24B shows the percentage of growth of a second population of DoHH2 ibrutinib resistant cells in the presence of ABT-199 and ibruitnib. FIGS. 24C and 24D show the synergy score of the ibrutinib and ABT-199 combination.

FIG. 25A shows plots of cell growth of TMD8, HBL1, and LY10 cells treated with ibrutinib in the presence or absence of ABT-199. FIG. 25B shows the drug dose matrix data of TMD8, HTML1, and LY10 cells. FIG. 25C shows the isobologram analysis of the data in FIG. 25B. FIG. 25D shows the combination index (C.I.) of ibrutinib and ABT-199 at the indicated concentrations in TMD8, HBL1, and LY10 cells.

FIG. 26A illustrates the adhesion of TMD8 cells treated with ibrutinib, ABT-199, or a combination thereof. FIG. 26B illustrates colony formation of HBL1 cells treated with ibrutinib, ABT-199, or a combination thereof. FIG. 26C shows PI uptake and annexin-V binding of TMD8 cells treated with ibrutinib, ABT-199, or a combination thereof. FIG. 26D shows changes in tumor size following treatment. FIG. 26E shows apoptotic cell populations of TMD8 tumor cells.

FIG. 27A shows plots of cell growth of GCB-DLBCL cells (DLCL-2, RL, and SU-DHL-4) treated with ibrutinib in the presence or absence of ABT-199. FIG. 27B shows plot of cell growth of FL cells (DoHH2 and WSU-FSCCL) treated with ibrutinib in the presence or absence of ABT-199. FIG. 27C shows the C.I. of different concentrations of ibrutinib combined with ABT-199 at 100 nM (DLCL-2, RL, and SU-DHL-4), 30 nM (DoHH2), and 100 nm (WSU-FSCCL).

FIG. 28A shows cell growth of LY10 (BTK-C481S) treated with ibrutinib in the presence or absence of ABT-100. FIG. 28B shows the drug dose matrix data of LY10 (BTK-C481S). FIG. 28C shows the isobologram analysis for the date in FIG. 28B. FIG. 28D shows the C.I. of ibrutinib and ABT-199 at indicated concentrations in LY10 (BTK-C481S) cells. FIG. 28E shows cell growth of HBL1-resistant and TMD8-resistant cells treated with ibrutinib or a combination of ibrutinib and ABT-100. FIG. 28F illustrates the adhesion of TMD8-resistant cells treated with ibrutinib, ABT-199, or a combination thereof. FIG. 28G shows cell growth of DoHH2 resistant cells treated with ibrutinib or a combination of ibrutinib and ABT-199. FIG. 28H shows the C.I. of ibrutinib and ABT-199 at indicated concentrates in DoHH2-resistant cells.

FIG. 29A shows the gene-expression profiles of apoptosis-related genes in TMD8-WT versus TMD8-resistant cells. FIG. 29B shows the gene expression levels of BAX, BCL-2, and MCL-1. FIG. 29C shows cell growth of TMD8-WT and TMD8-resistant cells treated with ABT-199. FIG. 29D shows BCL-2 gene expression in DoHH2-WT and DoHH2-resistant cells.

FIG. 30A shows BCL-2 gene expression from tumors from ABC-DLBCL and GCB-DLBCL patients. FIG. 30B shows BCL-2 gene expression from tumors from ABC-DLBCL patients with poorer response (PD+SD). FIG. 30C shows progression-free survival (PFS) for pateints with low and high BCL-2.

FIG. 31 illustrates the key molecules in BCR signaling pathway with targeted agents. Shown are the key molecules in BCR signaling pathway which involve in NF-κB activation and therapeutic agents targeting this pathway. BCR, B cell receptor; CD79A and CD79B, cluster of differentiation CD79A and CD79B; SYK, spleen tyrosine kinase; BTK, Bruton tyrosine kinase; PLCγ2, phospholipase Cγ2; PKCβ, protein kinase Cβ; IKK, IκB kinase; NF-κB, nuclear factor-κB; BCL-2, B-cell lymphoma 2.

FIG. 32A-FIG. 32C show plots of cell growth of DLCL-2 cells treated with ibrutinib in the presence or absence of varying concentrations of ABT-199, IPI-145, or a combination thereof. In FIG. 32A, ABT199 concentration was 10 nM, while IPI-145 concentration ranged from 10, 100, to 1000 nM. In FIG. 32B, ABT199 concentration was 30 nm, while IPI-145 concentration ranged from 10, 100, to 1000 nM. In FIG. 32C, ABT199 concentration was 100 nm, while IPI145 concentration ranged from 10, 100, to 1000 nM.

FIG. 33A-FIG. 33C show plots of cell growth of SUDHL4, SDHL10, and DLCL-2 cells treated with ibrutinib in the presence or absence of varying concentrations ABT-199, IPI-145, or a combination thereof. FIG. 33A shows SUDHL4 cells under varying concentrations of ibrutinib, ABT-199 (0, 10, or 30 nM) and IPI 145 (0, 10, 100, or 1000 nM). FIG. 33B shows the SUDHL10 cells under varying concentrations of ibrutinib, ABT-199 (0, 10, or 30 nM) and IPI-145 (0, 10, 100, or 1000 nM). FIG. 33C shows the DLCL-2 cells under varying concentrations of ibrutinib, ABT-199 (0, 10, or 30 nM) and IPI 145 (0, 10, 100, or 1000 nM).

FIG. 34 shows the C.I. values of the combination of ibrutinib, ABT-199, and IPI-145 at indicated concentrations in SUDHL4, SUDHL10, and DLCL-2 cells.

FIG. 35A-FIG. 35B show plots of cell growth of SUDHL4 and DLCL-2 cells treated with ibrutinib in the presence or absence of varying concentrations of ABT-199, dexamethasone, or a combination thereof. FIG. 35A shows SUDL4 cells under varying concentrations of ibrutinib, ABT-199 and dexamethasone. FIG. 35B shows DLCL-2 cells under varying concentrations of ibrutinib, ABT-199 and dexamethasone.

FIG. 36A-FIG. 36B show plots of cell growth of SUDHL6 and SUDHL10 cells treated with ibrutinib in the presence or absence of varying concentrations of ABT-199, dexamethasone, or a combination thereof. FIG. 36A shows SUDL6 cells under varying concentrations of ibrutinib, ABT-199 and dexamethasone. FIG. 36B shows SUDHL10 cells under varying concentrations of ibrutinib, ABT-199 and dexamethasone.

FIG. 37 shows plots of cell growth of SUDHL4 cells treated with increasing concentrations of ibrutinib in the presence or absence of varying concentrations of ABT-199, dexamethasone, or a combination thereof.

FIG. 38 shows plots of cell growth of DLCL-2 cells treated with increasing concentrations of ibrutinib in the presence or absence of varying concentrations of ABT-199, dexamethasone, or a combination thereof.

FIG. 39 shows plots of cell growth of SUDHL6 cells treated with increasing concentrations of ibrutinib in the presence or absence of varying concentrations of ABT-199, dexamethasone, or a combination thereof.

FIG. 40 shows plots of cell growth of SUDHL10 cells treated with increasing concentrations of ibrutinib in the presence or absence of varying concentrations of ABT-199, dexamethasone, or a combination thereof.

FIG. 41 shows the C.I. values of the combination of ibrutinib, ABT-199, and dexamethasone at indicated concentrations in SUDHL4, SUDHL6, and DLCL-2 cells.

### DETAILED DESCRIPTION OF THE INVENTION

Methods, systems, compositions, arrays, kits, reagents, computers software, and reports are provided herein for use in analyzing one or more of the biomarkers or biomarker genes disclosed herein. In one aspect, disclosed herein are methods for stratifying an individual having a hematological malignancy such as DLBCL for treatment, based on the presence or absence of modifications in one or more biomarker genes selected from EP300, MLL2, BCL-2, RB1, LRP1B, PIM1, TSC2, TNFRSF11A, SMAD4, PAX5, and CARD11. In some cases, the presence of modifications in one or more biomarker genes selected from EP300, MLL2, BCL-2, RB1, LRP1B, PIM1, TSC2, TNFRSF11A, SMAD4, PAX5, and CARD11 indicate that an individual has developed resistance, or is likely to develop resistance to therapy with a TEC inhibitor such as an ITK inhibitor or a BTK inhibitor (e.g. ibrutinib). In other cases, an individual's therapeutic regimen is optimized, e.g. modifying, discontinuing, or continuing the treatment, based on the presence or absence of modifications in the one or more biomarker genes selected from EP300, MLL2, BCL-2, RB1, LRP1B, PIM1, TSC2, TNFRSF11A, SMAD4, PAX5, and CARD11.

In another aspect, disclosed herein are methods for selecting an individual having a hematological malignancy such as DLBCL for treatment, based on the presence or absence of a modification to an aromatic residue in CD79B and at least one modification at amino acid positions 198 or 265 in MYD88. In some instances, the presence of the modification to an aromatic residue in CD79B and at least one modification at amino acid positions 198 or 265 in MYD88 indicates that the individual is responsive or is likely to respond to therapy with a TEC inhibitor such as an ITK inhibitor or a BTK inhibitor (e.g. ibrutinib). In other instances, an individual's therapeutic regimen is optimized, e.g. modifying, discontinuing, or continuing the treatment, based on the presence or absence of the modification to an aromatic residue at amino acid position 196 in CD79B and at least one modification at amino acid positions 198 or 265 in MYD88.

In some instances, disclosed herein are methods of selecting an individual having a hematological malignancy such as DLBCL for treatment, based on the presence or absence of a modification at amino acid position 15 in ROS1. In some cases, the presence of the modification at amino acid position 15 in ROS1 indicates that the individual has developed resistance or is likely to develop resistance to therapy with a TEC inhibitor such as an ITK inhibitor or a BTK inhibitor (e.g. ibrutinib). In other cases, an individual's therapeutic regimen is optimized, e.g. modifying, discontinuing, or continuing the treatment, based on the presence or absence of the modification at amino acid position 15 in ROS1.

In other instances, disclosed herein are methods of classifying an individual having a hematological malignancy such as DLBCL for treatment with a TEC inhibitor such as an ITK inhibitor or a BTK inhibitor (e.g. ibrutinib), based on the expression level of at least one biomarker gene selected from ACTG2, LOR, GAPT, CCND2, SELL, GEN1, and HDAC9. In some cases, an elevated expression level in at least one biomarker gene selected from ACTG2, LOR, GAPT, CCND2, SELL, GEN1, and HDAC9 relative to a control indicates that the individual has a stable DLBCL.

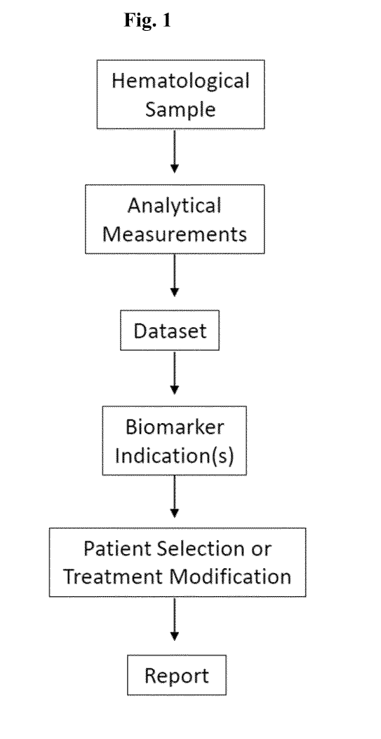

Also disclosed herein are systems for using biomarkers or biomarker genes disclosed herein for assessing an individual having a hematological malignancy such as DLBCL for treatment with a TEC inhibitor such as an ITK inhibitor or a BTK inhibitor (e.g. ibrutinib). In some cases, the systems comprise the analysis of a hematological sample such as a DLBCL sample by analytical techniques to derive biomarker data, or analytical measurements (FIG. 1). The biomarker data or analytical measurements are subsequently compiled by software into a dataset, which is then analyzed to determine one or more biomarker indications, such as the presence or absence of modifications in biomarker genes, or the expression level of biomarkers. The results are used to stratify patients prior to or during therapy regiments, to monitor the progress of a therapy regimen, or to optimize a therapy regimen. In some cases, the results are compiled into a report format for sending to a user.

Further disclosed herein are kits and arrays for using biomarkers or biomarker genes disclosed herein for use with the methods and systems disclosed above. In some embodiments, kits disclosed herein comprise one or more reagents for determining the presence or absence of modifications in one or more biomarker genes selected from BCL-2, RB1, LRP1B, PIM1, TSC2, TNFRSF11A, SMAD4, PAX5, and CARD11 in the sample, one or more reagents for determining the presence or absence of a modification to an aromatic residue at amino acid position 196 in CD79B and at least one modification at amino acid positions 198 or 265 in MYD88 in the sample, one or more reagents for determining the presence or absence of a modification at amino acid position 15 in ROS1 in the sample, or one or more reagents for determining the expression level of at least one biomarker gene selected from ACTG2, LOR, GAPT, CCND2, SELL, GEN1 and HDAC9 in the sample.

In some embodiments, a nucleic acid hybridization array comprising nucleic acid probes for evaluating an individual receiving a TEC inhibitor such as an ITK inhibitor or a BTK inhibitor (e.g. ibrutinib) for treatment of a hematological malignancy (e.g. DLBCL) has developed or is likely to develop resistance to the therapy, consisting essentially of nucleic acid probes which hybridize to biomarker genes selected from the group consisting of BCL-2, RB1, LRP1B, PIM1, TSC2, TNFRSF11A, SMAD4, PAX5, and CARD11. In some embodiments, a nucleic acid hybridization array comprising nucleic acid probes for evaluating whether an individual having a hematological malignancy (e.g. DLBCL) has a stable hematological malignancy (e.g. stable DLBCL), consisting essentially of nucleic acid probes which hybridize to biomarker genes selected from the group consisting of ACTG2, LOR, GAPT, CCND2, SELL, GEN1, and HDAC9.

### Certain Terminology

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

As used herein, ranges and amounts can be expressed as “about” a particular value or range. About also includes the exact amount. Hence “about 5 μL” means “about 5 μL” and also “5 μL.” Generally, the term “about” includes an amount that would be expected to be within experimental error.

The section headings used herein are for organizational purposes only and are not to be construed as limiting the subject matter described.

“Antibodies” and “immunoglobulins” (Igs) are glycoproteins having the same structural characteristics. The terms are used synonymously. In some instances the antigen specificity of the immunoglobulin is known.

The term “antibody” is used in the broadest sense and covers fully assembled antibodies, antibody fragments that can bind antigen (e.g., Fab, F(ab′)₂, Fv, single chain antibodies, diabodies, antibody chimeras, hybrid antibodies, bispecific antibodies, humanized antibodies, and the like), and recombinant peptides comprising the forgoing.

The terms “monoclonal antibody” and “mAb” as used herein refer to an antibody obtained from a substantially homogeneous population of antibodies, i.e., the individual antibodies comprising the population are identical except for possible naturally occurring mutations that may be present in minor amounts.

Native antibodies” and “native immunoglobulins” are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (V_{H}) followed by a number of constant domains. Each light chain has a variable domain at one end (V_{L}) and a constant domain at its other end; the constant domain of the light chain is aligned with the first constant domain of the heavy chain, and the light chain variable domain is aligned with the variable domain of the heavy chain. Particular amino acid residues are believed to form an interface between the light and heavy-chain variable domains.

The term “variable” refers to the fact that certain portions of the variable domains differ extensively in sequence among antibodies. Variable regions confer antigen-binding specificity. However, the variability is not evenly distributed throughout the variable domains of antibodies. It is concentrated in three segments called complementarity determining regions (CDRs) or hypervariable regions, both in the light chain and the heavy-chain variable domains. The more highly conserved portions of variable domains are celled in the framework (FR) regions. The variable domains of native heavy and light chains each comprise four FR regions, largely adopting a β-pleated-sheet configuration, connected by three CDRs, which form loops connecting, and in some cases forming part of, the β-pleated-sheet structure. The CDRs in each chain are held together in close proximity by the FR regions and, with the CDRs from the other chain, contribute to the formation of the antigen-binding site of antibodies (see, Kabat et al. (1991) NIH PubL. No. 91-3242, Vol. I, pages 647-669). The constant domains are not involved directly in binding an antibody to an antigen, but exhibit various effector functions, such as Fc receptor (FcR) binding, participation of the antibody in antibody-dependent cellular toxicity, initiation of complement dependent cytotoxicity, and mast cell degranulation.

The term “hypervariable region,” when used herein, refers to the amino acid residues of an antibody that are responsible for antigen-binding. The hypervariable region comprises amino acid residues from a “complementarily determining region” or “CDR” (i.e., residues 24-34 (L1), 50-56 (L2), and 89-97 (L3) in the light-chain variable domain and 31-35 (H1), 50-65 (H2), and 95-102 (H3) in the heavy-chain variable domain; Kabat et al. (1991) Sequences of Proteins of Immunological Interest, 5th Ed. Public Health Service, National Institute of Health, Bethesda, Md.) and/or those residues from a “hypervariable loop” (i.e., residues 26-32 (L1), 50-52 (L2), and 91-96 (L3) in the light-chain variable domain and (H1), 53-55 (H2), and 96-101 (13) in the heavy chain variable domain; Clothia and Lesk, (1987) J. Mol. Biol., 196:901-917). “Framework” or “FR” residues are those variable domain residues other than the hypervariable region residues, as herein deemed.

“Antibody fragments” comprise a portion of an intact antibody, preferably the antigen-binding or variable region of the intact antibody. Examples of antibody fragments include Fab, Fab, F(ab′)2, and Fv fragments; diabodies; linear antibodies (Zapata et al. (1995) Protein Eng. 10:1057-1062); single-chain antibody molecules; and multispecific antibodies formed from antibody fragments. Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)2 fragment that has two antigen-combining sites and is still capable of cross-linking antigen.

“Fv” is the minimum antibody fragment that contains a complete antigen recognition and binding site. This region consists of a dimer of one heavy- and one light-chain variable domain in tight, non-covalent association. It is in this configuration that the three CDRs of each variable domain interact to define an antigen-binding site on the surface of the V_{H}-V_{L} dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

The Fab fragment also contains the constant domain of the light chain and the first constant domain (C_{H1}) of the heavy chain. Fab fragments differ from Fab′ fragments by the addition of a few residues at the carboxy terminus of the heavy chain C_{H1} domain including one or more cysteines from the antibody hinge region. Fab′-SH is the designation herein for Fab′ in which the cysteine residue(s) of the constant domains bear a free thiol group. Fab′ fragments are produced by reducing the F(ab′)2 fragment's heavy chain disulfide bridge. Other chemical couplings of antibody fragments are also known.

The “light chains” of antibodies (immunoglobulins) from any vertebrate species can be assigned to one of two clearly distinct types, called kappa (κ) and lambda (λ), based on the amino acid sequences of their constant domains.

Depending on the amino acid sequence of the constant domain of their heavy chains, immunoglobulins can be assigned to different classes. There are five major classes of human immunoglobulins: IgA, IgD, IgE, IgG, and IgM, and several of these may be further divided into subclasses (isotypes), e.g., IgG1, IgG2, IgG3, IgG4, IgA1, and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known. Different isotypes have different effector functions. For example, human IgG1 and IgG3 isotypes have ADCC (antibody dependent cell-mediated cytotoxicity) activity.

As used herein, the terms “individual(s)”, “subject(s)” and “patient(s)” mean any mammal. In some embodiments, the mammal is a human. In some embodiments, the mammal is a non-human. None of the terms require or are limited to situations characterized by the supervision (e.g. constant or intermittent) of a health care worker (e.g. a doctor, a registered nurse, a nurse practitioner, a physician's assistant, an orderly or a hospice worker).

### Hematological Malignancies

Hematological malignancies are a diverse group of cancer that affects the blood, bone marrow, and lymph nodes. In some embodiments, the hematological malignancy is a leukemia, a lymphoma, a myeloma, a non-Hodgkin's lymphoma, a Hodgkin's lymphoma, T-cell malignancy, or a B-cell malignancy.

In some embodiments, the hematological malignancy is a T-cell malignancy. In some embodiments, T-cell malignancies include peripheral T-cell lymphoma not otherwise specified (PTCL-NOS), anaplastic large cell lymphoma, angioimmunoblastic lymphoma, cutaneous T-cell lymphoma, adult T-cell leukemia/lymphoma (ATLL), blastic NK-cell lymphoma, enteropathy-type T-cell lymphoma, hematosplenic gamma-delta T-cell lymphoma, lymphoblastic lymphoma, nasal NK/T-cell lymphomas, or treatment-related T-cell lymphomas.

In some embodiments, the hematological malignancy is a B-cell malignancy. In some embodiments, the B-cell malignancy is DLBCL. In some embodiments, additional B-cell malignancies include acute lymphoblastic leukemia (ALL), acute myelogenous leukemia (AML), chronic myelogenous leukemia (CML), acute monocytic leukemia (AMoL), chronic lymphocytic leukemia (CLL), high-risk chronic lymphocytic leukemia (CLL), small lymphocytic lymphoma (SLL), high-risk small lymphocytic lymphoma (SLL), follicular lymphoma (FL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis.

In some embodiments, the hematological malignancy is a non-Hodgkin's lymphoma. In some embodiments, a non-Hodgkin's lymphoma is formed from B-cells. In some embodiments, a non-Hodgkin's lymphoma is formed from T-cells. Exemplary non-Hodgkin's lymphoma include, but is not limited to, Burkitt lymphoma, CLL, SLL, DLBCL, FL, immunoblastic large cell lymphoma, precursor B-lymphoblastic lymphoma, mantle cell lymphoma, marginal zone B-cell lymphomas, Waldenstrom's macroglobulinemia, lymphoplasmacytic lymphoma, hairy cell leukemia, mediastinal large B-cell lymphoma, cutaneous lymphomas, mycosis fungoides, anaplastic large cell lymphoma, peripheral T-cell lymphomas, enteropathy associated T cell lymphoma (EATL), hepatosplenic gamma delta T cell lymphoma, and precursor T-lymphoblastic lymphoma.

In some embodiments, the non-Hodgkin's lymphoma is Burkitt lymphoma, CLL, SLL, DLBCL, FL, immunoblastic large cell lymphoma, precursor B-lymphoblastic lymphoma, mantle cell lymphoma, marginal zone B-cell lymphomas, Waldenstrom's macroglobulinemia, lymphoplasmacytic lymphoma, hairy cell leukemia, or mediastinal large B-cell lymphoma.

In some embodiments, the non-Hodgkin's lymphoma is Burkitt lymphoma. In some embodiments, the non-Hodgkin's lymphoma is CLL. In some embodiments, the non-Hodgkin's lymphoma is SLL. In some embodiments, the non-Hodgkin's lymphoma is DLBCL. In some embodiments, the non-Hodgkin's lymphoma is FL. In some embodiments, the non-Hodgkin's lymphoma is mantle cell lymphoma. In some embodiments, the non-Hodgkin's lymphoma is Waldenstrom's macroglobulinemia.

In some embodiments, the hematological malignancy is Burkitt lymphoma, CLL, SLL, DLBCL, FL, immunoblastic large cell lymphoma, precursor B-lymphoblastic lymphoma, mantle cell lymphoma, marginal zone B-cell lymphomas, Waldenstrom's macroglobulinemia, lymphoplasmacytic lymphoma, hairy cell leukemia, or mediastinal large B-cell lymphoma.

In some embodiments, the hematological malignancy is a relapsed or refractory hematological malignancy. In some embodiments, the hematological malignancy is a relapsed hematological malignancy. In some embodiments, the hematological malignancy is a refractory hematological malignancy. In some embodiments, the refractory hematological malignancy contains an acquired resistance to a Btk inhibitor. In some embodiments, the refractory hematological malignancy contains an acquired insensitivity to a Btk inhibitor. In some embodiments, the Btk inhibitor is ibrutinib. In some embodiments, the refractory hematological malignancy is Btk-resistant hematological malignancy. In some embodiments, the refractory hematological malignancy is Btk-insensitive hematological malignancy. In some embodiments, the hematological malignancy is Btk-resistant hematological malignancy. In some embodiments, the hematological malignancy is Btk-insensitive hematological malignancy.

In some embodiments, the relapsed or refractory hematological malignancy include DLBCL, acute lymphoblastic leukemia (ALL), acute myelogenous leukemia (AML), chronic myelogenous leukemia (CML), acute monocytic leukemia (AMoL), chronic lymphocytic leukemia (CLL), high-risk chronic lymphocytic leukemia (CLL), small lymphocytic lymphoma (SLL), high-risk small lymphocytic lymphoma (SLL), follicular lymphoma (FL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis.

In some embodiments, the relapsed or refractory hematological malignancy is a relapsed or refractory non-Hodgkin's lymphoma. In some embodiments, the relapsed or refractory non-Hodgkin's lymphoma include Burkitt lymphoma, CLL, SLL, DLBCL, FL, immunoblastic large cell lymphoma, precursor B-lymphoblastic lymphoma, mantle cell lymphoma, marginal zone B-cell lymphomas, Waldenstrom's macroglobulinemia, lymphoplasmacytic lymphoma, hairy cell leukemia, mediastinal large B-cell lymphoma, cutaneous lymphomas, mycosis fungoides, anaplastic large cell lymphoma, peripheral T-cell lymphomas, enteropathy associated T cell lymphoma (EATL), hepatosplenic gamma delta T cell lymphoma, and precursor T-lymphoblastic lymphoma.

In some embodiments, the relapsed or refractory non-Hodgkin's lymphoma is Burkitt lymphoma, CLL, SLL, DLBCL, FL, immunoblastic large cell lymphoma, precursor B-lymphoblastic lymphoma, mantle cell lymphoma, marginal zone B-cell lymphomas, Waldenstrom's macroglobulinemia, lymphoplasmacytic lymphoma, hairy cell leukemia, or mediastinal large B-cell lymphoma.

In some embodiments, the relapsed or refractory non-Hodgkin's lymphoma is a relapsed or refractory DLBCL. In some embodiments, the relapsed or refractory non-Hodgkin's lymphoma is a relapsed or refractory CLL. In some embodiments, the relapsed or refractory non-Hodgkin's lymphoma is a relapsed or refractory SLL. In some embodiments, the relapsed or refractory non-Hodgkin's lymphoma is a relapsed or refractory FL. In some embodiments, the relapsed or refractory non-Hodgkin's lymphoma is a relapsed or refractory Burkitt lymphoma. In some embodiments, the relapsed or refractory non-Hodgkin's lymphoma is a relapsed or refractory Waldenstrom's macroglobulinemia. In some embodiments, the relapsed or refractory non-Hodgkin's lymphoma is a relapsed or refractory mantle cell lymphoma.

In some embodiments, the hematological malignancy is a metastasized hematological malignancy. In some embodiments, the metastasized hematological malignancy contains an acquired resistance to a Btk inhibitor. In some embodiments, the metastasized hematological malignancy contains an acquired insensitivity to a Btk inhibitor. In some embodiments, the Btk inhibitor is ibrutinib. In some embodiments, the metastasized hematological malignancy is Btk-resistant hematological malignancy. In some embodiments, the metastasized hematological malignancy is Btk-insensitive hematological malignancy.

In some embodiments, the metastasized hematological malignancy include DLBCL, acute lymphoblastic leukemia (ALL), acute myelogenous leukemia (AML), chronic myelogenous leukemia (CML), acute monocytic leukemia (AMoL), chronic lymphocytic leukemia (CLL), high-risk chronic lymphocytic leukemia (CLL), small lymphocytic lymphoma (SLL), high-risk small lymphocytic lymphoma (SLL), follicular lymphoma (FL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis.

In some embodiments, the metastasized hematological malignancy is a metastasized non-Hodgkin's lymphoma. In some embodiments, the metastasized non-Hodgkin's lymphoma include Burkitt lymphoma, CLL, SLL, DLBCL, FL, immunoblastic large cell lymphoma, precursor B-lymphoblastic lymphoma, mantle cell lymphoma, marginal zone B-cell lymphomas, Waldenstrom's macroglobulinemia, lymphoplasmacytic lymphoma, hairy cell leukemia, mediastinal large B-cell lymphoma, cutaneous lymphomas, mycosis fungoides, anaplastic large cell lymphoma, peripheral T-cell lymphomas, enteropathy associated T cell lymphoma (EATL), hepatosplenic gamma delta T cell lymphoma, and precursor T-lymphoblastic lymphoma.

In some embodiments, the metastasized non-Hodgkin's lymphoma is Burkitt lymphoma, CLL, SLL, DLBCL, FL, immunoblastic large cell lymphoma, precursor B-lymphoblastic lymphoma, mantle cell lymphoma, marginal zone B-cell lymphomas, Waldenstrom's macroglobulinemia, lymphoplasmacytic lymphoma, hairy cell leukemia, or mediastinal large B-cell lymphoma.

In some embodiments, the metastasized non-Hodgkin's lymphoma is a metastasized DLBCL. In some embodiments, the metastasized non-Hodgkin's lymphoma is a metastasized CLL. In some embodiments, the metastasized non-Hodgkin's lymphoma is a metastasized SLL. In some embodiments, the metastasized non-Hodgkin's lymphoma is a metastasized FL. In some embodiments, the metastasized non-Hodgkin's lymphoma is a metastasized Burkitt lymphoma. In some embodiments, the metastasized non-Hodgkin's lymphoma is a metastasized Waldenstrom's macroglobulinemia. In some embodiments, the metastasized non-Hodgkin's lymphoma is a metastasized mantle cell lymphoma.

### Diffuse Large B Cell Lymphoma (DLBCL)

Diffuse large B cell lymphoma (DLBCL) is the most prevalent type of aggressive non-Hodgkin's lymphoma (NHL) in the United States. Clinical courses of patients with DLBCL are highly heterogeneous. While majority of the patients with DLBCL show response to the initial treatment, approximately one-third of patients have refractory disease or experience relapse after the standard therapies. DLBCL is a clinically and biologically heterogeneous disease, which can be demonstrated by several clinical and molecularly defined prognostic models. In certain instances, gene expression profiling (GEP) has been employed for dissecting the molecular heterogeneity and for predicting outcome in DLBCL. GEP can distinguish two prognostic subtypes, germinal center B cell-like (GCB) and activated B cell-like (ABC) DLBCL, among whose functional differences include activity of B cell receptor (BCR) signaling. ABC DLBCL cells have chronic active BCR signaling, upon which their survival is highly dependent.

One signaling pathway in the pathogenesis of ABC-DLBCL is the one mediated by the nuclear factor (NF)-κB transcription complex. The NF-κB family comprises 5 members (p50, p52, p65, c-rel and RelB) that form homo- and heterodimers and function as transcriptional factors to mediate a variety of proliferation, apoptosis, inflammatory and immune responses and are critical for normal B-cell development and survival. NF-κB is widely used by eukaryotic cells as a regulator of genes that control cell proliferation and cell survival. As such, many different types of human tumors have misregulated NF-κB: that is, NF-κB is constitutively active. Active NF-κB turns on the expression of genes that keep the cell proliferating and protect the cell from conditions that would otherwise cause it to die via apoptosis.

The dependence of ABC DLBCLs on NF-kB depends on a signaling pathway upstream of IkB kinase comprised of CARD11, BCL10 and MALT1 (the CBM complex). Interference with the CBM pathway extinguishes NF-kB signaling in ABC DLBCL cells and induces apoptosis. The molecular basis for constitutive activity of the NF-kB pathway is a subject of current investigation but some somatic alterations to the genome of ABC DLBCLs clearly invoke this pathway. For example, somatic mutations of the coiled-coil domain of CARD11 in DLBCL render this signaling scaffold protein able to spontaneously nucleate protein-protein interaction with MALT1 and BCL10, causing IKK activity and NF-kB activation. Constitutive activity of the B cell receptor signaling pathway has been implicated in the activation of NF-kB in ABC DLBCLs with wild type CARD11, and this is associated with mutations within the cytoplasmic tails of the B cell receptor subunits CD79A and CD79B. Oncogenic activating mutations in the signaling adapter MYD88 activate NF-kB and synergize with B cell receptor signaling in sustaining the survival of ABC DLBCL cells. In addition, inactivating mutations in a negative regulator of the NF-kB pathway, A20, occur almost exclusively in ABC DLBCL.

Early and effective treatment of DLBCL is a critical factor affecting the survival of DLBCL patients. The selection of treatment regimens against which DLBCL is resistant delays the onset of effective treatment of the cancer and can lead to growth and spread of the cancer. This, in turn, can have a negative effect on the patient's treatment outcome. Tumor-specific characteristics that are associated with responsiveness to an anti-cancer agent, e.g., a BTK inhibitor, such as the expression of one or more specific genes and/or encoded proteins are useful as a prognostic biomarker for identifying potential patients likely to respond or fail treatment with a BTK inhibitor at an earlier stage. As a result, patients suffering from DLBCL expressing such a biomarker can be selected for treatment with a BTK inhibitor. In addition, the biomarker can be employed for assessing the response to treatment with a BTK inhibitor.

Disclosed herein, are methods, systems, compositions, arrays, and kits for using biomarkers or biomarker genes disclosed herein for stratifying a patient having DLBCL for treatment. Also disclosed herein are methods, systems, compositions, arrays, and kits for using biomarkers or biomarker genes for monitoring a patient during treatment of DLBCL. Further disclosed herein are methods, systems, compositions, arrays, and kits for using biomarkers or biomarker genes for optimizing a treatment regimen with a TEC inhibitor. In some embodiments, DLBCL is ABC-DLBCL, GCB-DLBCL, double-hit (DH) DLBCL, triple hit (TH) DLBCL, or unclassified DLBCL. In some embodiments, the TEC inhibitor is an ITK inhibitor or a BTK inhibitor. In some embodiments, the BTK inhibitor is ibrutinib.

### Follicular Lymphoma

Follicular lymphoma (FL) is the most common indolent non-Hodgkin's lymphoma (NHL), and in some cases account for about 20 to about 30 percent of NHL cases. In some instances, common signs of disease include enlargement of the lymph nodes in the neck, underarm, stomach, or groin, as well as fatigue, shortness of breath, night sweats, and weight loss. In some embodiments, BCL-2 translocations (e.g. t(14;18)(q32;q21)) and BCL-6 translocations are observed in FL.

Disclosed herein, are methods, systems, compositions, arrays, and kits for using biomarkers or biomarker genes disclosed herein for stratifying a patient having FL for treatment. Also disclosed herein are methods, systems, compositions, arrays, and kits for using biomarkers or biomarker genes for monitoring a patient during treatment of FL. Additionally disclosed herein are methods, systems, compositions, arrays, and kits for measuring the biomarker expression levels or the biomarker mutation rates as means of diagnosing, evaluating, or monitoring development of insensitivity to a TEC inhibitor. Also disclosed herein are methods, systems, compositions, arrays, and kits for measuring the biomarker expression levels or the biomarker mutation rates as means of diagnosing, evaluating, or monitoring a patient's response to a TEC inhibitor. Further disclosed herein are methods, systems, compositions, arrays, and kits for using biomarkers or biomarker genes for optimizing a treatment regimen with a TEC inhibitor. In some embodiments, the TEC inhibitor is an ITK inhibitor or a BTK inhibitor. In some embodiments, the BTK inhibitor is ibrutinib.

### CLL/SLL

Chronic lymphocytic leukemia and small lymphocytic lymphoma (CLL/SLL) are commonly thought as the same disease with slightly different manifestations. Where the cancerous cells gather determines whether it is called CLL or SLL. When the cancer cells are primarily found in the lymph nodes, lima bean shaped structures of the lymphatic system (a system primarily of tiny vessels found in the body), it is called SLL. SLL accounts for about 5% to 10% of all lymphomas. When most of the cancer cells are in the bloodstream and the bone marrow, it is called CLL.

Both CLL and SLL are slow-growing diseases, although CLL, which is much more common, tends to grow slower. CLL and SLL are treated the same way. They are usually not considered curable with standard treatments, but depending on the stage and growth rate of the disease, most patients live longer than 10 years. Occasionally over time, these slow-growing lymphomas may transform into a more aggressive type of lymphoma.

Chronic lymphoid leukemia (CLL) is the most common type of leukemia. It is estimated that 100,760 people in the United States are living with or are in remission from CLL. Most (>75%) people newly diagnosed with CLL are over the age of 50. Currently CLL treatment focuses on controlling the disease and its symptoms rather than on an outright cure. CLL is treated by chemotherapy, radiation therapy, biological therapy, or bone marrow transplantation. Symptoms are sometimes treated surgically (splenectomy removal of enlarged spleen) or by radiation therapy (“de-bulking” swollen lymph nodes). Though CLL progresses slowly in most cases, it is considered generally incurable. Certain CLLs are classified as high-risk. As used herein, “high risk CLL” means CLL characterized by at least one of the following 1) 17p13−; 2) 11q22−; 3) unmutated IgVH together with ZAP-70+ and/or CD38+; or 4) trisomy 12.

CLL treatment is typically administered when the patient's clinical symptoms or blood counts indicate that the disease has progressed to a point where it may affect the patient's quality of life.

Small lymphocytic leukemia (SLL) is very similar to CLL described supra, and is also a cancer of B-cells. In SLL the abnormal lymphocytes mainly affect the lymph nodes. However, in CLL the abnormal cells mainly affect the blood and the bone marrow. The spleen may be affected in both conditions. SLL accounts for about 1 in 25 of all cases of non-Hodgkin lymphoma. It can occur at any time from young adulthood to old age, but is rare under the age of 50. SLL is considered an indolent lymphoma. This means that the disease progresses very slowly, and patients tend to live many years after diagnosis. However, most patients are diagnosed with advanced disease, and although SLL responds well to a variety of chemotherapy drugs, it is generally considered to be incurable. Although some cancers tend to occur more often in one gender or the other, cases and deaths due to SLL are evenly split between men and women. The average age at the time of diagnosis is 60 years.

Although SLL is indolent, it is persistently progressive. The usual pattern of this disease is one of high response rates to radiation therapy and/or chemotherapy, with a period of disease remission. This is followed months or years later by an inevitable relapse. Re-treatment leads to a response again, but again the disease will relapse. This means that although the short-term prognosis of SLL is quite good, over time, many patients develop fatal complications of recurrent disease. Considering the age of the individuals typically diagnosed with CLL and SLL, there is a need in the art for a simple and effective treatment of the disease with minimum side-effects that do not impede on the patient's quality of life. The instant invention fulfills this long standing need in the art.

Disclosed herein, are methods for using biomarkers or biomarker genes disclosed herein for stratifying a patient having CLL/SLL for treatment. Also disclosed herein are methods for using biomarkers or biomarker genes for monitoring a patient during treatment of CLL/SLL. Further disclosed herein are methods for using biomarkers or biomarker genes for optimizing a treatment regimen with a TEC inhibitor. In some embodiments, the TEC inhibitor is an ITK inhibitor or a BTK inhibitor. In some embodiments, the BTK inhibitor is ibrutinib.

### Mantle Cell Lymphoma

Mantle cell lymphoma is a subtype of B-cell lymphoma, due to CD5 positive antigen-naive pregerminal center B-cell within the mantle zone that surrounds normal germinal center follicles. MCL cells generally over-express cyclin D1 due to a t(11:14) chromosomal translocation in the DNA. More specifically, the translocation is at t(11;14)(q13;q32). Only about 5% of lymphomas are of this type. The cells are small to medium in size. Men are affected most often. The average age of patients is in the early 60s. The lymphoma is usually widespread when it is diagnosed, involving lymph nodes, bone marrow, and, very often, the spleen. Mantle cell lymphoma is not a very fast growing lymphoma, but is difficult to treat.

Disclosed herein, are methods for using biomarkers or biomarker genes disclosed herein for stratifying a patient having mantel cell lymphoma for treatment. Also disclosed herein are methods for using biomarkers or biomarker genes for monitoring a patient during treatment of mantel cell lymphoma. Further disclosed herein are methods for using biomarkers or biomarker genes for optimizing a treatment regimen with a TEC inhibitor. In some embodiments, the TEC inhibitor is an ITK inhibitor or a BTK inhibitor. In some embodiments, the BTK inhibitor is ibrutinib.

### Waldenstrom's Macroglobulinemia

Waldenstrom's macroglobulinemia, also known as lymphoplasmacytic lymphoma, is cancer involving a subtype of white blood cells called lymphocytes. It is characterized by an uncontrolled clonal proliferation of terminally differentiated B lymphocytes. It is also characterized by the lymphoma cells making an antibody called immunoglobulin M (IgM). The IgM antibodies circulate in the blood in large amounts, and cause the liquid part of the blood to thicken, like syrup. This can lead to decreased blood flow to many organs, which can cause problems with vision (because of poor circulation in blood vessels in the back of the eyes) and neurological problems (such as headache, dizziness, and confusion) caused by poor blood flow within the brain. Other symptoms can include feeling tired and weak, and a tendency to bleed easily. The underlying etiology is not fully understood but a number of risk factors have been identified, including the locus 6p21.3 on chromosome 6. There is a 2- to 3-fold risk increase of developing WM in people with a personal history of autoimmune diseases with autoantibodies and particularly elevated risks associated with hepatitis, human immunodeficiency virus, and rickettsiosis.

Disclosed herein, are methods for using biomarkers or biomarker genes disclosed herein for stratifying a patient having Waldenstrom's macroglobulinemia for treatment. Also disclosed herein are methods for using biomarkers or biomarker genes for monitoring a patient during treatment of Waldenstrom's macroglobulinemia. Further disclosed herein are methods for using biomarkers or biomarker genes for optimizing a treatment regimen with a TEC inhibitor. In some embodiments, the TEC inhibitor is an ITK inhibitor or a BTK inhibitor. In some embodiments, the BTK inhibitor is ibrutinib.

### Biomarkers

Disclosed herein, in certain embodiments, are methods of using biomarkers or biomarker genes disclosed herein for stratifying a patient having a hematological malignancy for treatment. Also disclosed herein are methods of using biomarkers or biomarker genes for monitoring a patient during treatment of a hematological malignancy. Further disclosed herein are methods of using biomarkers or biomarker genes for optimizing a treatment regimen. In some embodiments, the hematological malignancy is a leukemia, a lymphoma, a myeloma, a non-Hodgkin's lymphoma, a Hodgkin's lymphoma, T-cell malignancy, or a B-cell malignancy. In some embodiments, the hematological malignancy is a B-cell malignancy. In some embodiments, the B-cell malignancy is acute lymphoblastic leukemia (ALL), acute myelogenous leukemia (AML), chronic myelogenous leukemia (CML), acute monocytic leukemia (AMoL), chronic lymphocytic leukemia (CLL), high-risk chronic lymphocytic leukemia (CLL), small lymphocytic lymphoma (SLL), high-risk small lymphocytic lymphoma (SLL), diffuse large B cell lymphoma (DLBCL), follicular lymphoma (FL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis. In some embodiments, the hematological malignancy is chronic lymphocytic leukemia (CLL), high-risk chronic lymphocytic leukemia (CLL), small lymphocytic lymphoma (SLL), high-risk small lymphocytic lymphoma (SLL), diffuse large B cell lymphoma (DLBCL), mantle cell lymphoma (MCL), or Waldenstrom's macroglobulinemia. In some embodiments, the hematological malignancy is DLBCL. In some embodiments, the treatment comprises administration of a TEC inhibitor. In some embodiments, the TEC inhibitor is a BTK inhibitor, an ITK inhibitor, a TEC inhibitor, a RLK inhibitor, or a BMX inhibitor. In some embodiments, the TEC inhibitor is an ITK inhibitor. In some embodiments, the TEC inhibitor is a BTK inhibitor. In some embodiments, the BTK inhibitor is ibrutinib.

In some embodiments, the biomarker or biomarker genes are evaluated based on the presence or absence of modifications or mutations in the biomarkers or biomarker genes, or by expression level. In some embodiments, modifications are determined in genes selected from CDKN2A, CDKN2B, MYD88, PIK3C2G, CD79B, IRS2, BCL2, RB1, LRP1B, PIM1, TSC2, TNFRSF11A, SMAD4, PAX5, and CARD11. In some embodiments, modifications are determined in genes selected from BCL-2, RB1, LRP1B, PIM1, TSC2, TNFRSF11A, SMAD4, PAX5, and CARD11. In some embodiments, modifications are determined in genes selected from BCL-2, RB1, LRP1B, PIM1, and TSC2.

In some embodiments, the biomarker or biomarker genes are evaluated based on the expression level. In some instances, the expression level is compared to a reference level. In some instances, the expression level is an increased expression level. In some instances, the expression level is a decreased expression level. In some embodiments, expression levels of genes selected from CDKN2A, CDKN2B, MYD88, PIK3C2G, CD79B, IRS2, BCL2, RB1, LRP1B, PIM1, TSC2, TNFRSF11A, SMAD4, PAX5, and CARD11 are determined. In some embodiments, expression levels of genes selected from BCL-2, RB1, LRP1B, PIM1, TSC2, TNFRSF11A, SMAD4, PAX5, and CARD11 are determined. In some embodiments, expression levels of genes selected from BCL-2, RB1, LRP1B, PIM1, and TSC2 are determined. In some embodiments, the expression level of BCL-2 is determined.

In some embodiments, the presence or absence of modifications in one or more biomarker genes selected from EP300, MLL2, BCL-2, RB1, LRP1B, PIM1, TSC2, TNFRSF11A, SMAD4, PAX5, and CARD11 are used to select patients or individuals having a hematological malignancy for treatment with a TEC inhibitor if there is an absence of modifications in one or more of the biomarker genes. In some embodiments, the presence or absence of modifications in one or more biomarker genes selected from EP300, MLL2, BCL-2, RB1, LRP1B, PIM1, TSC2, TNFRSF11A, SMAD4, PAX5, and CARD11 are used to monitor an individual receiving a TEC inhibitor treatment for developing or likely to develop resistance to the therapy if the individual has modifications in one or more of the biomarker genes. In some embodiments, the presence or absence of modifications in one or more biomarker genes selected from EP300, MLL2, BCL-2, RB1, LRP1B, PIM1, TSC2, TNFRSF11A, SMAD4, PAX5, and CARD11 are used to optimize therapy of an individual receiving a TEC inhibitor. In some embodiments, the hematological malignancy is a leukemia, a lymphoma, a myeloma, a non-Hodgkin's lymphoma, a Hodgkin's lymphoma, T-cell malignancy, or a B-cell malignancy. In some embodiments, the hematological malignancy is a B-cell malignancy. In some embodiments, the B-cell malignancy is chronic lymphocytic leukemia (CLL), high-risk chronic lymphocytic leukemia (CLL), small lymphocytic lymphoma (SLL), high-risk small lymphocytic lymphoma (SLL), diffuse large B cell lymphoma (DLBCL), mantle cell lymphoma (MCL), or Waldenstrom's macroglobulinemia. In some embodiments, the B-cell malignancy is DLBCL. In some embodiments, the DLBCL is activated B-cell DLBCL (ABC-DLBCL), germinal center B-cell like DLBCL (GBC-DLBCL), double-hit (DH) DLBCL, triple-hit (TH) DLBCL, or unclassified DLBCL. In some embodiments, DLBCL is activated B-cell DLBCL (ABC-DLBCL), germinal center B-cell like DLBCL (GBC-DLBCL), or unclassified DLBCL. In some embodiments, the TEC inhibitor is a BTK inhibitor, an ITK inhibitor, a TEC inhibitor, a RLK inhibitor, or a BMX inhibitor. In some embodiments, the TEC inhibitor is an ITK inhibitor. In some embodiments, the TEC inhibitor is a BTK inhibitor. In some embodiments, the BTK inhibitor is ibrutinib.

In some embodiments, the presence or absence of modifications in one or more biomarker genes selected from EP300, MLL2, BCL-2, RB1, LRP1B, PIM1, TSC2, TNFRSF11A, SMAD4, PAX5, and CARD11 are used to select patients or individuals having DLBCL for treatment with an ITK inhibitor if there is an absence of modifications in one or more of the biomarker genes. In some embodiments, the presence or absence of modifications in one or more biomarker genes selected from EP300, MLL2, BCL-2, RB1, LRP1B, PIM1, TSC2, TNFRSF11A, SMAD4, PAX5, and CARD11 are used to monitor an individual receiving an ITK inhibitor treatment for developing or likely to develop resistance to the therapy if the individual has modifications in one or more of the biomarker genes. In some embodiments, the presence or absence of modifications in one or more biomarker genes selected from EP300, MLL2, BCL-2, RB1, LRP1B, PIM1, TSC2, TNFRSF11A, SMAD4, PAX5, and CARD11 are used to optimize therapy of an individual receiving an ITK inhibitor.

In some embodiments, the presence or absence of modifications in one or more biomarker genes selected from EP300, MLL2, BCL-2, RB1, LRP1B, PIM1, TSC2, TNFRSF11A, SMAD4, PAX5, and CARD11 are used to select patients or individuals having DLBCL for treatment with a BTK inhibitor if there is an absence of modifications in one or more of the biomarker genes. In some embodiments, the presence or absence of modifications in one or more biomarker genes selected from EP300, MLL2, BCL-2, RB1, LRP1B, PIM1, TSC2, TNFRSF11A, SMAD4, PAX5, and CARD11 are used to monitor an individual receiving a BTK inhibitor treatment for developing or likely to develop resistance to the therapy if the individual has modifications in one or more of the biomarker genes. In some embodiments, the presence or absence of modifications in one or more biomarker genes selected from EP300, MLL2, BCL-2, RB1, LRP1B, PIM1, TSC2, TNFRSF11A, SMAD4, PAX5, and CARD11 are used to optimize therapy of an individual receiving a BTK inhibitor. In some embodiments, the BTK inhibitor is selected from among ibrutinib (PCI-32765), PCI-45292, PCI-45466, AVL-101/CC-101 (Avila Therapeutics/Celgene Corporation), AVL-263/CC-263 (Avila Therapeutics/Celgene Corporation), AVL-292/CC-292 (Avila Therapeutics/Celgene Corporation), AVL-291/CC-291 (Avila Therapeutics/Celgene Corporation), CNX 774 (Avila Therapeutics), BMS-488516 (Bristol-Myers Squibb), BMS-509744 (Bristol-Myers Squibb), CGI-1746 (CGI Pharma/Gilead Sciences), CGI-560 (CGI Pharma/Gilead Sciences), CTA-056, GDC-0834 (Genentech), HY-11066 (also, CTK4I7891, HMS3265G21, HMS3265G22, HMS3265H21, HMS3265H22, 439574-61-5, AG-F-54930), ONO-4059 (Ono Pharmaceutical Co., Ltd.), ONO-WG37 (Ono Pharmaceutical Co., Ltd.), PLS-123 (Peking University), RN486 (Hoffmann-La Roche), HM71224 (Hanmi Pharmaceutical Company Limited), LFM-A13, BGB-3111 (Beigene), KBP-7536 (KBP BioSciences), ACP-196 (Acerta Pharma), JTE-051 (Japan Tobacco Inc), PRN1008 (Principia), CTP-730 (Concert Pharmaceuticals), or GDC-0853 (Genentech).

In some embodiments, the presence or absence of modifications in one or more biomarker genes selected from EP300, MLL2, BCL-2, RB1, LRP1B, PIM1, TSC2, TNFRSF11A, SMAD4, PAX5, and CARD11 are used to select patients or individuals having DLBCL for treatment with ibrutinib if there is an absence of modifications in one or more of the biomarker genes. In some embodiments, the presence or absence of modifications in one or more biomarker genes selected from EP300, MLL2, BCL-2, RB1, LRP1B, PIM1, TSC2, TNFRSF11A, SMAD4, PAX5, and CARD11 are used to monitor an individual receiving ibrutinib treatment for developing or likely to develop resistance to the therapy if the individual has modifications in one or more of the biomarker genes. In some embodiments, the presence or absence of modifications in one or more biomarker genes selected from EP300, MLL2, BCL-2, RB1, LRP1B, PIM1, TSC2, TNFRSF11A, SMAD4, PAX5, and CARD11 are used to optimize therapy of an individual receiving ibrutinib.

In some embodiments, the modifications or mutations associated with CARD11 include mutations at amino acid positions 117, 250, 248, 128, 249, and 232. In some embodiments, the modifications are T117P, S250P, N248S, T128M, Q249P, L232LL, L232IL, or L232LI.

In some embodiments, also disclosed herein are methods of selecting a patient having a hematological malignancy such as DLBCL for treatment with a TEC inhibitor, such as an ITK inhibitor or a BTK inhibitor (e.g. ibrutinib) by determining the presence or absence of a modification in one or more biomarker genes selected from EP300, MLL2, BCL-2, RB1, LRP1B, PIM1, TSC2, TNFRSF11A, SMAD4, PAX5, and CARD11, and one or more additional biomarkers. In some embodiments, disclosed herein are methods of monitoring whether an individual receiving a TEC inhibitor, such as an ITK inhibitor or a BTK inhibitor (e.g. ibrutinib) for treatment of a hematological malignancy such as diffuse large B cell lymphoma (DLBCL) has developed or is likely to develop resistance to the therapy, by determining the presence or absence of a modification in one or more biomarker genes selected from EP300, MLL2, BCL-2, RB1, LRP1B, PIM1, TSC2, TNFRSF11A, SMAD4, PAX5, and CARD11, and one or more additional biomarkers, and characterize the individual as resistant or likely to become resistant if the individual has a modification in one or more biomarker genes selected from EP300, MLL2, BCL-2, RB1, LRP1B, PIM1, TSC2, TNFRSF11A, SMAD4, PAX5, and CARD11, and the one or more additional biomarkers. Also disclosed herein, are methods of optimizing a therapeutic regiment based the presence or absence of a modification in one or more biomarker genes selected from EP300, MLL2, BCL-2, RB1, LRP1B, PIM1, TSC2, TNFRSF11A, SMAD4, PAX5, and CARD11, and one or more additional biomarkers.

In some embodiment, the one or more additional biomarkers include a mutation or modification in BTK. In some embodiments, the modification is a mutation at amino acid position 481 in BTK. In some embodiments, the mutation is C481S in BTK. In some embodiments, the C481 mutation in BTK is accompanied with additional mutations in BTK. In some embodiments, the additional mutations in BTK include substitutions at amino acid positions L11, K12, S14, K19, F25, K27, R28, R33, Y39, Y40, E41, I61, V64, R82, Q103, V113, S115, T117, Q127, C154, C155, T184, P189, P190, Y223, W251, 8288, L295, G302, R307, D308, V319, Y334, L358, Y361, H362, H364, N365, S366, L369, 1370M, R372, L408, G414, Y418, I429, K430, E445, G462, Y476, M477, C502, C506, A508, M509, L512, L518, R520, D521, A523, R525, N526, V535, L542, R544, Y551, F559, R562, W563, E567, S578, W581, A582, F583, M587, E589, S592, G594, Y598, A607, G613, Y617, P619, A622, V626, M630, C633, R641, F644, L647, L652, V1065, and A1185. In some embodiments, the additional modifications is selected from among L11P, K12R, S14F, K19E, F25S, K27R, R28H, R28C, R28P, T33P, Y3S9, Y40C, Y40N, E41K, I61N, V64F, V64D, R82K, Q103QSFSSVR, V113D, S115F, T117P, Q127H, C154S, C155G, T184P, P189A, Y223F, W251L, R288W, R288Q, L295P, G302E, R307K, R307G, R307T, D308E, V319A, Y334S, L358F, Y361C, H362Q, H364P, N365Y, S366F, L369F, I370M, R372G, L408P, G414R, Y418H, I429N, K430E, E445D, G462D, G462V, Y476D, M477R, C502F, C502W, C506Y, C506R, A508D, M509I, M509V, L512P, L512Q, L518R, R520Q, D521G, D521H, D521N, A523E, R525G, R525P, R525Q, N526K, V535F, L542P, R544G, R544K, Y551F, F559S, R562W, R562P, W563L, E567K, S578Y, W581R, A582V, F583S, M587L, E589D, E589K, E589G, S592P, G594E, Y598C, A607D, G613D, Y617E, P619A, P619S, A622P, V626G, M630I, M630K, M630T, C633Y, R641C, F644L, F644S, L647P, L652P, V1065I, and A1185V.

In some embodiments, the one or more additional biomarkers include a mutation in PLCγ2. In some embodiments, the mutation in PLCγ2 is a mutation at amino acid residue 665, 707, or a combination thereof. In some embodiments, the mutation is R665W and S707F.

In some embodiments, the one or more additional biomarkers include cytogenetic abnormalities such as del(17p13.1), del(13q14.3), del(11q22.3), del(11q23), unmutated IgVH together with ZAP-70+ and/or CD38+, trisomy 12, t(11;14)(q13;q32), t(14;19)(q32;q13), t(2;14)(p13;q32), del(13q14), +(12q21), del(6q21), ATM del, p53 del, t(15;17); t(8;21)(q22;q22), t(6;9), inv(16)(p13q22), del(16q); inv(16), t(16;16), del(11q), t(9;11), t(11;19), t(1;22), del(5q), +8, +21, +22, del(7q), del(9q), abnormal 11q23, -5, -7, abnormal 3q, complex karyotype, t(14;19), t(3:14), t(11;14), t(2;8)(p11;q24), t(1;8)(p36;q24), t(8:9)(q24;p13), t(9;14)(p13;q32), t(3:14)(q27;q32), or a combination thereof.

In some embodiments, also disclosed herein are methods of selecting a patient having a hematological malignancy such as DLBCL for treatment with a TEC inhibitor, such as an ITK inhibitor or a BTK inhibitor (e.g. ibrutinib) by determining the presence or absence of a modification in one or more biomarker genes selected from BCL-2, RB1, LRP1B, PIM1, TSC2, TNFRSF11A, SMAD4, PAX5, and CARD11, and a mutation in BTK at amino acid residue position 481. In some embodiments, the mutation is C481S. In some embodiments, disclosed herein are methods of monitoring whether an individual receiving a TEC inhibitor, such as an ITK inhibitor or a BTK inhibitor (e.g. ibrutinib) for treatment of a hematological malignancy such as diffuse large B cell lymphoma (DLBCL) has developed or is likely to develop resistance to the therapy, by determining the presence or absence of a modification in one or more biomarker genes selected from BCL-2, RB1, LRP1B, PIM1, TSC2, TNFRSF11A, SMAD4, PAX5, and CARD11, and a mutation in BTK at amino acid residue position 481, and characterize the individual as resistant or likely to become resistant if the individual has a modification in one or more biomarker genes selected from BCL-2, RB1, LRP1B, PIM1, TSC2, TNFRSF11A, SMAD4, PAX5, and CARD11, and the mutation in BTK at amino acid residue position 481. In some embodiments, the mutation is C481S. Also disclosed herein, are methods of optimizing a therapeutic regiment based the presence or absence of a modification in one or more biomarker genes selected from BCL-2, RB1, LRP1B, PIM1, TSC2, TNFRSF11A, SMAD4, PAX5, and CARD11, and a mutation in BTK at amino acid residue position 481. In some embodiments, the mutation is C481S.

In some embodiments, also disclosed herein are methods of selecting a patient having a hematological malignancy such as DLBCL for treatment with a TEC inhibitor, such as an ITK inhibitor or a BTK inhibitor (e.g. ibrutinib) by determining the presence or absence of a modification in one or more biomarker genes selected from BCL-2, RB1, LRP1B, PIM1, TSC2, TNFRSF11A, SMAD4, PAX5, and CARD11, and a mutation in PLCγ2 at amino acid residue position 665 and/or 707. In some embodiments, the mutations are R665W and S707F. In some embodiments, disclosed herein are methods of monitoring whether an individual receiving a TEC inhibitor, such as an ITK inhibitor or a BTK inhibitor (e.g. ibrutinib) for treatment of a hematological malignancy such as diffuse large B cell lymphoma (DLBCL) has developed or is likely to develop resistance to the therapy, by determining the presence or absence of a modification in one or more biomarker genes selected from BCL-2, RB1, LRP1B, PIM1, TSC2, TNFRSF11A, SMAD4, PAX5, and CARD11, and a mutation in PLCγ2 at amino acid residue position 665 and/or 707, and characterize the individual as resistant or likely to become resistant if the individual has a modification in one or more biomarker genes selected from BCL-2, RB1, LRP1B, PIM1, TSC2, TNFRSF11A, SMAD4, PAX5, and CARD11, and the mutation in PLCγ2 at amino acid residue position 665 and/or 707. In some embodiments, the mutations are R665W and S707F. Also disclosed herein, are methods of optimizing a therapeutic regiment based the presence or absence of a modification in one or more biomarker genes selected from BCL-2, RB1, LRP1B, PIM1, TSC2, TNFRSF11A, SMAD4, PAX5, and CARD11, and a mutation in PLCγ2 at amino acid residue position 665 and/or 707. In some embodiments, the mutations are R665W and S707F.

In some embodiments, also disclosed herein are methods of selecting a patient a hematological malignancy such as having DLBCL for treatment with a TEC inhibitor, such as an ITK inhibitor or a BTK inhibitor (e.g. ibrutinib) by determining the presence or absence of a modification in one or more biomarker genes selected from BCL-2, RB1, LRP1B, PIM1, TSC2, TNFRSF11A, SMAD4, PAX5, and CARD11, and one or more cytogenetic abnormalities. In some embodiments, disclosed herein are methods of monitoring whether an individual receiving a TEC inhibitor, such as an ITK inhibitor or a BTK inhibitor (e.g. ibrutinib) for treatment of a hematological malignancy such as diffuse large B cell lymphoma (DLBCL) has developed or is likely to develop resistance to the therapy, by determining the presence or absence of a modification in one or more biomarker genes selected from BCL-2, RB1, LRP1B, PIM1, TSC2, TNFRSF11A, SMAD4, PAX5, and CARD11, and one or more cytogenetic abnormalities, and characterize the individual as resistant or likely to become resistant if the individual has a modification in one or more biomarker genes selected from BCL-2, RB1, LRP1B, PIM1, TSC2, TNFRSF11A, SMAD4, PAX5, and CARD11, and the one or more cytogenetic abnormalities. Also disclosed herein, are methods of optimizing a therapeutic regiment based the presence or absence of a modification in one or more biomarker genes selected from BCL-2, RB1, LRP1B, PIM1, TSC2, TNFRSF11A, SMAD4, PAX5, and CARD11, and one or more cytogenetic abnormalities. In some embodiments, the one or more additional biomarkers include cytogenetic abnormalities such as del(17p13.1), del(13q14.3), del(11q22.3), del(11q23), unmutated IgVH together with ZAP-70+ and/or CD38+, trisomy 12, t(11;14)(q13;q32), t(14;19)(q32;q13), t(2;14)(p13;q32), del(13q14), +(12q21), del(6q21), ATM del, p53 del, t(15;17); t(8;21)(q22;q22), t(6;9), inv(16)(p13q22), del(16q); inv(16), t(16;16), del(11q), t(9;11), t(11;19), t(1;22), del(5q), +8, +21, +22, del(7q), del(9q), abnormal 11q23, -5, -7, abnormal 3q, complex karyotype, t(14;19), t(3:14), t(11;14), t(2;8)(p11;q24), t(1;8)(p36;q24), t(8:9)(q24;p13), t(9;14)(p13;q32), t(3:14)(q27;q32), or a combination thereof.

In some embodiments, the modifications in the one or more biomarker genes include base substitution, insertion, deletion, DNA rearrangement, copy number alteration, or a combination thereof. In some embodiments, CDKN2A, CDKN2B, MYD88, PIK3C2G, CD79B, IRS2, BCL2, RB1, LRP1B, PIM1, TSC2, TNFRSF11A, SMAD4, PAX5, and CARD11 comprise one or more modifications in each gene. In some embodiments, BCL-2, RB1, LRP1B, PIM1, TSC2, TNFRSF11A, SMAD4, PAX5, and CARD11 comprise one or more modifications in each gene. In some embodiments, BCL-2, RB1, LRP1B, PIM1, and TSC2 comprise one or more modifications in each gene. In some embodiments, modifications in the biomarker genes also correlate to modifications in the amino acid sequences. In some embodiments, modifications or mutations in the biomarker gene comprise base substitution, insertion, deletion, DNA rearrangement, copy number alteration, or a combination thereof. In some embodiments, these modifications result in missense mutation, nonsense mutation, or splice site mutation.

In some embodiments, additionally disclosed herein are methods of selecting an individual having a non-Hodgkin's lymphoma (e.g. DLBCL, CLL, SLL, FL) for treatment with a TEC inhibitor, such as an ITK inhibitor or a BTK inhibitor (e.g. ibrutinib) by determining the expression level of the biomarker gene BCL-2; and administering to the individual a therapeutically effective amount of ibrutinib if there is no increased expression level in the biomarker gene BCL-2 relative to a control. In some instances, disclosed herein are methods of monitoring the disease progression in an individual having a non-Hodgkin's lymphoma (e.g. DLBCL, CLL, SLL, FL) by determining the expression level of the biomarker gene BCL-2; and characterizing the individual as developed an insensitivity to ibrutinib if the individual shows an increase in expression level in the biomarker gene BCL-2 relative to a control. In some cases, disclosed herein are methods of monitoring the disease progression in an individual having a non-Hodgkin's lymphoma (e.g. DLBCL, CLL, SLL, FL) by determining the mutation rate of the biomarker gene BCL-2; and characterizing the individual as developed an insensitivity to ibrutinib or likely to develop an insensitivity to ibrutinib if the individual shows an increase in the mutation rate in the biomarker gene BCL-2 relative to a control.

In some cases, an increased expression level indicates an increase in insensitivity to a TEC inhibitor (e.g. a BTK inhibitor or an ITK inhibitor). In some cases, an increased expression level indicates an increase in insensitivity to a BTK inhibitor (e.g. ibrutinib). In some cases, an increased expression level indicates an increase in insensitivity to ibrutinib. In some embodiments, an increased expression level of the BCL-2 gene indicates an increase in insensitivity to a TEC inhibitor (e.g. a BTK inhibitor or an ITK inhibitor). In some embodiments, an increased expression level of the BCL-2 gene indicates an increase in insensitivity to a BTK inhibitor (e.g. ibrutinib). In some embodiments, an increased expression level of the BCL-2 gene indicates an increase in insensitivity to ibrutinib.

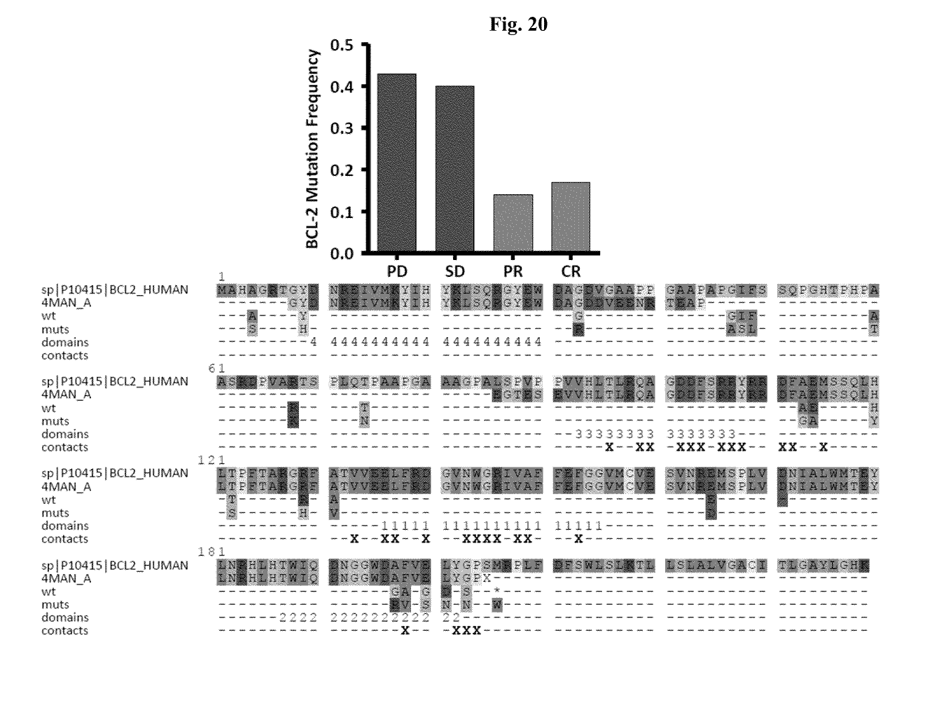

In some instances, an increase in mutation rate of the BCL-2 gene indicates a worse disease progression or a worse response to a treatment, e.g. with a TEC inhibitor (e.g. a BTK inhibitor or an ITK inhibitor) in an individual. In some cases, an increase in mutation rate of the BCL-2 gene indicates a worse disease progression or a worse response to a treatment, e.g. with a BTK inhibitor (e.g. ibrutinib) in an individual. In some instances, an increase in mutation rate of the BCL-2 gene indicates a worse disease progression or a worse response to a treatment, e.g. with ibrutinib in an individual. In some embodiments, the mutation is a mutation as shown in FIG. 20.

In some instances, an increase in mutation rate of the BCL-2 gene indicates the individual has a progression disease (PD) or stable diasease (SD) in response to a treatment, e.g. with a TEC inhibitor (e.g. a BTK inhibitor or an ITK inhibitor). In some instances, an increase in mutation rate of the BCL-2 gene indicates the individual has a progression disease (PD) or stable diasease (SD) in response to a treatment, e.g. with a BTK inhibitor (e.g. ibrutinib). In some instances, an increase in mutation rate of the BCL-2 gene indicates the individual has a progression disease (PD) or stable diasease (SD) in response to a treatment, e.g. ibrutinib. In some embodiments, the mutation is a mutation as shown in FIG. 20.

### BCL-2

B-cell lymphoma 2 (BCL-2) is a proto-oncogene that regulates apoptosis. In normal B cells, BCL-2 is located on chromosome 18 at position 21.3 (Gene ID: 596). However, in cancerous B cells, BCL-2 undergoes a reciprocal translocation with the immunoglobulin (IG) heavy chain (IGH) gene located on chromosome 14 as t(14;18)(q32;q21.3). This t(14;18) translocation subsequently places BCL-2 close to the heavy chain gene enhancer, which induces an increase in expression level of BCL-2 protein. B cells containing overexpression of the BCL-2 protein become apoptosis-resistant and proliferate in the germinal center where B cell development occurs.

In some embodiments, mutations or modifications of the BCL-2 gene comprise base substitution, insertion, deletion, DNA rearrangement, copy number alteration, or a combination thereof. In some embodiments, modifications of the BCL-2 gene comprise DNA rearrangements such as t(14;18)(q32;q21.3), t(2;18)(p11;q21.3), or t(18;22)(q21.3;q11). In some embodiments, mutations or modifications of the BCL-2 gene comprise base substitution, insertion, or deletion such as, but not limited to, modifications from thymine to cytosine at nucleic acid position 60985385, from guanine to cytosine at position 60985526, from guanine to adenine at position 60985730, from thymine to cytosine at position 60985412, from guanine to cytosine at position 60985644, from cytosine to thymine at position 60985803, from adenine to cytosine at position 60985840, from cytosine to guanine at position 60985900, from thymine to adenine at position 60985734, from cytosine to guanine at position 60985800, from cytosine to thymine at position 60985803, from thymine to guanine at position 60985854, or a combination thereof, on chromosome 18. In some embodiments, the base substitution, insertion, or deletions result in missense mutation, nonsense mutation, or splice site mutation. In some embodiments, the modifications on chromosome 18 are observed in an individual having DLBCL. In some embodiments, the modifications on chromosome 18 are observed in an individual having FL.

In some embodiments, the modifications associated with the BCL-2 gene further comprise modifications in the BCL-2 protein. In some embodiments, the modifications in the BCL-2 protein include, but are not limited to, modifications at positions corresponding to amino acid residues 2, 3, 4, 9, 11, 16, 20, 25, 33, 34, 45, 47, 48, 49, 56, 57, 59, 60, 68, 74, 86, 90, 108, 113, 114, 118, 119, 120, 122, 125, 129, 131, 157, 163, 165, 172, 180, 197, 198, 200, 201, 203, and/or 206. In some embodiments, the modifications include A2P, H3P, A4S, Y9H, N11Y, M16L, H20Q, Q25L, G33R, D34H, A45T, G47A, I48S, F49L, T56S, P57L, P59A, A60T, R68K, T74N, T74S, L86V, P90S, Y108H, Y108C, A113G, E114A, Q118H, L119V, H120Y, T122S, T125S, R129H, A131V, M157L, N163S, E165D, N172S, Y180F, Y180D, G197R, G197S, A198V, G200S, D201N, S203N, and/or 206W. In some embodiments, the BCL-2 protein comprise modifications at positions corresponding to amino acid residues 4, 9, 33, 47, 48, 49, 60, 68, 74, 113, 114, 120, 122, 129, 131, 165, 197, 198, 200, 201, 203, and/or 206. In some embodiments, the modifications include A4S, Y9H, G33R, G47A, I48S, F49L, A60T, R68K, T74N, T74S, A113G, E114A, H120Y, T122S, R129H, A131V, E165D, G197R, G197S, A198V, G200S, D201N, S203N, and/or 206W.

In some embodiments, the BCL-2 protein comprises modifications at one or more amino acid positions as shown in the sequence alignment in FIG. 20.

In some embodiments, the modifications of these amino acid residues are observed in an individual having DLBCL. In some embodiments, the modifications of these amino acid residues are observed in an individual having FL.

As used herein and throughout, the term “proto-oncogene” refers to a cellular gene that when mutated or abnormally expressed, induces the cell to become cancerous.

### RB1

RB1, or retinoblastoma protein, is a tumor suppressor protein that inhibits transcription of genes necessary for the transition from G1 to S phase. For example, RB1 stalls cells containing damaged DNA in G1 phase by binding to the E2 promoter-binding-protein-dimerization partners (E2F-DP) complex which is central for the G1 to S phase transition, thereby inactivating the E2F-DP complex. In addition, the Rb-E2F/DP complex also attracts HDAC proteins to the chromatin, thereby further suppresses DNA synthesis.

The RB1 gene is located on chromosome 13, at position 14.2 (Gene ID: 5925). Mutations or modifications in RB1 are heterogeneous in nature. In some embodiments, there are more than 1600 distinct mutations comprising from base substitutions, insertions, deletions, copy number alterations, or DNA rearrangements. In some embodiments, the mutations or modifications include a deletion at 13q14. In some embodiments, the mutations or modifications of RB1 include modification from thymine to cytosine at nucleic acid position 48934213 on chromosome 13. In some embodiments, the base substitution results in missense mutation. In some embodiments, the modification at nucleic acid position 48934213 on chromosome 13 is observed in an individual having DLBCL.

In some embodiments, the modifications associated with the RB1 gene further comprise modifications in the RB1 protein. In some embodiments, the modifications in the RB1 protein include modification at positions corresponding to amino acid residue 223. In some embodiments, the modification is L223P. In some embodiments, the modification at amino acid residue 223 is observed in an individual having DLBCL.

### LRP1B

Low density lipoprotein-related protein 1B (LRP1B) belong to the family of low density lipoprotein receptors. Similar to other members of the lipoprotein receptor family, LRP1B can associate with other membrane bound receptors, such as integrins and receptor tyrosine kinases and intracellular signaling molecules. In addition, LRP1B modulates cell migration and invasive capacity through the regulation of the urokinase plasminogen system. Further, LRP1B modulates the extracellular microenvironment through clearance of extracellular ligands by endocytosis. Inactivation of LRP1B results in alterations in the cellular environment which in some cases, confer increased cell growth and invasive capacity.

The LRP1B gene is located on chromosome 2 at position 21.2 (Gene ID: 53353). In some embodiments, modifications of the LRP1B gene comprise base substitution, insertion, deletion, DNA rearrangement, copy number alteration, or a combination thereof. In some embodiments, modifications of LRP1B include, but are not limited to, modification from adenine to cytosine at nucleic acid position 141122343, from cytosine to adenine at nucleic acid position 141819760, from adenine to guanine at nucleic acid position 142888255, from cytosine to guanine at nucleic acid position 141299498, from thymine to guanine at nucleic acid position 142004875, from cytosine to thymine at nucleic acid position 141122349, from adenine to guanine at nucleic acid position 141128768, from guanine to thymine at nucleic acid position 141202004, from adenine to guanine at nucleic acid position 141202135, from cytosine to thymine at nucleic acid position 141232883, from adenine to guanine at nucleic acid position 141242979, from guanine to adenine at nucleic acid position 141643757, from thymine to guanine at nucleic acid position 141771142, from guanine to adenine at nucleic acid position 141986994, or a combination thereof, on chromosome 2. In some embodiments, the base substitution, insertion, or deletions result in missense mutation, nonsense mutation, or splice site mutation. In some embodiments, the modifications on chromosome 2 are observed in an individual having DLBCL.

In some embodiments, the modifications associated with the LRP1B gene further comprise modifications in the LRP1B protein. In some embodiments, the modifications in the LRP1B protein include modification at positions corresponding to amino acid residue 15, 171, 203, 366, 778, 846, 1305, 1452, 2205, 2413, 2567, 3120, 3150, 3352, 3391, 3397, 3619, 3671, 3673, and/or 4436. In some embodiments, the modifications include L15S, N171T, P203L, D366Y, D778A, K846E, T13051, A1452P, C2205F, V2413L, C2567S, Y3120H, C3150Y, V3150I, S3352P, C3391R, L3397M, C3619R, G3671E, I3673R, and/or Y4436F. In some embodiments, the modifications at these amino acid residues are observed in an individual having DLBCL.

### PIM1

PIM1 is a proto-oncogene that encodes for serine or threonine kinases. In some cases, it has been described in relation to murine T-cell lymphomas, but has since been found to be highly expressed in other tumor cells. PIM1 is involved in cell cycle progression, apoptosis, transcriptional activations, and signal transduction pathways. In DLBCL, PIM1 has been shown to be a target of aberrant hypermutation, leading to base pair substitutions and amino acid substitutions. The PIM1 gene is located on chromosome 6 at location 21.2 (Gene ID: 5292).

In some embodiments, modifications of the PIM1 gene comprise base substitution, insertion, deletion, DNA rearrangement, copy number alteration, or a combination thereof. In some embodiments, modifications of PIM1 include, but are not limited to, modification from cytosine to thymine at nucleic acid position 37138962, from guanine to thymine at nucleic acid position 37138549, from thymine to adenine at nucleic acid position 37138906, from cytosine to guanine at nucleic acid position 37139045, from cytosine to thymine at nucleic acid position 37139210, from thymine to cytosine at nucleic acid position 37138359, from cytosine to thymine at nucleic acid position 37138355, from thymine to guanine at nucleic acid position 37138400, from cytosine to adenine at nucleic acid position 37139033, from cytosine to thymine at nucleic acid position 37139204, from cytosine to thymine at nucleic acid position 37139210, from guanine to adenine at nucleic acid position 37138549, or a combination thereof, on chromosome 6. In some embodiments, the base substitution, insertion, or deletions result in missense mutation, nonsense mutation, or splice site mutation. In some embodiments, the modifications on chromosome 6 are observed in an individual having DLBCL.

In some embodiments, the modifications associated with the PIM1 gene further comprise modifications in the PIM1 protein. In some embodiments, the modifications in the PIM1 protein include modification at positions corresponding to amino acid residue 2, 3, 17, 24, 28, 81, 82, 101, 109, 125, 129, 164, 172, 182 and/or 184. In some embodiments, the modifications include K2F, K3S, C17G, K24N, G28-splice, P81-splice, N82K, S101F, W109-nonsense, P125S, P125T, L129V, L164F, N172S, L182F, and/or L184F. In some embodiments, the modifications at these amino acid residues are observed in an individual having DLBCL.

### TSC2

Tuberous sclerosis complex 2 (TSC2) is a tumor suppressor protein that together with hamartin encoded by the TSC1 gene, modulates cellular growth, proliferation, and protein synthesis. The TSC2 gene is located on chromosome 6 at location 13.3 (Gene ID: 7249).

In some embodiments, modifications of the TSC2 gene comprise base substitution, insertion, deletion, DNA rearrangement, copy number alteration, or a combination thereof. In some embodiments, modifications of TSC2 include, but are not limited to, modification from guanine to adenine at nucleic acid position 2127694, from cytosine to thymine at nucleic acid position 2122880, from guanine to adenine at nucleic acid position 2121583, from cytosine to thymine at nucleic acid position 2110779, or a combination thereof, on chromosome 6. In some embodiments, the base substitution, insertion, or deletions result in missense mutation, nonsense mutation, or splice site mutation. In some embodiments, the modifications on chromosome 6 are observed in an individual having DLBCL.

In some embodiments, the modifications associated with the TSC2 gene further comprise modifications in the TSC2 protein. In some embodiments, the modifications in the TSC2 protein include modification at positions corresponding to amino acid residue 638, 751, and/or 978. In some embodiments, the modifications include V638M, R751-nonsense, and/or R978H. In some embodiments, the modifications at these amino acid residues are observed in an individual having DLBCL.

### Co-Mutation in CD79B and MYD88

Disclosed herein, in certain embodiments, are methods of selecting an individual having a hematological malignancy for treatment with a TEC inhibitor, monitoring an individual during therapy, or optimizing a therapeutic regimen based on the presence or absence of a co-mutation in CD79B and MYD88. In some embodiments, the presence of the combination of the modifications in CD79B and MYD88 indicates the individual is responsive or is likely to be responsive to treatment with the TEC inhibitor. In some embodiments, the modifications comprise a modification to an aromatic residue at amino acid position 196 in CD79B (Gene ID: 974; BC002975.1) and at least one modification at amino acid positions 198 or 265 in MYD88 (Gene ID: 4615; U84408.1). In some embodiments, the aromatic residue is phenylalanine or tryptophan. In some embodiments, the modification at amino acid position 196 in CD79B is Y196F. In some embodiments, the modification at amino acid position 198 in MYD88 is S198N. In some embodiments, the modification at amino acid position 265 in MYD88 is L265P. In some embodiments, the combination of the modifications in CD79B and MYD88 is Y196F and S198N or Y196F and L265P.

In some embodiments, additional co-mutations in CD79B and MYD88 are observed. In some embodiments, additional modifications in CD79B occur at positions corresponding to amino acid residue 149, 196 and/or 192. In some embodiments, the additional modifications include A149P, Y196S, E192D, and/or Y196C. In some embodiments, MYD88 comprise additional modifications at positions corresponding to amino acid residue 232, 169, 172 and/or 220. In some embodiments, the modifications are M232T, G169R, V172F, and L220P. In some embodiments, additional co-mutations in CD79B and MYD88 comprise Y196C (CD79B) and L265P (MYD88), and E192D (CD79B), Y196C (CD79B) and L265P (MYD88).

In some embodiments, the presence of additional co-mutations disclosed above in an individual also indicates the individual as responsive or is likely to be responsive to treatment with a TEC inhibitor. In some embodiments, the presence of additional co-mutations is less likely to indicate the individual as responsive or is likely to be responsive to treatment with a TEC inhibitor. In some embodiments, the presence of additional co-mutations does not indicate the individual as responsive or is likely to be responsive to treatment with a TEC inhibitor.

In some embodiments, the TEC inhibitor is a BTK inhibitor, an ITK inhibitor, a TEC inhibitor, a RLK inhibitor, or a BMX inhibitor. In some embodiments, the TEC inhibitor is an ITK inhibitor. In some embodiments, the TEC inhibitor is a BTK inhibitor. In some embodiments, the BTK inhibitor is ibrutinib.

In some embodiments, the hematological malignancy is a leukemia, a lymphoma, a myeloma, a non-Hodgkin's lymphoma, a Hodgkin's lymphoma, T-cell malignancy, or a B-cell malignancy. In some embodiments, the hematological malignancy is a B-cell malignancy. In some embodiments, the B-cell malignancy is chronic lymphocytic leukemia (CLL), high-risk chronic lymphocytic leukemia (CLL), small lymphocytic lymphoma (SLL), high-risk small lymphocytic lymphoma (SLL), diffuse large B cell lymphoma (DLBCL), mantle cell lymphoma (MCL), or Waldenstrom's macroglobulinemia. In some embodiments, the B-cell malignancy is DLBCL. In some embodiments, the DLBCL is activated B-cell DLBCL (ABC-DLBCL), germinal center B-cell like DLBCL (GBC-DLBCL), double-hit (DH) DLBCL, triple-hit (TH) DLBCL, or unclassified DLBCL. In some embodiments, the DLBCL is activated B-cell DLBCL (ABC-DLBCL), or unclassified DLBCL.

In some embodiments, the presence of a co-mutation in CD79B and MYD88 as Y196F and S198N or Y196F and L265P characterize the individual having DLBCL as responsive or is likely to be responsive to treatment with a TEC inhibitor. In some embodiments, the presence of a co-mutation in CD79B and MYD88 as Y196F and S198N or Y196F and L265P characterize the individual having DLBCL as responsive or is likely to be responsive to treatment with an ITK inhibitor. In some embodiments, the presence of a co-mutation in CD79B and MYD88 as Y196F and S198N or Y196F and L265P characterize the individual having DLBCL as responsive or is likely to be responsive to treatment with a BTK inhibitor. In some embodiments, the BTK inhibitor is selected from among ibrutinib (PCI-32765), PCI-45292, PCI-45466, AVL-101/CC-101 (Avila Therapeutics/Celgene Corporation), AVL-263/CC-263 (Avila Therapeutics/Celgene Corporation), AVL-292/CC-292 (Avila Therapeutics/Celgene Corporation), AVL-291/CC-291 (Avila Therapeutics/Celgene Corporation), CNX 774 (Avila Therapeutics), BMS-488516 (Bristol-Myers Squibb), BMS-509744 (Bristol-Myers Squibb), CGI-1746 (CGI Pharma/Gilead Sciences), CGI-560 (CGI Pharma/Gilead Sciences), CTA-056, GDC-0834 (Genentech), HY-11066 (also, CTK4I7891, HMS3265G21, HMS3265G22, HMS3265H21, HMS3265H22, 439574-61-5, AG-F-54930), ONO-4059 (Ono Pharmaceutical Co., Ltd.), ONO-WG37 (Ono Pharmaceutical Co., Ltd.), PLS-123 (Peking University), RN486 (Hoffmann-La Roche), HM71224 (Hanmi Pharmaceutical Company Limited), LFM-A13, BGB-3111 (Beigene), KBP-7536 (KBP BioSciences), ACP-196 (Acerta Pharma), JTE-051 (Japan Tobacco Inc), PRN1008 (Principia), CTP-730 (Concert Pharmaceuticals), or GDC-0853 (Genentech). In some embodiments, the presence of a co-mutation in CD79B and MYD88 as Y196F and S198N or Y196F and L265P characterize the individual having DLBCL as responsive or is likely to be responsive to treatment with ibrutinib.

In some embodiments, also disclosed herein are methods of selecting an individual having a hematological malignancy such as diffuse large B cell lymphoma (DLBCL) for treatment with a TEC inhibitor, such as an ITK inhibitor or a BTK inhibitor (e.g. ibrutinib), based on the presence or absence of a modification to an aromatic residue at amino acid position 196 in CD79B, at least one modification at amino acid positions 198 or 265 in MYD88, and one or more additional biomarkers. In some embodiments, also disclosed herein are methods of monitoring whether an individual receiving a TEC inhibitor, such as an ITK inhibitor or a BTK inhibitor (e.g. ibrutinib) for treatment of a hematological malignancy such as diffuse large B cell lymphoma (DLBCL) is responsive or is likely to respond to therapy, based on the presence or absence of a modification to an aromatic residue at amino acid position 196 in CD79B, at least one modification at amino acid positions 198 or 265 in MYD88, and one or more additional biomarkers, and characterize the individual as responsive or is likely to respond to therapy with ibrutinib if the individual has the modification to an aromatic residue at amino acid position 196 in CD79B, at least one modification at amino acid positions 198 or 265 in MYD88, and one or more additional biomarkers. In some embodiments, further disclosed herein are methods of optimizing the therapy based on the presence or absence of a modification to an aromatic residue at amino acid position 196 in CD79B, at least one modification at amino acid positions 198 or 265 in MYD88, and one or more additional biomarkers.

In some embodiment, the one or more additional biomarkers include a mutation or modification in BTK. In some embodiments, the modification is a mutation at amino acid position 481 in BTK. In some embodiments, the mutation is C481S in BTK. In some embodiments, the C481 mutation in BTK is accompanied with additional mutations in BTK. In some embodiments, the additional mutations in BTK include substitutions at amino acid positions L11, K12, S14, K19, F25, K27, R28, R33, Y39, Y40, E41, I61, V64, R82, Q103, V113, S115, T117, Q127, C154, C155, T184, P189, P190, Y223, W251, 8288, L295, G302, R307, D308, V319, Y334, L358, Y361, H362, H364, N365, S366, L369, 1370M, R372, L408, G414, Y418, 1429, K430, E445, G462, Y476, M477, C502, C506, A508, M509, L512, L518, R520, D521, A523, R525, N526, V535, L542, R544, Y551, F559, R562, W563, E567, S578, W581, A582, F583, M587, E589, S592, G594, Y598, A607, G613, Y617, P619, A622, V626, M630, C633, R641, F644, L647, L652, V1065, and A1185. In some embodiments, the additional modifications is selected from among L11P, K12R, S14F, K19E, F25S, K27R, R28H, R28C, R28P, T33P, Y3S9, Y40C, Y40N, E41K, I61N, V64F, V64D, R82K, Q103QSFSSVR, V113D, S115F, T117P, Q127H, C154S, C155G, T184P, P189A, Y223F, W251L, R288W, R288Q, L295P, G302E, R307K, R307G, R307T, D308E, V319A, Y334S, L358F, Y361C, H362Q, H364P, N365Y, S366F, L369F, I370M, R372G, L408P, G414R, Y418H, I429N, K430E, E445D, G462D, G462V, Y476D, M477R, C502F, C502W, C506Y, C506R, A508D, M509I, M509V, L512P, L512Q, L518R, R520Q, D521G, D521H, D521N, A523E, R525G, R525P, R525Q, N526K, V535F, L542P, R544G, R544K, Y551F, F559S, R562W, R562P, W563L, E567K, S578Y, W581R, A582V, F583S, M587L, E589D, E589K, E589G, S592P, G594E, Y598C, A607D, G613D, Y617E, P619A, P619S, A622P, V626G, M630I, M630K, M630T, C633Y, R641C, F644L, F644S, L647P, L652P, V1065I, and A1185V.

In some embodiments, the one or more additional biomarkers include a mutation in PLCγ2. In some embodiments, the mutation in PLCγ2 is a mutation at amino acid residue 665, 707, or a combination thereof. In some embodiments, the mutation is R665W and S707F.

In some embodiments, the one or more additional biomarkers include cytogenetic abnormalities such as del(17p13.1), del(13q14.3), del(11q22.3), del(11q23), unmutated IgVH together with ZAP-70+ and/or CD38+, trisomy 12, t(11;14)(q13;q32), t(14;19)(q32;q13), t(2;14)(p13;q32), del(13q14), +(12q21), del(6q21), ATM del, p53 del, t(15;17); t(8;21)(q22;q22), t(6;9), inv(16)(p13q22), del(16q); inv(16), t(16;16), del(11q), t(9;11), t(11;19), t(1;22), del(5q), +8, +21, +22, del(7q), del(9q), abnormal 11q23, -5, -7, abnormal 3q, complex karyotype, t(14;19), t(3:14), t(11;14), t(2;8)(p11;q24), t(1;8)(p36;q24), t(8:9)(q24;p13), t(9;14)(p13;q32), t(3:14)(q27;q32), or a combination thereof.

In some embodiments, also disclosed herein are methods of selecting an individual having a hematological malignancy such as diffuse large B cell lymphoma (DLBCL) for treatment with a TEC inhibitor, such as an ITK inhibitor or a BTK inhibitor (e.g. ibrutinib), based on the presence or absence of a modification to an aromatic residue at amino acid position 196 in CD79B, at least one modification at amino acid positions 198 or 265 in MYD88, and a mutation in BTK at amino acid residue position 481. In some embodiments, the mutation is C481S. In some embodiments, also disclosed herein are methods of monitoring whether an individual receiving a TEC inhibitor, such as an ITK inhibitor or a BTK inhibitor (e.g. ibrutinib) for treatment of a hematological malignancy such as diffuse large B cell lymphoma (DLBCL) is responsive or is likely to respond to therapy, based on the presence or absence of a modification to an aromatic residue at amino acid position 196 in CD79B, at least one modification at amino acid positions 198 or 265 in MYD88, and a mutation in BTK at amino acid residue position 481, and characterize the individual as responsive or is likely to respond to therapy with ibrutinib if the individual has the modification to an aromatic residue at amino acid position 196 in CD79B, at least one modification at amino acid positions 198 or 265 in MYD88, and the mutation in BTK at amino acid residue position 481. In some embodiments, the mutation is C481S. In some embodiments, further disclosed herein are methods of optimizing the therapy based on the presence or absence of a modification to an aromatic residue at amino acid position 196 in CD79B, at least one modification at amino acid positions 198 or 265 in MYD88, and a mutation in BTK at amino acid residue position 481. In some embodiments, the mutation is C481S.

In some embodiments, also disclosed herein are methods of selecting an individual having a hematological malignancy such as diffuse large B cell lymphoma (DLBCL) for treatment with a TEC inhibitor, such as an ITK inhibitor or a BTK inhibitor (e.g. ibrutinib), based on the presence or absence of a modification to an aromatic residue at amino acid position 196 in CD79B, at least one modification at amino acid positions 198 or 265 in MYD88, and a mutation in PLCγ2 at amino acid residue position 665 and/or 707. In some embodiments, the mutations are R665W and S707F. In some embodiments, also disclosed herein are methods of monitoring whether an individual receiving a TEC inhibitor, such as an ITK inhibitor or a BTK inhibitor (e.g. ibrutinib) for treatment of a hematological malignancy such as diffuse large B cell lymphoma (DLBCL) is responsive or is likely to respond to therapy, based on the presence or absence of a modification to an aromatic residue at amino acid position 196 in CD79B, at least one modification at amino acid positions 198 or 265 in MYD88, and a mutation in PLCγ2 at amino acid residue position 665 and/or 707, and characterize the individual as responsive or is likely to respond to therapy with ibrutinib if the individual has the modification to an aromatic residue at amino acid position 196 in CD79B, at least one modification at amino acid positions 198 or 265 in MYD88, and the mutation in PLCγ2 at amino acid residue position 665 and/or 707. In some embodiments, the mutations are R665W and S707F. In some embodiments, further disclosed herein are methods of optimizing the therapy based on the presence or absence of a modification to an aromatic residue at amino acid position 196 in CD79B, at least one modification at amino acid positions 198 or 265 in MYD88, and a mutation in PLCγ2 at amino acid residue position 665 and/or 707. In some embodiments, the mutations are R665W and S707F.

In some embodiments, also disclosed herein are methods of selecting an individual having a hematological malignancy such as diffuse large B cell lymphoma (DLBCL) for treatment with a TEC inhibitor, such as an ITK inhibitor or a BTK inhibitor (e.g. ibrutinib), based on the presence or absence of a modification to an aromatic residue at amino acid position 196 in CD79B, at least one modification at amino acid positions 198 or 265 in MYD88, and one or more cytogenetic abnormalities. In some embodiments, also disclosed herein are methods of monitoring whether an individual receiving a TEC inhibitor, such as an ITK inhibitor or a BTK inhibitor (e.g. ibrutinib) for treatment of a hematological malignancy such as diffuse large B cell lymphoma (DLBCL) is responsive or is likely to respond to therapy, based on the presence or absence of a modification to an aromatic residue at amino acid position 196 in CD79B, at least one modification at amino acid positions 198 or 265 in MYD88, and one or more cytogenetic abnormalities, and characterize the individual as responsive or is likely to respond to therapy with ibrutinib if the individual has the modification to an aromatic residue at amino acid position 196 in CD79B, at least one modification at amino acid positions 198 or 265 in MYD88, and one or more cytogenetic abnormalities. In some embodiments, further disclosed herein are methods of optimizing the therapy based on the presence or absence of a modification to an aromatic residue at amino acid position 196 in CD79B, at least one modification at amino acid positions 198 or 265 in MYD88, and one or more cytogenetic abnormalities. In some embodiments, the one or more additional biomarkers include cytogenetic abnormalities such as del(17p13.1), del(13q14.3), del(11q22.3), del(11q23), unmutated IgVH together with ZAP-70+ and/or CD38+, trisomy 12, t(11;14)(q13;q32), t(14;19)(q32;q13), t(2;14)(p13;q32), del(13q14), +(12q21), del(6q21), ATM del, p53 del, t(15;17); t(8;21)(q22;q22), t(6;9), inv(16)(p13q22), del(16q); inv(16), t(16;16), del(11q), t(9;11), t(11;19), t(1;22), del(5q), +8, +21, +22, del(7q), del(9q), abnormal 11q23, -5, -7, abnormal 3q, complex karyotype, t(14;19), t(3:14), t(11;14), t(2;8)(p11;q24), t(1;8)(p36;q24), t(8:9)(q24;p13), t(9;14)(p13;q32), t(3:14)(q27;q32), or a combination thereof.

### ROS1

Disclosed herein, in certain embodiments, are methods of selecting an individual having a hematological malignancy for treatment with a TEC inhibitor, monitoring an individual during therapy, or optimizing a therapeutic regimen based on the presence or absence of a modification at amino acid position 15 in ROS1. ROS1 is a proto-oncogene tyrosine-protein kinase belonging to the sevenless subfamily of tyrosine kinase insulin receptors. The ROS1 gene is located on chromosome 6 (Gene ID: 6098; 1611455A). The makeup of the ROS1 protein consists of a glycoprotein-rich extracellular domain, a transmembrane domain, and an intracellular tyrosine kinase. ROS1 rearrangements involve a diverse repertoire of partners, increasing fusion partners such as FIG, SLC34A2, CD74, SDC4, EZR, KDELR2, CCDC6, TPM3 and LRIG3. Despite the diversity of fusion partners, ROS1 rearrangements generally involve a conserved ROS1 breakpoint that preserves the tyrosine kinase domain. The preservation of the tyrosine kinase domain may lead to constitutive kinase activation, which is proposed to drive oncogenic transformation. Further, ROS1 fusion leads to upregulation of SHP-1 and SHP2, and activation of the phosphoinositide-3 kinase (PI3K)/AKT/mTOR, JAK/STAT, and MAPK/ERK pathways, in which these downstream signals promote cell survival and proliferation.

In some embodiments, modifications of the ROS1 gene comprise base substitution, insertion, deletion, DNA rearrangement, copy number alteration, or a combination thereof. In some embodiments, modifications of ROS1 include, but are not limited to, modification from guanine to adenine at nucleic acid position 117710558, from cytosine to thymine at nucleic acid position 117641128, from adenine to guanine at nucleic acid position 117708161, from adenine to guanine at nucleic acid position 117746695, or a combination thereof, on chromosome 6.

In some embodiments, the modifications associated with the ROS1 gene further comprise modifications in the ROS1 protein. In some embodiments, the modifications in the ROS1 protein include modification at positions corresponding to amino acid residue 15, 572, 672 and/or 1948. In some embodiments, the modifications include A15G, Q572-nonsense, A672-splice, and/or R1948H. In some embodiments, the modification is A15G.

In some embodiments, an individual having a hematological malignancy is characterized as resistant or is likely to become resistant to therapy with a TEC inhibitor if the individual has the modification at amino acid position 15 in ROS1. In some embodiments, the A15G modification in ROS1 further indicates the individual has developed or likely to develop a progressive hematological malignancy. In some embodiments, the hematological malignancy is a leukemia, a lymphoma, a myeloma, a non-Hodgkin's lymphoma, a Hodgkin's lymphoma, T-cell malignancy, or a B-cell malignancy. In some embodiments, the hematological malignancy is a B-cell malignancy. In some embodiments, the B-cell malignancy is chronic lymphocytic leukemia (CLL), high-risk chronic lymphocytic leukemia (CLL), small lymphocytic lymphoma (SLL), high-risk small lymphocytic lymphoma (SLL), diffuse large B cell lymphoma (DLBCL), mantle cell lymphoma (MCL), or Waldenstrom's macroglobulinemia. In some embodiments, the B-cell malignancy is DLBCL. In some embodiments, the DLBCL is activated B-cell DLBCL (ABC-DLBCL), germinal center B-cell like DLBCL (GBC-DLBCL), double-hit (DH) DLBCL, triple-hit (TH) DLBCL, or unclassified DLBCL. In some embodiments, the DLBCL is activated B-cell DLBCL (ABC-DLBCL), or unclassified DLBCL. In some embodiments, the DLBCL is a progressive DLBCL. In some embodiments, an individual having DLBCL is characterized as resistant or is likely to become resistant to therapy with a TEC inhibitor if the individual has the modification at amino acid position 15 in ROS1. In some embodiments, the A15G modification in ROS1 further indicates the individual has developed or likely to develop a progressive DLBCL.

In some embodiments, the TEC inhibitor is a BTK inhibitor, an ITK inhibitor, a TEC inhibitor, a RLK inhibitor, or a BMX inhibitor. In some embodiments, the TEC inhibitor is an ITK inhibitor. In some embodiments, the TEC inhibitor is a BTK inhibitor. In some embodiments, the BTK inhibitor is ibrutinib.

In some embodiments, an individual having DLBCL is characterized as resistant or is likely to become resistant to therapy with an ITK inhibitor if the individual has the modification at amino acid position 15 in ROS1. In some embodiments, the A15G modification in ROS1 further indicates the individual has developed or likely to develop a progressive DLBCL.

In some embodiments, an individual having DLBCL is characterized as resistant or is likely to become resistant to therapy with a BTK inhibitor if the individual has the modification at amino acid position 15 in ROS1. In some embodiments, the A15G modification in ROS1 further indicates the individual has developed or likely to develop a progressive DLBCL. In some embodiments, the BTK inhibitor is selected from among ibrutinib (PCI-32765), PCI-45292, PCI-45466, AVL-101/CC-101 (Avila Therapeutics/Celgene Corporation), AVL-263/CC-263 (Avila Therapeutics/Celgene Corporation), AVL-292/CC-292 (Avila Therapeutics/Celgene Corporation), AVL-291/CC-291 (Avila Therapeutics/Celgene Corporation), CNX 774 (Avila Therapeutics), BMS-488516 (Bristol-Myers Squibb), BMS-509744 (Bristol-Myers Squibb), CGI-1746 (CGI Pharma/Gilead Sciences), CGI-560 (CGI Pharma/Gilead Sciences), CTA-056, GDC-0834 (Genentech), HY-11066 (also, CTK4I7891, HMS3265G21, HMS3265G22, HMS3265H21, HMS3265H22, 439574-61-5, AG-F-54930), ONO-4059 (Ono Pharmaceutical Co., Ltd.), ONO-WG37 (Ono Pharmaceutical Co., Ltd.), PLS-123 (Peking University), RN486 (Hoffmann-La Roche), HM71224 (Hanmi Pharmaceutical Company Limited), LFM-A13, BGB-3111 (Beigene), KBP-7536 (KBP BioSciences), ACP-196 (Acerta Pharma), JTE-051 (Japan Tobacco Inc), PRN1008 (Principia), CTP-730 (Concert Pharmaceuticals), or GDC-0853 (Genentech).

In some embodiments, an individual having DLBCL is characterized as resistant or is likely to become resistant to therapy with ibrutinib if the individual has the modification at amino acid position 15 in ROS1. In some embodiments, the A15G modification in ROS1 further indicates the individual has developed or likely to develop a progressive DLBCL.

In some embodiments, also disclosed herein are methods of selecting an individual having a hematological malignancy such as diffuse large B cell lymphoma (DLBCL) for treatment with a TEC inhibitor such as an ITK inhibitor or a BTK inhibitor (e.g. ibrutinib) based on the presence or absence of a modification at amino acid position 15 in ROS1 and one or more additional biomarkers. In some embodiments, further disclosed herein are methods of monitoring whether an individual receiving a TEC inhibitor such as an ITK inhibitor or a BTK inhibitor (e.g. ibrutinib) for treatment of a hematological malignancy such as diffuse large B cell lymphoma (DLBCL) has developed or is likely to develop resistance to the therapy, based on the presence or absence of a modification at amino acid position 15 in ROS1 and one or more additional biomarkers, and characterize the individual as resistant or is likely to become resistant to therapy with ibrutinib if the individual has the modification at amino acid position 15 in ROS1 and one or more biomarkers. In some embodiments, also disclosed herein are methods of optimizing a therapy based on the presence or absence of a modification at amino acid position 15 in ROS1 and one or more additional biomarkers.

In some embodiment, the one or more additional biomarkers include a mutation or modification in BTK. In some embodiments, the modification is a mutation at amino acid position 481 in BTK. In some embodiments, the mutation is C481S in BTK. In some embodiments, the C481 mutation in BTK is accompanied with additional mutations in BTK. In some embodiments, the additional mutations in BTK include substitutions at amino acid positions L11, K12, S14, K19, F25, K27, R28, R33, Y39, Y40, E41, I61, V64, R82, Q103, V113, S115, T117, Q127, C154, C155, T184, P189, P190, Y223, W251, 8288, L295, G302, R307, D308, V319, Y334, L358, Y361, H362, H364, N365, S366, L369, 1370M, R372, L408, G414, Y418, 1429, K430, E445, G462, Y476, M477, C502, C506, A508, M509, L512, L518, R520, D521, A523, R525, N526, V535, L542, R544, Y551, F559, R562, W563, E567, S578, W581, A582, F583, M587, E589, S592, G594, Y598, A607, G613, Y617, P619, A622, V626, M630, C633, R641, F644, L647, L652, V1065, and A1185. In some embodiments, the additional modifications is selected from among L11P, K12R, S14F, K19E, F25S, K27R, R28H, R28C, R28P, T33P, Y3S9, Y40C, Y40N, E41K, I61N, V64F, V64D, R82K, Q103QSFSSVR, V113D, S115F, T117P, Q127H, C154S, C155G, T184P, P189A, Y223F, W251L, R288W, R288Q, L295P, G302E, R307K, R307G, R307T, D308E, V319A, Y334S, L358F, Y361C, H362Q, H364P, N365Y, S366F, L369F, I370M, R372G, L408P, G414R, Y418H, I429N, K430E, E445D, G462D, G462V, Y476D, M477R, C502F, C502W, C506Y, C506R, A508D, M509I, M509V, L512P, L512Q, L518R, R520Q, D521G, D521H, D521N, A523E, R525G, R525P, R525Q, N526K, V535F, L542P, R544G, R544K, Y551F, F559S, R562W, R562P, W563L, E567K, S578Y, W581R, A582V, F583S, M587L, E589D, E589K, E589G, S592P, G594E, Y598C, A607D, G613D, Y617E, P619A, P619S, A622P, V626G, M630I, M630K, M630T, C633Y, R641C, F644L, F644S, L647P, L652P, V1065I, and A1185V.

In some embodiments, the one or more additional biomarkers include a mutation in PLCγ2. In some embodiments, the mutation in PLCγ2 is a mutation at amino acid residue 665, 707, or a combination thereof. In some embodiments, the mutation is R665W and S707F.

In some embodiments, the one or more additional biomarkers include cytogenetic abnormalities such as del(17p13.1), del(13q14.3), del(11q22.3), del(11q23), unmutated IgVH together with ZAP-70+ and/or CD38+, trisomy 12, t(11;14)(q13;q32), t(14;19)(q32;q13), t(2;14)(p13;q32), del(13q14), +(12q21), del(6q21), ATM del, p53 del, t(15;17); t(8;21)(q22;q22), t(6;9), inv(16)(p13q22), del(16q); inv(16), t(16;16), del(11q), t(9;11), t(11;19), t(1;22), del(5q), +8, +21, +22, del(7q), del(9q), abnormal 11q23, -5, -7, abnormal 3q, complex karyotype, t(14;19), t(3:14), t(11;14), t(2;8)(p11;q24), t(1;8)(p36;q24), t(8:9)(q24;p13), t(9;14)(p13;q32), t(3:14)(q27;q32), or a combination thereof.

In some embodiments, also disclosed herein are methods of selecting an individual having a hematological malignancy such as diffuse large B cell lymphoma (DLBCL) for treatment with a TEC inhibitor such as an ITK inhibitor or a BTK inhibitor (e.g. ibrutinib) based on the presence or absence of a modification at amino acid position 15 in ROS1 and a mutation in BTK at amino acid residue position 481. In some embodiments, the mutation is C481S. In some embodiments, further disclosed herein are methods of monitoring whether an individual receiving a TEC inhibitor such as an ITK inhibitor or a BTK inhibitor (e.g. ibrutinib) for treatment of a hematological malignancy such as diffuse large B cell lymphoma (DLBCL) has developed or is likely to develop resistance to the therapy, based on the presence or absence of a modification at amino acid position 15 in ROS1 and a mutation in BTK at amino acid residue position 481, and characterize the individual as resistant or is likely to become resistant to therapy with ibrutinib if the individual has the modification at amino acid position 15 in ROS1 and the mutation in BTK at amino acid residue position 481. In some embodiments, the mutation is C481S. In some embodiments, also disclosed herein are methods of optimizing a therapy based on the presence or absence of a modification at amino acid position 15 in ROS1 and a mutation in BTK at amino acid residue position 481. In some embodiments, the mutation is C481S.

In some embodiments, also disclosed herein are methods of selecting an individual having a hematological malignancy such as diffuse large B cell lymphoma (DLBCL) for treatment with a TEC inhibitor such as an ITK inhibitor or a BTK inhibitor (e.g. ibrutinib) based on the presence or absence of a modification at amino acid position 15 in ROS1 and a mutation in PLCγ2 at amino acid residue position 665 and/or 707. In some embodiments, the mutations are R665W and S707F. In some embodiments, further disclosed herein are methods of monitoring whether an individual receiving a TEC inhibitor such as an ITK inhibitor or a BTK inhibitor (e.g. ibrutinib) for treatment of a hematological malignancy such as diffuse large B cell lymphoma (DLBCL) has developed or is likely to develop resistance to the therapy, based on the presence or absence of a modification at amino acid position 15 in ROS1 and a mutation in PLCγ2 at amino acid residue position 665 and/or 707, and characterize the individual as resistant or is likely to become resistant to therapy with ibrutinib if the individual has the modification at amino acid position 15 in ROS1 and the mutation in PLCγ2 at amino acid residue position 665 and/or 707. In some embodiments, the mutations are R665W and S707F. In some embodiments, also disclosed herein are methods of optimizing a therapy based on the presence or absence of a modification at amino acid position 15 in ROS1 and a mutation in PLCγ2 at amino acid residue position 665 and/or 707. In some embodiments, the mutations are R665W and S707F.

In some embodiments, also disclosed herein are methods of selecting an individual having a hematological malignancy such as diffuse large B cell lymphoma (DLBCL) for treatment with a TEC inhibitor such as an ITK inhibitor or a BTK inhibitor (e.g. ibrutinib) based on the presence or absence of a modification at amino acid position 15 in ROS1 and one or more cytogenetic abnormalities. In some embodiments, further disclosed herein are methods of monitoring whether an individual receiving a TEC inhibitor such as an ITK inhibitor or a BTK inhibitor (e.g. ibrutinib) for treatment of a hematological malignancy such as diffuse large B cell lymphoma (DLBCL) has developed or is likely to develop resistance to the therapy, based on the presence or absence of a modification at amino acid position 15 in ROS1 and one or more cytogenetic abnormalities, and characterize the individual as resistant or is likely to become resistant to therapy with ibrutinib if the individual has the modification at amino acid position 15 in ROS1 and cytogenetic abnormalities. In some embodiments, also disclosed herein are methods of optimizing a therapy based on the presence or absence of a modification at amino acid position 15 in ROS1 and one or more cytogenetic abnormalities. In some embodiments, the one or more additional biomarkers include cytogenetic abnormalities such as del(17p13.1), del(13q14.3), del(11q22.3), del(11q23), unmutated IgVH together with ZAP-70+ and/or CD38+, trisomy 12, t(11;14)(q13;q32), t(14;19)(q32;q13), t(2;14)(p13;q32), del(13q14), +(12q21), del(6q21), ATM del, p53 del, t(15;17); t(8;21)(q22;q22), t(6;9), inv(16)(p13q22), del(16q); inv(16), t(16;16), del(11q), t(9;11), t(11;19), t(1;22), del(5q), +8, +21, +22, del(7q), del(9q), abnormal 11q23, -5, -7, abnormal 3q, complex karyotype, t(14;19), t(3:14), t(11;14), t(2;8)(p11;q24), t(1;8)(p36;q24), t(8:9)(q24;p13), t(9;14)(p13;q32), t(3:14)(q27;q32), or a combination thereof.

### Biomarkers ACTG2, LOR, GAPT, CCND2, SELL, GEN1 and HDAC9

Disclosed herein, in certain embodiments, are methods of selecting an individual having a hematological malignancy for treatment with a TEC inhibitor, or monitoring the disease progression of an individual based on the expression level of at least one biomarker gene selected from ACTG2, LOR, GAPT, CCND2, SELL, GEN1, HDAC9, FGR, and IGHA1. In some embodiments, the biomarker gene is selected from ACTG2, LOR, GAPT, CCND2, SELL, GEN1, and HDAC9. ACTG2 (actin, gamma2, smooth muscle, enteric) is ubiquitously expressed highly conserved protein involved in cell motility and maintenance of the cytoskeleton. LOR encodes the protein loricrin, a major protein component of the stratum corneum, the outermost layer of the epidermis. GAPT (GRB2-binding adaptor protein, transmembrane) negatively regulates B-cell proliferation following stimulation through the B-cell receptor. CCND2 (cyclin D2) is a regulator of cyclin-dependent kinases and is involved in cell cycle regulation. SELL (selectin L or CD62L) is a cell adhesion molecule found on lymphocytes and is involved in lymphocyte-endothelial cell interactions. GEN1 (Gen endonuclease homolog 1) encodes endonucleases which resolves Holliday junctions during homologous recombination and DNA repair. HDAC9, or histone deacetylase 9, is an enzyme involved in transcriptional regulation, cell cycle progression, and developmental events.

In some embodiments, an individual is administered a therapeutically effective amount of a TEC inhibitor if there is an increase in expression level in at least one biomarker gene selected from ACTG2, LOR, GAPT, CCND2, SELL, GEN1, and HDAC9 relative to a control. In some embodiments, an individual is characterized as having a stable hematological malignancy if the individual shows an increase in expression level in at least one biomarker gene selected from ACTG2, LOR, GAPT, CCND2, SELL, GEN1, and HDAC9 relative to a control.

In some embodiments, the expression level of the at least one biomarker gene selected from ACTG2, LOR, GAPT, CCND2, SELL, GEN1, and HDAC9 increase by 0.5-fold, 1-fold, 1.5-fold, 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, 10-fold, 15-fold, 20-fold, 50-fold, 75-fold, 100-fold, 200-fold, 500-fold, 1000-fold, or more compared to the control. In some embodiments, the expression level of the at least one biomarker gene selected from ACTG2, LOR, GAPT, CCND2, SELL, GEN1, and HDAC9 increase by 0.5-fold, 1-fold, 1.5-fold, 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, 10-fold, 15-fold, 20-fold, 50-fold, or more compared to the control.

In some embodiments, the control is the expression levels of the ACTG2, LOR, GAPT, CCND2, SELL, GEN1, and HDAC9 genes in an individual who as a progressive hematological malignancy. In some embodiments, the control is the expression levels of the ACTG2, LOR, GAPT, CCND2, SELL, GEN1, and HDAC9 genes in the individual prior to treatment with a TEC inhibitor. In some embodiments, the control is the expression levels of the ACTG2, LOR, GAPT, CCND2, SELL, GEN1, and HDAC9 genes in the individual who does not have a hematological malignancy.

In some embodiments, the hematological malignancy is a leukemia, a lymphoma, a myeloma, a non-Hodgkin's lymphoma, a Hodgkin's lymphoma, T-cell malignancy, or a B-cell malignancy. In some embodiments, the hematological malignancy is a B-cell malignancy. In some embodiments, the B-cell malignancy is chronic lymphocytic leukemia (CLL), high-risk chronic lymphocytic leukemia (CLL), small lymphocytic lymphoma (SLL), high-risk small lymphocytic lymphoma (SLL), diffuse large B cell lymphoma (DLBCL), mantle cell lymphoma (MCL), or Waldenstrom's macroglobulinemia. In some embodiments, the B-cell malignancy is DLBCL. In some embodiments, the DLBCL is activated B-cell DLBCL (ABC-DLBCL), germinal center B-cell like DLBCL (GBC-DLBCL), double-hit (DH) DLBCL, triple-hit (TH) DLBCL, or unclassified DLBCL. In some embodiments, the DLBCL is activated B-cell DLBCL (ABC-DLBCL).

In some embodiments, the TEC inhibitor is a BTK inhibitor, an ITK inhibitor, a TEC inhibitor, a RLK inhibitor, or a BMX inhibitor. In some embodiments, the TEC inhibitor is an ITK inhibitor. In some embodiments, the TEC inhibitor is a BTK inhibitor. In some embodiments, the BTK inhibitor is ibrutinib.

In some embodiments, an individual having DLBCL is administered a therapeutically effective amount of an ITK inhibitor if there is an increase in expression level in at least one biomarker gene selected from ACTG2, LOR, GAPT, CCND2, SELL, GEN1, and HDAC9 relative to a control. In some embodiments, an individual is characterized as having a stable DLBCL if the individual shows an increase in expression level in at least one biomarker gene selected from ACTG2, LOR, GAPT, CCND2, SELL, GEN1, and HDAC9 relative to a control.

In some embodiments, an individual having DLBCL is administered a therapeutically effective amount of a BTK inhibitor if there is an increase in expression level in at least one biomarker gene selected from ACTG2, LOR, GAPT, CCND2, SELL, GEN1, and HDAC9 relative to a control. In some embodiments, an individual is characterized as having a stable DLBCL if the individual shows an increase in expression level in at least one biomarker gene selected from ACTG2, LOR, GAPT, CCND2, SELL, GEN1, and HDAC9 relative to a control. In some embodiments, the BTK inhibitor is selected from among ibrutinib (PCI-32765), PCI-45292, PCI-45466, AVL-101/CC-101 (Avila Therapeutics/Celgene Corporation), AVL-263/CC-263 (Avila Therapeutics/Celgene Corporation), AVL-292/CC-292 (Avila Therapeutics/Celgene Corporation), AVL-291/CC-291 (Avila Therapeutics/Celgene Corporation), CNX 774 (Avila Therapeutics), BMS-488516 (Bristol-Myers Squibb), BMS-509744 (Bristol-Myers Squibb), CGI-1746 (CGI Pharma/Gilead Sciences), CGI-560 (CGI Pharma/Gilead Sciences), CTA-056, GDC-0834 (Genentech), HY-11066 (also, CTK4I7891, HMS3265G21, HMS3265G22, HMS3265H21, HMS3265H22, 439574-61-5, AG-F-54930), ONO-4059 (Ono Pharmaceutical Co., Ltd.), ONO-WG37 (Ono Pharmaceutical Co., Ltd.), PLS-123 (Peking University), RN486 (Hoffmann-La Roche), HM71224 (Hanmi Pharmaceutical Company Limited), LFM-A13, BGB-3111 (Beigene), KBP-7536 (KBP BioSciences), ACP-196 (Acerta Pharma), JTE-051 (Japan Tobacco Inc), PRN1008 (Principia), CTP-730 (Concert Pharmaceuticals), or GDC-0853 (Genentech).

In some embodiments, an individual having DLBCL is administered a therapeutically effective amount of ibrutinib if there is an increase in expression level in at least one biomarker gene selected from ACTG2, LOR, GAPT, CCND2, SELL, GEN1, and HDAC9 relative to a control. In some embodiments, an individual is characterized as having a stable DLBCL if the individual shows an increase in expression level in at least one biomarker gene selected from ACTG2, LOR, GAPT, CCND2, SELL, GEN1, and HDAC9 relative to a control.

In some embodiments, also disclosed herein are methods of assessing an individual having a hematological malignancy such as diffuse large B cell lymphoma (DLBCL) for treatment with a TEC inhibitor such as an ITK inhibitor or a BTK inhibitor (e.g. ibrutinib) by determining the expression level of at least one biomarker gene selected from ACTG2, LOR, GAPT, CCND2, SELL, GEN1, and HDAC9; and one or more additional biomarkers; and administer to the individual a therapeutically effective amount of a TEC inhibitor such as an ITK inhibitor or a BTK inhibitor (e.g. ibrutinib) if there is an increase in expression level in at least one biomarker gene selected from ACTG2, LOR, GAPT, CCND2, SELL, GEN1, and HDAC9; and one or more additional biomarkers. In some embodiments, the one or more additional biomarkers include CCL3, CCL4, miR155, or a combination thereof.

In some embodiments, further disclosed herein are methods of monitoring the disease progression in an individual having a hematological malignancy such as diffuse large B cell lymphoma (DLBCL) by determining the expression level of at least one biomarker gene selected from ACTG2, LOR, GAPT, CCND2, SELL, GEN1, and HDAC9; and one or more additional biomarkers; and characterize the individual as having a stable hematological malignancy such as a stable DLBCL if the individual shows an increase in expression level in at least one biomarker gene selected from ACTG2, LOR, GAPT, CCND2, SELL, GEN1, and HDAC9, and one or more additional biomarkers. In some embodiments, the one or more additional biomarkers include CCL3, CCL4, miR155, or a combination thereof.

### Additional Biomarkers

Disclosed herein, in certain embodiments, are methods of selecting an individual having a hematological malignancy for treatment with a TEC inhibitor, or monitoring the disease progression of an individual based on the expression level of at least one biomarker selected from osteopontin, MMP-7, aldose reductase, and HGF. Osteopontin is an extracellular structural protein mainly expressed in bones but also expressed in immune cells including macrophages, neutrophiles, dendritic cells, T and B cells. In some embodiments, osteopontin participates in biomineralization, bone remodeling, apoptosis, and mediates cell activation, and cytokine production. MMP-7, matrix metalloproteinase-7, is an enzyme that breaks down extracellular matrix by degrading macromolecules including casein, type 1, II, IV and V gelatins, fibronectin, and proteoglycan. In some cases, elevated expression of MMP-7 facilitates cancer invasion and angiogenesis. Aldose reductase is an NADPH-dependent oxidoreductase that catalyzes the reduction of aldehydes and carbonyls, such as the reduction of toxic lipid aldehyde hydroxyl-trans-2-nonenol (HNE) to 1,4-dihydroxynonene (DHN) and its glutathione conjugate, GS-HNE, to GS-DHN. In some cases, aldose reductase is shown to be involved in growth factors-induced proliferation of certain cancer cells, as well as in cell cycle progression and expression of cell cycle-related proteins such as E2F-1, cyclins and cdks through AKT/PI3K pathway. Hepatocyte growth factor (HGF) is a paracrine cellular growth, motility, and morphogenic factor. HGF participates in cell growth regulation, motility, and morphogenesis via its interaction with the proto-oncogenic c-Met receptor. c-Met is constitutively expressed by several lymphoma cell lines such as Burkitt's lymphoma cell lines. HGF induces c-Met phosphorylation which leads to enhanced integrin-mediated adhesion to fibronectin, and promotes invasion into the fibroblast monolayers.

In some embodiments, an individual having a hematological malignancy is administered a therapeutically effective amount of a TEC inhibitor if there is a decrease in expression level in at least one biomarker selected from osteopontin, MMP-7, aldose reductase, and HGF relative to a reference level. In some embodiments, an individual having a hematological malignancy is not administered a therapeutically effective amount of a TEC inhibitor if there is an elevated expression level in at least one biomarker selected from osteopontin, MMP-7, aldose reductase, and HGF relative to a reference level. In some embodiments, a therapeutic regimen is continued if there is a decrease in expression level in at least one biomarker selected from osteopontin, MMP-7, aldose reductase, and HGF relative to a reference level. In some embodiments, a therapeutic regimen is discontinued if there is an elevated expression level in at least one biomarker selected from osteopontin, MMP-7, aldose reductase, and HGF relative to a reference level. In some embodiments, elevated level of osteopontin is further correlated with shorter overall survival and event-free survival.

In some embodiments, the expression levels of osteopontin, MMP-7, aldose reductase, and HGF are 0.5-fold, 1-fold, 1.5-fold, 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, 10-fold, 15-fold, 20-fold, 50-fold, 75-fold, 100-fold, 200-fold, 500-fold, 1000-fold, or more compared to the reference levels of osteopontin, MMP-7, aldose reductase, and HGF.

In some embodiments, the reference level is the expression levels of the osteopontin, MMP-7, aldose reductase, and HGF in the individual who does not have a hematological malignancy. In some embodiments, the reference level is the expression levels of the osteopontin, MMP-7, aldose reductase, and HGF in the individual prior to treatment with a TEC inhibitor. In some embodiments, the reference level is the expression levels of osteopontin, MMP-7, aldose reductase, and HGF in an individual who as a stable hematological malignancy.

In some embodiments, the hematological malignancy is a leukemia, a lymphoma, a myeloma, a non-Hodgkin's lymphoma, a Hodgkin's lymphoma, T-cell malignancy, or a B-cell malignancy. In some embodiments, the hematological malignancy is a B-cell malignancy. In some embodiments, the B-cell malignancy is chronic lymphocytic leukemia (CLL), high-risk chronic lymphocytic leukemia (CLL), small lymphocytic lymphoma (SLL), high-risk small lymphocytic lymphoma (SLL), diffuse large B cell lymphoma (DLBCL), mantle cell lymphoma (MCL), or Waldenstrom's macroglobulinemia. In some embodiments, the B-cell malignancy is DLBCL. In some embodiments, the DLBCL is activated B-cell DLBCL (ABC-DLBCL), germinal center B-cell like DLBCL (GBC-DLBCL), double-hit (DH) DLBCL, triple-hit (TH) DLBCL, or unclassified DLBCL. In some embodiments, the DLBCL is activated B-cell DLBCL (ABC-DLBCL).

In some embodiments, the TEC inhibitor is a BTK inhibitor, an ITK inhibitor, a TEC inhibitor, a RLK inhibitor, or a BMX inhibitor. In some embodiments, the TEC inhibitor is an ITK inhibitor. In some embodiments, the TEC inhibitor is a BTK inhibitor. In some embodiments, the BTK inhibitor is ibrutinib.

In some embodiments, an individual having DLBCL is administered a therapeutically effective amount of an ITK inhibitor if there is a decrease in expression level in at least one biomarker selected from osteopontin, MMP-7, aldose reductase, and HGF relative to a reference level. In some embodiments, an individual having DLBCL is not administered a therapeutically effective amount of an ITK inhibitor if there is an elevated expression level in at least one biomarker selected from osteopontin, MMP-7, aldose reductase, and HGF relative to a reference level. In some embodiments, a therapeutic regimen is continued if there is a decrease in expression level in at least one biomarker selected from osteopontin, MMP-7, aldose reductase, and HGF relative to a reference level. In some embodiments, a therapeutic regimen is discontinued if there is an elevated expression level in at least one biomarker selected from osteopontin, MMP-7, aldose reductase, and HGF relative to a reference level.

In some embodiments, an individual having DLBCL is administered a therapeutically effective amount of a BTK inhibitor if there is a decrease in expression level in at least one biomarker selected from osteopontin, MMP-7, aldose reductase, and HGF relative to a reference level. In some embodiments, an individual having DLBCL is not administered a therapeutically effective amount of a BTK inhibitor if there is an elevated expression level in at least one biomarker selected from osteopontin, MMP-7, aldose reductase, and HGF relative to a reference level. In some embodiments, a therapeutic regimen is continued if there is a decrease in expression level in at least one biomarker selected from osteopontin, MMP-7, aldose reductase, and HGF relative to a reference level. In some embodiments, a therapeutic regimen is discontinued if there is an elevated expression level in at least one biomarker selected from osteopontin, MMP-7, aldose reductase, and HGF relative to a reference level. In some embodiments, the BTK inhibitor is selected from among ibrutinib (PCI-32765), PCI-45292, PCI-45466, AVL-101/CC-101 (Avila Therapeutics/Celgene Corporation), AVL-263/CC-263 (Avila Therapeutics/Celgene Corporation), AVL-292/CC-292 (Avila Therapeutics/Celgene Corporation), AVL-291/CC-291 (Avila Therapeutics/Celgene Corporation), CNX 774 (Avila Therapeutics), BMS-488516 (Bristol-Myers Squibb), BMS-509744 (Bristol-Myers Squibb), CGI-1746 (CGI Pharma/Gilead Sciences), CGI-560 (CGI Pharma/Gilead Sciences), CTA-056, GDC-0834 (Genentech), HY-11066 (also, CTK4I7891, HMS3265G21, HMS3265G22, HMS3265H21, HMS3265H22, 439574-61-5, AG-F-54930), ONO-4059 (Ono Pharmaceutical Co., Ltd.), ONO-WG37 (Ono Pharmaceutical Co., Ltd.), PLS-123 (Peking University), RN486 (Hoffmann-La Roche), HM71224 (Hanmi Pharmaceutical Company Limited), LFM-A13, BGB-3111 (Beigene), KBP-7536 (KBP BioSciences), ACP-196 (Acerta Pharma), JTE-051 (Japan Tobacco Inc), PRN1008 (Principia), CTP-730 (Concert Pharmaceuticals), or GDC-0853 (Genentech).

In some embodiments, an individual having DLBCL is administered a therapeutically effective amount of ibrutinib if there is a decrease in expression level in at least one biomarker selected from osteopontin, MMP-7, aldose reductase, and HGF relative to a reference level. In some embodiments, an individual having DLBCL is not administered a therapeutically effective amount of ibrutinib if there is an elevated expression level in at least one biomarker selected from osteopontin, MMP-7, aldose reductase, and HGF relative to a reference level. In some embodiments, a therapeutic regimen is continued if there is a decrease in expression level in at least one biomarker selected from osteopontin, MMP-7, aldose reductase, and HGF relative to a reference level. In some embodiments, a therapeutic regimen is discontinued if there is an elevated expression level in at least one biomarker selected from osteopontin, MMP-7, aldose reductase, and HGF relative to a reference level.

### Diagnostic and Therapeutic Methods

### Diagnostic Methods

Methods for determining the expression or presence of biomarker genes such as EP300, MLL2, BCL-2, RB1, LRP1B, PIM1, TSC2, TNFRSF11A, SMAD4, PAX5, CARD11, ACTG2, LOR, GAPT, CCND2, SELL, GEN1, and HDAC9 and biomarkers such as CD79B, MYD88, and ROS1 are well known in the art. Mutations or modifications and expression levels of biomarkers are measured by RT-PCR, Qt-PCR, microarray, Northern blot, or other similar technologies. Circulating levels of biomarkers in a blood sample obtained from a candidate subject are measured, for example, by ELISA, radioimmunoassay (RIA), electrochemiluminescence (ECL), Western blot, multiplexing technologies, or other similar methods. Cell surface expression of biomarkers are measured, for example, by flow cytometry, immunohistochemistry, Western Blot, immunoprecipitation, magnetic bead selection, and quantification of cells expressing either of these cell surface markers.

As disclosed herein, determining the presence, modifications, or expression of the biomarker of interest at the protein or nucleotide level are accomplished using any detection method known to those of skill in the art. By “determining the modification(s)” is intended to determine a mutation within the biomarker gene or a biomarker protein. As used herein, “modification” and “mutation” are used interchangeably. The term “biomarker” refers to in some cases the protein of interest. In some cases, “biomarker” refers to the gene of interest. In some cases, the terms “biomarker” and “biomarker gene” are used interchangeably. By “detecting expression” or “detecting the level of” is intended determining the expression level or presence of a biomarker protein or gene in the biological sample. Thus, “detecting expression” encompasses instances where a biomarker is determined not to be expressed, not to be detectably expressed, expressed at a low level, expressed at a normal level, or overexpressed.

In certain aspects of the method provided herein, the one or more subpopulation of lymphocytes are isolated, detected or measured. In certain embodiments, the one or more subpopulation of lymphocytes are isolated, detected or measured using immunophenotyping techniques. In other embodiments, the one or more subpopulation of lymphocytes are isolated, detected or measured using fluorescence activated cell sorting (FACS) techniques.

In certain aspects, the modifications, expression, or presence of these various biomarkers and any clinically useful prognostic markers in a biological sample are detected at the protein or nucleic acid level, using, for example, immunohistochemistry techniques or nucleic acid-based techniques such as in situ hybridization and RT-PCR. In one embodiments, the modifications, expression, or presence of one or more biomarkers is carried out by a means for nucleic acid amplification, a means for nucleic acid sequencing, a means utilizing a nucleic acid microarray (DNA and RNA), or a means for in situ hybridization using specifically labeled probes.

In some embodiments, the determining the modification, expression, or presence of one or more biomarkers is carried out through gel electrophoresis. In one embodiment, the determination is carried out through transfer to a membrane and hybridization with a specific probe.

In other embodiments, the determining the modification, expression, or presence of one or more biomarkers carried out by a diagnostic imaging technique.

In still other embodiments, the determining the modification, expression, or presence of one or more biomarkers carried out by a detectable solid substrate. In one embodiment, the detectable solid substrate is paramagnetic nanoparticles functionalized with antibodies.

In another aspect, provided herein are methods for detecting or measuring residual lymphoma following a course of treatment in order to guide continuing or discontinuing treatment or changing from one therapeutic regimen to another comprising determining the expression or presence of one or more biomarkers from one or more subpopulation of lymphocytes in a subject wherein the course of treatment is treatment with a Btk inhibitor (e.g., ibrutinib).

Methods for detecting the modification and expression of the biomarkers described herein, within the test and control biological samples comprise any methods that determine the quantity or the presence of these markers either at the nucleic acid or protein level. Such methods are well known in the art and include but are not limited to western blots, northern blots, ELISA, immunoprecipitation, immunofluorescence, flow cytometry, immunohistochemistry, nucleic acid hybridization techniques, nucleic acid reverse transcription methods, and nucleic acid amplification methods. In some embodiments, expression of a biomarker is detected on a protein level using, for example, antibodies that are directed against specific biomarker proteins. These antibodies are used in various methods such as Western blot, ELISA, multiplexing technologies, immunoprecipitation, or immunohistochemistry techniques. In some embodiments, detection of biomarkers is accomplished by ELISA. In some embodiments, detection of biomarkers is accomplished by electrochemiluminescence (ECL).

In some embodiments, the modification, expression, or presence of one or more of the biomarkers described herein are determined at the nucleic acid level. Nucleic acid-based techniques for assessing expression are well known in the art and include, for example, determining the level of biomarker mRNA in a biological sample. Many expression detection methods use isolated RNA. Any RNA isolation technique that does not select against the isolation of mRNA is utilized for the purification of RNA (see, e.g., Ausubel et al., ed. (1987-1999) Current Protocols in Molecular Biology (John Wiley & Sons, New York). Additionally, large numbers of tissue samples are readily processed using techniques well known to those of skill in the art, such as, for example, the single-step RNA isolation process disclosed in U.S. Pat. No. 4,843,155.

Thus, in some embodiments, the detection of a biomarker or other protein of interest is assayed at the nucleic acid level using nucleic acid probes. The term “nucleic acid probe” refers to any molecule that is capable of selectively binding to a specifically intended target nucleic acid molecule, for example, a nucleotide transcript. Probes are synthesized by one of skill in the art, or derived from appropriate biological preparations. Probes are specifically designed to be labeled, for example, with a radioactive label, a fluorescent label, an enzyme, a chemiluminescent tag, a colorimetric tag, or other labels or tags that are discussed above or that are known in the art. Examples of molecules that are utilized as probes include, but are not limited to, RNA and DNA.

For example, isolated mRNA are used in hybridization or amplification assays that include, but are not limited to, Southern or Northern analyses, polymerase chain reaction analyses and probe arrays. One method for the detection of mRNA levels involves contacting the isolated mRNA with a nucleic acid molecule (probe) that hybridize to the mRNA encoded by the gene being detected. The nucleic acid probe comprises of, for example, a full-length cDNA, or a portion thereof, such as an oligonucleotide of at least 7, 15, 30, 50, 100, 250 or 500 nucleotides in length and sufficient to specifically hybridize under stringent conditions to an mRNA or genomic DNA encoding a biomarker, biomarker described herein above. Hybridization of an mRNA with the probe indicates that the biomarker or other target protein of interest is being expressed.

In one embodiment, the mRNA is immobilized on a solid surface and contacted with a probe, for example by running the isolated mRNA on an agarose gel and transferring the mRNA from the gel to a membrane, such as nitrocellulose. In an alternative embodiment, the probe(s) are immobilized on a solid surface and the mRNA is contacted with the probe(s), for example, in a gene chip array. A skilled artisan readily adapts known mRNA detection methods for use in detecting the level of mRNA encoding the biomarkers or other proteins of interest.

An alternative method for determining the level of an mRNA of interest in a sample involves the process of nucleic acid amplification, e.g., by RT-PCR (see, for example, U.S. Pat. No. 4,683,202), ligase chain reaction (Barany (1991) Proc. Natl. Acad. Sci. USA 88:189 193), self-sustained sequence replication (Guatelli et al. (1990) Proc. Natl. Acad. Sci. USA 87:1874-1878), transcriptional amplification system (Kwoh et al. (1989) Proc. Natl. Acad. Sci. USA 86:1173-1177), Q-Beta Replicase (Lizardi et al. (1988) Bio/Technology 6:1197), rolling circle replication (U.S. Pat. No. 5,854,033) or any other nucleic acid amplification method, followed by the detection of the amplified molecules using techniques well known to those of skill in the art. These detection schemes are especially useful for the detection of nucleic acid molecules if such molecules are present in very low numbers. In particular aspects of the invention, biomarker expression is assessed by quantitative fluorogenic RT-PCR (i.e., the TaqMan0 System).

Modifications or expression levels of an RNA of interest are monitored using a membrane blot (such as used in hybridization analysis such as Northern, dot, and the like), or microwells, sample tubes, gels, beads or fibers (or any solid support comprising bound nucleic acids). See U.S. Pat. Nos. 5,770,722, 5,874,219, 5,744,305, 5,677,195 and 5,445,934, which are incorporated herein by reference. The detection of expression also comprises using nucleic acid probes in solution.

In some embodiments, microarrays are used to determine expression or presence of one or more biomarkers. Microarrays are particularly well suited for this purpose because of the reproducibility between different experiments. DNA microarrays provide one method for the simultaneous measurement of the expression levels of large numbers of genes. Each array consists of a reproducible pattern of capture probes attached to a solid support. Labeled RNA or DNA is hybridized to complementary probes on the array and then detected by laser scanning Hybridization intensities for each probe on the array are determined and converted to a quantitative value representing relative gene expression levels. See, U.S. Pat. Nos. 6,040,138, 5,800,992, 6,020,135, 6,033,860, 6,344,316, and U.S. Pat. Application 20120208706. High-density oligonucleotide arrays are particularly useful for determining the gene expression profile for a large number of RNA's in a sample. Exemplary microarray chips include FoundationOne and FoundationOne Heme from Foundation Medicine, Inc; GeneChip® Human Genome U133 Plus 2.0 array from Affymetrix; and Human DiscoveryMAP® 250+ v. 2.0 from Myraid RBM.

Techniques for the synthesis of these arrays using mechanical synthesis methods are described in, e.g., U.S. Pat. No. 5,384,261. In some embodiments, an array is fabricated on a surface of virtually any shape or even a multiplicity of surfaces. In some embodiments, an array is a planar array surface. In some embodiments, arrays include peptides or nucleic acids on beads, gels, polymeric surfaces, fibers such as fiber optics, glass or any other appropriate substrate, see U.S. Pat. Nos. 5,770,358, 5,789,162, 5,708,153, 6,040,193 and 5,800,992, each of which is hereby incorporated in its entirety for all purposes. In some embodiments, arrays are packaged in such a manner as to allow for diagnostics or other manipulation of an all-inclusive device.

Any means for specifically identifying and quantifying a biomarker (for example, biomarker, a biomarker of cell survival or proliferation, a biomarker of apoptosis, a biomarker of a Btk-mediated signaling pathway) in the biological sample of a candidate subject is contemplated. Thus, in some embodiments, expression level of a biomarker protein of interest in a biological sample is detected by means of a binding protein capable of interacting specifically with that biomarker protein or a biologically active variant thereof. In some embodiments, labeled antibodies, binding portions thereof, or other binding partners are used. The word “label” when used herein refers to a detectable compound or composition that is conjugated directly or indirectly to the antibody so as to generate a “labeled” antibody. In some embodiments, the label is detectable by itself (e.g., radioisotope labels or fluorescent labels) or, in the case of an enzymatic label, catalyzes chemical alteration of a substrate compound or composition that is detectable.

The antibodies for detection of a biomarker protein are either monoclonal or polyclonal in origin, or are synthetically or recombinantly produced. The amount of complexed protein, for example, the amount of biomarker protein associated with the binding protein, for example, an antibody that specifically binds to the biomarker protein, is determined using standard protein detection methodologies known to those of skill in the art. A detailed review of immunological assay design, theory and protocols are found in numerous texts in the art (see, for example, Ausubel et al., eds. (1995) Current Protocols in Molecular Biology) (Greene Publishing and Wiley-Interscience, NY)); Coligan et al., eds. (1994) Current Protocols in Immunology (John Wiley & Sons, Inc., New York, N.Y.).

The choice of marker used to label the antibodies will vary depending upon the application. However, the choice of the marker is readily determinable to one skilled in the art. These labeled antibodies are used in immunoassays as well as in histological applications to detect the presence of any biomarker or protein of interest. The labeled antibodies are either polyclonal or monoclonal. Further, the antibodies for use in detecting a protein of interest are labeled with a radioactive atom, an enzyme, a chromophoric or fluorescent moiety, or a colorimetric tag as described elsewhere herein. The choice of tagging label also will depend on the detection limitations desired. Enzyme assays (ELISAs) typically allow detection of a colored product formed by interaction of the enzyme-tagged complex with an enzyme substrate. Radionuclides that serve as detectable labels include, for example, 1-131, 1-123, 1-125, Y-90, Re-188, Re-186, At-211, Cu-67, Bi-212, and Pd-109. Examples of enzymes that serve as detectable labels include, but are not limited to, horseradish peroxidase, alkaline phosphatase, beta-galactosidase, and glucose-6-phosphate dehydrogenase. Chromophoric moieties include, but are not limited to, fluorescein and rhodamine. The antibodies are conjugated to these labels by methods known in the art. For example, enzymes and chromophoric molecules are conjugated to the antibodies by means of coupling agents, such as dialdehydes, carbodiimides, dimaleimides, and the like. Alternatively, conjugation occurs through a ligand-receptor pair. Examples of suitable ligand-receptor pairs are biotin-avidin or biotin-streptavidin, and antibody-antigen.

In certain embodiments, expression or presence of one or more biomarkers or other proteins of interest within a biological sample, for example, a sample of bodily fluid, is determined by radioimmunoassays or enzyme-linked immunoassays (ELISAs), competitive binding enzyme-linked immunoassays, dot blot (see, for example, Promega Protocols and Applications Guide, Promega Corporation (1991), Western blot (see, for example, Sambrook et al. (1989) Molecular Cloning, A Laboratory Manual, Vol. 3, Chapter 18 (Cold Spring Harbor Laboratory Press, Plainview, N.Y.), chromatography such as high performance liquid chromatography (HPLC), or other assays known in the art. Thus, the detection assays involve steps such as, but not limited to, immunoblotting, immunodiffusion, immunoelectrophoresis, or immunoprecipitation.

In certain other embodiments, the methods disclosed herein are useful for identifying and treating a hematological malignancy, including those listed herein, that are refractory to (i.e., resistant to, or have become resistant to) first-line oncotherapeutic treatments.

### Samples

In some embodiments, the sample for use in the methods is obtained from cells of a hematological malignant cell line. In some embodiments, the sample is obtained from cells of a acute lymphoblastic leukemia (ALL), acute myelogenous leukemia (AML), chronic myelogenous leukemia (CML), acute monocytic leukemia (AMoL), chronic lymphocytic leukemia (CLL), high risk CLL, small lymphocytic lymphoma (SLL), high risk SLL, follicular lymphoma (FL), diffuse large B-cell lymphoma (DLBCL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis cell line. In some embodiments, the sample is obtained from cells of a DLBCL cell line.

In some embodiments, the sample is a DLBCL cell or population of DLBCL cells. In some embodiments, the DLBCL cell line is an activated B-cell-like (ABC)-DLBCL cell line. In some embodiments, the DLBCL cell line is a germinal center B-cell-like (GCB)-DLBCL cell line. In some embodiments, the DLBCL cell line is OCI-Ly1, OCI-Ly2, OCI-Ly3, OCI-Ly4, OCI-Ly6, OCI-Ly7, OCI-Ly10, OCI-Ly18, OCI-Ly19, U2932, DB, HBL-1, RIVA, SUDHL2, or TMD8. In some embodiments, the DLBCL cell line that is sensitive to treatment with a BTK inhibitor is TMD8, HBL-1 or OCI-Ly10. In some embodiments, the DLBCL cell line that is resistant to treatment with a BTK inhibitor is OCI-Ly3, DB or OCI-Ly19.

In some embodiments, the sample for use in the methods is from any tissue or fluid from a patient. Samples include, but are not limited, to whole blood, dissociated bone marrow, bone marrow aspirate, pleural fluid, peritoneal fluid, central spinal fluid, abdominal fluid, pancreatic fluid, cerebrospinal fluid, brain fluid, ascites, pericardial fluid, urine, saliva, bronchial lavage, sweat, tears, ear flow, sputum, hydrocele fluid, semen, vaginal flow, milk, amniotic fluid, and secretions of respiratory, intestinal or genitourinary tract. In particular embodiments, the sample is a blood serum sample. In particular embodiments, the sample is from a fluid or tissue that is part of, or associated with, the lymphatic system or circulatory system. In some embodiments, the sample is a blood sample that is a venous, arterial, peripheral, tissue, cord blood sample. In particular embodiments, the sample is a blood cell sample containing one or more peripheral blood mononuclear cells (PBMCs). In some embodiments, the sample contains one or more circulating tumor cells (CTCs). In some embodiments, the sample contains one or more disseminated tumor cells (DTC, e.g., in a bone marrow aspirate sample).

In some embodiments, the samples are obtained from the individual by any suitable means of obtaining the sample using well-known and routine clinical methods. Procedures for obtaining fluid samples from an individual are well known. For example, procedures for drawing and processing whole blood and lymph are well-known and can be employed to obtain a sample for use in the methods provided. Typically, for collection of a blood sample, an anti-coagulation agent (e.g., EDTA, or citrate and heparin or CPD (citrate, phosphate, dextrose) or comparable substances) is added to the sample to prevent coagulation of the blood. In some examples, the blood sample is collected in a collection tube that contains an amount of EDTA to prevent coagulation of the blood sample.

In some embodiments, the collection of a sample from the individual is performed at regular intervals, such as, for example, one day, two days, three days, four days, five days, six days, one week, two weeks, weeks, four weeks, one month, two months, three months, four months, five months, six months, one year, daily, weekly, bimonthly, quarterly, biyearly or yearly.

In some embodiments, the collection of a sample is performed at a predetermined time or at regular intervals relative to treatment with a TEC inhibitor. In some embodiments, the TEC inhibitor is a BTK inhibitor, an ITK inhibitor, a TEC inhibitor, a RLK inhibitor, or a BMX inhibitor. In some embodiments, the TEC inhibitor is an ITK inhibitor. In some embodiments, the TEC inhibitor is a BTK inhibitor.

In some embodiments, the collection of a sample is performed at a predetermined time or at regular intervals relative to treatment with an ITK inhibitor. For example, a sample is collected from a patient at a predetermined time or at regular intervals prior to, during, or following treatment or between successive treatments with an ITK inhibitor. In particular examples, a sample is obtained from a patient prior to administration of an ITK inhibitor, and then again at regular intervals after treatment with the ITK inhibitor has been effected. In some embodiments, the patient is administered an ITK inhibitor and one or more additional therapeutic agents. In some embodiments, the ITK inhibitor is an irreversible ITK inhibitor. In some embodiments, the ITK inhibitor is a reversible ITK inhibitor.

In some embodiments, the collection of a sample is performed at a predetermined time or at regular intervals relative to treatment with a BTK inhibitor. For example, a sample is collected from a patient at a predetermined time or at regular intervals prior to, during, or following treatment or between successive treatments with a BTK inhibitor. In particular examples, a sample is obtained from a patient prior to administration of a BTK inhibitor, and then again at regular intervals after treatment with the BTK inhibitor has been effected. In some embodiments, the patient is administered a BTK inhibitor and one or more additional therapeutic agents. In some embodiments, the BTK inhibitor is an irreversible BTK inhibitor. In some embodiments, the BTK inhibitor is a reversible BTK inhibitor. In some embodiments, the BTK inhibitor is ibrutinib. In some embodiments, the BTK inhibitor is selected from among ibrutinib (PCI-32765), PCI-45292, PCI-45466, AVL-101/CC-101 (Avila Therapeutics/Celgene Corporation), AVL-263/CC-263 (Avila Therapeutics/Celgene Corporation), AVL-292/CC-292 (Avila Therapeutics/Celgene Corporation), AVL-291/CC-291 (Avila Therapeutics/Celgene Corporation), CNX 774 (Avila Therapeutics), BMS-488516 (Bristol-Myers Squibb), BMS-509744 (Bristol-Myers Squibb), CGI-1746 (CGI Pharma/Gilead Sciences), CGI-560 (CGI Pharma/Gilead Sciences), CTA-056, GDC-0834 (Genentech), HY-11066 (also, CTK4I7891, HMS3265G21, HMS3265G22, HMS3265H21, HMS3265H22, 439574-61-5, AG-F-54930), ONO-4059 (Ono Pharmaceutical Co., Ltd.), ONO-WG37 (Ono Pharmaceutical Co., Ltd.), PLS-123 (Peking University), RN486 (Hoffmann-La Roche), HM71224 (Hanmi Pharmaceutical Company Limited), LFM-A13, BGB-3111 (Beigene), KBP-7536 (KBP BioSciences), ACP-196 (Acerta Pharma), JTE-051 (Japan Tobacco Inc), PRN1008 (Principia), CTP-730 (Concert Pharmaceuticals), or GDC-0853 (Genentech).

In some embodiments, the collection of a sample is performed at a predetermined time or at regular intervals relative to treatment with ibrutinib. For example, a sample is collected from a patient at a predetermined time or at regular intervals prior to, during, or following treatment or between successive treatments with ibrutinib. In particular examples, a sample is obtained from a patient prior to administration of ibrutinib, and then again at regular intervals after treatment with ibrutinib has been effected. In some embodiments, the patient is administered ibrutinib and one or more additional therapeutic agents.

### TEC Family Kinase Inhibitors

BTK is a member of the Tyrosine-protein kinase (TEC) family of kinases. In some embodiments, the TEC family comprises BTK, ITK, TEC, RLK and BMX. In some embodiments, a covalent TEC family kinase inhibitor inhibits the kinase activity of BTK, ITK, TEC, RLK and BMX. In some embodiments, a covalent TEC family kinase inhibitor is a BTK inhibitor. In some embodiments, a covalent TEC family kinase inhibitor is an ITK inhibitor. In some embodiments, a covalent TEC family kinase inhibitor is a TEC inhibitor. In some embodiments, a covalent TEC family kinase inhibitor is a RLK inhibitor. In some embodiments, a covalent TEC family kinase inhibitor is a BMK inhibitor.

### BTK Inhibitor Compounds Including Ibrutinib, and Pharmaceutically Acceptable Salts Thereof

The BTK inhibitor compound described herein (i.e., Ibrutinib) is selective for BTK and kinases having a cysteine residue in an amino acid sequence position of the tyrosine kinase that is homologous to the amino acid sequence position of cysteine 481 in BTK. The BTK inhibitor compound can form a covalent bond with Cys 481 of BTK (e.g., via a Michael reaction).

In some embodiments, the BTK inhibitor is a compound of Formula (A) having the structure:

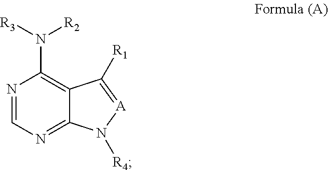

wherein:

A is N;

R₁ is phenyl-O-phenyl or phenyl-S-phenyl;

R₂ and R₃ are independently H;

R₄ is L₃-X-L₄-G, wherein,

L₃ is optional, and when present is a bond, optionally substituted or unsubstituted alkyl, optionally substituted or unsubstituted cycloalkyl, optionally substituted or unsubstituted alkenyl, optionally substituted or unsubstituted alkynyl;

X is optional, and when present is a bond, —O—, —C(═O)—, —S—, —S(═O)—, —S(═O)₂—, —NH—, —NR₉—, —NHC(O)—, —C(O)NH—, —NR₉C(O)—, —C(O)NR₉—, —S(═O)₂NH—, —NHS(═O)₂—, —S(═O)₂NR₉—, —NR₉S(═O)₂—, —OC(O)NH—, —NHC(O)O—, —OC(O)NR₉—, —NR₉C(O)O—, —CH═NO—, —ON═CH—, —NR₁₀C(O)NR₁₀—, heteroaryl-, aryl-, —NR₁₀C(═NR₁₁)NR₁₀—, —NR₁₀C(═NR₁₁)—, —C(═NR₁₁)NR₁₀—, —OC(═NR₁₁)—, or —C(═NR₁₁)O—;

L₄ is optional, and when present is a bond, substituted or unsubstituted alkyl, substituted or unsubstituted cycloalkyl, substituted or unsubstituted alkenyl, substituted or unsubstituted alkynyl, substituted or unsubstituted aryl, substituted or unsubstituted heteroaryl, substituted or unsubstituted heterocycle;

or L₃, X and L₄ taken together form a nitrogen containing heterocyclic ring;

G is

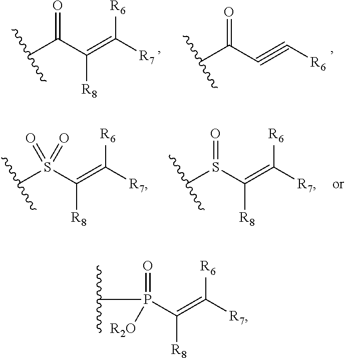

wherein,

R₆, R₇ and R₈ are independently selected from among H, halogen, CN, OH, substituted or unsubstituted alkyl or substituted or unsubstituted heteroalkyl or substituted or unsubstituted cycloalkyl, substituted or unsubstituted heterocycloalkyl, substituted or unsubstituted aryl, substituted or unsubstituted heteroaryl;

each R₉ is independently selected from among H, substituted or unsubstituted lower alkyl, and substituted or unsubstituted lower cycloalkyl;

each R₁₀ is independently H, substituted or unsubstituted lower alkyl, or substituted or unsubstituted lower cycloalkyl; or

two R₁₀ groups can together form a 5-, 6-, 7-, or 8-membered heterocyclic ring; or

R₁₀ and R₁₁ can together form a 5-, 6-, 7-, or 8-membered heterocyclic ring; or each R₁₁ is independently selected from H or substituted or unsubstituted alkyl; or a pharmaceutically acceptable salt thereof. In some embodiments, L₃, X and L₄ taken together form a nitrogen containing heterocyclic ring. In some embodiments, the nitrogen containing heterocyclic ring is a piperidine group. In some embodiments, G is

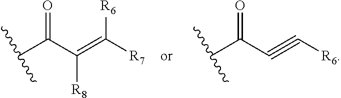

In some embodiments, the compound of Formula (A) is 1-[(3R)-3-[4-amino-3-(4-phenoxyphenyl)pyrazolo[3,4-d]pyrimidin-1-yl]piperidin-1-yl]prop-2-en-1-one.

In some embodiments, the BTK inhibitor is a compound having the structure of Formula (A1):

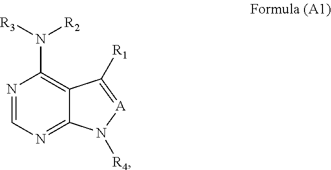

wherein
A is independently selected from N or CR₅;
R₁ is H, L₂-(substituted or unsubstituted alkyl), L₂-(substituted or unsubstituted cycloalkyl), L₂-(substituted or unsubstituted alkenyl), L₂-(substituted or unsubstituted cycloalkenyl), L₂-(substituted or unsubstituted heterocycle), L₂-(substituted or unsubstituted heteroaryl), or L₂-(substituted or unsubstituted aryl), where L₂ is a bond, O, S, —S(═O), —S(═O)₂, C(═O), -(substituted or unsubstituted C₁-C₆ alkylene), or -(substituted or unsubstituted C₂-C₆ alkenylene);
R₂ and R₃ are independently selected from H, lower alkyl and substituted lower alkyl;
R₄ is L₃-X-L₄-G, wherein,
   L₃ is optional, and when present is a bond, or an optionally substituted group selected from alkylene, heteroalkylene, arylene, heteroarylene, alkylarylene, alkylheteroarylene, or alkylheterocycloalkylene;
   X is optional, and when present is a bond, O, —C(═O), S, —S(═O), —S(═O)₂, —NH, —NR₉, —NHC(O), —C(O)NH, —NR₉C(O), —C(O)NR₉, —S(═O)₂NH, —NHS(═O)₂, —S(═O)₂NR₉—, —NR₉S(═O)₂, —OC(O)NH—, —NHC(O)O—, —OC(O)NR₉—, —NR₉C(O)O—, —CH═NO—, —ON═CH—, —NR₁₀C(O)NR₁₀—, heteroarylene, arylene, —NR₁₀C(═NR₁₁)NR₁₀—, —NR₁₀C(═NR₁₁)—, —C(═NR₁₁)NR₁₀—, —OC(═NR₁₁)—, or —C(═NR₁₁)O—;
   L₄ is optional, and when present is a bond, substituted or unsubstituted alkylene, substituted or unsubstituted cycloalkylene, substituted or unsubstituted alkenylene, substituted or unsubstituted alkynylene, substituted or unsubstituted arylene, substituted or unsubstituted heteroarylene, substituted or unsubstituted heterocyclene;
   or L₃, X and L₄ taken together form a nitrogen containing heterocyclic ring, or an optionally substituted group selected from alkyl, heteroalkyl, aryl, heteroaryl, alkylaryl, alkylheteroaryl, or alkylheterocycloalkyl;
      G is

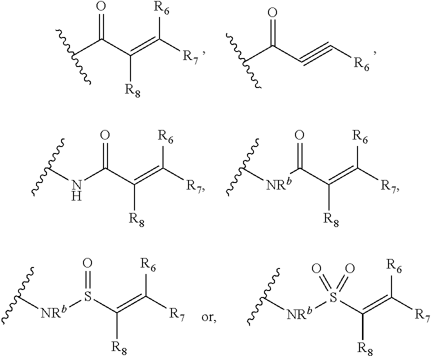

where R^{b} is H, substituted or unsubstituted alkyl, substituted or unsubstituted cycloalkyl; and either R₇ and R₈ are H;
R₆ is H, substituted or unsubstituted C₁-C₄alkyl, substituted or unsubstituted C₁-C₄heteroalkyl, C₁-C₈alkylaminoalkyl, C₁-C₈hydroxyalkylaminoalkyl, C₁-C₈alkoxyalkylaminoalkyl, substituted or unsubstituted C₃-C₆cycloalkyl, substituted or unsubstituted C₁-C₈alkylC₃-C₆cycloalkyl, substituted or unsubstituted aryl, substituted or unsubstituted C₂-C₈heterocycloalkyl, substituted or unsubstituted heteroaryl, C₁-C₄alkyl(aryl), C₁-C₄alkyl(heteroaryl), C₁-C₈alkylethers, C₁-C₈alkylamides, or C₁-C₄alkyl(C₂-C₈heterocycloalkyl);
R₆ and R₈ are H;
   R₇ is H, substituted or unsubstituted C₁-C₄alkyl, substituted or unsubstituted C₁-C₄heteroalkyl, C₁-C₈alkylaminoalkyl, C₁-C₈hydroxyalkylaminoalkyl, C₁-C₈alkoxyalkylaminoalkyl, substituted or unsubstituted C₃-C₆cycloalkyl, substituted or unsubstituted C₁-C₈alkylC₃-C₆cycloalkyl, substituted or unsubstituted aryl, substituted or unsubstituted C₂-C₈heterocycloalkyl, substituted or unsubstituted heteroaryl, C₁-C₄alkyl(aryl), C₁-C₄alkyl(heteroaryl), C₁-C₈alkylethers, C₁-C₈ alkylamides, or C₁-C₄alkyl(C₂-C₈heterocycloalkyl); or
R₇ and R₈ taken together form a bond;
   R₆ is H, substituted or unsubstituted C₁-C₄alkyl, substituted or unsubstituted C₁-C₄heteroalkyl, C₁-C₈alkylaminoalkyl, C₁-C₈hydroxyalkylaminoalkyl, C₁-C₈alkoxyalkylaminoalkyl, substituted or unsubstituted C₃-C₆cycloalkyl, substituted or unsubstituted C₁-C₈alkylC₃-C₆cycloalkyl, substituted or unsubstituted aryl, substituted or unsubstituted C₂-C₈heterocycloalkyl, substituted or unsubstituted heteroaryl, C₁-C₄alkyl(aryl), C₁-C₄alkyl(heteroaryl), C₁-C₈alkylethers, C₁-C₈alkylamides, or C₁-C₄alkyl(C₂-C₈heterocycloalkyl); or
R₅ is H, halogen, -L₆-(substituted or unsubstituted C₁-C₃ alkyl), -L₆-(substituted or unsubstituted C₂-C₄ alkenyl), -L₆-(substituted or unsubstituted heteroaryl), or -L₆-(substituted or unsubstituted aryl), wherein L₆ is a bond, O, S, —S(═O), S(═O)₂, NH, C(O), —NHC(O)O, —OC(O)NH, —NHC(O), or —C(O)NH;
R₉ is selected from among H, substituted or unsubstituted lower alkyl, and substituted or unsubstituted lower cycloalkyl;
each R₁₀ is independently H, substituted or unsubstituted lower alkyl, or substituted or unsubstituted lower cycloalkyl; or
two R₁₀ groups can together form a 5-, 6-, 7-, or 8-membered heterocyclic ring; or
R₁₀ and R₁₁ can together form a 5-, 6-, 7-, or 8-membered heterocyclic ring; or
R₁₁ is selected from H, —S(═O)₂R₈, —S(═O)₂NH₂, —C(O)R₈, —CN, —NO₂, heteroaryl, or heteroalkyl; and pharmaceutically active metabolites, pharmaceutically acceptable solvates, pharmaceutically acceptable salts, or pharmaceutically acceptable prodrugs thereof.

In some embodiments, A is independently selected from N. In some embodiments R₁ is L₂-(substituted or unsubstituted heteroaryl), or L₂-(substituted or unsubstituted aryl), where L₂ is a bond, O, S, —S(═O), —S(═O)₂, C(═O), -(substituted or unsubstituted C₁-C₆ alkylene), or -(substituted or unsubstituted C₂-C₆ alkenylene). In a further embodiment, R₁ is L₂-(substituted or unsubstituted aryl) and L₂ is a bond. In a further embodiment, R₁ is L₂-(substituted aryl) wherein L₂ is a bond and aryl is substituted with L3-(substituted or unsubstituted heteroaryl) or L₃-(substituted or unsubstituted aryl). In a further embodiment, L₃ is a bond, O, S, NHC(O), C(O)NH.

In some embodiments, L₃, X and L₄ taken together form a nitrogen containing heterocyclic ring. In a further embodiment L₃, X and L₄ taken together form a pyrrolidine ring or a piperidine ring. In yet a further embodiment L₃, X and L₄ taken together form a piperidine ring. In some embodiments, G is

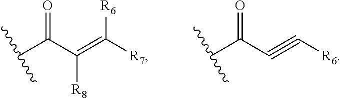

In some embodiments G is

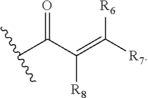

In some embodiments, R₆, R₇ and R₈ are H.

In some embodiments, examples of covalent Btk inhibitors are found in the following patents and patent applications, all of which are incorporated herein in their entirety by reference: U.S. Pat. No. 7,514,444; U.S. Pat. No. 7,960,396; U.S. Pat. No. 8,236,812; U.S. Pat. No. 8,497,277; U.S. Pat. No. 8,563,563; U.S. Pat. No. 8,399,470; U.S. Pat. No. 8,088,781; U.S. Pat. No. 8,501,751; U.S. Pat. No. 8,008,309; U.S. Pat. No. 8,552,010; U.S. Pat. No. 7,732,454; U.S. Pat. No. 7,825,118; U.S. Pat. No. 8,377,946; U.S. Pat. No. 8,501,724; US Patent Pub. No. 2011-0039868; U.S. Pat. No. 8,232,280; U.S. Pat. No. 8,158,786; US Patent Pub. No. 2011-0281322; US Patent Pub. No. 2012-0088912; US Patent Pub. No. 2012-0108612; US Patent Pub. No. 2012-0115889; US Patent Pub. No. 2013-0005745; US Patent Pub. No. 2012-0122894; US Patent Pub. No. 2012-0135944; US Patent Pub. No. 2012-0214826; US Patent Pub. No. 2012-0252821; US Patent Pub. No. 2012-0252822; US Patent Pub. No. 2012-0277254; US Patent Pub. No. 2010-0022561; US Patent Pub. No. 2010-0324050; US Patent Pub. No. 2012-0283276; US Patent Pub. No. 2012-0065201; US Patent Pub. No. 2012-0178753; US Patent Pub. No. 2012-0101113; US Patent Pub. No. 2012-0101114; US Patent Pub. No. 2012-0165328; US Patent Pub. No. 2012-0184013; US Patent Pub. No. 2012-0184567; US Patent Pub. No. 2012-0202264; US Patent Pub. No. 2012-0277225; US Patent Pub. No. 2012-0277255; US Patent Pub. No. 2012-0296089; US Patent Pub. No. 2013-0035334; US Patent Pub. No. 2012-0329130; US Patent Pub. No. 2013-0018060; US Patent Pub. No. 2010-0254905; U.S. Patent App. No. 60/826,720; U.S. Patent App. No. 60/828,590; U.S. patent application Ser. No. 13/654,173; U.S. patent application Ser. No. 13/849,399; U.S. patent application Ser. No. 13/890,498; U.S. patent application Ser. No. 13/952,531; U.S. patent application Ser. No. 14/033,344; U.S. patent application Ser. No. 14/073,543; U.S. patent application Ser. No. 14/073,594; U.S. patent application Ser. No. 14/079,508; U.S. patent application Ser. No. 14/080,640; U.S. patent application Ser. No. 14/080,649; U.S. patent application Ser. No. 14/069,222; PCT App. No. PCT/US2008/58528; PCT App. No. PCT/US2012/046779; U.S. Patent App. No. 61/582,199; U.S. patent application Ser. No. 13/619,466; PCT App. No. PCT/US2012/72043; U.S. Patent App. No. 61/593,146; U.S. Patent App. No. 61/637,765; PCT App. No. PCT/US2013/23918; U.S. Patent App. No. 61/781,975; U.S. Patent App. No. 61/727,031; PCT App. No. PCT/US2013/7016; U.S. Patent App. No. 61/647,956; PCT App. No. PCT/US2013/41242; U.S. Patent App. No. 61/769,103; U.S. Patent App. No. 61/842,321; and U.S. Patent App. No. 61/884,888.

“Ibrutinib” or “1-((R)-3-(4-amino-3-(4-phenoxyphenyl)-1H-pyrazolo[3,4-d]pyrimidin-1-yl)piperidin-1-yl)prop-2-en-1-one” or “1-{(3R)-3-[4-amino-3-(4-phenoxyphenyl)-1H-pyrazolo[3,4-d]pyrimidin-1-yl]piperidin-1-yl}prop-2-en-1-one” or “2-Propen-1-one, 1-[(3R)-3-[4-amino-3-(4-phenoxyphenyl)-1H-pyrazolo[3,4-d]pyrimidin-1-yl]-1-piperidinyl-” or Ibrutinib or any other suitable name refers to the compound with the following structure:

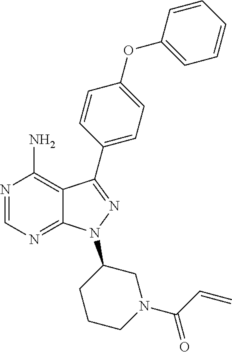 [SMILES: C=CC(=O)N1CCC[C@@H](n2nc(-c3ccc(Oc4ccccc4)cc3)c3c(N)ncnc32)C1]

A wide variety of pharmaceutically acceptable salts is formed from Ibrutinib and includes:
acid addition salts formed by reacting Ibrutinib with an organic acid, which includes aliphatic mono- and dicarboxylic acids, phenyl-substituted alkanoic acids, hydroxyl alkanoic acids, alkanedioic acids, aromatic acids, aliphatic and aromatic sulfonic acids, amino acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like;
acid addition salts formed by reacting Ibrutinib with an inorganic acid, which includes hydrochloric acid, hydrobromic acid, sulfuric acid, nitric acid, phosphoric acid, hydroiodic acid, hydrofluoric acid, phosphorous acid, and the like.

The term “pharmaceutically acceptable salts” in reference to Ibrutinib refers to a salt of Ibrutinib, which does not cause significant irritation to a mammal to which it is administered and does not substantially abrogate the biological activity and properties of the compound.

It should be understood that a reference to a pharmaceutically acceptable salt includes the solvent addition forms (solvates). Solvates contain either stoichiometric or non-stoichiometric amounts of a solvent, and are formed during the process of product formation or isolation with pharmaceutically acceptable solvents such as water, ethanol, methanol, methyl tert-butyl ether (MTBE), diisopropyl ether (DIPE), ethyl acetate, isopropyl acetate, isopropyl alcohol, methyl isobutyl ketone (MIBK), methyl ethyl ketone (MEK), acetone, nitromethane, tetrahydrofuran (THF), dichloromethane (DCM), dioxane, heptanes, toluene, anisole, acetonitrile, and the like. In one aspect, solvates are formed using, but limited to, Class 3 solvent(s). Categories of solvents are defined in, for example, the International Conference on Harmonization of Technical Requirements for Registration of Pharmaceuticals for Human Use (ICH), “Impurities: Guidelines for Residual Solvents, Q3C(R3), (November 2005). Hydrates are formed when the solvent is water, or alcoholates are formed when the solvent is alcohol. In some embodiments, solvates of Ibrutinib, or pharmaceutically acceptable salts thereof, are conveniently prepared or formed during the processes described herein. In some embodiments, solvates of Ibrutinib are anhydrous. In some embodiments, Ibrutinib, or pharmaceutically acceptable salts thereof, exist in unsolvated form. In some embodiments, Ibrutinib, or pharmaceutically acceptable salts thereof, exist in unsolvated form and are anhydrous.

In yet other embodiments, Ibrutinib, or a pharmaceutically acceptable salt thereof, is prepared in various forms, including but not limited to, amorphous phase, crystalline forms, milled forms and nano-particulate forms. In some embodiments, Ibrutinib, or a pharmaceutically acceptable salt thereof, is amorphous. In some embodiments, Ibrutinib, or a pharmaceutically acceptable salt thereof, is amorphous and anhydrous. In some embodiments, Ibrutinib, or a pharmaceutically acceptable salt thereof, is crystalline. In some embodiments, Ibrutinib, or a pharmaceutically acceptable salt thereof, is crystalline and anhydrous.

In some embodiments, Ibrutinib is prepared as outlined in U.S. Pat. No. 7,514,444.

In some embodiments, the Btk inhibitor is PCI-45292, PCI-45466, AVL-101/CC-101 (Avila Therapeutics/Celgene Corporation), AVL-263/CC-263 (Avila Therapeutics/Celgene Corporation), AVL-292/CC-292 (Avila Therapeutics/Celgene Corporation), AVL-291/CC-291 (Avila Therapeutics/Celgene Corporation), CNX 774 (Avila Therapeutics), BMS-488516 (Bristol-Myers Squibb), BMS-509744 (Bristol-Myers Squibb), CGI-1746 (CGI Pharma/Gilead Sciences), CGI-560 (CGI Pharma/Gilead Sciences), CTA-056, GDC-0834 (Genentech), HY-11066 (also, CTK4I7891, HMS3265G21, HMS3265G22, HMS3265H21, HMS3265H22, 439574-61-5, AG-F-54930), ONO-4059 (Ono Pharmaceutical Co., Ltd.), ONO-WG37 (Ono Pharmaceutical Co., Ltd.), PLS-123 (Peking University), RN486 (Hoffmann-La Roche), HM71224 (Hanmi Pharmaceutical Company Limited), LFM-A13, BGB-3111 (Beigene), KBP-7536 (KBP BioSciences), ACP-196 (Acerta Pharma), JTE-051 (Japan Tobacco Inc), PRN1008 (Principia), CTP-730 (Concert Pharmaceuticals), or GDC-0853 (Genentech).

In some embodiments, the BTK inhibitor is 4-(tert-butyl)-N-(2-methyl-3-(4-methyl-6-((4-(morpholine-4-carbonyl)phenyl)amino)-5-oxo-4,5-dihydropyrazin-2-yl)phenyl)benzamide (CGI-1746); 7-benzyl-1-(3-(piperidin-1-yl)propyl)-2-(4-(pyridin-4-yl)phenyl)-1H-imidazo[4,5-g]quinoxalin-6(5H)-one (CTA-056); (R)—N-(3-(6-(4-(1,4-dimethyl-3-oxopiperazin-2-yl)phenylamino)-4-methyl-5-oxo-4,5-dihydropyrazin-2-yl)-2-methylphenyl)-4,5,6,7-tetrahydrobenzo[b]thiophene-2-carboxamide (GDC-0834); 6-cyclopropyl-8-fluoro-2-(2-hydroxymethyl-3-{1-methyl-5-[5-(4-methyl-piperazin-1-yl)-pyridin-2-ylamino]-6-oxo-1,6-dihydro-pyridin-3-yl}-phenyl)-2H-isoquinolin-1-one (RN-486); N-[5-[5-(4-acetylpiperazine-1-carbonyl)-4-methoxy-2-methylphenyl]sulfanyl-1,3-thiazol-2-yl]-4-[(3,3-dimethylbutan-2-ylamino)methyl]benzamide (BMS-509744, HY-11092); or N-(5-((5-(4-Acetylpiperazine-1-carbonyl)-4-methoxy-2-methylphenyl)thio)thiazol-2-yl)-4-(((3-methylbutan-2-yl)amino)methyl)benzamide (HY11066); or a pharmaceutically acceptable salt thereof.

In some embodiments, the BTK inhibitor is:

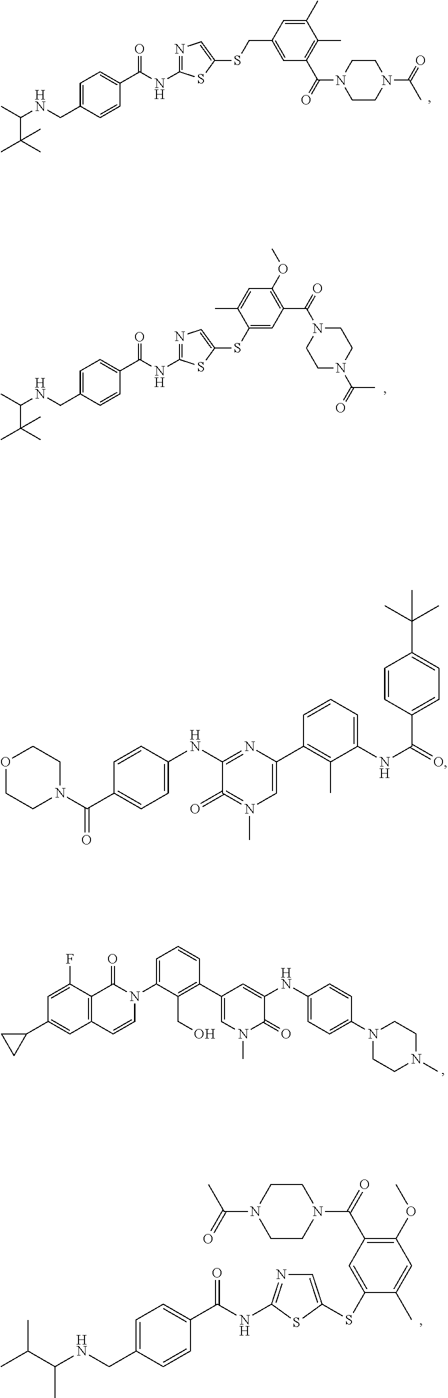

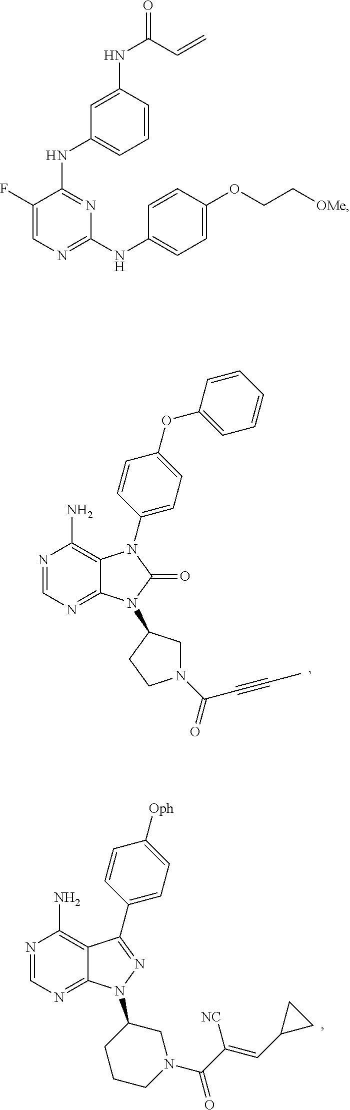

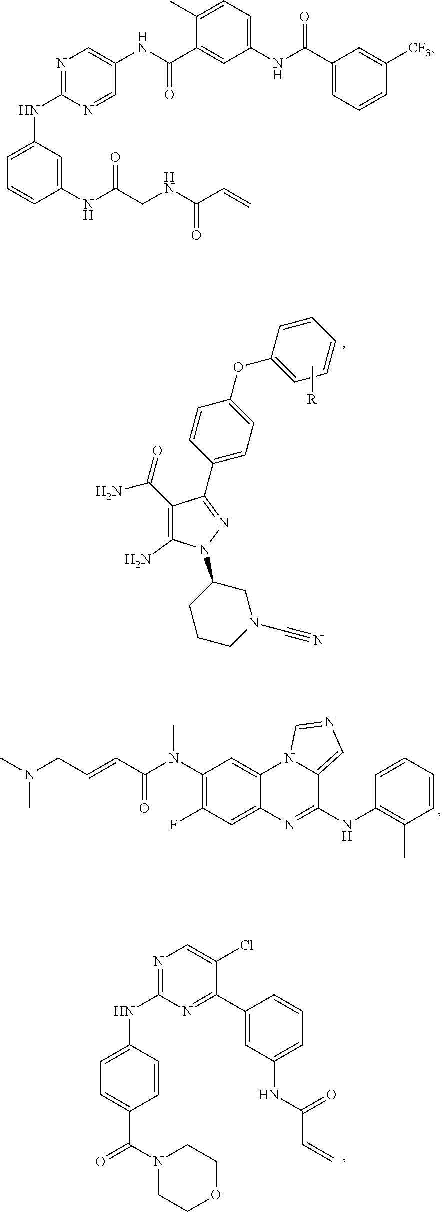

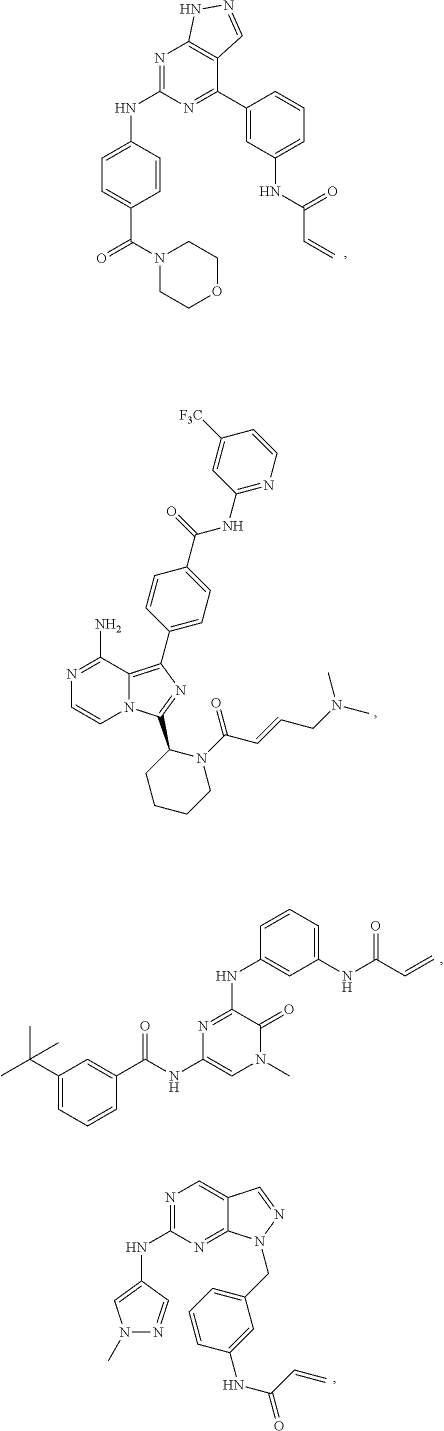

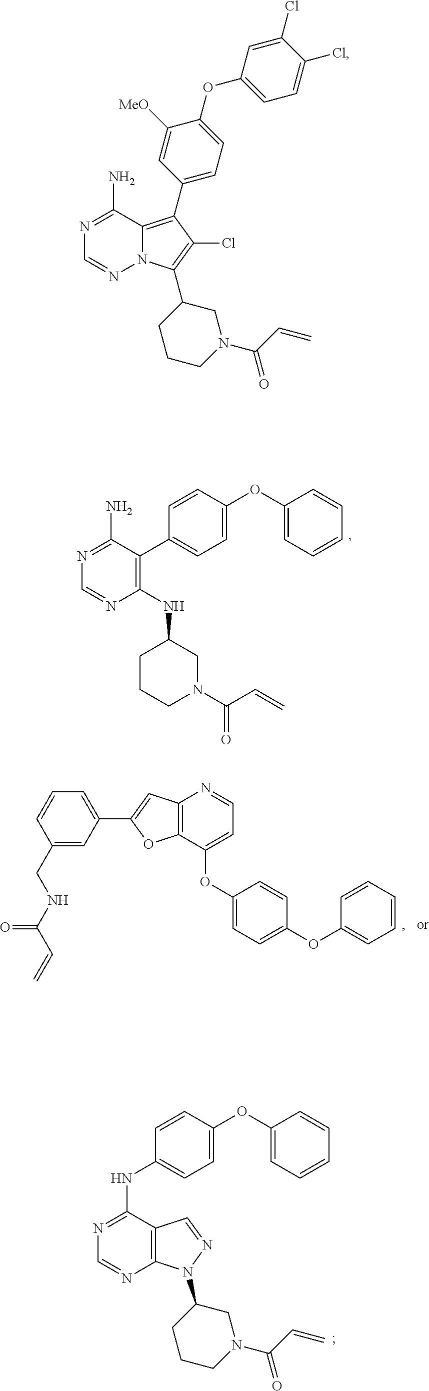 [SMILES: C=CC(=O)N1CCCC(c2c(Cl)c(-c3ccc(Oc4ccc(Cl)c(Cl)c4)c(OC)c3)c3c(N)ncnn23)C1 ; C=CC(=O)N1CCC[C@@H](Nc2ncnc(N)c2-c2ccc(Oc3ccccc3)cc2)C1 ; C=CC(=O)NCc1cccc(-c2cc3nccc(Oc4ccc(Oc5ccccc5)cc4)c3o2)c1 ; C=CC(=O)N1CCC[C@@H](n2ncc3c(Nc4ccc(Oc5ccccc5)cc4)ncnc32)C1]

or a pharmaceutically acceptable salt thereof.

### ITK Inhibitors

In some embodiments, the ITK inhibitor covalently binds to Cysteine 442 of ITK. In some embodiments, the ITK inhibitor is an ITK inhibitor compound described in WO2002/0500071, which is incorporated by reference in its entirety. In some embodiments, the ITK inhibitor is an ITK inhibitor compound described in WO2005/070420, which is incorporated by reference in its entirety. In some embodiments, the ITK inhibitor is an ITK inhibitor compound described in WO2005/079791, which is incorporated by reference in its entirety. In some embodiments, the ITK inhibitor is an ITK inhibitor compound described in WO2007/076228, which is incorporated by reference in its entirety. In some embodiments, the ITK inhibitor is an ITK inhibitor compound described in WO2007/058832, which is incorporated by reference in its entirety. In some embodiments, the ITK inhibitor is an ITK inhibitor compound described in WO2004/016610, which is incorporated by reference in its entirety. In some embodiments, the ITK inhibitor is an ITK inhibitor compound described in WO2004/016611, which is incorporated by reference in its entirety. In some embodiments, the ITK inhibitor is an ITK inhibitor compound described in WO2004/016600, which is incorporated by reference in its entirety. In some embodiments, the ITK inhibitor is an ITK inhibitor compound described in WO2004/016615, which is incorporated by reference in its entirety. In some embodiments, the ITK inhibitor is an ITK inhibitor compound described in WO2005/026175, which is incorporated by reference in its entirety. In some embodiments, the ITK inhibitor is an ITK inhibitor compound described in WO2006/065946, which is incorporated by reference in its entirety. In some embodiments, the ITK inhibitor is an ITK inhibitor compound described in WO2007/027594, which is incorporated by reference in its entirety. In some embodiments, the ITK inhibitor is an ITK inhibitor compound described in WO2007/017455, which is incorporated by reference in its entirety. In some embodiments, the ITK inhibitor is an ITK inhibitor compound described in WO2008/025820, which is incorporated by reference in its entirety. In some embodiments, the ITK inhibitor is an ITK inhibitor compound described in WO2008/025821, which is incorporated by reference in its entirety. In some embodiments, the ITK inhibitor is an ITK inhibitor compound described in WO2008/025822, which is incorporated by reference in its entirety. In some embodiments, the ITK inhibitor is an ITK inhibitor compound described in WO2011/017219, which is incorporated by reference in its entirety. In some embodiments, the ITK inhibitor is an ITK inhibitor compound described in WO2011/090760, which is incorporated by reference in its entirety. In some embodiments, the ITK inhibitor is an ITK inhibitor compound described in WO2009/158571, which is incorporated by reference in its entirety. In some embodiments, the ITK inhibitor is an ITK inhibitor compound described in WO2009/051822, which is incorporated by reference in its entirety. In some embodiments, the Itk inhibitor is an Itk inhibitor compound described in US 20110281850, which is incorporated by reference in its entirety. In some embodiments, the Itk inhibitor is an Itk inhibitor compound described in WO2014/082085, which is incorporated by reference in its entirety. In some embodiments, the Itk inhibitor is an Itk inhibitor compound described in WO2014/093383, which is incorporated by reference in its entirety. In some embodiments, the Itk inhibitor is an Itk inhibitor compound described in U.S. Pat. No. 8,759,358, which is incorporated by reference in its entirety. In some embodiments, the Itk inhibitor is an Itk inhibitor compound described in WO2014/105958, which is incorporated by reference in its entirety. In some embodiments, the Itk inhibitor is an Itk inhibitor compound described in US2014/0256704, which is incorporated by reference in its entirety. In some embodiments, the Itk inhibitor is an Itk inhibitor compound described in US20140315909, which is incorporated by reference in its entirety. In some embodiments, the Itk inhibitor is an Itk inhibitor compound described in US20140303161, which is incorporated by reference in its entirety. In some embodiments, the Itk inhibitor is an Itk inhibitor compound described in WO2014/145403, which is incorporated by reference in its entirety.

In some embodiments, the ITK inhibitor has a structure selected from:

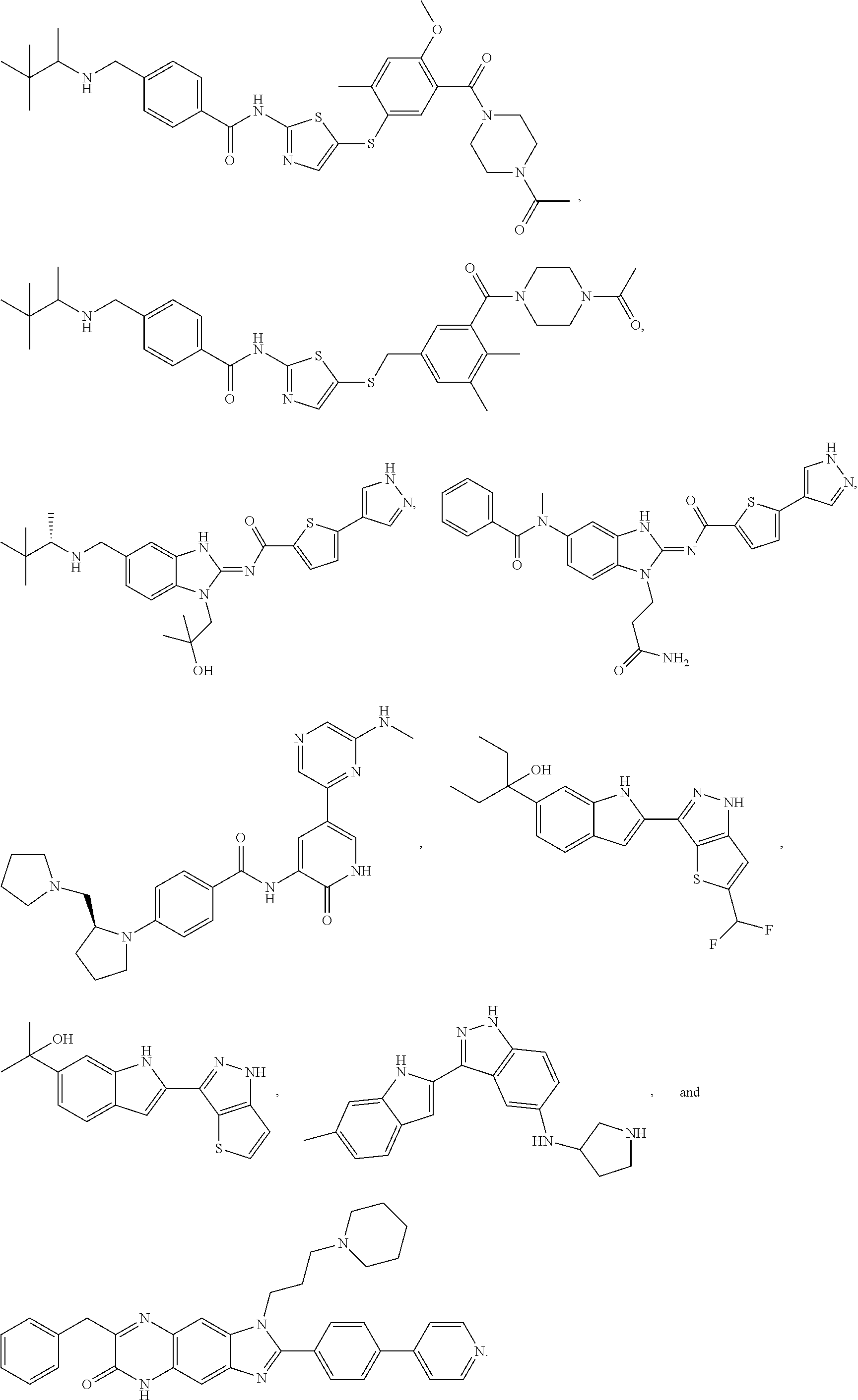

### Combination Therapy

In some embodiments, a TEC inhibitor is administered in combination with an additional therapeutic agent for the treatment of a hematological malignancy. In some embodiments, the TEC inhibitor is a BTK inhibitor, an ITK inhibitor, a TEC inhibitor, a RLK inhibitor, or a BMX inhibitor. In certain embodiments, an ITK inhibitor is administered in combination with an additional therapeutic agent for the treatment of a hematological malignancy. In certain embodiments, a BTK inhibitor (e.g. ibrutinib) is administered in combination with an additional therapeutic agent for the treatment of a hematological malignancy. In some embodiments, the additional therapeutic agent is a B cell receptor pathway inhibitor. In some embodiments, the B cell receptor pathway inhibitor is a CD79A inhibitor, a CD79B inhibitor, a CD19 inhibitor, a Lyn inhibitor, a Syk inhibitor, a PI3K inhibitor, a Blnk inhibitor, a PLCγ inhibitor, a PKCβ inhibitor, or a combination thereof. In some embodiments, the additional therapeutic agent is an antibody, B cell receptor signaling inhibitor, a PI3K inhibitor, an IAP inhibitor, an mTOR inhibitor, a radioimmunotherapeutic, a DNA damaging agent, a proteosome inhibitor, a histone deacetylase inhibitor, a protein kinase inhibitor, a hedgehog inhibitor, an Hsp90 inhibitor, a telomerase inhibitor, a Jak1/2 inhibitor, a protease inhibitor, a PKC inhibitor, a PARP inhibitor, or a combination thereof. In some embodiments, the additional therapeutic agent is an inhibitor of LYN, SYK, JAK, PI3K, PLCγ, MAPK, HDAC, NF_{K}B, or MEK. In some embodiments, the additional therapeutic agent is selected from a chemotherapeutic agent, a biologic agent, radiation therapy, bone marrow transplant or surgery.

In some embodiments, the additional therapeutic agent is selected from among a chemotherapeutic agent, a biologic agent, radiation therapy, bone marrow transplant or surgery. In some embodiments, the chemotherapeutic agent is selected from among chlorambucil, ifosfamide, doxorubicin, mesalazine, thalidomide, lenalidomide, temsirolimus, everolimus, fludarabine, fostamatinib, paclitaxel, docetaxel, ofatumumab, rituximab, dexamethasone, prednisone, CAL-101, ibritumomab, tositumomab, bortezomib, pentostatin, endostatin, or a combination thereof.

In some embodiments, the additional therapeutic agent comprises an agent selected from: bendamustine, bortezomib, lenalidomide, idelalisib (GS-1101), vorinostat, everolimus, panobinostat, temsirolimus, romidepsin, vorinostat, fludarabine, cyclophosphamide, mitoxantrone, pentostatine, prednisone, etopside, procarbazine, and thalidomide.

In some embodiments, the additional therapeutic agent is rituximab. In some embodiments, rituximab is further administered as a maintenance therapy.

In some embodiments the additional therapeutic agent is bendamustine. In some embodiments, bortezomib is administered in combination with rituximab.

In some embodiments, the additional therapeutic agent is bortezomib. In some embodiments, bendamustine is administered in combination with rituximab.

In some embodiments, the additional therapeutic agent is lenalidomide. In some embodiments, lenalidomide is administered in combination with rituximab.

In some embodiments, the additional therapeutic agent is a multi-agent therapeutic regimen. In some embodiments the additional therapeutic agent comprises the HyperCVAD regimen (cyclophosphamide, vincristine, doxorubicin, dexamethasone alternating with methotrexate and cytarabine). In some embodiments, the HyperCVAD regimen is administered in combination with rituximab.

In some embodiments the additional therapeutic agent comprises the R-CHOP regiment (rituximab, cyclophosphamide, doxorubicin, vincristine, and prednisone).

In some embodiments the additional therapeutic agent comprises bortezomib and rituximab.

In some embodiments the additional therapeutic agent comprises cladribine and rituximab.

In some embodiments the additional therapeutic agent comprises the FCR regimen (FCR (fludarabine, cyclophosphamide, rituximab).

In some embodiments the additional therapeutic agent comprises the FCMR regimen (fludarabine, cyclophosphamide, mitoxantrone, rituximab).

In some embodiments the additional therapeutic agent comprises the FMR regimen (fludarabine, mitoxantrone, rituximab).

In some embodiments the additional therapeutic agent comprises the PCR regimen (pentostatin, cyclophosphamide, rituximab).

In some embodiments the additional therapeutic agent comprises the PEPC regimen (prednisone, etoposide, procarbazine, cyclophosphamide).

In some embodiments the additional therapeutic agent comprises radioimmunotherapy with ⁹⁰Y-ibritumomab tiuxetan or ¹³¹I-tositumomab.

In some embodiments, the additional therapeutic agent is an autologous stem cell transplant.

In some embodiments, the additional therapeutic agent is selected from: Nitrogen Mustards such as for example, bendamustine, chlorambucil, chlormethine, cyclophosphamide, ifosfamide, melphalan, prednimustine, trofosfamide; Alkyl Sulfonates like busulfan, mannosulfan, treosulfan; Ethylene Imines like carboquone, thiotepa, triaziquone; Nitrosoureas like carmustine, fotemustine, lomustine, nimustine, ranimustine, semustine, streptozocin; Epoxides such as for example, etoglucid; Other Alkylating Agents such as for example dacarbazine, mitobronitol, pipobroman, temozolomide; Folic Acid Analogues such as for example methotrexate, permetrexed, pralatrexate, raltitrexed; Purine Analogs such as for example cladribine, clofarabine, fludarabine, mercaptopurine, nelarabine, tioguanine; Pyrimidine Analogs such as for example azacitidine, capecitabine, carmofur, cytarabine, decitabine, fluorouracil, gemcitabine, tegafur; Vinca Alkaloids such as for example vinblastine, vincristine, vindesine, vinflunine, vinorelbine; Podophyllotoxin Derivatives such as for example etoposide, teniposide; Colchicine derivatives such as for example demecolcine; Taxanes such as for example docetaxel, paclitaxel, paclitaxel poliglumex; Other Plant Alkaloids and Natural Products such as for example trabectedin; Actinomycines such as for example dactinomycin; Antracyclines such as for example aclarubicin, daunorubicin, doxorubicin, epirubicin, idarubicin, mitoxantrone, pirarubicin, valrubicin, zorubincin; Other Cytotoxic Antibiotics such as for example bleomycin, ixabepilone, mitomycin, plicamycin; Platinum Compounds such as for example carboplatin, cisplatin, oxaliplatin, satraplatin; Methylhydrazines such as for example procarbazine; Sensitizers such as for example aminolevulinic acid, efaproxiral, methyl aminolevulinate, porfimer sodium, temoporfin; Protein Kinase Inhibitors such as for example dasatinib, erlotinib, everolimus, gefitinib, imatinib, lapatinib, nilotinib, pazonanib, sorafenib, sunitinib, temsirolimus; Other Antineoplastic Agents such as for example alitretinoin, altretamine, amzacrine, anagrelide, arsenic trioxide, asparaginase, bexarotene, bortezomib, celecoxib, denileukin diftitox, estramustine, hydroxycarbamide, irinotecan, lonidamine, masoprocol, miltefosein, mitoguazone, mitotane, oblimersen, pegaspargase, pentostatin, romidepsin, sitimagene ceradenovec, tiazofurine, topotecan, tretinoin, vorinostat; Estrogens such as for example diethylstilbenol, ethinylestradiol, fosfestrol, polyestradiol phosphate; Progestogens such as for example gestonorone, medroxyprogesterone, megestrol; Gonadotropin Releasing Hormone Analogs such as for example buserelin, goserelin, leuprorelin, triptorelin; Anti-Estrogens such as for example fulvestrant, tamoxifen, toremifene; Anti-Androgens such as for example bicalutamide, flutamide, nilutamide, Enzyme Inhibitors, aminoglutethimide, anastrozole, exemestane, formestane, letrozole, vorozole; Other Hormone Antagonists such as for example abarelix, degarelix; Immunostimulants such as for example histamine dihydrochloride, mifamurtide, pidotimod, plerixafor, roquinimex, thymopentin; Immunosuppressants such as for example everolimus, gusperimus, leflunomide, mycophenolic acid, sirolimus; Calcineurin Inhibitors such as for example ciclosporin, tacrolimus; Other Immunosuppressants such as for example azathioprine, lenalidomide, methotrexate, thalidomide; and Radiopharmaceuticals such as for example, iobenguane.

In some embodiments, the additional therapeutic agent is selected from: interferons, interleukins, Tumor Necrosis Factors, Growth Factors, or the like.

In some embodiments, the additional therapeutic agent is selected from: ancestim, filgrastim, lenograstim, molgramostim, pegfilgrastim, sargramostim; Interferons such as for example interferon alfa natural, interferon alfa-2a, interferon alfa-2b, interferon alfacon-1, interferon alfa-n1, interferon beta natural, interferon beta-1a, interferon beta-1b, interferon gamma, peginterferon alfa-2a, peginterferon alfa-2b; Interleukins such as for example aldesleukin, oprelvekin; Other Immunostimulants such as for example BCG vaccine, glatiramer acetate, histamine dihydrochloride, immunocyanin, lentinan, melanoma vaccine, mifamurtide, pegademase, pidotimod, plerixafor, poly I:C, poly ICLC, roquinimex, tasonermin, thymopentin; Immunosuppressants such as for example abatacept, abetimus, alefacept, antilymphocyte immunoglobulin (horse), antithymocyte immunoglobulin (rabbit), eculizumab, efalizumab, everolimus, gusperimus, leflunomide, muromab-CD3, mycophenolic acid, natalizumab, sirolimus; TNF alpha Inhibitors such as for example adalimumab, afelimomab, certolizumab pegol, etanercept, golimumab, infliximab; Interleukin Inhibitors such as for example anakinra, basiliximab, canakinumab, daclizumab, mepolizumab, rilonacept, tocilizumab, ustekinumab; Calcineurin Inhibitors such as for example ciclosporin, tacrolimus; Other Immunosuppressants such as for example azathioprine, lenalidomide, methotrexate, thalidomide.

In some embodiments, the additional therapeutic agent is selected from: Adalimumab, Alemtuzumab, Basiliximab, Bevacizumab, Cetuximab, Certolizumab pegol, Daclizumab, Eculizumab, Efalizumab, Gemtuzumab, Ibritumomab tiuxetan, Infliximab, Muromonab-CD3, Natalizumab, Panitumumab, Ranibizumab, Rituximab, Tositumomab, Trastuzumab, or the like, or a combination thereof.

In some embodiments, the additional therapeutic agent is selected from: Monoclonal Antibodies such as for example alemtuzumab, bevacizumab, catumaxomab, cetuximab, edrecolomab, gemtuzumab, panitumumab, rituximab, trastuzumab; Immunosuppressants, eculizumab, efalizumab, muromab-CD3, natalizumab; TNF alpha Inhibitors such as for example adalimumab, afelimomab, certolizumab pegol, golimumab, infliximab; Interleukin Inhibitors, basiliximab, canakinumab, daclizumab, mepolizumab, tocilizumab, ustekinumab; Radiopharmaceuticals, ibritumomab tiuxetan, tositumomab; Others Monoclonal Antibodies such as for example abagovomab, adecatumumab, alemtuzumab, anti-CD30 monoclonal antibody Xmab2513, anti-MET monoclonal antibody MetMab, apolizumab, apomab, arcitumomab, basiliximab, bispecific antibody 2B1, blinatumomab, brentuximab vedotin, capromab pendetide, cixutumumab, claudiximab, conatumumab, dacetuzumab, denosumab, eculizumab, epratuzumab, epratuzumab, ertumaxomab, etaracizumab, figitumumab, fresolimumab, galiximab, ganitumab, gemtuzumab ozogamicin, glembatumumab, ibritumomab, inotuzumab ozogamicin, ipilimumab, lexatumumab, lintuzumab, lintuzumab, lucatumumab, mapatumumab, matuzumab, milatuzumab, monoclonal antibody CC49, necitumumab, nimotuzumab, oregovomab, pertuzumab, ramacurimab, ranibizumab, siplizumab, sonepcizumab, tanezumab, tositumomab, trastuzumab, tremelimumab, tucotuzumab celmoleukin, veltuzumab, visilizumab, volociximab, zalutumumab.

In some embodiments, the additional therapeutic agent is selected from: agents that affect the tumor micro-environment such as cellular signaling network (e.g. phosphatidylinositol 3-kinase (PI3K) signaling pathway, signaling from the B-cell receptor and the IgE receptor). In some embodiments, the additional therapeutic agent is a PI3K signaling inhibitor or a syc kinase inhibitor. In one embodiment, the syk inhibitor is R788. In another embodiment is a PKCγ inhibitor such as by way of example only, enzastaurin.

Examples of agents that affect the tumor micro-environment include PI3K signaling inhibitor, syc kinase inhibitor, Protein Kinase Inhibitors such as for example dasatinib, erlotinib, everolimus, gefitinib, imatinib, lapatinib, nilotinib, pazonanib, sorafenib, sunitinib, temsirolimus; Other Angiogenesis Inhibitors such as for example GT-111, JI-101, R1530; Other Kinase Inhibitors such as for example AC220, AC480, ACE-041, AMG 900, AP24534, Arry-614, AT7519, AT9283, AV-951, axitinib, AZD1152, AZD7762, AZD8055, AZD8931, bafetinib, BAY 73-4506, BGJ398, BGT226, BI 811283, BI6727, BIBF 1120, BIBW 2992, BMS-690154, BMS-777607, BMS-863233, BSK-461364, CAL-101, CEP-11981, CYC116, DCC-2036, dinaciclib, dovitinib lactate, E7050, EMD 1214063, ENMD-2076, fostamatinib disodium, GSK2256098, GSK690693, INCB18424, INNO-406, JNJ-26483327, JX-594, KX2-391, linifanib, LY2603618, MGCD265, MK-0457, MK1496, MLN8054, MLN8237, MP470, NMS-1116354, NMS-1286937, ON 01919.Na, OSI-027, OSI-930, Btk inhibitor, PF-00562271, PF-02341066, PF-03814735, PF-04217903, PF-04554878, PF-04691502, PF-3758309, PHA-739358, PLC3397, progenipoietin, R547, R763, ramucirumab, regorafenib, RO5185426, SAR103168, SCH 727965, SGI-1176, SGX523, SNS-314, TAK-593, TAK-901, TKI258, TLN-232, TTP607, XL147, XL228, XL281RO5126766, XL418, XL765.

In some embodiments, the additional therapeutic agent is selected from inhibitors of mitogen-activated protein kinase signaling, e.g., U0126, PD98059, PD184352, PD0325901, ARRY-142886, SB239063, SP600125, BAY 43-9006, wortmannin, or LY294002; Syk inhibitors; mTOR inhibitors; and antibodies (e.g., rituxan).

In some embodiments, the additional therapeutic agent is selected from: Adriamycin, Dactinomycin, Bleomycin, Vinblastine, Cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; hydroxyurea; idarubicin hydrochloride; ifosfamide; iimofosine; interleukin I1 (including recombinant interleukin II, or r1L2), interferon alfa-2a; interferon alfa-2b; interferon alfa-n1; interferon alfa-n3; interferon beta-1 a; interferon gamma-1 b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride.

In some embodiments, the additional therapeutic agent is selected from: 20-epi-1, 25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; 9-dioxamycin; diphenyl spiromustine; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-such as for example growth factor-1 receptor inhibitor; interferon agonists; interferons; interleukins; iobenguane; iododoxorubicin; ipomeanol, 4−; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylerie conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen-binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer.

In some embodiments, the additional therapeutic agent is selected from: alkylating agents, antimetabolites, natural products, or hormones, e.g., nitrogen mustards (e.g., mechloroethamine, cyclophosphamide, chlorambucil, etc.), alkyl sulfonates (e.g., busulfan), nitrosoureas (e.g., carmustine, lomustine, etc.), or triazenes (decarbazine, etc.). Examples of antimetabolites include but are not limited to folic acid analog (e.g., methotrexate), or pyrimidine analogs (e.g., Cytarabine), purine analogs (e.g., mercaptopurine, thioguanine, pentostatin).

In some embodiments, the additional therapeutic agent is selected from: nitrogen mustards (e.g., mechloroethamine, cyclophosphamide, chlorambucil, melphalan, etc.), ethylenimine and methylmelamines (e.g., hexamethylmelamine, thiotepa), alkyl sulfonates (e.g., busulfan), nitrosoureas (e.g., carmustine, lomustine, semustine, streptozocin, etc.), or triazenes (decarbazine, etc.). Examples of antimetabolites include, but are not limited to folic acid analog (e.g., methotrexate), or pyrimidine analogs (e.g., fluorouracil, floxuridine, Cytarabine), purine analogs (e.g., mercaptopurine, thioguanine, pentostatin.

In some embodiments, the additional therapeutic agent is selected from: agents which act by arresting cells in the G2-M phases due to stabilized microtubules, e.g., Erbulozole (also known as R-55104), Dolastatin 10 (also known as DLS-10 and NSC-376128), Mivobulin isethionate (also known as CI-980), Vincristine, NSC-639829, Discodermolide (also known as NVP-XX-A-296), ABT-751 (Abbott, also known as E-7010), Altorhyrtins (such as Altorhyrtin A and Altorhyrtin C), Spongistatins (such as Spongistatin 1, Spongistatin 2, Spongistatin 3, Spongistatin 4, Spongistatin 5, Spongistatin 6, Spongistatin 7, Spongistatin 8, and Spongistatin 9), Cemadotin hydrochloride (also known as LU-103793 and NSC-D-669356), Epothilones (such as Epothilone A, Epothilone B, Epothilone C (also known as desoxyepothilone A or dEpoA), Epothilone D (also referred to as KOS-862, dEpoB, and desoxyepothilone B), Epothilone E, Epothilone F, Epothilone B N-oxide, Epothilone A N-oxide, 16-aza-epothilone B, 21-aminoepothilone B (also known as BMS-310705), 21-hydroxyepothilone D (also known as Desoxyepothilone F and dEpoF), 26-fluoroepothilone), Auristatin PE (also known as NSC-654663), Soblidotin (also known as TZT-1027), LS-4559-P (Pharmacia, also known as LS-4577), LS-4578 (Pharmacia, also known as LS-477-P), LS-4477 (Pharmacia), LS-4559 (Pharmacia), RPR-112378 (Aventis), Vincristine sulfate, DZ-3358 (Daiichi), FR-182877 (Fujisawa, also known as WS-9885B), GS-164 (Takeda), GS-198 (Takeda), KAR-2 (Hungarian Academy of Sciences), BSF-223651 (BASF, also known as ILX-651 and LU-223651), SAH-49960 (Lilly/Novartis), SDZ-268970 (Lilly/Novartis), AM-97 (Armad/Kyowa Hakko), AM-132 (Armad), AM-138 (Armad/Kyowa Hakko), IDN-5005 (Indena), Cryptophycin 52 (also known as LY-355703), AC-7739 (Ajinomoto, also known as AVE-8063A and CS-39.HCl), AC-7700 (Ajinomoto, also known as AVE-8062, AVE-8062A, CS-39-L-Ser.HCl, and RPR-258062A), Vitilevuamide, Tubulysin A, Canadensol, Centaureidin (also known as NSC-106969), T-138067 (Tularik, also known as T-67, TL-138067 and TI-138067), COBRA-1 (Parker Hughes Institute, also known as DDE-261 and WHI-261), H10 (Kansas State University), H16 (Kansas State University), Oncocidin A1 (also known as BTO-956 and DIME), DDE-313 (Parker Hughes Institute), Fijianolide B, Laulimalide, SPA-2 (Parker Hughes Institute), SPA-1 (Parker Hughes Institute, also known as SPIKET-P), 3-IAABU (Cytoskeleton/Mt. Sinai School of Medicine, also known as MF-569), Narcosine (also known as NSC-5366), Nascapine, D-24851 (Asta Medica), A-105972 (Abbott), Hemiasterlin, 3-BAABU (Cytoskeleton/Mt. Sinai School of Medicine, also known as MF-191), TMPN (Arizona State University), Vanadocene acetylacetonate, T-138026 (Tularik), Monsatrol, lnanocine (also known as NSC-698666), 3-1AABE (Cytoskeleton/Mt. Sinai School of Medicine), A-204197 (Abbott), T-607 (Tuiarik, also known as T-900607), RPR-115781 (Aventis), Eleutherobins (such as Desmethyleleutherobin, Desaetyleleutherobin, lsoeleutherobin A, and Z-Eleutherobin), Caribaeoside, Caribaeolin, Halichondrin B, D-64131 (Asta Medica), D-68144 (Asta Medica), Diazonamide A, A-293620 (Abbott), NPI-2350 (Nereus), Taccalonolide A, TUB-245 (Aventis), A-259754 (Abbott), Diozostatin, (−)-Phenylahistin (also known as NSCL-96F037), D-68838 (Asta Medica), D-68836 (Asta Medica), Myoseverin B, D-43411 (Zentaris, also known as D-81862), A-289099 (Abbott), A-318315 (Abbott), HTI-286 (also known as SPA-110, trifluoroacetate salt) (Wyeth), D-82317 (Zentaris), D-82318 (Zentaris), SC-12983 (NCI), Resverastatin phosphate sodium, BPR-OY-007 (National Health Research Institutes), and SSR-250411 (Sanofi).

### Pharmaceutical Compositions/Formulations

In some embodiments, pharmaceutical compositions are formulated in a conventional manner using one or more physiologically acceptable carriers including excipients and auxiliaries which facilitate processing of the active compounds into preparations which can be used pharmaceutically. Proper formulation is dependent upon the route of administration chosen. Any of the well-known techniques, carriers, and excipients can be used as suitable and as understood in the art. A summary of pharmaceutical compositions described herein can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference in their entirety.

A pharmaceutical composition, as used herein, refers to a mixture of a compound described herein, such as, for example, ibrutinib, with other chemical components, such as carriers, stabilizers, diluents, dispersing agents, suspending agents, thickening agents, and/or excipients. The pharmaceutical composition facilitates administration of the compound to an organism. In practicing the methods of treatment or use provided herein, therapeutically effective amounts of compounds described herein are administered in a pharmaceutical composition to a mammal having a disease, disorder, or condition to be treated. Preferably, the mammal is a human. A therapeutically effective amount can vary widely depending on the severity of the disease, the age and relative health of the subject, the potency of the compound used and other factors. The compounds can be used singly or in combination with one or more therapeutic agents as components of mixtures.

In certain embodiments, compositions also include one or more pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

In other embodiments, compositions also include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

The term “pharmaceutical combination” as used herein, means a product that results from the mixing or combining of more than one active ingredient and includes both fixed and non-fixed combinations of the active ingredients. The term “fixed combination” means that the active ingredients, e.g. a compound described herein and a co-agent, are both administered to a patient simultaneously in the form of a single entity or dosage. The term “non-fixed combination” means that the active ingredients, e.g. a compound described herein and a co-agent, are administered to a patient as separate entities either simultaneously, concurrently or sequentially with no specific intervening time limits, wherein such administration provides effective levels of the two compounds in the body of the patient. The latter also applies to cocktail therapy, e.g. the administration of three or more active ingredients.

The pharmaceutical formulations described herein can be administered to a subject by multiple administration routes, including but not limited to, oral, parenteral (e.g., intravenous, subcutaneous, intramuscular), intranasal, buccal, topical, rectal, or transdermal administration routes. The pharmaceutical formulations described herein include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations, and mixed immediate and controlled release formulations.

In some embodiments, pharmaceutical compositions including a compound described herein are manufactured in a conventional manner, such as, by way of example only, by means of conventional mixing, dissolving, granulating, dragee-making, levigating, emulsifying, encapsulating, entrapping or compression processes.

“Antifoaming agents” reduce foaming during processing which can result in coagulation of aqueous dispersions, bubbles in the finished film, or generally impair processing. Exemplary anti-foaming agents include silicon emulsions or sorbitan sesquioleate.

“Antioxidants” include, for example, butylated hydroxytoluene (BHT), sodium ascorbate, ascorbic acid, sodium metabisulfite and tocopherol. In certain embodiments, antioxidants enhance chemical stability where required.

In certain embodiments, compositions provided herein also include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

In some embodiments, formulations described herein benefit from antioxidants, metal chelating agents, thiol containing compounds and other general stabilizing agents. Examples of such stabilizing agents, include, but are not limited to: (a) about 0.5% to about 2% w/v glycerol, (b) about 0.1% to about 1% w/v methionine, (c) about 0.1% to about 2% w/v monothioglycerol, (d) about 1 mM to about 10 mM EDTA, (e) about 0.01% to about 2% w/v ascorbic acid, (f) 0.003% to about 0.02% w/v polysorbate 80, (g) 0.001% to about 0.05% w/v. polysorbate 20, (h) arginine, (i) heparin, (j) dextran sulfate, (k) cyclodextrins, (l) pentosan polysulfate and other heparinoids, (m) divalent cations such as magnesium and zinc; or (n) combinations thereof.

“Binders” impart cohesive qualities and include, e.g., alginic acid and salts thereof; cellulose derivatives such as carboxymethylcellulose, methylcellulose (e.g., Methocel®), hydroxypropylmethylcellulose, hydroxyethylcellulose, hydroxypropylcellulose (e.g., Klucel®), ethylcellulose (e.g., Ethocel®), and microcrystalline cellulose (e.g., Avicel®); microcrystalline dextrose; amylose; magnesium aluminum silicate; polysaccharide acids; bentonites; gelatin; polyvinylpyrrolidone/vinyl acetate copolymer; crospovidone; povidone; starch; pregelatinized starch; tragacanth, dextrin, a sugar, such as sucrose (e.g., Dipac®), glucose, dextrose, molasses, mannitol, sorbitol, xylitol (e.g., Xylitab®), and lactose; a natural or synthetic gum such as acacia, tragacanth, ghatti gum, mucilage of isapol husks, polyvinylpyrrolidone (e.g., Polyvidone® CL, Kollidon® CL, Polyplasdone® XL-10), larch arabogalactan, Veegum®, polyethylene glycol, waxes, sodium alginate, and the like.

A “carrier” or “carrier materials” include any commonly used excipients in pharmaceutics and should be selected on the basis of compatibility with compounds disclosed herein, such as, compounds of ibrutinib, and the release profile properties of the desired dosage form. Exemplary carrier materials include, e.g., binders, suspending agents, disintegration agents, filling agents, surfactants, solubilizers, stabilizers, lubricants, wetting agents, diluents, and the like. “Pharmaceutically compatible carrier materials” include, but are not limited to, acacia, gelatin, colloidal silicon dioxide, calcium glycerophosphate, calcium lactate, maltodextrin, glycerine, magnesium silicate, polyvinylpyrrollidone (PVP), cholesterol, cholesterol esters, sodium caseinate, soy lecithin, taurocholic acid, phosphotidylcholine, sodium chloride, tricalcium phosphate, dipotassium phosphate, cellulose and cellulose conjugates, sugars sodium stearoyl lactylate, carrageenan, monoglyceride, diglyceride, pregelatinized starch, and the like. See, e.g., Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

“Dispersing agents,” and/or “viscosity modulating agents” include materials that control the diffusion and homogeneity of a drug through liquid media or a granulation method or blend method. In some embodiments, these agents also facilitate the effectiveness of a coating or eroding matrix. Exemplary diffusion facilitators/dispersing agents include, e.g., hydrophilic polymers, electrolytes, Tween® 60 or 80, PEG, polyvinylpyrrolidone (PVP; commercially known as Plasdone®), and the carbohydrate-based dispersing agents such as, for example, hydroxypropyl celluloses (e.g., HPC, HPC-SL, and HPC-L), hydroxypropyl methylcelluloses (e.g., HPMC K100, HPMC K4M, HPMC K15M, and HPMC K100M), carboxymethylcellulose sodium, methylcellulose, hydroxyethylcellulose, hydroxypropylcellulose, hydroxypropylmethylcellulose phthalate, hydroxypropylmethylcellulose acetate stearate (HPMCAS), noncrystalline cellulose, magnesium aluminum silicate, triethanolamine, polyvinyl alcohol (PVA), vinyl pyrrolidone/vinyl acetate copolymer (S630), 4-(1,1,3,3-tetramethylbutyl)-phenol polymer with ethylene oxide and formaldehyde (also known as tyloxapol), poloxamers (e.g., Pluronics F68®, F88®, and F108®, which are block copolymers of ethylene oxide and propylene oxide); and poloxamines (e.g., Tetronic 908®, also known as Poloxamine 908®, which is a tetrafunctional block copolymer derived from sequential addition of propylene oxide and ethylene oxide to ethylenediamine (BASF Corporation, Parsippany, N.J.)), polyvinylpyrrolidone K12, polyvinylpyrrolidone K17, polyvinylpyrrolidone K25, or polyvinylpyrrolidone K30, polyvinylpyrrolidone/vinyl acetate copolymer (S-630), polyethylene glycol, e.g., the polyethylene glycol can have a molecular weight of about 300 to about 6000, or about 3350 to about 4000, or about 7000 to about 5400, sodium carboxymethylcellulose, methylcellulose, polysorbate-80, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, polysorbate-80, sodium alginate, polyethoxylated sorbitan monolaurate, polyethoxylated sorbitan monolaurate, povidone, carbomers, polyvinyl alcohol (PVA), alginates, chitosans and combinations thereof. Plasticizers such as cellulose or triethyl cellulose can also be used as dispersing agents. Dispersing agents particularly useful in liposomal dispersions and self-emulsifying dispersions are dimyristoyl phosphatidyl choline, natural phosphatidyl choline from eggs, natural phosphatidyl glycerol from eggs, cholesterol and isopropyl myristate.

Combinations of one or more erosion facilitator with one or more diffusion facilitator can also be used in the present compositions.

The term “diluent” refers to chemical compounds that are used to dilute the compound of interest prior to delivery. Diluents can also be used to stabilize compounds because they can provide a more stable environment. Salts dissolved in buffered solutions (which also can provide pH control or maintenance) are utilized as diluents in the art, including, but not limited to a phosphate buffered saline solution. In certain embodiments, diluents increase bulk of the composition to facilitate compression or create sufficient bulk for homogenous blend for capsule filling. Such compounds include e.g., lactose, starch, mannitol, sorbitol, dextrose, microcrystalline cellulose such as Avicel®; dibasic calcium phosphate, dicalcium phosphate dihydrate; tricalcium phosphate, calcium phosphate; anhydrous lactose, spray-dried lactose; pregelatinized starch, compressible sugar, such as Di-Pac® (Amstar); mannitol, hydroxypropylmethylcellulose, hydroxypropylmethylcellulose acetate stearate, sucrose-based diluents, confectioner's sugar; monobasic calcium sulfate monohydrate, calcium sulfate dihydrate; calcium lactate trihydrate, dextrates; hydrolyzed cereal solids, amylose; powdered cellulose, calcium carbonate; glycine, kaolin; mannitol, sodium chloride; inositol, bentonite, and the like.

The term “disintegrate” includes both the dissolution and dispersion of the dosage form when contacted with gastrointestinal fluid. “Disintegration agents or disintegrants” facilitate the breakup or disintegration of a substance. Examples of disintegration agents include a starch, e.g., a natural starch such as corn starch or potato starch, a pregelatinized starch such as National 1551 or Amijel®, or sodium starch glycolate such as Promogel® or Explotab®, a cellulose such as a wood product, methylcrystalline cellulose, e.g., Avicel®, Avicel® PH101, Avicel® PH102, Avicel® PH105, Elcema® P100, Emcocel®, Vivacel®, Ming Tia®, and Solka-Floc®, methylcellulose, croscarmellose, or a cross-linked cellulose, such as cross-linked sodium carboxymethylcellulose (Ac-Di-Sol®), cross-linked carboxymethylcellulose, or cross-linked croscarmellose, a cross-linked starch such as sodium starch glycolate, a cross-linked polymer such as crospovidone, a cross-linked polyvinylpyrrolidone, alginate such as alginic acid or a salt of alginic acid such as sodium alginate, a clay such as Veegum® HV (magnesium aluminum silicate), a gum such as agar, guar, locust bean, Karaya, pectin, or tragacanth, sodium starch glycolate, bentonite, a natural sponge, a surfactant, a resin such as a cation-exchange resin, citrus pulp, sodium lauryl sulfate, sodium lauryl sulfate in combination starch, and the like.

“Drug absorption” or “absorption” typically refers to the process of movement of drug from site of administration of a drug across a barrier into a blood vessel or the site of action, e.g., a drug moving from the gastrointestinal tract into the portal vein or lymphatic system.

An “enteric coating” is a substance that remains substantially intact in the stomach but dissolves and releases the drug in the small intestine or colon. Generally, the enteric coating comprises a polymeric material that prevents release in the low pH environment of the stomach but that ionizes at a higher pH, typically a pH of 6 to 7, and thus dissolves sufficiently in the small intestine or colon to release the active agent therein.

“Erosion facilitators” include materials that control the erosion of a particular material in gastrointestinal fluid. Erosion facilitators are generally known to those of ordinary skill in the art. Exemplary erosion facilitators include, e.g., hydrophilic polymers, electrolytes, proteins, peptides, and amino acids.

“Filling agents” include compounds such as lactose, calcium carbonate, calcium phosphate, dibasic calcium phosphate, calcium sulfate, microcrystalline cellulose, cellulose powder, dextrose, dextrates, dextran, starches, pregelatinized starch, sucrose, xylitol, lactitol, mannitol, sorbitol, sodium chloride, polyethylene glycol, and the like.

“Flavoring agents” and/or “sweeteners” useful in the formulations described herein, include, e.g., acacia syrup, acesulfame K, alitame, anise, apple, aspartame, banana, Bavarian cream, berry, black currant, butterscotch, calcium citrate, camphor, caramel, cherry, cherry cream, chocolate, cinnamon, bubble gum, citrus, citrus punch, citrus cream, cotton candy, cocoa, cola, cool cherry, cool citrus, cyclamate, cylamate, dextrose, eucalyptus, eugenol, fructose, fruit punch, ginger, glycyrrhetinate, glycyrrhiza (licorice) syrup, grape, grapefruit, honey, isomalt, lemon, lime, lemon cream, monoammonium glyrrhizinate (MagnaSweet®), maltol, mannitol, maple, marshmallow, menthol, mint cream, mixed berry, neohesperidine DC, neotame, orange, pear, peach, peppermint, peppermint cream, Prosweet® Powder, raspberry, root beer, rum, saccharin, safrole, sorbitol, spearmint, spearmint cream, strawberry, strawberry cream, stevia, sucralose, sucrose, sodium saccharin, saccharin, aspartame, acesulfame potassium, mannitol, talin, sylitol, sucralose, sorbitol, Swiss cream, tagatose, tangerine, thaumatin, tutti fruitti, vanilla, walnut, watermelon, wild cherry, wintergreen, xylitol, or any combination of these flavoring ingredients, e.g., anise-menthol, cherry-anise, cinnamon-orange, cherry-cinnamon, chocolate-mint, honey-lemon, lemon-lime, lemon-mint, menthol-eucalyptus, orange-cream, vanilla-mint, and mixtures thereof.

“Lubricants” and “glidants” are compounds that prevent, reduce or inhibit adhesion or friction of materials. Exemplary lubricants include, e.g., stearic acid, calcium hydroxide, talc, sodium stearyl fumerate, a hydrocarbon such as mineral oil, or hydrogenated vegetable oil such as hydrogenated soybean oil (Sterotex®), higher fatty acids and their alkali-metal and alkaline earth metal salts, such as aluminum, calcium, magnesium, zinc, stearic acid, sodium stearates, glycerol, talc, waxes, Stearowet®, boric acid, sodium benzoate, sodium acetate, sodium chloride, leucine, a polyethylene glycol (e.g., PEG-4000) or a methoxypolyethylene glycol such as Carbowax™, sodium oleate, sodium benzoate, glyceryl behenate, polyethylene glycol, magnesium or sodium lauryl sulfate, colloidal silica such as Syloid™, Cab-O-Sil®, a starch such as corn starch, silicone oil, a surfactant, and the like.

A “measurable serum concentration” or “measurable plasma concentration” describes the blood serum or blood plasma concentration, typically measured in mg, μg, or ng of therapeutic agent per mL, dL, or L of blood serum, absorbed into the bloodstream after administration. As used herein, measurable plasma concentrations are typically measured in ng/ml or μg/ml.

“Pharmacodynamics” refers to the factors which determine the biologic response observed relative to the concentration of drug at a site of action.

“Pharmacokinetics” refers to the factors which determine the attainment and maintenance of the appropriate concentration of drug at a site of action.

“Plasticizers” are compounds used to soften the microencapsulation material or film coatings to make them less brittle. Suitable plasticizers include, e.g., polyethylene glycols such as PEG 300, PEG 400, PEG 600, PEG 1450, PEG 3350, and PEG 800, stearic acid, propylene glycol, oleic acid, triethyl cellulose and triacetin. In some embodiments, plasticizers can also function as dispersing agents or wetting agents.

“Solubilizers” include compounds such as triacetin, triethylcitrate, ethyl oleate, ethyl caprylate, sodium lauryl sulfate, sodium doccusate, vitamin E TPGS, dimethylacetamide, N-methylpyrrolidone, N-hydroxyethylpyrrolidone, polyvinylpyrrolidone, hydroxypropylmethyl cellulose, hydroxypropyl cyclodextrins, ethanol, n-butanol, isopropyl alcohol, cholesterol, bile salts, polyethylene glycol 200-600, glycofurol, transcutol, propylene glycol, and dimethyl isosorbide and the like.

“Stabilizers” include compounds such as any antioxidation agents, buffers, acids, preservatives and the like.

“Steady state,” as used herein, is when the amount of drug administered is equal to the amount of drug eliminated within one dosing interval resulting in a plateau or constant plasma drug exposure.

“Suspending agents” include compounds such as polyvinylpyrrolidone, e.g., polyvinylpyrrolidone K12, polyvinylpyrrolidone K17, polyvinylpyrrolidone K25, or polyvinylpyrrolidone K30, vinyl pyrrolidone/vinyl acetate copolymer (S630), polyethylene glycol, e.g., the polyethylene glycol can have a molecular weight of about 300 to about 6000, or about 3350 to about 4000, or about 7000 to about 5400, sodium carboxymethylcellulose, methylcellulose, hydroxypropylmethylcellulose, hydroxymethylcellulose acetate stearate, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monolaurate, povidone and the like.

“Surfactants” include compounds such as sodium lauryl sulfate, sodium docusate, Tween 60 or 80, triacetin, vitamin E TPGS, sorbitan monooleate, polyoxyethylene sorbitan monooleate, polysorbates, polaxomers, bile salts, glyceryl monostearate, copolymers of ethylene oxide and propylene oxide, e.g., Pluronic® (BASF), and the like. Some other surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40. In some embodiments, surfactants are included to enhance physical stability or for other purposes.

“Viscosity enhancing agents” include, e.g., methyl cellulose, xanthan gum, carboxymethyl cellulose, hydroxypropyl cellulose, hydroxypropylmethyl cellulose, hydroxypropylmethyl cellulose acetate stearate, hydroxypropylmethyl cellulose phthalate, carbomer, polyvinyl alcohol, alginates, acacia, chitosans and combinations thereof

“Wetting agents” include compounds such as oleic acid, glyceryl monostearate, sorbitan monooleate, sorbitan monolaurate, triethanolamine oleate, polyoxyethylene sorbitan monooleate, polyoxyethylene sorbitan monolaurate, sodium docusate, sodium oleate, sodium lauryl sulfate, sodium doccusate, triacetin, Tween 80, vitamin E TPGS, ammonium salts and the like.

### Dosage Forms

The compositions described herein can be formulated for administration to a subject via any conventional means including, but not limited to, oral, parenteral (e.g., intravenous, subcutaneous, or intramuscular), buccal, intranasal, rectal or transdermal administration routes. As used herein, the term “subject” is used to mean an animal, preferably a mammal, including a human or non-human. As used herein, the terms patient and subject are used interchangeably.

Moreover, the pharmaceutical compositions described herein, which include ibrutinib can be formulated into any suitable dosage form, including but not limited to, aqueous oral dispersions, liquids, gels, syrups, elixirs, slurries, suspensions and the like, for oral ingestion by a patient to be treated, solid oral dosage forms, aerosols, controlled release formulations, fast melt formulations, effervescent formulations, lyophilized formulations, tablets, powders, pills, dragees, capsules, delayed release formulations, extended release formulations, pulsatile release formulations, multiparticulate formulations, and mixed immediate release and controlled release formulations.

Pharmaceutical preparations for oral use can be obtained by mixing one or more solid excipient with one or more of the compounds described herein, optionally grinding the resulting mixture, and processing the mixture of granules, after adding suitable auxiliaries, if desired, to obtain tablets or dragee cores. Suitable excipients include, for example, fillers such as sugars, including lactose, sucrose, mannitol, or sorbitol; cellulose preparations such as, for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. In some embodiments, disintegrating agents are added, such as the cross-linked croscarmellose sodium, polyvinylpyrrolidone, agar, or alginic acid or a salt thereof such as sodium alginate.

Dragee cores are provided with suitable coatings. For this purpose, in some embodiments, concentrated sugar solutions are used, which, in particular embodiments, optionally contain gum arabic, talc, polyvinylpyrrolidone, carbopol gel, polyethylene glycol, and/or titanium dioxide, lacquer solutions, and suitable organic solvents or solvent mixtures. In some embodiments, dyestuffs or pigments are added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In some embodiments, in soft capsules, the active compounds are dissolved or suspended in suitable liquids, such as fatty oils, liquid paraffin, or liquid polyethylene glycols. In addition, in some embodiments, stabilizers are added. All formulations for oral administration should be in dosages suitable for such administration.

In some embodiments, the solid dosage forms disclosed herein are in the form of a tablet, (including a suspension tablet, a fast-melt tablet, a bite-disintegration tablet, a rapid-disintegration tablet, an effervescent tablet, or a caplet), a pill, a powder (including a sterile packaged powder, a dispensable powder, or an effervescent powder) a capsule (including both soft or hard capsules, e.g., capsules made from animal-derived gelatin or plant-derived HPMC, or “sprinkle capsules”), solid dispersion, solid solution, bioerodible dosage form, controlled release formulations, pulsatile release dosage forms, multiparticulate dosage forms, pellets, granules, or an aerosol. In other embodiments, the pharmaceutical formulation is in the form of a powder. In still other embodiments, the pharmaceutical formulation is in the form of a tablet, including but not limited to, a fast-melt tablet. Additionally, in some embodiments, pharmaceutical formulations described herein are administered as a single capsule or in multiple capsule dosage form. In some embodiments, the pharmaceutical formulation is administered in two, or three, or four, capsules or tablets.

In some embodiments, solid dosage forms, e.g., tablets, effervescent tablets, and capsules, are prepared by mixing particles of ibrutinib, with one or more pharmaceutical excipients to form a bulk blend composition. When referring to these bulk blend compositions as homogeneous, it is meant that the particles of ibrutinib are dispersed evenly throughout the composition so that the composition can be readily subdivided into equally effective unit dosage forms, such as tablets, pills, and capsules. In some embodiments, the individual unit dosages also include film coatings, which disintegrate upon oral ingestion or upon contact with diluent. These formulations can be manufactured by conventional pharmacological techniques.

Conventional pharmacological techniques include, e.g., one or a combination of methods: (1) dry mixing, (2) direct compression, (3) milling, (4) dry or non-aqueous granulation, (5) wet granulation, or (6) fusion. See, e.g., Lachman et al., The Theory and Practice of Industrial Pharmacy (1986). Other methods include, e.g., spray drying, pan coating, melt granulation, granulation, fluidized bed spray drying or coating (e.g., wurster coating), tangential coating, top spraying, tableting, extruding and the like.

The pharmaceutical solid dosage forms described herein can include a compound described herein and one or more pharmaceutically acceptable additives such as a compatible carrier, binder, filling agent, suspending agent, flavoring agent, sweetening agent, disintegrating agent, dispersing agent, surfactant, lubricant, colorant, diluent, solubilizer, moistening agent, plasticizer, stabilizer, penetration enhancer, wetting agent, anti-foaming agent, antioxidant, preservative, or one or more combination thereof. In still other aspects, using standard coating procedures, such as those described in Remington's Pharmaceutical Sciences, 20th Edition (2000), a film coating is provided around the formulation of ibrutinib. In another embodiment, some or all of the particles of ibrutinib, are not microencapsulated and are uncoated.

Suitable carriers for use in the solid dosage forms described herein include, but are not limited to, acacia, gelatin, colloidal silicon dioxide, calcium glycerophosphate, calcium lactate, maltodextrin, glycerine, magnesium silicate, sodium caseinate, soy lecithin, sodium chloride, tricalcium phosphate, dipotassium phosphate, sodium stearoyl lactylate, carrageenan, monoglyceride, diglyceride, pregelatinized starch, hydroxypropylmethylcellulose, hydroxypropylmethylcellulose acetate stearate, sucrose, microcrystalline cellulose, lactose, mannitol and the like.

Suitable filling agents for use in the solid dosage forms described herein include, but are not limited to, lactose, calcium carbonate, calcium phosphate, dibasic calcium phosphate, calcium sulfate, microcrystalline cellulose, cellulose powder, dextrose, dextrates, dextran, starches, pregelatinized starch, hydroxypropylmethycellulose (HPMC), hydroxypropylmethycellulose phthalate, hydroxypropylmethylcellulose acetate stearate (HPMCAS), sucrose, xylitol, lactitol, mannitol, sorbitol, sodium chloride, polyethylene glycol, and the like.

In order to release the compound of ibrutinib, from a solid dosage form matrix as efficiently as possible, disintegrants are often used in the formulation, especially when the dosage forms are compressed with binder. Disintegrants help rupturing the dosage form matrix by swelling or capillary action when moisture is absorbed into the dosage form. Suitable disintegrants for use in the solid dosage forms described herein include, but are not limited to, natural starch such as corn starch or potato starch, a pregelatinized starch such as National 1551 or Amijel®, or sodium starch glycolate such as Promogel® or Explotab®, a cellulose such as a wood product, methylcrystalline cellulose, e.g., Avicel®, Avicel® PH101, Avicel® PH102, Avicel® PH105, Elcema® P100, Emcocel®, Vivacel®, Ming Tia®, and Solka-Floc®, methylcellulose, croscarmellose, or a cross-linked cellulose, such as cross-linked sodium carboxymethylcellulose (Ac-Di-Sol®), cross-linked carboxymethylcellulose, or cross-linked croscarmellose, a cross-linked starch such as sodium starch glycolate, a cross-linked polymer such as crospovidone, a cross-linked polyvinylpyrrolidone, alginate such as alginic acid or a salt of alginic acid such as sodium alginate, a clay such as Veegum® HV (magnesium aluminum silicate), a gum such as agar, guar, locust bean, Karaya, pectin, or tragacanth, sodium starch glycolate, bentonite, a natural sponge, a surfactant, a resin such as a cation-exchange resin, citrus pulp, sodium lauryl sulfate, sodium lauryl sulfate in combination starch, and the like.

Binders impart cohesiveness to solid oral dosage form formulations: for powder filled capsule formulation, they aid in plug formation that can be filled into soft or hard shell capsules and for tablet formulation, they ensure the tablet remaining intact after compression and help assure blend uniformity prior to a compression or fill step. Materials suitable for use as binders in the solid dosage forms described herein include, but are not limited to, carboxymethylcellulose, methylcellulose (e.g., Methocel®), hydroxypropylmethylcellulose (e.g. Hypromellose USP Pharmacoat-603, hydroxypropylmethylcellulose acetate stearate (Aqoate HS-LF and HS), hydroxyethylcellulose, hydroxypropylcellulose (e.g., Klucel®), ethylcellulose (e.g., Ethocel®), and microcrystalline cellulose (e.g., Avicel®), microcrystalline dextrose, amylose, magnesium aluminum silicate, polysaccharide acids, bentonites, gelatin, polyvinylpyrrolidone/vinyl acetate copolymer, crospovidone, povidone, starch, pregelatinized starch, tragacanth, dextrin, a sugar, such as sucrose (e.g., Dipac®), glucose, dextrose, molasses, mannitol, sorbitol, xylitol (e.g., Xylitab®), lactose, a natural or synthetic gum such as acacia, tragacanth, ghatti gum, mucilage of isapol husks, starch, polyvinylpyrrolidone (e.g., Povidone® CL, Kollidon® CL, Polyplasdone® XL-10, and Povidone® K-12), larch arabogalactan, Veegum®, polyethylene glycol, waxes, sodium alginate, and the like.

In general, binder levels of 20-70% are used in powder-filled gelatin capsule formulations. Binder usage level in tablet formulations varies whether direct compression, wet granulation, roller compaction, or usage of other excipients such as fillers which itself can act as moderate binder. Formulators skilled in art can determine the binder level for the formulations, but binder usage level of up to 70% in tablet formulations is common.

Suitable lubricants or glidants for use in the solid dosage forms described herein include, but are not limited to, stearic acid, calcium hydroxide, talc, corn starch, sodium stearyl fumerate, alkali-metal and alkaline earth metal salts, such as aluminum, calcium, magnesium, zinc, stearic acid, sodium stearates, magnesium stearate, zinc stearate, waxes, Stearowet®, boric acid, sodium benzoate, sodium acetate, sodium chloride, leucine, a polyethylene glycol or a methoxypolyethylene glycol such as Carbowax™, PEG 4000, PEG 5000, PEG 6000, propylene glycol, sodium oleate, glyceryl behenate, glyceryl palmitostearate, glyceryl benzoate, magnesium or sodium lauryl sulfate, and the like.

Suitable diluents for use in the solid dosage forms described herein include, but are not limited to, sugars (including lactose, sucrose, and dextrose), polysaccharides (including dextrates and maltodextrin), polyols (including mannitol, xylitol, and sorbitol), cyclodextrins and the like.

The term “non water-soluble diluent” represents compounds typically used in the formulation of pharmaceuticals, such as calcium phosphate, calcium sulfate, starches, modified starches and microcrystalline cellulose, and microcellulose (e.g., having a density of about 0.45 g/cm³, e.g. Avicel, powdered cellulose), and talc.

Suitable wetting agents for use in the solid dosage forms described herein include, for example, oleic acid, glyceryl monostearate, sorbitan monooleate, sorbitan monolaurate, triethanolamine oleate, polyoxyethylene sorbitan monooleate, polyoxyethylene sorbitan monolaurate, quaternary ammonium compounds (e.g., Polyquat 10®), sodium oleate, sodium lauryl sulfate, magnesium stearate, sodium docusate, triacetin, vitamin E TPGS and the like.

Suitable surfactants for use in the solid dosage forms described herein include, for example, sodium lauryl sulfate, sorbitan monooleate, polyoxyethylene sorbitan monooleate, polysorbates, polaxomers, bile salts, glyceryl monostearate, copolymers of ethylene oxide and propylene oxide, e.g., Pluronic® (BASF), and the like.

Suitable suspending agents for use in the solid dosage forms described here include, but are not limited to, polyvinylpyrrolidone, e.g., polyvinylpyrrolidone K12, polyvinylpyrrolidone K17, polyvinylpyrrolidone K25, or polyvinylpyrrolidone K30, polyethylene glycol, e.g., the polyethylene glycol can have a molecular weight of about 300 to about 6000, or about 3350 to about 4000, or about 7000 to about 5400, vinyl pyrrolidone/vinyl acetate copolymer (S630), sodium carboxymethylcellulose, methylcellulose, hydroxy-propylmethylcellulose, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monolaurate, povidone and the like.

Suitable antioxidants for use in the solid dosage forms described herein include, for example, e.g., butylated hydroxytoluene (BHT), sodium ascorbate, and tocopherol.

It should be appreciated that there is considerable overlap between additives used in the solid dosage forms described herein. Thus, the above-listed additives should be taken as merely exemplary, and not limiting, of the types of additives that can be included in solid dosage forms described herein. The amounts of such additives can be readily determined by one skilled in the art, according to the particular properties desired.

In other embodiments, one or more layers of the pharmaceutical formulation are plasticized. Illustratively, a plasticizer is generally a high boiling point solid or liquid. Suitable plasticizers can be added from about 0.01% to about 50% by weight (w/w) of the coating composition. Plasticizers include, but are not limited to, diethyl phthalate, citrate esters, polyethylene glycol, glycerol, acetylated glycerides, triacetin, polypropylene glycol, polyethylene glycol, triethyl citrate, dibutyl sebacate, stearic acid, stearol, stearate, and castor oil.

Compressed tablets are solid dosage forms prepared by compacting the bulk blend of the formulations described above. In various embodiments, compressed tablets which are designed to dissolve in the mouth will include one or more flavoring agents. In other embodiments, the compressed tablets will include a film surrounding the final compressed tablet. In some embodiments, the film coating can provide a delayed release of ibrutinib or the second agent, from the formulation. In other embodiments, the film coating aids in patient compliance (e.g., Opadry® coatings or sugar coating). Film coatings including Opadry® typically range from about 1% to about 3% of the tablet weight. In other embodiments, the compressed tablets include one or more excipients.

In some embodiments, a capsule is prepared, for example, by placing the bulk blend of the formulation of ibrutinib or the second agent, described above, inside of a capsule. In some embodiments, the formulations (non-aqueous suspensions and solutions) are placed in a soft gelatin capsule. In other embodiments, the formulations are placed in standard gelatin capsules or non-gelatin capsules such as capsules comprising HPMC. In other embodiments, the formulation is placed in a sprinkle capsule, wherein the capsule can be swallowed whole or the capsule can be opened and the contents sprinkled on food prior to eating. In some embodiments, the therapeutic dose is split into multiple (e.g., two, three, or four) capsules. In some embodiments, the entire dose of the formulation is delivered in a capsule form.

In various embodiments, the particles of ibrutinib, and one or more excipients are dry blended and compressed into a mass, such as a tablet, having a hardness sufficient to provide a pharmaceutical composition that substantially disintegrates within less than about 30 minutes, less than about 35 minutes, less than about 40 minutes, less than about 45 minutes, less than about 50 minutes, less than about 55 minutes, or less than about 60 minutes, after oral administration, thereby releasing the formulation into the gastrointestinal fluid.

In another aspect, in some embodiments, dosage forms include microencapsulated formulations. In some embodiments, one or more other compatible materials are present in the microencapsulation material. Exemplary materials include, but are not limited to, pH modifiers, erosion facilitators, anti-foaming agents, antioxidants, flavoring agents, and carrier materials such as binders, suspending agents, disintegration agents, filling agents, surfactants, solubilizers, stabilizers, lubricants, wetting agents, and diluents.

Materials useful for the microencapsulation described herein include materials compatible with ibrutinib, which sufficiently isolate the compound of any of ibrutinib, from other non-compatible excipients. Materials compatible with compounds of any of ibrutinib, are those that delay the release of the compounds of any of ibrutinib, in vivo.

Exemplary microencapsulation materials useful for delaying the release of the formulations including compounds described herein, include, but are not limited to, hydroxypropyl cellulose ethers (HPC) such as Klucel® or Nisso HPC, low-substituted hydroxypropyl cellulose ethers (L-HPC), hydroxypropyl methyl cellulose ethers (HPMC) such as Seppifilm-LC, Pharmacoat®, Metolose SR, Methocel®-E, Opadry YS, PrimaFlo, Benecel MP824, and Benecel MP843, methylcellulose polymers such as Methocel®-A, hydroxypropylmethylcellulose acetate stearate Aqoat (HF-LS, HF-LG, HF-MS) and Metolose®, Ethylcelluloses (EC) and mixtures thereof such as E461, Ethocel®, Aqualon®-EC, Surelease®, Polyvinyl alcohol (PVA) such as Opadry AMB, hydroxyethylcelluloses such as Natrosol®, carboxymethylcelluloses and salts of carboxymethylcelluloses (CMC) such as Aqualon®-CMC, polyvinyl alcohol and polyethylene glycol co-polymers such as Kollicoat IR®, monoglycerides (Myverol), triglycerides (KLX), polyethylene glycols, modified food starch, acrylic polymers and mixtures of acrylic polymers with cellulose ethers such as Eudragit® EPO, Eudragit® L30D-55, Eudragit® FS 30D Eudragit® L100-55, Eudragit® L100, Eudragit® S100, Eudragit® RD100, Eudragit® E100, Eudragit® L12.5, Eudragit® S12.5, Eudragit® NE30D, and Eudragit® NE 40D, cellulose acetate phthalate, sepifilms such as mixtures of HPMC and stearic acid, cyclodextrins, and mixtures of these materials.

In still other embodiments, plasticizers such as polyethylene glycols, e.g., PEG 300, PEG 400, PEG 600, PEG 1450, PEG 3350, and PEG 800, stearic acid, propylene glycol, oleic acid, and triacetin are incorporated into the microencapsulation material. In other embodiments, the microencapsulating material useful for delaying the release of the pharmaceutical compositions is from the USP or the National Formulary (NF). In yet other embodiments, the microencapsulation material is Klucel. In still other embodiments, the microencapsulation material is methocel.

In some embodiments, microencapsulated compounds of any of ibrutinib, are formulated by methods known by one of ordinary skill in the art. Such known methods include, e.g., spray drying processes, spinning disk-solvent processes, hot melt processes, spray chilling methods, fluidized bed, electrostatic deposition, centrifugal extrusion, rotational suspension separation, polymerization at liquid-gas or solid-gas interface, pressure extrusion, or spraying solvent extraction bath. In addition to these, several chemical techniques, e.g., complex coacervation, solvent evaporation, polymer-polymer incompatibility, interfacial polymerization in liquid media, in situ polymerization, in-liquid drying, and desolvation in liquid media could also be used. Furthermore, in some embodiments, other methods such as roller compaction, extrusion/spheronization, coacervation, or nanoparticle coating are used.

In one embodiment, the particles of compounds of any of ibrutinib, are microencapsulated prior to being formulated into one of the above forms. In still another embodiment, some or most of the particles are coated prior to being further formulated by using standard coating procedures, such as those described in Remington's Pharmaceutical Sciences, 20th Edition (2000).

In other embodiments, the solid dosage formulations of the compounds of any of ibrutinib, are plasticized (coated) with one or more layers. Illustratively, a plasticizer is generally a high boiling point solid or liquid. Suitable plasticizers can be added from about 0.01% to about 50% by weight (w/w) of the coating composition. Plasticizers include, but are not limited to, diethyl phthalate, citrate esters, polyethylene glycol, glycerol, acetylated glycerides, triacetin, polypropylene glycol, polyethylene glycol, triethyl citrate, dibutyl sebacate, stearic acid, stearol, stearate, and castor oil.

In other embodiments, a powder including the formulations with a compound of any of ibrutinib, described herein, is formulated to include one or more pharmaceutical excipients and flavors. In some embodiments, such a powder is prepared, for example, by mixing the formulation and optional pharmaceutical excipients to form a bulk blend composition. Additional embodiments also include a suspending agent and/or a wetting agent. This bulk blend is uniformly subdivided into unit dosage packaging or multi-dosage packaging units.

In still other embodiments, effervescent powders are also prepared in accordance with the present disclosure. Effervescent salts have been used to disperse medicines in water for oral administration. Effervescent salts are granules or coarse powders containing a medicinal agent in a dry mixture, usually composed of sodium bicarbonate, citric acid and/or tartaric acid. When salts of the compositions described herein are added to water, the acids and the base react to liberate carbon dioxide gas, thereby causing “effervescence.” Examples of effervescent salts include, e.g., the following ingredients: sodium bicarbonate or a mixture of sodium bicarbonate and sodium carbonate, citric acid and/or tartaric acid. Any acid-base combination that results in the liberation of carbon dioxide can be used in place of the combination of sodium bicarbonate and citric and tartaric acids, as long as the ingredients were suitable for pharmaceutical use and result in a pH of about 6.0 or higher.

In some embodiments, the solid dosage forms described herein can be formulated as enteric coated delayed release oral dosage forms, i.e., as an oral dosage form of a pharmaceutical composition as described herein which utilizes an enteric coating to affect release in the small intestine of the gastrointestinal tract. In some embodiments, the enteric coated dosage form is a compressed or molded or extruded tablet/mold (coated or uncoated) containing granules, powder, pellets, beads or particles of the active ingredient and/or other composition components, which are themselves coated or uncoated. In some embodiments, the enteric coated oral dosage form is a capsule (coated or uncoated) containing pellets, beads or granules of the solid carrier or the composition, which are themselves coated or uncoated.

The term “delayed release” as used herein refers to the delivery so that the release can be accomplished at some generally predictable location in the intestinal tract more distal to that which would have been accomplished if there had been no delayed release alterations. In some embodiments the method for delay of release is coating. Any coatings should be applied to a sufficient thickness such that the entire coating does not dissolve in the gastrointestinal fluids at pH below about 5, but does dissolve at pH about 5 and above. It is expected that any anionic polymer exhibiting a pH-dependent solubility profile can be used as an enteric coating in the methods and compositions described herein to achieve delivery to the lower gastrointestinal tract. In some embodiments the polymers described herein are anionic carboxylic polymers. In other embodiments, the polymers and compatible mixtures thereof, and some of their properties, include, but are not limited to:

Shellac, also called purified lac, a refined product obtained from the resinous secretion of an insect. This coating dissolves in media of pH>7;

Acrylic polymers. The performance of acrylic polymers (primarily their solubility in biological fluids) can vary based on the degree and type of substitution. Examples of suitable acrylic polymers include methacrylic acid copolymers and ammonium methacrylate copolymers. The Eudragit series E, L, S, RL, RS and NE (Rohm Pharma) are available as solubilized in organic solvent, aqueous dispersion, or dry powders. The Eudragit series RL, NE, and RS are insoluble in the gastrointestinal tract but are permeable and are used primarily for colonic targeting. The Eudragit series E dissolve in the stomach. The Eudragit series L, L-30D and S are insoluble in stomach and dissolve in the intestine;

Cellulose Derivatives. Examples of suitable cellulose derivatives are: ethyl cellulose; reaction mixtures of partial acetate esters of cellulose with phthalic anhydride. The performance can vary based on the degree and type of substitution. Cellulose acetate phthalate (CAP) dissolves in pH>6. Aquateric (FMC) is an aqueous based system and is a spray dried CAP psuedolatex with particles <1 μm. Other components in Aquateric can include pluronics, Tweens, and acetylated monoglycerides. Other suitable cellulose derivatives include: cellulose acetate trimellitate (Eastman); methylcellulose (Pharmacoat, Methocel); hydroxypropylmethyl cellulose phthalate (HPMCP); hydroxypropylmethyl cellulose succinate (HPMCS); and hydroxypropylmethylcellulose acetate succinate (e.g., AQOAT (Shin Etsu)). The performance can vary based on the degree and type of substitution. For example, HPMCP such as, HP-50, HP-55, HP-555, HP-55F grades are suitable. The performance can vary based on the degree and type of substitution. For example, suitable grades of hydroxypropylmethylcellulose acetate succinate include, but are not limited to, AS-LG (LF), which dissolves at pH 5, AS-MG (MF), which dissolves at pH 5.5, and AS-HG (HF), which dissolves at higher pH. These polymers are offered as granules, or as fine powders for aqueous dispersions; Poly Vinyl Acetate Phthalate (PVAP). PVAP dissolves in pH>5, and it is much less permeable to water vapor and gastric fluids.

In some embodiments, the coating can, and usually does, contain a plasticizer and possibly other coating excipients such as colorants, talc, and/or magnesium stearate, which are well known in the art. Suitable plasticizers include triethyl citrate (Citroflex 2), triacetin (glyceryl triacetate), acetyl triethyl citrate (Citroflec A2), Carbowax 400 (polyethylene glycol 400), diethyl phthalate, tributyl citrate, acetylated monoglycerides, glycerol, fatty acid esters, propylene glycol, and dibutyl phthalate. In particular, anionic carboxylic acrylic polymers usually will contain 10-25% by weight of a plasticizer, especially dibutyl phthalate, polyethylene glycol, triethyl citrate and triacetin. Conventional coating techniques such as spray or pan coating are employed to apply coatings. The coating thickness must be sufficient to ensure that the oral dosage form remains intact until the desired site of topical delivery in the intestinal tract is reached.

In some embodiments, colorants, detackifiers, surfactants, antifoaming agents, lubricants (e.g., carnuba wax or PEG) are added to the coatings besides plasticizers to solubilize or disperse the coating material, and to improve coating performance and the coated product.

In other embodiments, the formulations described herein, which include ibrutinib, are delivered using a pulsatile dosage form. A pulsatile dosage form is capable of providing one or more immediate release pulses at predetermined time points after a controlled lag time or at specific sites. Many other types of controlled release systems known to those of ordinary skill in the art and are suitable for use with the formulations described herein. Examples of such delivery systems include, e.g., polymer-based systems, such as polylactic and polyglycolic acid, polyanhydrides and polycaprolactone; porous matrices, nonpolymer-based systems that are lipids, including sterols, such as cholesterol, cholesterol esters and fatty acids, or neutral fats, such as mono-, di- and triglycerides; hydrogel release systems; silastic systems; peptide-based systems; wax coatings, bioerodible dosage forms, compressed tablets using conventional binders and the like. See, e.g., Liberman et al., Pharmaceutical Dosage Forms, 2 Ed., Vol. 1, pp. 209-214 (1990); Singh et al., Encyclopedia of Pharmaceutical Technology, 2^{nd} Ed., pp. 751-753 (2002); U.S. Pat. Nos. 4,327,725, 4,624,848, 4,968,509, 5,461,140, 5,456,923, 5,516,527, 5,622,721, 5,686,105, 5,700,410, 5,977,175, 6,465,014 and 6,932,983.

In some embodiments, pharmaceutical formulations are provided that include particles of ibrutinib, described herein and at least one dispersing agent or suspending agent for oral administration to a subject. In some embodiments, the formulations are a powder and/or granules for suspension, and upon admixture with water, a substantially uniform suspension is obtained.

Liquid formulation dosage forms for oral administration can be aqueous suspensions selected from the group including, but not limited to, pharmaceutically acceptable aqueous oral dispersions, emulsions, solutions, elixirs, gels, and syrups. See, e.g., Singh et al., Encyclopedia of Pharmaceutical Technology, 2^{nd} Ed., pp. 754-757 (2002). In addition, in some embodiments, the liquid dosage forms include additives, such as: (a) disintegrating agents; (b) dispersing agents; (c) wetting agents; (d) at least one preservative, (e) viscosity enhancing agents, (f) at least one sweetening agent, and (g) at least one flavoring agent. In some embodiments, the aqueous dispersions can further include a crystalline inhibitor.

The aqueous suspensions and dispersions described herein can remain in a homogenous state, as defined in The USP Pharmacists' Pharmacopeia (2005 edition, chapter 905), for at least 4 hours. The homogeneity should be determined by a sampling method consistent with regard to determining homogeneity of the entire composition. In one embodiment, an aqueous suspension can be re-suspended into a homogenous suspension by physical agitation lasting less than 1 minute. In another embodiment, an aqueous suspension can be re-suspended into a homogenous suspension by physical agitation lasting less than 45 seconds. In yet another embodiment, an aqueous suspension can be re-suspended into a homogenous suspension by physical agitation lasting less than 30 seconds. In still another embodiment, no agitation is necessary to maintain a homogeneous aqueous dispersion.

Examples of disintegrating agents for use in the aqueous suspensions and dispersions include, but are not limited to, a starch, e.g., a natural starch such as corn starch or potato starch, a pregelatinized starch such as National 1551 or Amijel®, or sodium starch glycolate such as Promogel® or Explotab®; a cellulose such as a wood product, methylcrystalline cellulose, e.g., Avicel®, Avicel® PH101, Avicel® PH102, Avicel® PH105, Elcema® P100, Emcocel®, Vivacel®, Ming Tia®, and Solka-Floc®, methylcellulose, croscarmellose, or a cross-linked cellulose, such as cross-linked sodium carboxymethylcellulose (Ac-Di-Sol®), cross-linked carboxymethylcellulose, or cross-linked croscarmellose; a cross-linked starch such as sodium starch glycolate; a cross-linked polymer such as crospovidone; a cross-linked polyvinylpyrrolidone; alginate such as alginic acid or a salt of alginic acid such as sodium alginate; a clay such as Veegum® HV (magnesium aluminum silicate); a gum such as agar, guar, locust bean, Karaya, pectin, or tragacanth; sodium starch glycolate; bentonite; a natural sponge; a surfactant; a resin such as a cation-exchange resin; citrus pulp; sodium lauryl sulfate; sodium lauryl sulfate in combination starch; and the like.

In some embodiments, the dispersing agents suitable for the aqueous suspensions and dispersions described herein are known in the art and include, for example, hydrophilic polymers, electrolytes, Tween® 60 or 80, PEG, polyvinylpyrrolidone (PVP; commercially known as Plasdone®), and the carbohydrate-based dispersing agents such as, for example, hydroxypropylcellulose and hydroxypropyl cellulose ethers (e.g., HPC, HPC-SL, and HPC-L), hydroxypropyl methylcellulose and hydroxypropyl methylcellulose ethers (e.g. HPMC K100, HPMC K4M, HPMC K15M, and HPMC K100M), carboxymethylcellulose sodium, methylcellulose, hydroxyethylcellulose, hydroxypropylmethyl-cellulose phthalate, hydroxypropylmethyl-cellulose acetate stearate, noncrystalline cellulose, magnesium aluminum silicate, triethanolamine, polyvinyl alcohol (PVA), polyvinylpyrrolidone/vinyl acetate copolymer (Plasdone®, e.g., S-630), 4-(1,1,3,3-tetramethylbutyl)-phenol polymer with ethylene oxide and formaldehyde (also known as tyloxapol), poloxamers (e.g., Pluronics F68®, F88®, and F108®, which are block copolymers of ethylene oxide and propylene oxide); and poloxamines (e.g., Tetronic 908®, also known as Poloxamine 908®, which is a tetrafunctional block copolymer derived from sequential addition of propylene oxide and ethylene oxide to ethylenediamine (BASF Corporation, Parsippany, N.J.)). In other embodiments, the dispersing agent is selected from a group not comprising one of the following agents: hydrophilic polymers; electrolytes; Tween® 60 or 80; PEG; polyvinylpyrrolidone (PVP); hydroxypropylcellulose and hydroxypropyl cellulose ethers (e.g., HPC, HPC-SL, and HPC-L); hydroxypropyl methylcellulose and hydroxypropyl methylcellulose ethers (e.g. HPMC K100, HPMC K4M, HPMC K15M, HPMC K100M, and Pharmacoat® USP 2910 (Shin-Etsu)); carboxymethylcellulose sodium; methylcellulose; hydroxyethylcellulose; hydroxypropylmethyl-cellulose phthalate; hydroxypropylmethyl-cellulose acetate stearate; non-crystalline cellulose; magnesium aluminum silicate; triethanolamine; polyvinyl alcohol (PVA); 4-(1,1,3,3-tetramethylbutyl)-phenol polymer with ethylene oxide and formaldehyde; poloxamers (e.g., Pluronics F68®, F88®, and F108®, which are block copolymers of ethylene oxide and propylene oxide); or poloxamines (e.g., Tetronic 908®, also known as Poloxamine 908®).

Wetting agents suitable for the aqueous suspensions and dispersions described herein are known in the art and include, but are not limited to, cetyl alcohol, glycerol monostearate, polyoxyethylene sorbitan fatty acid esters (e.g., the commercially available Tweens such as e.g., Tween 20® and Tween 80® (ICI Specialty Chemicals)), and polyethylene glycols (e.g., Carbowaxs 3350® and 1450®, and Carbopol 934® (Union Carbide)), oleic acid, glyceryl monostearate, sorbitan monooleate, sorbitan monolaurate, triethanolamine oleate, polyoxyethylene sorbitan monooleate, polyoxyethylene sorbitan monolaurate, sodium oleate, sodium lauryl sulfate, sodium docusate, triacetin, vitamin E TPGS, sodium taurocholate, simethicone, phosphotidylcholine and the like.

Suitable preservatives for the aqueous suspensions or dispersions described herein include, for example, potassium sorbate, parabens (e.g., methylparaben and propylparaben), benzoic acid and its salts, other esters of parahydroxybenzoic acid such as butylparaben, alcohols such as ethyl alcohol or benzyl alcohol, phenolic compounds such as phenol, or quaternary compounds such as benzalkonium chloride. Preservatives, as used herein, are incorporated into the dosage form at a concentration sufficient to inhibit microbial growth.

Suitable viscosity enhancing agents for the aqueous suspensions or dispersions described herein include, but are not limited to, methyl cellulose, xanthan gum, carboxymethyl cellulose, hydroxypropyl cellulose, hydroxypropylmethyl cellulose, Plasdon® S-630, carbomer, polyvinyl alcohol, alginates, acacia, chitosans and combinations thereof. The concentration of the viscosity enhancing agent will depend upon the agent selected and the viscosity desired.

Examples of sweetening agents suitable for the aqueous suspensions or dispersions described herein include, for example, acacia syrup, acesulfame K, alitame, anise, apple, aspartame, banana, Bavarian cream, berry, black currant, butterscotch, calcium citrate, camphor, caramel, cherry, cherry cream, chocolate, cinnamon, bubble gum, citrus, citrus punch, citrus cream, cotton candy, cocoa, cola, cool cherry, cool citrus, cyclamate, cylamate, dextrose, eucalyptus, eugenol, fructose, fruit punch, ginger, glycyrrhetinate, glycyrrhiza (licorice) syrup, grape, grapefruit, honey, isomalt, lemon, lime, lemon cream, monoammonium glyrrhizinate (MagnaSweet®), maltol, mannitol, maple, marshmallow, menthol, mint cream, mixed berry, neohesperidine DC, neotame, orange, pear, peach, peppermint, peppermint cream, Prosweet® Powder, raspberry, root beer, rum, saccharin, safrole, sorbitol, spearmint, spearmint cream, strawberry, strawberry cream, stevia, sucralose, sucrose, sodium saccharin, saccharin, aspartame, acesulfame potassium, mannitol, talin, sucralose, sorbitol, swiss cream, tagatose, tangerine, thaumatin, tutti fruitti, vanilla, walnut, watermelon, wild cherry, wintergreen, xylitol, or any combination of these flavoring ingredients, e.g., anise-menthol, cherry-anise, cinnamon-orange, cherry-cinnamon, chocolate-mint, honey-lemon, lemon-lime, lemon-mint, menthol-eucalyptus, orange-cream, vanilla-mint, and mixtures thereof. In one embodiment, the aqueous liquid dispersion can comprise a sweetening agent or flavoring agent in a concentration ranging from about 0.001% to about 1.0% the volume of the aqueous dispersion. In another embodiment, the aqueous liquid dispersion can comprise a sweetening agent or flavoring agent in a concentration ranging from about 0.005% to about 0.5% the volume of the aqueous dispersion. In yet another embodiment, the aqueous liquid dispersion can comprise a sweetening agent or flavoring agent in a concentration ranging from about 0.01% to about 1.0% the volume of the aqueous dispersion.

In addition to the additives listed above, the liquid formulations can also include inert diluents commonly used in the art, such as water or other solvents, solubilizing agents, and emulsifiers. Exemplary emulsifiers are ethyl alcohol, isopropyl alcohol, ethyl carbonate, ethyl acetate, benzyl alcohol, benzyl benzoate, propyleneglycol, 1,3-butyleneglycol, dimethylformamide, sodium lauryl sulfate, sodium doccusate, cholesterol, cholesterol esters, taurocholic acid, phosphotidylcholine, oils, such as cottonseed oil, groundnut oil, corn germ oil, olive oil, castor oil, and sesame oil, glycerol, tetrahydrofurfuryl alcohol, polyethylene glycols, fatty acid esters of sorbitan, or mixtures of these substances, and the like.

In some embodiments, the pharmaceutical formulations described herein can be self-emulsifying drug delivery systems (SEDDS). Emulsions are dispersions of one immiscible phase in another, usually in the form of droplets. Generally, emulsions are created by vigorous mechanical dispersion. SEDDS, as opposed to emulsions or microemulsions, spontaneously form emulsions when added to an excess of water without any external mechanical dispersion or agitation. An advantage of SEDDS is that only gentle mixing is required to distribute the droplets throughout the solution. Additionally, water or the aqueous phase can be added just prior to administration, which ensures stability of an unstable or hydrophobic active ingredient. Thus, the SEDDS provides an effective delivery system for oral and parenteral delivery of hydrophobic active ingredients. In some embodiments, SEDDS provide improvements in the bioavailability of hydrophobic active ingredients. Methods of producing self-emulsifying dosage forms are known in the art and include, but are not limited to, for example, U.S. Pat. Nos. 5,858,401, 6,667,048, and 6,960,563, each of which is specifically incorporated by reference.

It is to be appreciated that there is overlap between the above-listed additives used in the aqueous dispersions or suspensions described herein, since a given additive is often classified differently by different practitioners in the field, or is commonly used for any of several different functions. Thus, the above-listed additives should be taken as merely exemplary, and not limiting, of the types of additives that can be included in formulations described herein. The amounts of such additives can be readily determined by one skilled in the art, according to the particular properties desired.

### Intranasal Formulations

Intranasal formulations are known in the art and are described in, for example, U.S. Pat. Nos. 4,476,116, 5,116,817 and 6,391,452, each of which is specifically incorporated by reference. Formulations that include ibrutinib, which are prepared according to these and other techniques well-known in the art are prepared as solutions in saline, employing benzyl alcohol or other suitable preservatives, fluorocarbons, and/or other solubilizing or dispersing agents known in the art. See, for example, Ansel, H. C. et al., Pharmaceutical Dosage Forms and Drug Delivery Systems, Sixth Ed. (1995). Preferably these compositions and formulations are prepared with suitable nontoxic pharmaceutically acceptable ingredients. These ingredients are known to those skilled in the preparation of nasal dosage forms and some of these can be found in Remington: The Science and Practice of Pharmacy, 21st edition, 2005, a standard reference in the field. The choice of suitable carriers is highly dependent upon the exact nature of the nasal dosage form desired, e.g., solutions, suspensions, ointments, or gels. Nasal dosage forms generally contain large amounts of water in addition to the active ingredient. In some embodiments, minor amounts of other ingredients such as pH adjusters, emulsifiers or dispersing agents, preservatives, surfactants, gelling agents, or buffering and other stabilizing and solubilizing agents are also present. The nasal dosage form should be isotonic with nasal secretions.

In some embodiments, for administration by inhalation described herein, the pharmaceutical compositions are in a form as an aerosol, a mist or a powder. Pharmaceutical compositions described herein are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In some embodiments, in the case of a pressurized aerosol, the dosage unit is determined by providing a valve to deliver a metered amount. In some embodiments, capsules and cartridges of, such as, by way of example only, gelatin for use in an inhaler or insufflator are formulated containing a powder mix of the compound described herein and a suitable powder base such as lactose or starch.

### Buccal Formulations

In some embodiments, buccal formulations are administered using a variety of formulations known in the art. For example, such formulations include, but are not limited to, U.S. Pat. Nos. 4,229,447, 4,596,795, 4,755,386, and 5,739,136, each of which is specifically incorporated by reference. In addition, the buccal dosage forms described herein can further include a bioerodible (hydrolysable) polymeric carrier that also serves to adhere the dosage form to the buccal mucosa. The buccal dosage form is fabricated so as to erode gradually over a predetermined time period, wherein the delivery is provided essentially throughout. Buccal drug delivery, as will be appreciated by those skilled in the art, avoids the disadvantages encountered with oral drug administration, e.g., slow absorption, degradation of the active agent by fluids present in the gastrointestinal tract and/or first-pass inactivation in the liver. With regard to the bioerodible (hydrolysable) polymeric carrier, it will be appreciated that virtually any such carrier can be used, so long as the desired drug release profile is not compromised, and the carrier is compatible with ibrutinib, and any other components that are present in the buccal dosage unit. Generally, the polymeric carrier comprises hydrophilic (water-soluble and water-swellable) polymers that adhere to the wet surface of the buccal mucosa. Examples of polymeric carriers useful herein include acrylic acid polymers and co, e.g., those known as “carbomers” (Carbopol®, which can be obtained from B.F. Goodrich, is one such polymer). In some embodiments, other components are also incorporated into the buccal dosage forms described herein include, but are not limited to, disintegrants, diluents, binders, lubricants, flavoring, colorants, preservatives, and the like. In some embodiments, for buccal or sublingual administration, the compositions are in the form of tablets, lozenges, or gels formulated in a conventional manner.

### Transdermal Formulations

In some embodiments, transdermal formulations described herein are administered using a variety of devices which have been described in the art. For example, such devices include, but are not limited to, U.S. Pat. Nos. 3,598,122, 3,598,123, 3,710,795, 3,731,683, 3,742,951, 3,814,097, 3,921,636, 3,972,995, 3,993,072, 3,993,073, 3,996,934, 4,031,894, 4,060,084, 4,069,307, 4,077,407, 4,201,211, 4,230,105, 4,292,299, 4,292,303, 5,336,168, 5,665,378, 5,837,280, 5,869,090, 6,923,983, 6,929,801 and 6,946,144, each of which is specifically incorporated by reference in its entirety.

In some embodiments, the transdermal dosage forms described herein incorporate certain pharmaceutically acceptable excipients which are conventional in the art. In one embodiments, the transdermal formulations described herein include at least three components: (1) a formulation of a compound of ibrutinib; (2) a penetration enhancer; and (3) an aqueous adjuvant. In addition, transdermal formulations can include additional components such as, but not limited to, gelling agents, creams and ointment bases, and the like. In some embodiments, the transdermal formulation can further include a woven or non-woven backing material to enhance absorption and prevent the removal of the transdermal formulation from the skin. In other embodiments, the transdermal formulations described herein can maintain a saturated or supersaturated state to promote diffusion into the skin.

In some embodiments, formulations suitable for transdermal administration of compounds described herein employ transdermal delivery devices and transdermal delivery patches and can be lipophilic emulsions or buffered, aqueous solutions, dissolved and/or dispersed in a polymer or an adhesive. In some embodiments, such patches are constructed for continuous, pulsatile, or on demand delivery of pharmaceutical agents. Still further, transdermal delivery of the compounds described herein can be accomplished by means of iontophoretic patches and the like. Additionally, transdermal patches can provide controlled delivery of ibrutinib. The rate of absorption can be slowed by using rate-controlling membranes or by trapping the compound within a polymer matrix or gel. Conversely, absorption enhancers can be used to increase absorption. An absorption enhancer or carrier can include absorbable pharmaceutically acceptable solvents to assist passage through the skin. For example, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin.

### Injectable Formulations

In some embodiments, formulations that include a compound of ibrutinib, suitable for intramuscular, subcutaneous, or intravenous injection include physiologically acceptable sterile aqueous or non-aqueous solutions, dispersions, suspensions or emulsions, and sterile powders for reconstitution into sterile injectable solutions or dispersions. Examples of suitable aqueous and non-aqueous carriers, diluents, solvents, or vehicles including water, ethanol, polyols (propyleneglycol, polyethylene-glycol, glycerol, cremophor and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions, and by the use of surfactants. In some embodiments, formulations suitable for subcutaneous injection also contain additives such as preserving, wetting, emulsifying, and dispensing agents. Prevention of the growth of microorganisms can be ensured by various antibacterial and antifungal agents, such as parabens, chlorobutanol, phenol, sorbic acid, and the like. In some embodiments, it is also desirable to include isotonic agents, such as sugars, sodium chloride, and the like. Prolonged absorption of the injectable pharmaceutical form can be brought about by the use of agents delaying absorption, such as aluminum monostearate and gelatin.

In some embodiments, for intravenous injections, compounds described herein are formulated in aqueous solutions, preferably in physiologically compatible buffers such as Hank's solution, Ringer's solution, or physiological saline buffer. For transmucosal administration, penetrants appropriate to the barrier to be permeated are used in the formulation. Such penetrants are generally known in the art. In some embodiments, for other parenteral injections, appropriate formulations include aqueous or nonaqueous solutions, preferably with physiologically compatible buffers or excipients. Such excipients are generally known in the art.

In some embodiments, parenteral injections involve bolus injection or continuous infusion. In some embodiments, formulations for injection are presented in unit dosage form, e.g., in ampoules or in multi-dose containers, with an added preservative. In some embodiments, the pharmaceutical composition described herein is in a form suitable for parenteral injection as a sterile suspensions, solutions or emulsions in oily or aqueous vehicles, and contains formulatory agents such as suspending, stabilizing and/or dispersing agents. Pharmaceutical formulations for parenteral administration include aqueous solutions of the active compounds in water-soluble form. Additionally, in some embodiments, suspensions of the active compounds are prepared as appropriate oily injection suspensions. Suitable lipophilic solvents or vehicles include fatty oils such as sesame oil, or synthetic fatty acid esters, such as ethyl oleate or triglycerides, or liposomes. In some embodiments, aqueous injection suspensions contain substances which increase the viscosity of the suspension, such as sodium carboxymethyl cellulose, sorbitol, or dextran. Optionally, in some embodiments, the suspension also contains suitable stabilizers or agents which increase the solubility of the compounds to allow for the preparation of highly concentrated solutions. Alternatively, in some embodiments, the active ingredient is in powder form for constitution with a suitable vehicle, e.g., sterile pyrogen-free water, before use.

### Other Formulations

In certain embodiments, delivery systems for pharmaceutical compounds are employed, such as, for example, liposomes and emulsions. In certain embodiments, compositions provided herein can also include an mucoadhesive polymer, selected from among, for example, carboxymethylcellulose, carbomer (acrylic acid polymer), poly(methylmethacrylate), polyacrylamide, polycarbophil, acrylic acid/butyl acrylate copolymer, sodium alginate and dextran.

In some embodiments, the compounds described herein are administered topically and can be formulated into a variety of topically administrable compositions, such as solutions, suspensions, lotions, gels, pastes, medicated sticks, balms, creams or ointments. Such pharmaceutical compounds can contain solubilizers, stabilizers, tonicity enhancing agents, buffers and preservatives.

In some embodiments, the compounds described herein are formulated in rectal compositions such as enemas, rectal gels, rectal foams, rectal aerosols, suppositories, jelly suppositories, or retention enemas, containing conventional suppository bases such as cocoa butter or other glycerides, as well as synthetic polymers such as polyvinylpyrrolidone, PEG, and the like. In suppository forms of the compositions, a low-melting wax such as, but not limited to, a mixture of fatty acid glycerides, optionally in combination with cocoa butter is first melted.

### Dosing and Treatment Regiments

In some embodiments, the amount of a TEC inhibitor that is administered from 10 mg/day up to, and including, 1000 mg/day. In some embodiments, the amount of a TEC inhibitor that is administered is from about 40 mg/day to 70 mg/day. In some embodiments, the amount of a TEC inhibitor that is administered per day is about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 110 mg, about 120 mg, about 125 mg, about 130 mg, about 135 mg, or about 140 mg.

In some embodiments, the amount of an ITK inhibitor that is administered from 10 mg/day up to, and including, 1000 mg/day. In some embodiments, the amount of an ITK inhibitor that is administered is from about 40 mg/day to 70 mg/day. In some embodiments, the amount of an ITK inhibitor that is administered per day is about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 110 mg, about 120 mg, about 125 mg, about 130 mg, about 135 mg, or about 140 mg.

In some embodiments, the amount of a BTK inhibitor that is administered from 10 mg/day up to, and including, 1000 mg/day. In some embodiments, the amount of a BTK inhibitor that is administered is from about 40 mg/day to 70 mg/day. In some embodiments, the amount of a BTK inhibitor that is administered per day is about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 110 mg, about 120 mg, about 125 mg, about 130 mg, about 135 mg, or about 140 mg.

In some embodiments, the amount of ibrutinib that is administered from 10 mg/day up to, and including, 1000 mg/day. In some embodiments, the amount of Ibrutinib that is administered is from about 40 mg/day to 70 mg/day. In some embodiments, the amount of Ibrutinib that is administered per day is about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 110 mg, about 120 mg, about 125 mg, about 130 mg, about 135 mg, or about 140 mg. In some embodiments, the amount of Ibrutinib that is administered is about 40 mg/day. In some embodiments, the amount of Ibrutinib that is administered is about 50 mg/day. In some embodiments, the amount of Ibrutinib that is administered is about 60 mg/day. In some embodiments, the amount of Ibrutinib that is administered is about 70 mg/day.

In some embodiments, Ibrutinib is administered once per day, twice per day, or three times per day. In some embodiments, Ibrutinib is administered once per day. In some embodiments, Ibrutinib is administered as a maintenance therapy.

In some embodiments, the compositions disclosed herein are administered for prophylactic, therapeutic, or maintenance treatment. In some embodiments, the compositions disclosed herein are administered for therapeutic applications. In some embodiments, the compositions disclosed herein are administered for therapeutic applications. In some embodiments, the compositions disclosed herein are administered as a maintenance therapy, for example for a patient in remission.

In some embodiments, in the case wherein the patient's status does improve, upon the doctor's discretion the administration of the compounds is given continuously; alternatively, the dose of drug being administered may be temporarily reduced or temporarily suspended for a certain length of time (i.e., a “drug holiday”). The length of the drug holiday can vary between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. In some embodiments, the dose reduction during a drug holiday is from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, can be reduced, as a function of the symptoms, to a level at which the improved disease, disorder or condition is retained. Patients can, however, require intermittent treatment on a long-term basis upon any recurrence of symptoms.

The amount of a given agent that will correspond to such an amount will vary depending upon factors such as the particular compound, the severity of the disease, the identity (e.g., weight) of the subject or host in need of treatment, but can nevertheless be routinely determined in a manner known in the art according to the particular circumstances surrounding the case, including, e.g., the specific agent being administered, the route of administration, and the subject or host being treated. In general, however, doses employed for adult human treatment will typically be in the range of 0.02-5000 mg per day, or from about 1-1500 mg per day. In some embodiments, the desired dose is conveniently presented in a single dose or as divided doses administered simultaneously (or over a short period of time) or at appropriate intervals, for example as two, three, four or more sub-doses per day.

In some embodiments, the pharmaceutical composition described herein is in unit dosage forms suitable for single administration of precise dosages. In unit dosage form, the formulation is divided into unit doses containing appropriate quantities of one or more compound. In some embodiments, the unit dosage is in the form of a package containing discrete quantities of the formulation. Non-limiting examples are packaged tablets or capsules, and powders in vials or ampoules. Aqueous suspension compositions can be packaged in single-dose non-reclosable containers. Alternatively, multiple-dose reclosable containers can be used, in which case it is typical to include a preservative in the composition. By way of example only, in some embodiments, formulations for parenteral injection are presented in unit dosage form, which include, but are not limited to ampoules, or in multi-dose containers, with an added preservative.

The foregoing ranges are merely suggestive, as the number of variables in regard to an individual treatment regime is large, and considerable excursions from these recommended values are not uncommon. In some embodiments, such dosages are altered depending on a number of variables, not limited to the activity of the compound used, the disease or condition to be treated, the mode of administration, the requirements of the individual subject, the severity of the disease or condition being treated, and the judgment of the practitioner.

Toxicity and therapeutic efficacy of such therapeutic regimens can be determined by standard pharmaceutical procedures in cell cultures or experimental animals, including, but not limited to, the determination of the LD50 (the dose lethal to 50% of the population) and the ED50 (the dose therapeutically effective in 50% of the population). The dose ratio between the toxic and therapeutic effects is the therapeutic index and it can be expressed as the ratio between LD50 and ED50. Compounds exhibiting high therapeutic indices are preferred. The data obtained from cell culture assays and animal studies can be used in formulating a range of dosage for use in human. The dosage of such compounds lies preferably within a range of circulating concentrations that include the ED50 with minimal toxicity. In some embodiments, the dosage is varied within this range depending upon the dosage form employed and the route of administration utilized.

### Maintenance Therapy

Provided herein are methods for maintenance therapy of subject having a hematological malignancy such as DLBCL. In some embodiments, disclosed herein are methods of monitoring a patient during treatment and optimizing a therapeutic regimen of a patient having a hematological malignancy such as DLBCL. In some embodiments, if the individual has modifications in the one or more biomarker genes selected from EP300, MLL2, BCL-2, RB1, LRP1B, PIM1, TSC2, TNFRSF11A, SMAD4, PAX5, and CARD11, the individual is characterized as having developed resistance or is likely to develop resistance to therapy with a TEC inhibitor. In some embodiments, treatment regimen is modified based on the presence or absence of modifications in the one or more biomarker genes selected from EP300, MLL2, BCL-2, RB1, LRP1B, PIM1, TSC2, TNFRSF11A, SMAD4, PAX5, and CARD11, in an individual receiving therapy. In some embodiments, if the individual has the modification to an aromatic residue at amino acid position 196 in CD79B and at least one modification at amino acid positions 198 or 265 in MYD88, the individual is characterized has responsive or is likely to be responsive to therapy with a TEC inhibitor. In some embodiments, treatment regimen is modified based on the presence or absence of the modification to an aromatic residue at amino acid position 196 in CD79B and at least one modification at amino acid positions 198 or 265 in MYD88. In some embodiments, if the individual has the modification at amino acid position 15 in ROS1, the individual is characterized as resistant or is likely to become resistant to therapy with a TEC inhibitor. In some embodiments, treatment regimen is modified based on the presence or absence of the modification at amino acid position 15 in ROS1. In some embodiments, if the individual shows an increase in expression level in at least one biomarker gene selected from ACTG2, LOR, GAPT, CCND2, SELL, GEN1, and HDAC9 relative to a control, the individual is characterized as having a stable hematological malignancy.

In some embodiments, the methods for maintenance therapy comprise treating DLBCL with a TEC inhibitor, such as an ITK inhibitor or a BTK inhibitor (e.g. ibrutinib) for a period of six months or longer, such as, for example, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 25 months, 26 months, 27 months, 28 months, 29 months, 30 months, 31 months, 32 months, 33 months, 34 months, 35 months, 3 years, 4 years, 5 years, 6 years, 7 years, 8 years, 9 years, 10 years or longer. In some embodiments, the methods for maintenance therapy comprise treating a hematological malignancy such as DLBCL with a TEC inhibitor, such as an ITK inhibitor or a BTK inhibitor (e.g. ibrutinib) for a period of six months or longer, such as, for example, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 25 months, 26 months, 27 months, 28 months, 29 months, 30 months, 31 months, 32 months, 33 months, 34 months, 35 months, 3 years, 4 years, 5 years, 6 years, 7 years, 8 years, 9 years, 10 years or longer.

In some embodiments, the subject is monitored every month, every 2 months, every 3 months, every 4 months, every 5 months, every 6 months, every 7 months, every 8 months, every 9 months, every 10 months, every 11 months, or every year to determine the modifications or the expression levels of the biomarkers disclosed herein.

In some embodiments, maintenance therapy comprises multiple cycles of administration of a TEC inhibitor, such as an ITK inhibitor or a BTK inhibitor. In some embodiments, a cycle of administration is one month, 2 months, 3 months, 4 months, 6 months, 6 months, 7 months, 8 months, 9 months, 10 months, 11 months, 12 months or longer. In some embodiments, a cycle of administration comprises administration of a single therapeutic dosage of a TEC inhibitor, such as an ITK inhibitor or a BTK inhibitor over the cycle. In some embodiments, a cycle of administration comprises two or more different dosages of a TEC inhibitor, such as an ITK inhibitor or a BTK inhibitor over the cycle. In some embodiments, the dosage of a TEC inhibitor, such as an ITK inhibitor or a BTK inhibitor differs over consecutive cycles. In some embodiments, the dosage of a TEC inhibitor, such as an ITK inhibitor or a BTK inhibitor increases over consecutive cycles. In some embodiments, the dosage of a TEC inhibitor, such as an ITK inhibitor or a BTK inhibitor is the same over consecutive cycles. In some embodiments, the BTK inhibitor is ibrutinib.

In some embodiments, maintenance therapy comprises multiple cycles of administration of ibrutinib. In some embodiments, a cycle of administration is one month, 2 months, 3 months, 4 months, 6 months, 6 months, 7 months, 8 months, 9 months, 10 months, 11 months, 12 months or longer. In some embodiments, a cycle of administration comprises administration of a single therapeutic dosage of ibrutinib over the cycle. In some embodiments, a cycle of administration comprises two or more different dosages of ibrutinib over the cycle. In some embodiments, the dosage of ibrutinib differs over consecutive cycles. In some embodiments, the dosage of ibrutinib increases over consecutive cycles. In some embodiments, the dosage of ibrutinib is the same over consecutive cycles.

In some embodiments, maintenance therapy comprises administration of a daily dosage of a TEC inhibitor, such as an ITK inhibitor or a BTK inhibitor. In some embodiments, the daily dosage of a TEC inhibitor, such as an ITK inhibitor or administered is at or about 10 mg per day to about 2000 mg per day, such as for example, about 50 mg per day to about 1500 mg per day, such as for example about 100 mg per day to about 1000 mg per day, such as for example about 250 mg per day to about 850 mg per day, such as for example about 300 mg per day to about 600 mg per day. In a particular embodiment, the maintenance dosage of a TEC inhibitor, such as an ITK inhibitor or a BTK inhibitor is about 840 mg per day. In a particular embodiment, the maintenance dosage of a TEC inhibitor, such as an ITK inhibitor or a BTK inhibitor is about 560 mg per day. In a particular embodiment, the maintenance dosage of a TEC inhibitor, such as an ITK inhibitor or a BTK inhibitor is about 420 mg per day. In a particular embodiment, the maintenance dosage of a TEC inhibitor, such as an ITK inhibitor or a BTK inhibitor is about 140 mg per day.

In some embodiments, maintenance therapy comprises administration of a daily dosage of ibrutinib. In some embodiments, the daily dosage of ibrutinib administered is at or about 10 mg per day to about 2000 mg per day, such as for example, about 50 mg per day to about 1500 mg per day, such as for example about 100 mg per day to about 1000 mg per day, such as for example about 250 mg per day to about 850 mg per day, such as for example about 300 mg per day to about 600 mg per day. In a particular embodiment, the maintenance dosage of ibrutinib is about 840 mg per day. In a particular embodiment, the maintenance dosage of ibrutinib is about 560 mg per day. In a particular embodiment, the maintenance dosage of ibrutinib is about 420 mg per day. In a particular embodiment, the maintenance dosage of ibrutinib is about 140 mg per day.

In some embodiments, a TEC inhibitor, such as an ITK inhibitor or a BTK inhibitor is administered once per day, two times per day, three times per day or more frequent. In a particular embodiment, a TEC inhibitor, such as an ITK inhibitor or a BTK inhibitor is administered once per day.

In some embodiments, ibrutinib is administered once per day, two times per day, three times per day or more frequent. In a particular embodiment, ibrutinib is administered once per day.

In some embodiments, the dosage of a TEC inhibitor, such as an ITK inhibitor or a BTK inhibitor is escalated over time. In some embodiments, the dosage of a TEC inhibitor, such as an ITK inhibitor or a BTK inhibitor is escalated from at or about 1.25 mg/kg/day to at or about 12.5 mg/kg/day over a predetermined period of time. In some embodiments the predetermined period of time is over 1 month, over 2 months, over 3 months, over 4 months, over 5 months, over 6 months, over 7 months, over 8 months, over 9 months, over 10 months, over 11 months, over 12 months, over 18 months, over 24 months or longer.

In some embodiments, the dosage of ibrutinib is escalated over time. In some embodiments, the dosage of ibrutinib is escalated from at or about 1.25 mg/kg/day to at or about 12.5 mg/kg/day over a predetermined period of time. In some embodiments the predetermined period of time is over 1 month, over 2 months, over 3 months, over 4 months, over 5 months, over 6 months, over 7 months, over 8 months, over 9 months, over 10 months, over 11 months, over 12 months, over 18 months, over 24 months or longer.

In some embodiments, a cycle of administration comprises administration of a TEC inhibitor, such as an ITK inhibitor or a BTK inhibitor in combination with an additional therapeutic agent. In some embodiments the additional therapeutic is administered simultaneously, sequentially, or intermittently with a TEC inhibitor, such as an ITK inhibitor or a BTK inhibitor. In some embodiments the additional therapeutic agent is an anti-cancer agent. In some embodiments the additional therapeutic agent is an anti-cancer agent for the treatment of a leukemia, lymphoma or a myeloma. Exemplary anti-cancer agents for administration in a combination with a BTK inhibitor are provided elsewhere herein. In a particular embodiment, the anti-cancer agent is an anti-CD 20 antibody (e.g. Rituxan). In a particular embodiment, the anti-cancer agent bendamustine. In some embodiments, the additional anti-cancer agent is a reversible Btk inhibitor.

In some embodiments, a cycle of administration comprises administration of ibrutinib in combination with an additional therapeutic agent. In some embodiments the additional therapeutic is administered simultaneously, sequentially, or intermittently with ibrutinib. In some embodiments the additional therapeutic agent is an anti-cancer agent. In some embodiments the additional therapeutic agent is an anti-cancer agent for the treatment of a leukemia, lymphoma or a myeloma. Exemplary anti-cancer agents for administration in a combination with ibrutinib are provided elsewhere herein. In a particular embodiment, the anti-cancer agent is an anti-CD 20 antibody (e.g. Rituxan). In a particular embodiment, the anti-cancer agent bendamustine. In some embodiments, the additional anti-cancer agent is a reversible Btk inhibitor.

### Compositions, Kits, and Arrays

Disclosed herein, in certain embodiments, are compositions, kits, and nucleic acid hybridization arrays, for use with one or more methods described herein. In some embodiments, kits disclosed herein comprise one or more reagents for determining the presence or absence of modifications in one or more biomarker genes selected from EP300, MLL2, BCL-2, RB1, LRP1B, PIM1, TSC2, TNFRSF11A, SMAD4, PAX5, and CARD11 in the sample, one or more reagents for determining the presence or absence of a modification to an aromatic residue at amino acid position 196 in CD79B and at least one modification at amino acid positions 198 or 265 in MYD88 in the sample, one or more reagents for determining the presence or absence of a modification at amino acid position 15 in ROS1 in the sample, or one or more reagents for determining the expression level of at least one biomarker gene selected from ACTG2, LOR, GAPT, CCND2, SELL, GEN1 and HDAC9 in the sample.

In some embodiments, a nucleic acid hybridization array comprising nucleic acid probes for evaluating an individual receiving ibrutinib for treatment of diffuse large B cell lymphoma (DLBCL) has developed or is likely to develop resistance to the therapy, consisting essentially of nucleic acid probes which hybridize to biomarker genes selected from the group consisting of BCL-2, RB1, LRP1B, PIM1, TSC2, TNFRSF11A, SMAD4, PAX5, and CARD11. In some embodiments, a nucleic acid hybridization array comprising nucleic acid probes for evaluating whether an individual having diffuse large B cell lymphoma (DLBCL) has a stable DLBCL, consisting essentially of nucleic acid probes which hybridize to biomarker genes selected from the group consisting of ACTG2, LOR, GAPT, CCND2, SELL, GEN1, and HDAC9.

In some cases, the compositions comprise any component, reaction mixture and/or intermediate described herein, as well as any combination thereof. For example, the disclosure provides detection reagents for use with the methods provided herein. In some embodiments, any suitable detection reagents are provided, including a primers, probes, enzymes, antibodies, as described elsewhere herein.

In some instances, kits and nucleic acid hybridization arrays include, a carrier, package, or container that are compartmentalized to receive one or more containers such as vials, tubes, and the like, each of the container(s) comprising one of the separate elements to be used in a method described herein. Suitable containers include, for example, bottles, vials, syringes, and test tubes. In one embodiment, the containers are formed from a variety of materials such as glass or plastic.

In some cases, kits and nucleic acid hybridization arrays provided herein contain packaging materials. Examples of pharmaceutical packaging materials include, but are not limited to, blister packs, bottles, tubes, bags, containers, bottles, and any packaging material suitable for a selected formulation and intended mode of administration and treatment.

For example, the container(s) include Ibrutinib, optionally in a composition or in combination with an additional therapeutic agent as disclosed herein. Such kits optionally include an identifying description or label or instructions relating to its use in the methods described herein.

A kit typically includes labels listing contents and/or instructions for use, and package inserts with instructions for use. A set of instructions will also typically be included.

In one embodiment, a label is on or associated with the container. In one embodiment, a label is on a container when letters, numbers or other characters forming the label are attached, molded or etched into the container itself; a label is associated with a container when it is present within a receptacle or carrier that also holds the container, e.g., as a package insert. In one embodiment, a label is used to indicate that the contents are to be used for a specific therapeutic application. The label also indicates directions for use of the contents, such as in the methods described herein.

In certain embodiments, the pharmaceutical compositions are presented in a pack or dispenser device which contains one or more unit dosage forms containing a compound provided herein. The pack, for example, contains metal or plastic foil, such as a blister pack. In one embodiment, the pack or dispenser device is accompanied by instructions for administration. In one embodiment, the pack or dispenser is also accompanied with a notice associated with the container in form prescribed by a governmental agency regulating the manufacture, use, or sale of pharmaceuticals, which notice is reflective of approval by the agency of the form of the drug for human or veterinary administration. Such notice, for example, is the labeling approved by the U.S. Food and Drug Administration for prescription drugs, or the approved product insert. In one embodiment, compositions containing a compound provided herein formulated in a compatible pharmaceutical carrier are also prepared, placed in an appropriate container, and labeled for treatment of an indicated condition.

### Digital Processing Device

Disclosed herein, in certain embodiments, are systems of assessing an individual having diffuse large B cell lymphoma (DLBCL) for treatment comprising: (a) a digital processing device comprising an operating system configured to perform executable instructions, and an electronic memory; (b) a dataset stored in the electronic memory, wherein the dataset comprises data for one or more biomarker genes in a sample, wherein the biomarker genes are selected from the group consisting of EP300, MLL2, BCL-2, RB1, LRP1B, PIM1, TSC2, TNFRSF11A, SMAD4, PAX5, and CARD11; and (c) a computer program including instructions executable by the digital processing device to create an application comprising: (i) a first software module configured to analyze the dataset to determine the presence or absence of modifications in one or more biomarker genes; and (ii) a second software module to assign the individual as a candidate for treatment with ibrutinib if there is an absence of modifications in the one or more biomarker genes.

Also disclosed herein, in certain embodiments, are systems of assessing an individual having diffuse large B cell lymphoma (DLBCL) for treatment comprising: (a) a digital processing device comprising an operating system configured to perform executable instructions, and an electronic memory; (b) a dataset stored in the electronic memory, wherein the dataset comprises data for one or more biomarker genes in a sample, wherein the biomarker genes are selected from the group consisting of ACTG2, LOR, GAPT, CCND2, SELL, GEN1, and HDAC9; and (c) a computer program including instructions executable by the digital processing device to create an application comprising: (i) a third software module configured to analyze the dataset to determine the expression level of one or more biomarker genes; (ii) a forth software module configured to match the expression level of one or more biomarker genes to a control; and (iii) a fifth software module to assign the individual as a candidate to treatment with ibrutinib if there is an increase in expression level in the one or more biomarker genes relative to the control.

In some embodiments, the systems and methods described herein include a digital processing device, or use of the same. In further embodiments, the digital processing device includes one or more hardware central processing units (CPU) that carry out the device's functions. In still further embodiments, the digital processing device further comprises an operating system configured to perform executable instructions. In some embodiments, the digital processing device is optionally connected to a computer network. In further embodiments, the digital processing device is optionally connected to the Internet such that it accesses the World Wide Web. In still further embodiments, the digital processing device is optionally connected to a cloud computing infrastructure. In other embodiments, the digital processing device is optionally connected to an intranet. In other embodiments, the digital processing device is optionally connected to a data storage device.

In accordance with the description herein, suitable digital processing devices include, by way of non-limiting examples, server computers, desktop computers, laptop computers, notebook computers, sub-notebook computers, netbook computers, netpad computers, set-top computers, media streaming devices, handheld computers, Internet appliances, mobile smartphones, tablet computers, personal digital assistants, video game consoles, and vehicles. Those of skill in the art will recognize that many smartphones are suitable for use in the system described herein. Those of skill in the art will also recognize that select televisions, video players, and digital music players with optional computer network connectivity are suitable for use in the system described herein. Suitable tablet computers include those with booklet, slate, and convertible configurations, known to those of skill in the art.

In some embodiments, the digital processing device includes an operating system configured to perform executable instructions. The operating system is, for example, software, including programs and data, which manages the device's hardware and provides services for execution of applications. Those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft® Windows®, Apple® Mac OS X®, UNIX®, and UNIX-like operating systems such as GNU/Linux®. In some embodiments, the operating system is provided by cloud computing. Those of skill in the art will also recognize that suitable mobile smart phone operating systems include, by way of non-limiting examples, Nokia® Symbian® OS, Apple® iOS®, Research In Motion® BlackBerry OS®, Google® Android®, Microsoft® Windows Phone® OS, Microsoft® Windows Mobile® OS, Linux®, and Palm® WebOS®. Those of skill in the art will also recognize that suitable media streaming device operating systems include, by way of non-limiting examples, Apple TV®, Roku®, Boxee®, Google TV®, Google Chromecast®, Amazon Fire®, and Samsung® HomeSync®. Those of skill in the art will also recognize that suitable video game console operating systems include, by way of non-limiting examples, Sony® PS3®, Sony® PS4®, Microsoft Xbox 360®, Microsoft Xbox One, Nintendo® Wii®, Nintendo® Wii U®, and Ouya®.

In some embodiments, the device includes a storage and/or memory device. The storage and/or memory device is one or more physical apparatuses used to store data or programs on a temporary or permanent basis. In some embodiments, the device is volatile memory and requires power to maintain stored information. In some embodiments, the device is non-volatile memory and retains stored information when the digital processing device is not powered. In further embodiments, the non-volatile memory comprises flash memory. In some embodiments, the non-volatile memory comprises dynamic random-access memory (DRAM). In some embodiments, the non-volatile memory comprises ferroelectric random access memory (FRAM). In some embodiments, the non-volatile memory comprises phase-change random access memory (PRAM). In other embodiments, the device is a storage device including, by way of non-limiting examples, CD-ROMs, DVDs, flash memory devices, magnetic disk drives, magnetic tapes drives, optical disk drives, and cloud computing based storage. In further embodiments, the storage and/or memory device is a combination of devices such as those disclosed herein.

In some embodiments, the digital processing device includes a display to send visual information to a user. In some embodiments, the display is a cathode ray tube (CRT). In some embodiments, the display is a liquid crystal display (LCD). In further embodiments, the display is a thin film transistor liquid crystal display (TFT-LCD). In some embodiments, the display is an organic light emitting diode (OLED) display. In various further embodiments, on OLED display is a passive-matrix OLED (PMOLED) or active-matrix OLED (AMOLED) display. In some embodiments, the display is a plasma display. In other embodiments, the display is a video projector. In still further embodiments, the display is a combination of devices such as those disclosed herein.

In some embodiments, the digital processing device includes an input device to receive information from a user. In some embodiments, the input device is a keyboard. In some embodiments, the input device is a pointing device including, by way of non-limiting examples, a mouse, trackball, track pad, joystick, game controller, or stylus. In some embodiments, the input device is a touch screen or a multi-touch screen. In other embodiments, the input device is a microphone to capture voice or other sound input. In other embodiments, the input device is a video camera or other sensor to capture motion or visual input. In further embodiments, the input device is a Kinect™, Leap Motion™, or the like. In still further embodiments, the input device is a combination of devices such as those disclosed herein.

### Non-Transitory Computer Readable Storage Medium

In some embodiments, the systems and methods disclosed herein include one or more non-transitory computer readable storage media encoded with a program including instructions executable by the operating system of an optionally networked digital processing device. In further embodiments, a computer readable storage medium is a tangible component of a digital processing device. In still further embodiments, a computer readable storage medium is optionally removable from a digital processing device. In some embodiments, a computer readable storage medium includes, by way of non-limiting examples, CD-ROMs, DVDs, flash memory devices, solid state memory, magnetic disk drives, magnetic tape drives, optical disk drives, cloud computing systems and services, and the like. In some cases, the program and instructions are permanently, substantially permanently, semi-permanently, or non-transitorily encoded on the media.

### Computer Program

In some embodiments, the systems and methods disclosed herein include at least one computer program, or use of the same. A computer program includes a sequence of instructions, executable in the digital processing device's CPU, written to perform a specified task. In some embodiments, computer readable instructions are implemented as program modules, such as functions, objects, Application Programming Interfaces (APIs), data structures, and the like, that perform particular tasks or implement particular abstract data types. In light of the disclosure provided herein, those of skill in the art will recognize that a computer program, in certain embodiments, is written in various versions of various languages.

In some embodiments, the functionality of the computer readable instructions are combined or distributed as desired in various environments. In some embodiments, a computer program comprises one sequence of instructions. In some embodiments, a computer program comprises a plurality of sequences of instructions. In some embodiments, a computer program is provided from one location. In other embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof.

### Web Application

In some embodiments, a computer program includes a web application. In light of the disclosure provided herein, those of skill in the art will recognize that a web application, in various embodiments, utilizes one or more software frameworks and one or more database systems. In some embodiments, a web application is created upon a software framework such as Microsoft® .NET or Ruby on Rails (RoR). In some embodiments, a web application utilizes one or more database systems including, by way of non-limiting examples, relational, non-relational, object oriented, associative, and XML database systems. In further embodiments, suitable relational database systems include, by way of non-limiting examples, Microsoft® SQL Server, mySQL™, and Oracle®. Those of skill in the art will also recognize that a web application, in various embodiments, is written in one or more versions of one or more languages. In some embodiments, a web application is written in one or more markup languages, presentation definition languages, client-side scripting languages, server-side coding languages, database query languages, or combinations thereof. In some embodiments, a web application is written to some extent in a markup language such as Hypertext Markup Language (HTML), Extensible Hypertext Markup Language (XHTML), or eXtensible Markup Language (XML). In some embodiments, a web application is written to some extent in a presentation definition language such as Cascading Style Sheets (CSS). In some embodiments, a web application is written to some extent in a client-side scripting language such as Asynchronous Javascript and XML (AJAX), Flash® Actionscript, Javascript, or Silverlight®. In some embodiments, a web application is written to some extent in a server-side coding language such as Active Server Pages (ASP), ColdFusion®, Perl, Java™, JavaServer Pages (JSP), Hypertext Preprocessor (PHP), Python™, Ruby, Tcl, Smalltalk, WebDNA®, or Groovy. In some embodiments, a web application is written to some extent in a database query language such as Structured Query Language (SQL). In some embodiments, a web application integrates enterprise server products such as IBM® Lotus Domino®. In some embodiments, a web application includes a media player element. In various further embodiments, a media player element utilizes one or more of many suitable multimedia technologies including, by way of non-limiting examples, Adobe® Flash®, HTML 5, Apple® QuickTime®, Microsoft Silverlight®, Java™, and Unity®.

### Mobile Application

In some embodiments, a computer program includes a mobile application provided to a mobile digital processing device. In some embodiments, the mobile application is provided to a mobile digital processing device at the time it is manufactured. In other embodiments, the mobile application is provided to a mobile digital processing device via the computer network described herein.

In view of the disclosure provided herein, a mobile application is created by techniques known to those of skill in the art using hardware, languages, and development environments known to the art. Those of skill in the art will recognize that mobile applications are written in several languages. Suitable programming languages include, by way of non-limiting examples, C, C++, C#, Objective-C, Java™, Javascript, Pascal, Object Pascal, Python™, Ruby, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appcelerator®, Celsius, Bedrock, Flash Lite, .NET Compact Framework, Rhomobile, and WorkLight Mobile Platform. Other development environments are available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Those of skill in the art will recognize that several commercial forums are available for distribution of mobile applications including, by way of non-limiting examples, Apple® App Store, Android™ Market, BlackBerry® App World, App Store for Palm devices, App Catalog for webOS, Windows® Marketplace for Mobile, Ovi Store for Nokia® devices, Samsung® Apps, and Nintendo® DSi Shop.

### Standalone Application

In some embodiments, a computer program includes a standalone application, which is a program that is run as an independent computer process, not an add-on to an existing process, e.g., not a plug-in. Those of skill in the art will recognize that standalone applications are often compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and VB .NET, or combinations thereof. Compilation is often performed, at least in part, to create an executable program. In some embodiments, a computer program includes one or more executable complied applications.

### Web Browser Plug-in

In some embodiments, the computer program includes a web browser plug-in. In computing, a plug-in is one or more software components that add specific functionality to a larger software application. Makers of software applications support plug-ins to enable third-party developers to create abilities which extend an application, to support easily adding new features, and to reduce the size of an application. When supported, plug-ins enable customizing the functionality of a software application. For example, plug-ins are commonly used in web browsers to play video, generate interactivity, scan for viruses, and display particular file types. Those of skill in the art will be familiar with several web browser plug-ins including, Adobe® Flash® Player, Microsoft® Silverlight®, and Apple® QuickTime®. In some embodiments, the toolbar comprises one or more web browser extensions, add-ins, or add-ons. In some embodiments, the toolbar comprises one or more explorer bars, tool bands, or desk bands.

In view of the disclosure provided herein, those of skill in the art will recognize that several plug-in frameworks are available that enable development of plug-ins in various programming languages, including, by way of non-limiting examples, C++, Delphi, Java™, PHP, Python™, and VB .NET, or combinations thereof.

Web browsers (also called Internet browsers) are software applications, designed for use with network-connected digital processing devices, for retrieving, presenting, and traversing information resources on the World Wide Web. Suitable web browsers include, by way of non-limiting examples, Microsoft® Internet Explorer®, Mozilla® Firefox®, Google® Chrome, Apple® Safari®, Opera Software® Opera®, and KDE Konqueror. In some embodiments, the web browser is a mobile web browser. Mobile web browsers (also called microbrowsers, mini-browsers, and wireless browsers) are designed for use on mobile digital processing devices including, by way of non-limiting examples, handheld computers, tablet computers, netbook computers, subnotebook computers, smartphones, music players, personal digital assistants (PDAs), and handheld video game systems. Suitable mobile web browsers include, by way of non-limiting examples, Google® Android® browser, RIM BlackBerry® Browser, Apple® Safari®, Palm® Blazer, Palm® WebOS® Browser, Mozilla® Firefox® for mobile, Microsoft® Internet Explorer® Mobile, Amazon® Kindle® Basic Web, Nokia® Browser, Opera Software® Opera® Mobile, and Sony® PSP™ browser.

### Software Modules

In some embodiments, the systems and methods disclosed herein include software, server, and/or database modules, or use of the same. In view of the disclosure provided herein, software modules are created by techniques known to those of skill in the art using machines, software, and languages known to the art. The software modules disclosed herein are implemented in a multitude of ways. In various embodiments, a software module comprises a file, a section of code, a programming object, a programming structure, or combinations thereof. In further various embodiments, a software module comprises a plurality of files, a plurality of sections of code, a plurality of programming objects, a plurality of programming structures, or combinations thereof. In various embodiments, the one or more software modules comprise, by way of non-limiting examples, a web application, a mobile application, and a standalone application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on cloud computing platforms. In some embodiments, software modules are hosted on one or more machines in one location. In other embodiments, software modules are hosted on one or more machines in more than one location.

### Databases

In some embodiments, the methods and systems disclosed herein include one or more databases, or use of the same. In view of the disclosure provided herein, those of skill in the art will recognize that many databases are suitable for storage and retrieval of analytical information described elsewhere herein. In various embodiments, suitable databases include, by way of non-limiting examples, relational databases, non-relational databases, object oriented databases, object databases, entity-relationship model databases, associative databases, and XML databases. In some embodiments, a database is internet-based. In further embodiments, a database is web-based. In still further embodiments, a database is cloud computing-based. In other embodiments, a database is based on one or more local computer storage devices.

### Services

Disclosed herein in certain embodiments, are methods and systems performed as a service. In some embodiments, a service provider obtains a DLBCL samples that a customer wishes to analyze. In some embodiments, the service provider then encodes each DLBCL sample to be analyzed by any of the methods described herein, performs the analysis and provides a report to the customer. In some embodiments, the customer also performs the analysis and provides the results to the service provider for decoding. In some embodiments, the service provider then provides the decoded results to the customer. In some embodiments, the customer also encodes the DLBCL samples, analyzes the samples and decodes the results by interacting with software installed locally (at the customer's location) or remotely (e.g. on a server reachable through a network). In some embodiments, the software generates a report and transmit the report to the customer. Exemplary customers include clinical laboratories, hospitals, and the like. In some embodiments, a customer or party is any suitable customer or party with a need or desire to use the methods, systems, compositions, and kits of the invention.

### Server

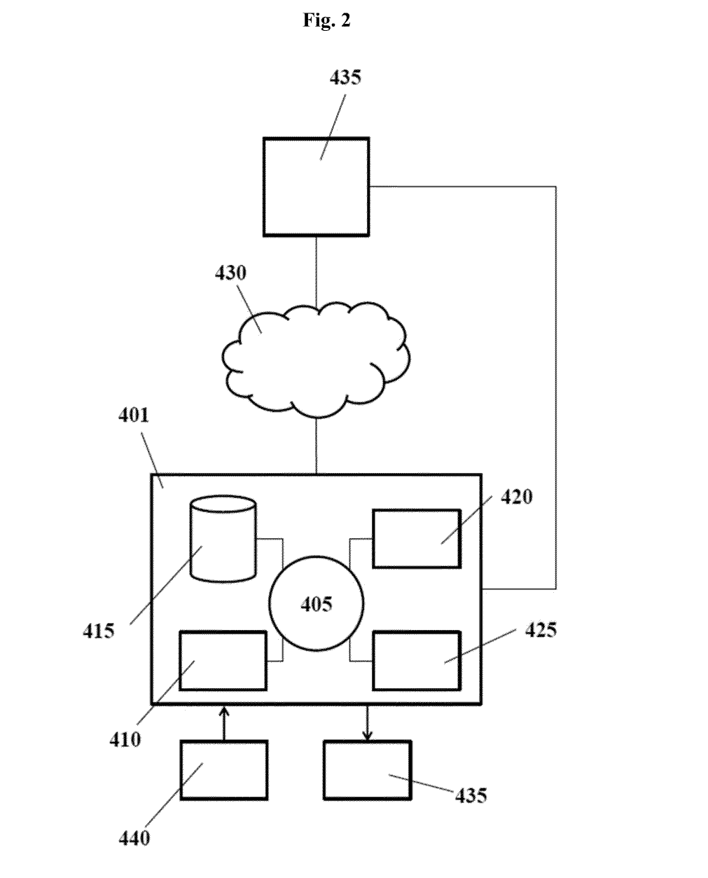

In some embodiments, the methods provided herein are processed on a server or a computer server (FIG. 2). In some embodiments, the server 401 includes a central processing unit (CPU, also “processor”) 405 which is a single core processor, a multi core processor, or plurality of processors for parallel processing. In some embodiments, a processor used as part of a control assembly is a microprocessor. In some embodiments, the server 401 also includes memory 410 (e.g. random access memory, read-only memory, flash memory); electronic storage unit 415 (e.g. hard disk); communications interface 420 (e.g. network adaptor) for communicating with one or more other systems; and peripheral devices 425 which includes cache, other memory, data storage, and/or electronic display adaptors. The memory 410, storage unit 415, interface 420, and peripheral devices 425 are in communication with the processor 405 through a communications bus (solid lines), such as a motherboard. In some embodiments, the storage unit 415 is a data storage unit for storing data. The server 401 is operatively coupled to a computer network (“network”) 430 with the aid of the communications interface 420. In some embodiments, a processor with the aid of additional hardware is also operatively coupled to a network. In some embodiments, the network 430 is the Internet, an intranet and/or an extranet, an intranet and/or extranet that is in communication with the Internet, a telecommunication or data network. In some embodiments, the network 430 with the aid of the server 401, implements a peer-to-peer network, which enables devices coupled to the server 401 to behave as a client or a server. In some embodiments, the server is capable of transmitting and receiving computer-readable instructions (e.g., device/system operation protocols or parameters) or data (e.g., sensor measurements, raw data obtained from detecting metabolites, analysis of raw data obtained from detecting metabolites, interpretation of raw data obtained from detecting metabolites, etc.) via electronic signals transported through the network 430. Moreover, in some embodiments, a network is used, for example, to transmit or receive data across an international border.

In some embodiments, the server 401 is in communication with one or more output devices 435 such as a display or printer, and/or with one or more input devices 440 such as, for example, a keyboard, mouse, or joystick. In some embodiments, the display is a touch screen display, in which case it functions as both a display device and an input device. In some embodiments, different and/or additional input devices are present such an enunciator, a speaker, or a microphone. In some embodiments, the server uses any one of a variety of operating systems, such as for example, any one of several versions of Windows®, or of MacOS®, or of Unix®, or of Linux®.

In some embodiments, the storage unit 415 stores files or data associated with the operation of a device, systems or methods described herein.

In some embodiments, the server communicates with one or more remote computer systems through the network 430. In some embodiments, the one or more remote computer systems include, for example, personal computers, laptops, tablets, telephones, Smart phones, or personal digital assistants.

In some embodiments, a control assembly includes a single server 401. In other situations, the system includes multiple servers in communication with one another through an intranet, extranet and/or the Internet.

In some embodiments, the server 401 is adapted to store device operation parameters, protocols, methods described herein, and other information of potential relevance. In some embodiments, such information is stored on the storage unit 415 or the server 401 and such data is transmitted through a network.

### EXAMPLES

These examples are provided for illustrative purposes only and not to limit the scope of the claims provided herein.

### Example 1

### Patient Cohorts for Genomic Mutation Analysis, Gene Expression Profile and Analyte Expression Analysis

Three DLBCL patient cohorts were analyzed. They are 1106 cohorts 1 and 2, and 04753.

### Example 2

### Effect of Mutations on DLBCL

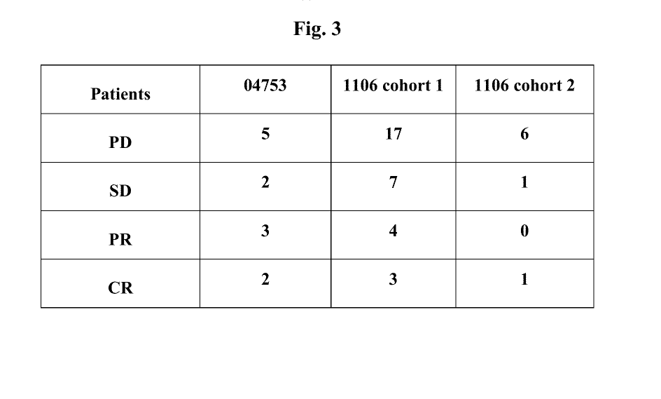

DNA mutations were analyzed for a total of 51 patients, of which 12 were from the 04753 cohort, 31 from the 1106 cohort 1 and 8 from the 1106 cohort 2. The patients were also grouped based on the disease progression, 28 were PD (progression of disease), 10 were SD (stable disease), 7 were PR (partial response) and 6 were CR (complete remission) (FIG. 3). Tumor biopsies were collected from all patients prior to any ibrutinib dose. Tissue sections obtained from the tumor biopsies of all the patients were formalin fixed and paraformaldehyde embedded (FFPE). DNA was extracted from the FFPE tissue sections and hybridized with the FoundationOne T5, T6 and Heme panels (Foundation Medicine, Inc., Cambridge, Mass.) containing 374 cancer related genes and 23 other genes commonly rearranged in cancer.

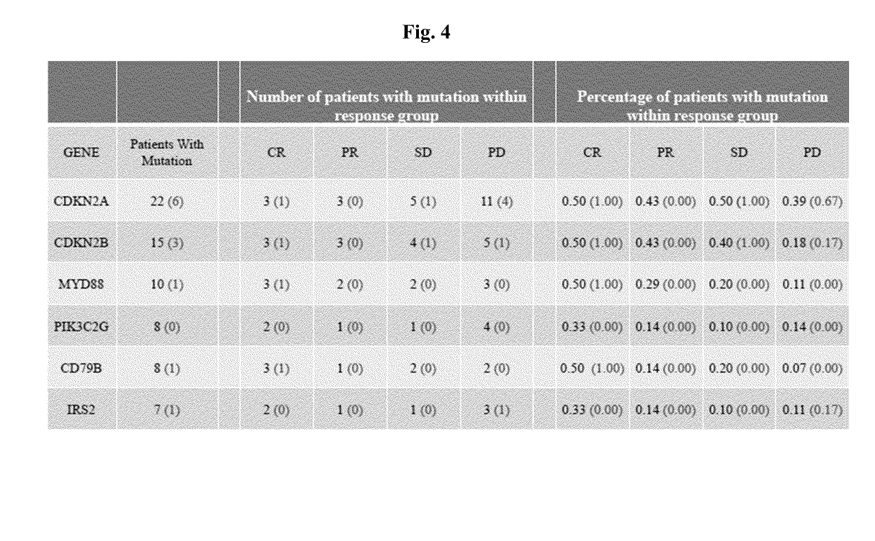

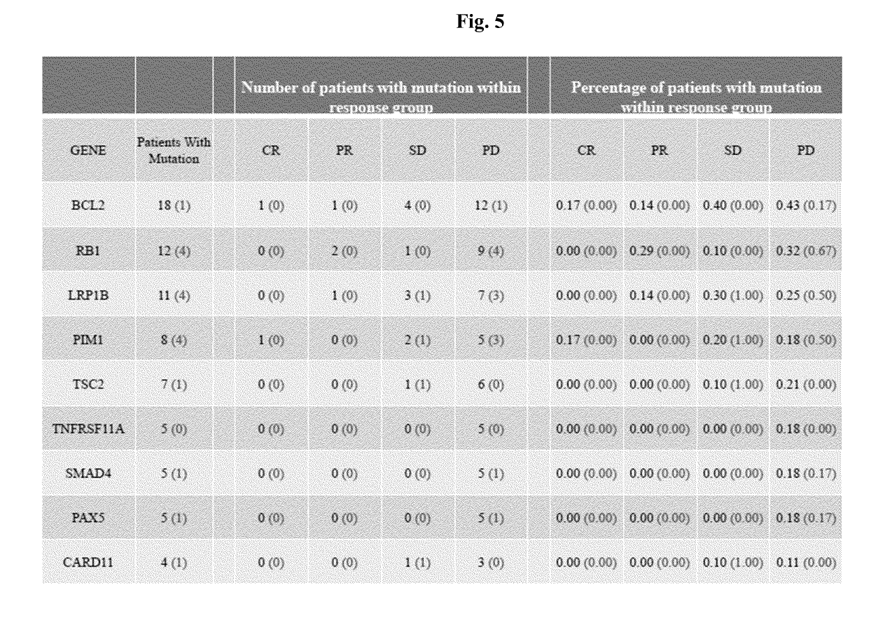

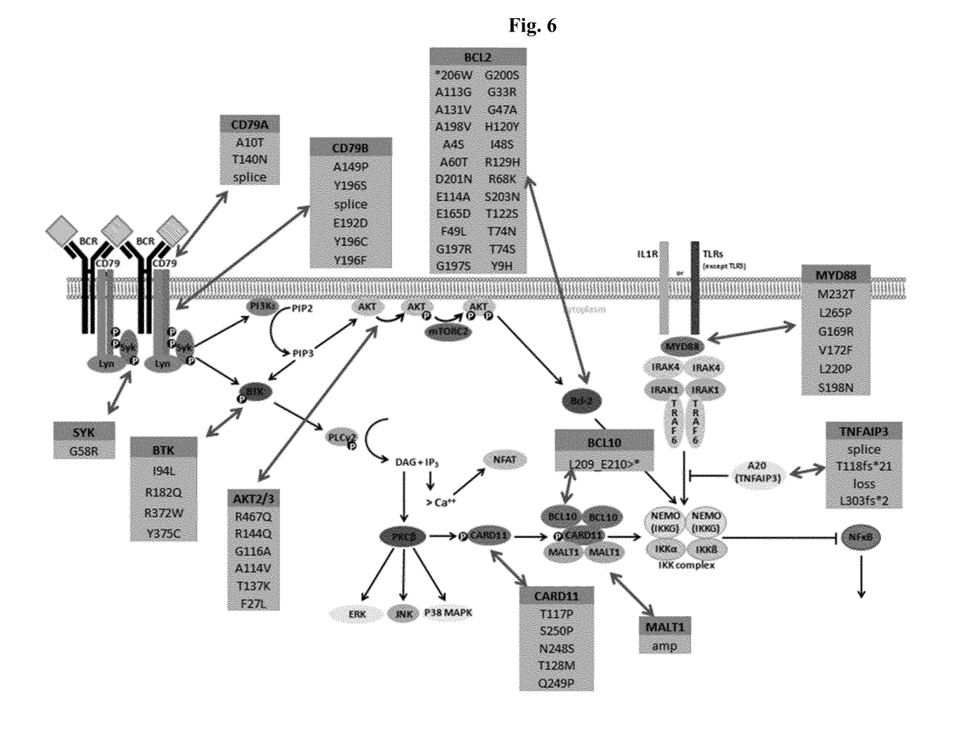

Mutations or modifications analyzed were separated into two groups based on whether their correlation with resistance or likelihood of indicating resistance in patients receiving ibrutinib. The biomarkers did not affect the CR or PR groups include CDKN2A/B, MYD88, PIK3C2G, CD79B, and IRS2 (FIG. 4). The biomarkers that affected the response to ibrutinib include BCL2, RB1, LRP1B, PIM1, TSC2, TNFR, SF11A, SMAD4, PAX5, and CARD11 (FIG. 5). These mutations were prevalent within the PD population of patients.

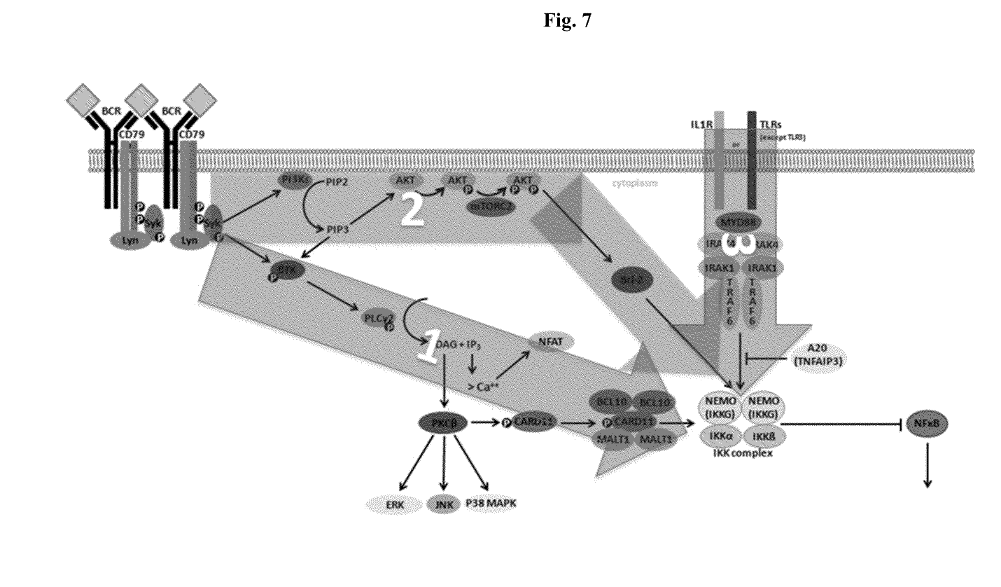

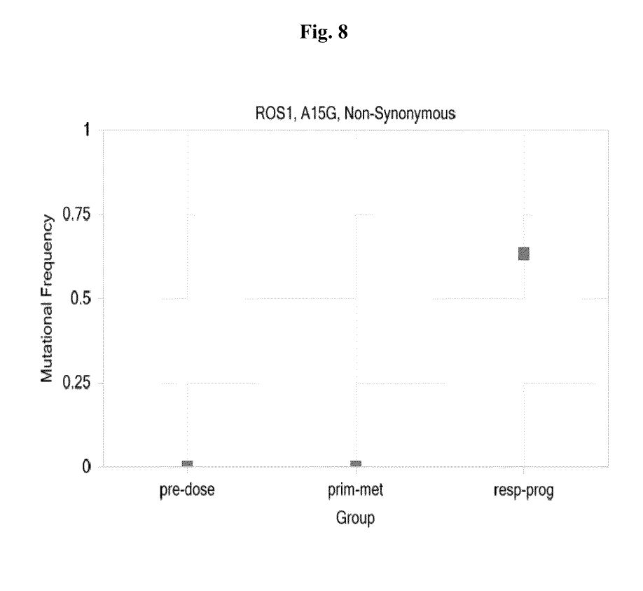

One CR patient, from 1106 cohort 2 (ABC subtype DLBCL), had co-mutations in MYD88 and CD79B. Both of the genes are involved in signaling pathways which ultimately regulate the activation or inhibition of NF-κB gene transcription via NF-κB signaling pathways (FIGS. 6 and 7). This co-mutation in CD79B and MYD88 is Y196F and S198N or Y196F and L265P.

### Example 3

### ROS1 Mutation A15G

A single patient from 1106 cohort, 11096-091-201, had a cutaneous type of DLBCL. The patient responded to ibrutinib treatment but then relapsed. Tumor biopsies were collected at three different stages, pre-dose stage, i.e. prior to any treatment with ibrutinib (biopsy sample from arm), metastasis stage (biopsy sample from leg) and refractory to drug/relapsed stage (biopsy sample from arm). The tissue sections obtained from the tumor biopsies were formalin fixed and paraformaldehyde embedded (FFPE). DNA was extracted from the FFPE tissue sections, subjected to hybridization capture by the FoundationOne panels (Foundation Medicine, Inc., Cambridge, Mass.) containing 374 cancer related genes and 23 other genes commonly rearranged in cancer, and sequenced to high and uniform coverage. A single mutation A15G, in the signal peptide region of ROS1 had a higher mutational frequency in the biopsy sample collected from the patient's arm during the refractory to drug/relapsed stage, when compared to the mutational frequencies in the biopsies collected from the patient's arm during the pre-dose stage and patient's leg during the metastasis stage (FIG. 8).

### Example 4

### Effect of Gene Expression Profile on Progression Free Survival (PFS) in DLBCL Patients

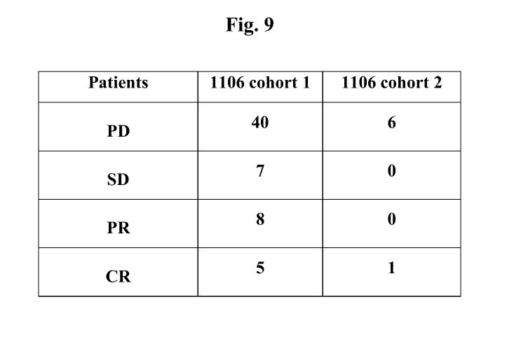

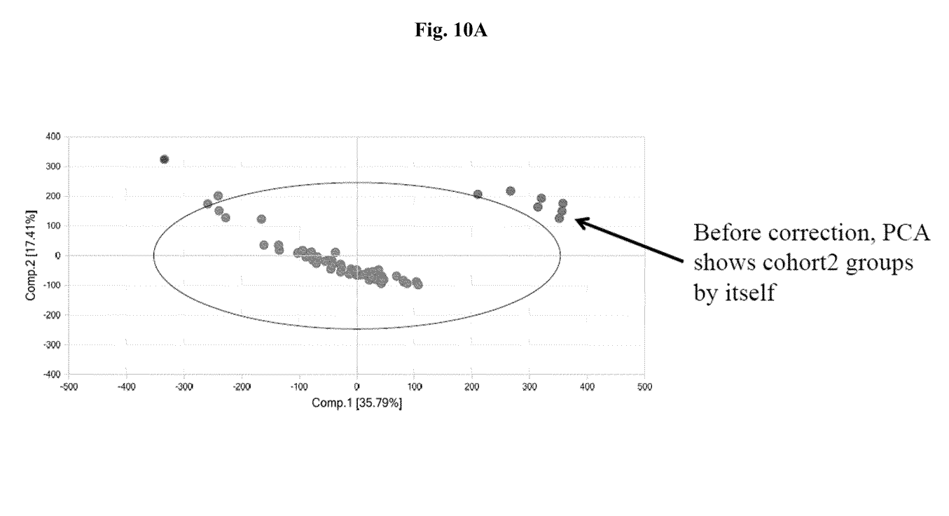

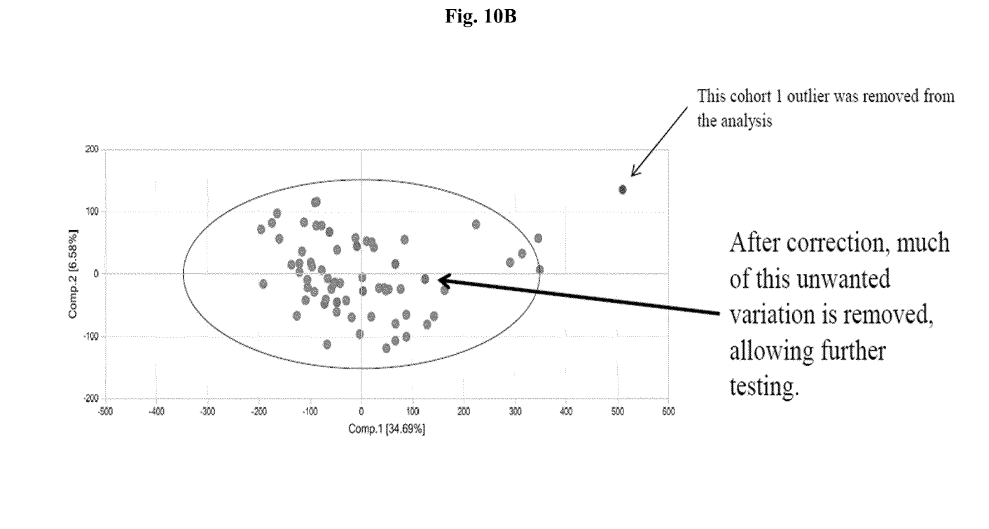

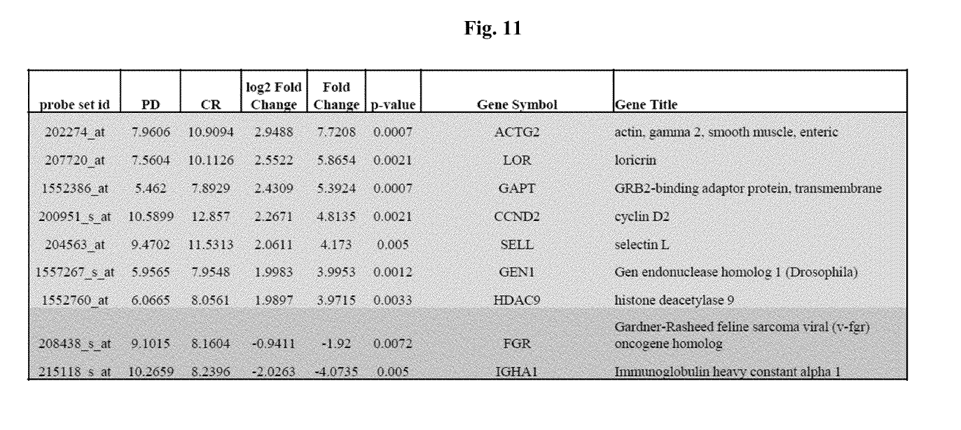

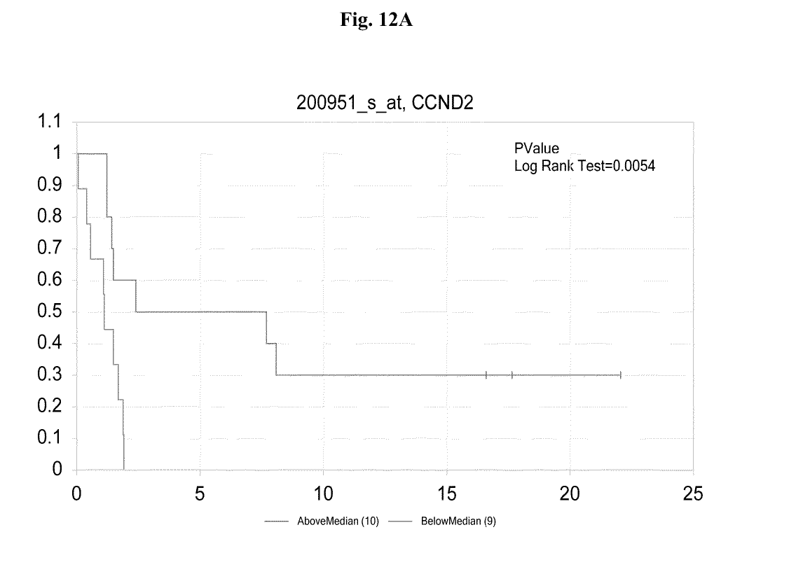

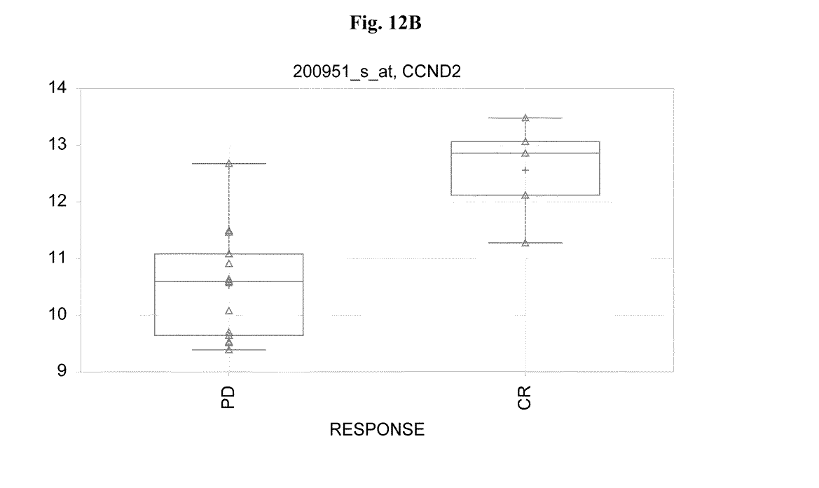

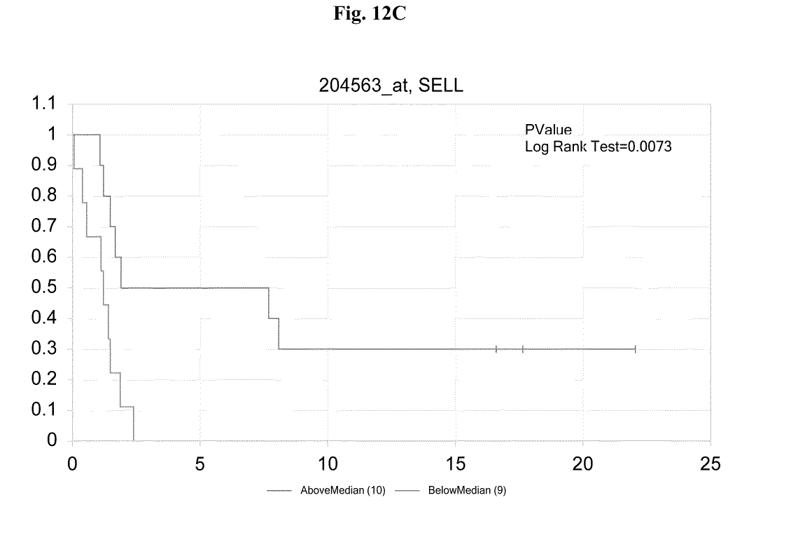

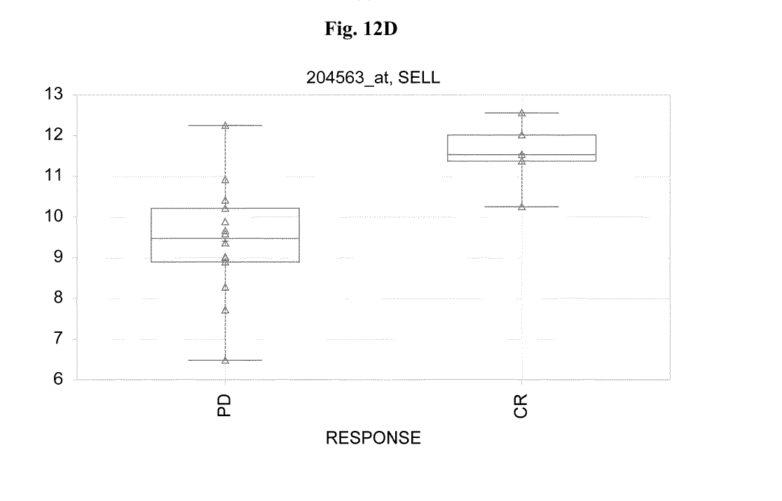

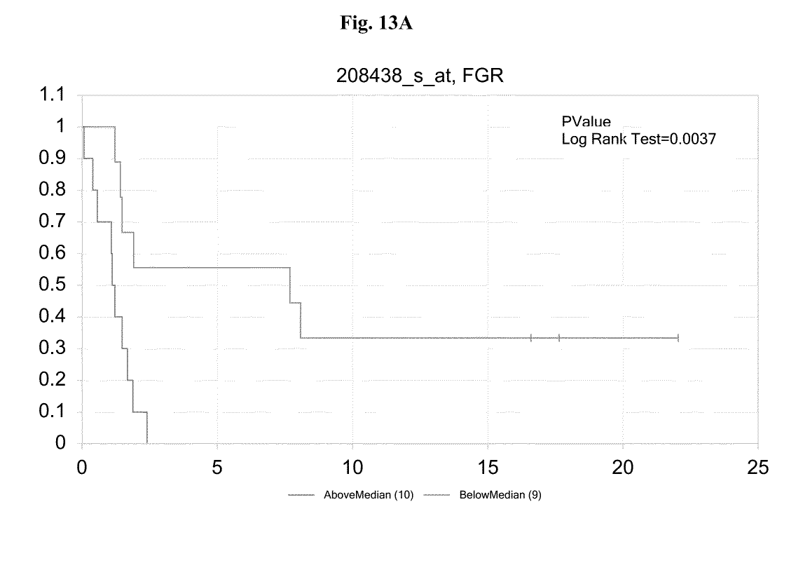

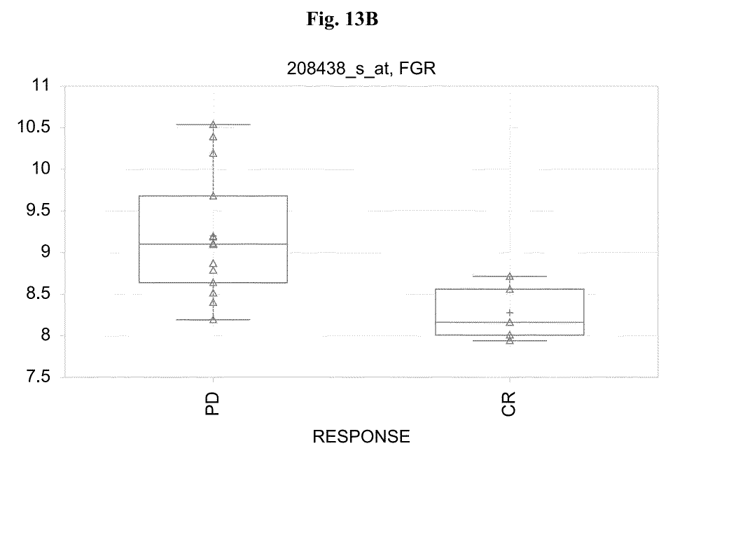

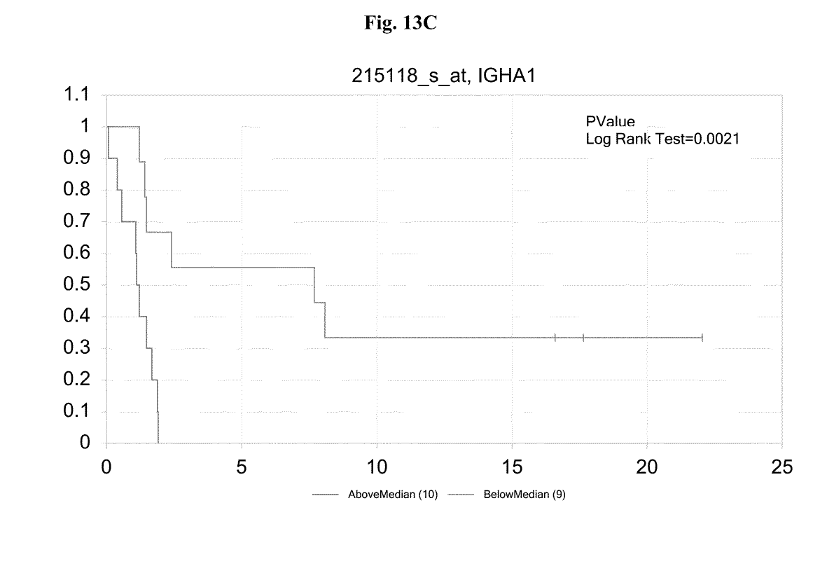

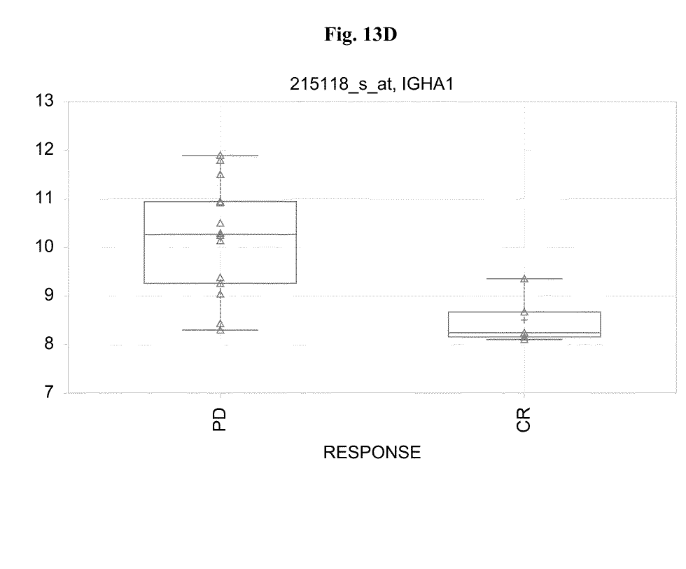

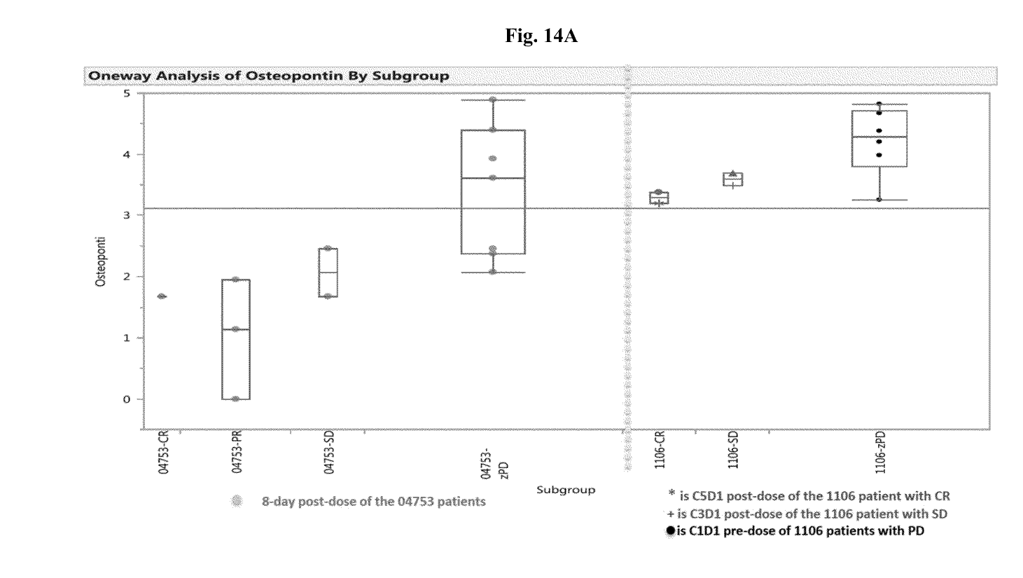

Expression profiles were analyzed for tumor samples collected, prior to any dose of ibrutinib, from 60 patients belonging to 1106 cohort 1 and 7 patients belonging to 1106 cohort 2. The 1106 cohort 1 patients were grouped based on the disease progression, 40 were PD (progression of disease), 7 were SD (stable disease), 8 were PR (partial response), and 5 were CR (complete remission). The 1106 cohort 2 patients were also grouped based on the disease progression, 6 were PD (progression of disease) and 1 was CR (complete remission). The patient stratification is shown in FIG. 9. RNA was extracted from the pre-dose tumor samples and hybridized against Affymetrix U133 Plus 2.0 gene array chips, which analyze the relative expression level of more than 47,000 transcripts. One CEL file was generated per patient, from the Affymetrix U133 Plus 2.0 gene array analysis. Normalized expression levels were computed by processing the CEL files using the Robust Multichip Average (RMA) scheme, which yields log (base 2) expression values. The RMA-Estimated expression values were then correlated with progression free survival (PFS) by using “Survival” and “simPH” packages available in R libraries. The survival data was used to calculate the Cox proportional hazard coefficient for each gene. The coefficient is a quantitative measure of PFS. The gene expression profiling data of cohort 2 initially demonstrated a batch effect, which was subsequently corrected to remove unwanted variation between the 1106 cohorts 1 and 2 (FIGS. 10A and 10B), by removal of batch effect. The cohort 2 samples were also found to be of the ABC subtype when classified using a machine learning approach. Using the calculation methods described above, 7 genes were identified to be positively correlated with PFS. The expression level of the 7 genes, ACTG2, LOR, GAPT, CCND2, SELL, GEN1, and HDAC9 are 3-7 fold higher in CR patients than in PD patients (FIG. 9). Expression levels of CCND2 (FIGS. 12 A-B) and SELL (FIGS. 12C-D) are about 4 fold higher in CR patients than in PD patients. There were 2 other genes, FGR and IGHA1, which negatively correlated with PFS. Expression level of FGR (FIGS. 13A-B) and IGHA1 (FIGS. 13C-D) are 2-4 fold lower in CR patients than in PD patients.

### Example 5

### Effect of Ibrutinib Treatment on Protein Expression Levels in 1106 Cohort 2 DLBCL Patients

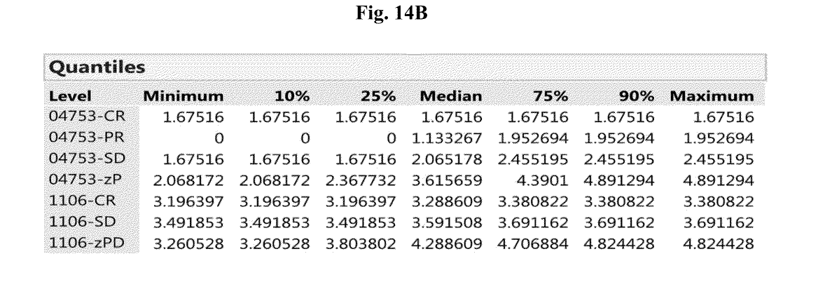

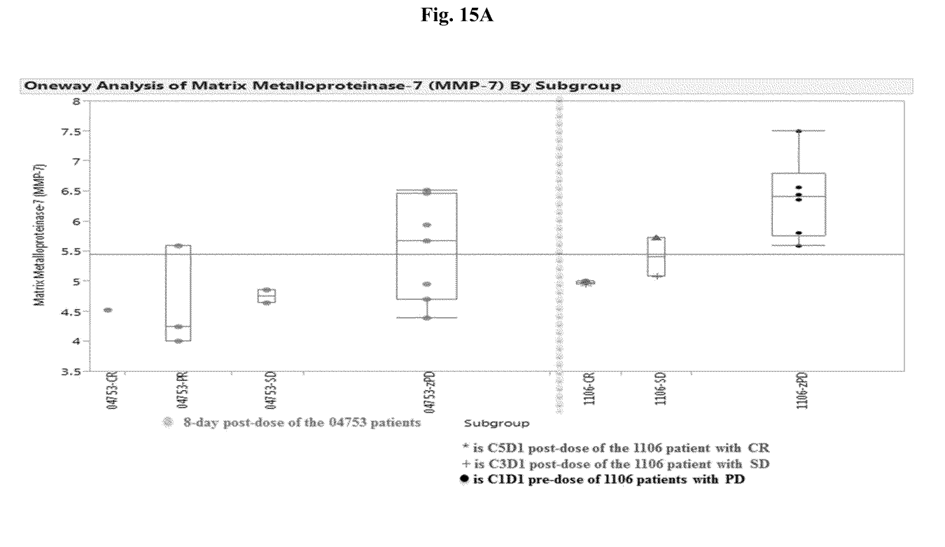

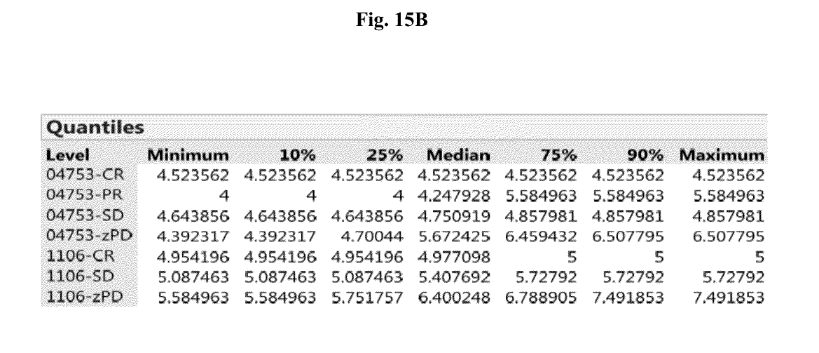

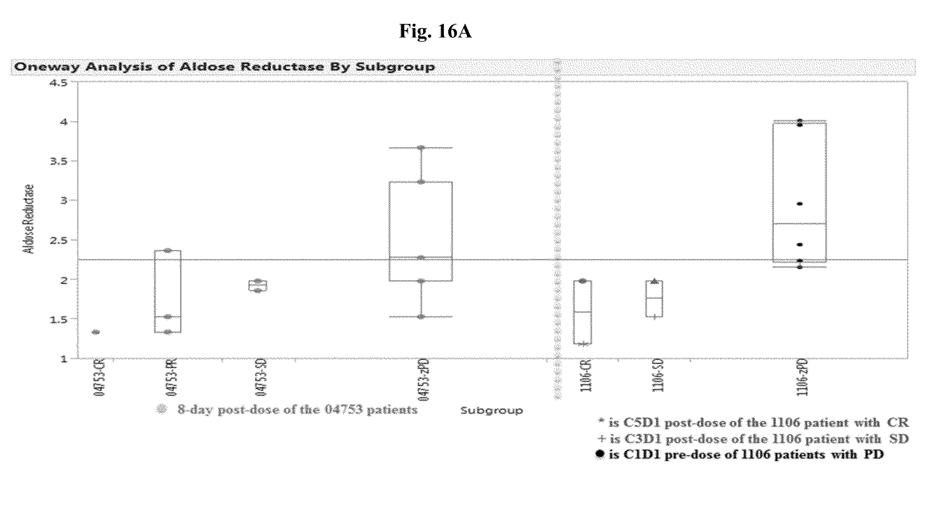

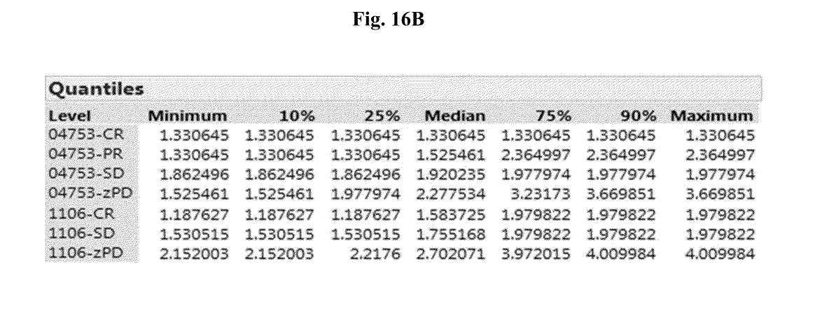

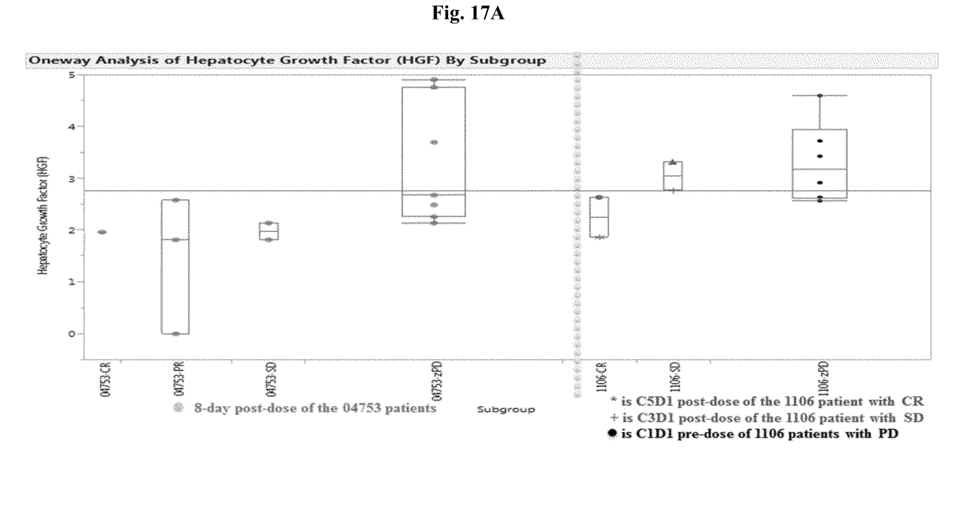

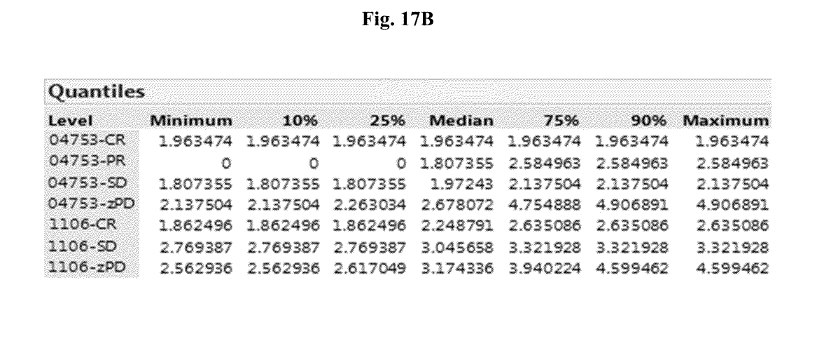

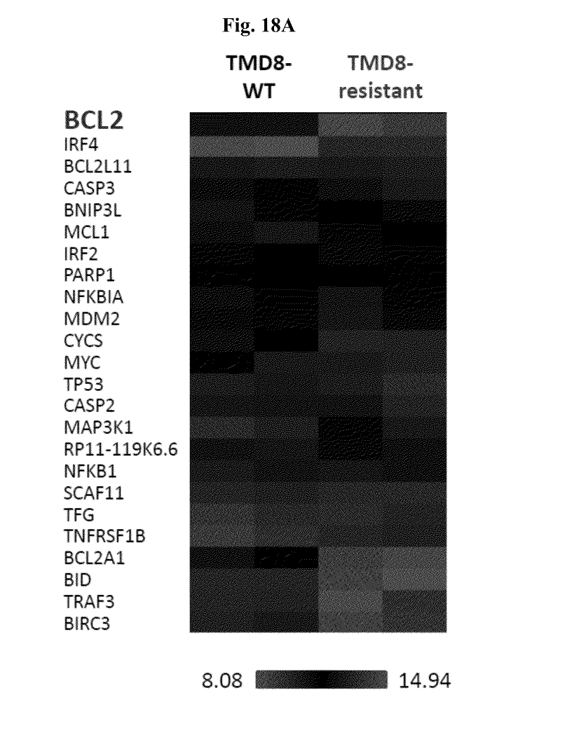

Expression profiles of analytes were determined from serum sample of 8 patients from the 1106 cohort 2, using the Myriad RBM Human DiscoveryMAP250+ v2.0 immunoassay platform (Myriad RBM, Inc., Austin, Tex.). Also, using the HANS-IHC algorithm, it was determined that none of the 8 patients had the germinal center B-subtype of DBLCL. The patients were also grouped based on disease progression, 6 were PD (progression of disease), 1 was SD (stable disease) and 1 was complete remission (CR). The serum samples for the PD patients were collected prior to any dose of ibrutinib (pre-dose), but serum samples for the SD and CR patients were collected both pre-dose and post-dose. Although the Human DiscoveryMAP250+ v2.0 is equipped to assay 240 analytes, 59 analytes were excluded from the analysis as their concentrations were below the lower limit of quantitation (LLOQ). The raw analyte levels were normalized by taking log (base 2) of the ratio between the level of analyte and lower limit of quantitation. The normalized levels of the 180 analytes were then subjected to OneWay analysis. It was observed that the expression levels of the 180 analytes from the 8 patients tested, could be grouped into 16 different patterns. Analytes with elevated levels in the 6 PD patients were Osteopontin (OPN) (FIG. 14A), Matrixmetalloproteinase 7 (MMP-7) (FIG. 15A), Aldose Reductase (ALDR) (FIG. 16A), and Hepatocyte Growth Factor (HGF) (FIG. 17A). The levels of OPN (FIG. 14B), MMP-7 (FIG. 15B), ALDR (FIG. 16B), and HGF (FIG. 17B) were higher in the PD patients (1106-PD) than in SD (1106-SD) and CR patients (1106-CR).

### Example 6

### Effect of Ibrutinib Treatment on Protein Expression Levels in 04753 Cohort DLBCL Patients

Expression profiles of analytes were determined from serum sample of 13 patients from the 04753 cohort using the Myriad RBM Human DiscoveryMAP250+ v2.0 immunoassay platform (Myriad RBM, Inc., Austin, Tex.). The serum samples were obtained eight days post ibrutinib dose. The patients were grouped based on their response to the ibrutinib treatment. The groups were progressive disease (04753-PD), stable disease (047530-SD), partial response (04753-PR), and complete remission (04753-CR). The raw analyte levels were normalized by taking log (base 2) of the ratio between the level of analyte and lower limit of quantitation. The normalized levels of the analytes were then subjected to OneWay analysis. Analytes with elevated levels in the 04753-PD patients were Osteopontin (OPN) (FIG. 14A), Matrixmetalloproteinase 7 (MMP-7) (FIG. 15A), Aldose Reductase (ALDR) (FIG. 16A), and Hepatocyte Growth Factor (HGF) (FIG. 17A). The levels of OPN (FIG. 14B), MMP-7 (FIG. 15B), ALDR (FIG. 16B), and HGF (FIG. 17B) were higher in the PD patients (04753-PD) than in SD (04753-SD) and CR patients (04753-CR).

### Example 7

### BCL-2 Gene Expression

### Gene Expression Analysis in TMD8 Cells

Gene expressions of wild type TMD8 cells and ibrutinib-resistant TMD8 cells were analyzed using GeneChip Human Transcriptome Array 2.0. Transcriptome Analysis Console v2.0 was used to generate the heatmap which illustrates a list of apoptosis related genes.

Gene expressions of BAX, BCL-2, and MCL-1 were measured by qPCR. Expression data were normalized to a GAPDH reference gene. All data were presented as fold change over the wild type TMD8 sample.

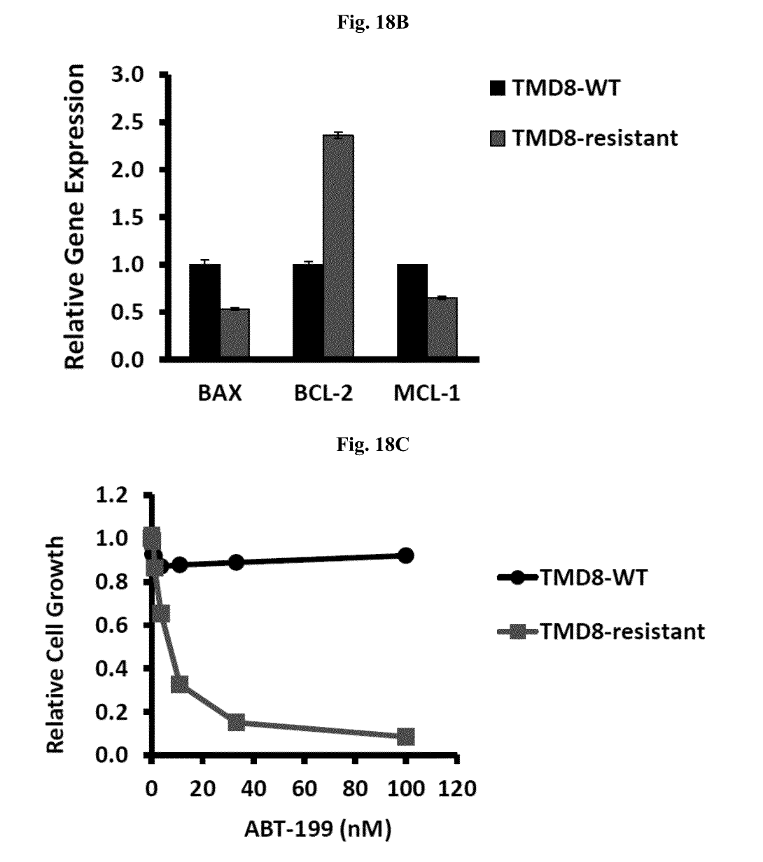

Wild type TMD8 cells and ibrutinib-resistant TMD8 cells were treated with indicated concentrations (FIG. 18C) of ABT-199 for 3 days and the drug effect on cell growth was determined using a CellTiter-Glo luminescent cell viability assay.

FIG. 18A-FIG. 18C show comparison of BCL-2 gene expressions in either ibrutinib-resistant TMD8 cells or wild-type TMD8 cells. BCL-2 gene expression is higher in ibrutinib-resistant TMD8 cells than in wild-type TMD8 cells.

BCL-2 Gene Expression Analysis of Tumor Samples from DLBCL Subtypes

Affymetrix HG-U133 Plus 2 data was normalized using the Robust Multi-array Average (RMA) algorithm. This normalization method was based on the classification algorithm of Wright, G. et al. PNAS 2003; 100(17):9991-6. Subtypes of DLBCL were analyzed at the National Cancer Institute. For the ABC-DLBCL subtype analysis, only the samples having a gene expression profiling (GEP) call of ABC-DLBCL were used and normalized separately. A test for differential expression of genes between ABC-DLBCL responders (CR+PR) and non-responders (SD+PD) to Ibrutinib was performed using the rank product statistic (RankProd R package). For the ABC-DLBCL vs GCB-DLBCL comparison plot and heatmap, all subtypes were normalized together. The data were plotted in linear scale.

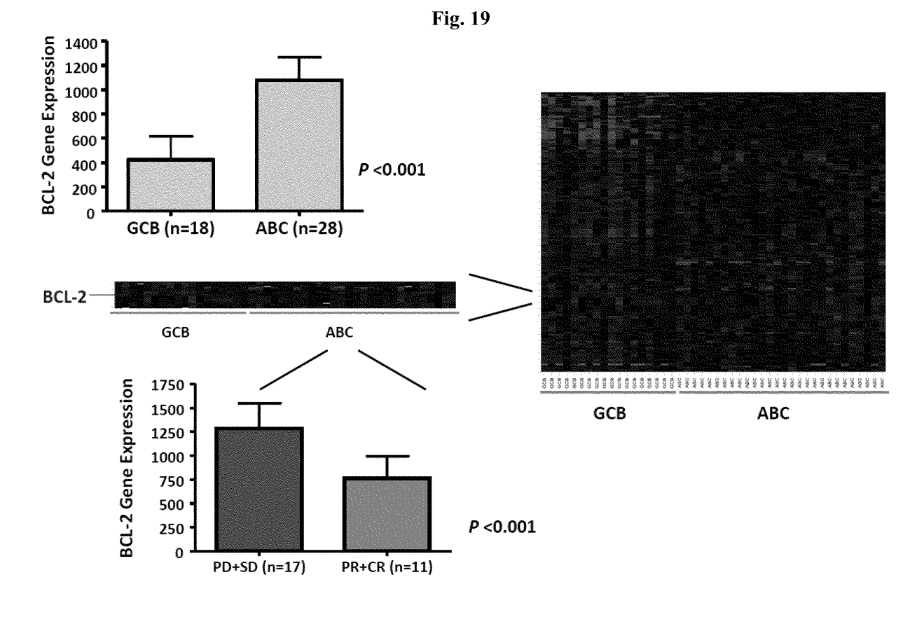

FIG. 19 illustrates BCL-2 gene expressions in different subspecies of DLBCL tumor samples. Lower BCL-2 gene expression was observed in the tumor samples from patients with better responses to ibrutinib.

BCL-2 Gene Expression Analysis of Tumor Samples from Patients with Different Ibrutinib Responses

FIG. 20 top panel illustrates BCL-2 mutation frequency identified in tumor samples from patients with different response to ibrutinib.

FIG. 20 bottom panel illustrates a sequence alignment between the BCL2 protein sequence (Uniprot accession P10415) and the sequence corresponding to the crystal structure 4MAN_A. The sequence alignment was performed using ClustalW and visualized with Seaview (physical properties of the amino acids were color coded by default Seaview scheme documented at http://pageperso.lif.univ-mrs.fr/˜ michel.vancaneghem/optionBio2/documents/seaview.help. This PDB entry is a co-crystal structure of BCL2 with the inhibitor 4-[4-({4′-chloro-3-[2-(dimethylamino)ethoxy]biphenyl-2-yl}methyl)piperazin-1-yl]-2-(1H-indol-5-yloxy)-N-({3-nitro-4-[(tetrahydro-2H-pyran-4-ylmethyl)amino]phenyl}sulfonyl)benzamide.

Mutation in the BCL2 coding region gives rise to amino acid substitutions that are noted on the alignment (muts) and denoted from wild type (wt). Additionally, structural domains of BCL2 are annotated on the alignment with the domain indicated by a number. Contacts between the inhibitor and the protein are marked with an X. The contacts were mapped by performing a 12 ns molecular dynamics simulation of the complex in water at 300K using the Schrodinger Suite version 2014-2, and then analyzing the resulting trajectory data within Schrodinger's Maestro. A contact is defined as a physical interaction between a protein residue and the inhibitor. Such interactions can be H-bonding, pi-pi stacking, hydrophobic, and electrostatic. It is noted that this is a modeling result that was performed on an actual crystal structure of the complex.

### Example 8

### Combination Effect of BCL-2 Inhibitor and Ibrutinib in DoHH2 Cells

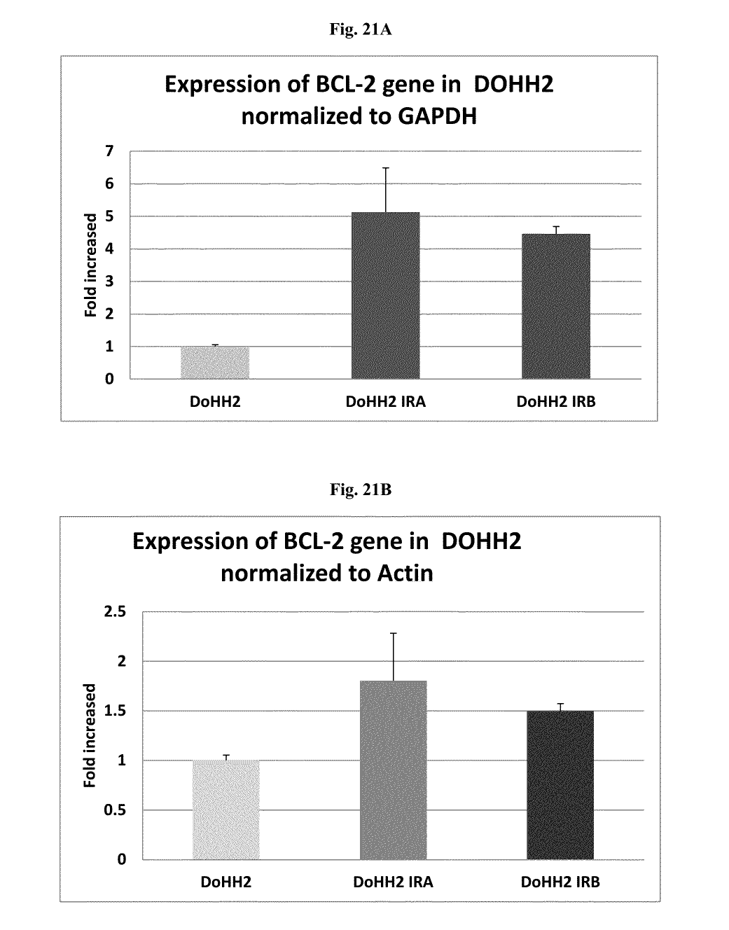

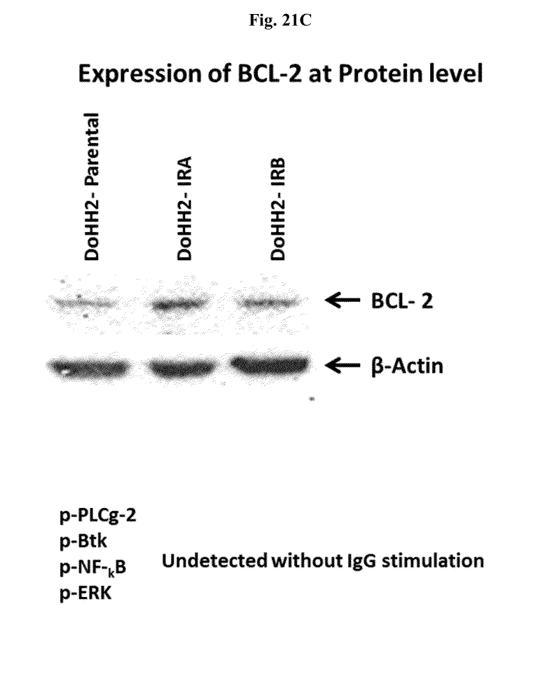

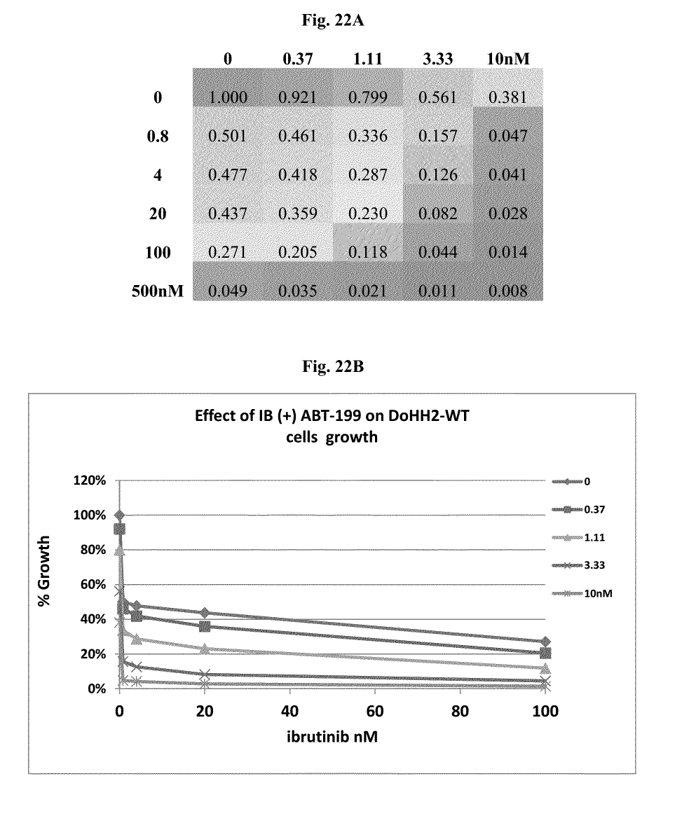

FIG. 21 illustrates BCL-2 expression in DoHH2 cell-lines. FIG. 21A and FIG. 21B show the expression of BCL-2 gene in DoHH2, a non-Hodgkin's B-cell line, normalized to GAPDH and Actin, respectively. FIG. 21C shows the expressin of BCL-2 at the protein level. BCL-2 is expressed at a higher level in ibrutinib resistant DoHH2 cells compared to the wild-type DoHH2 cells.

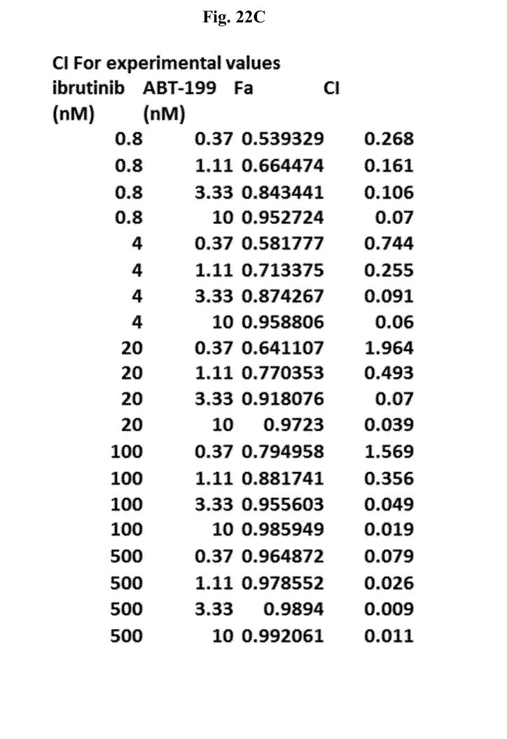

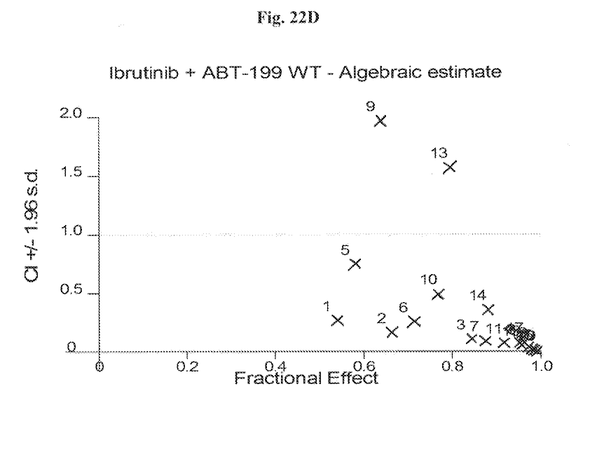

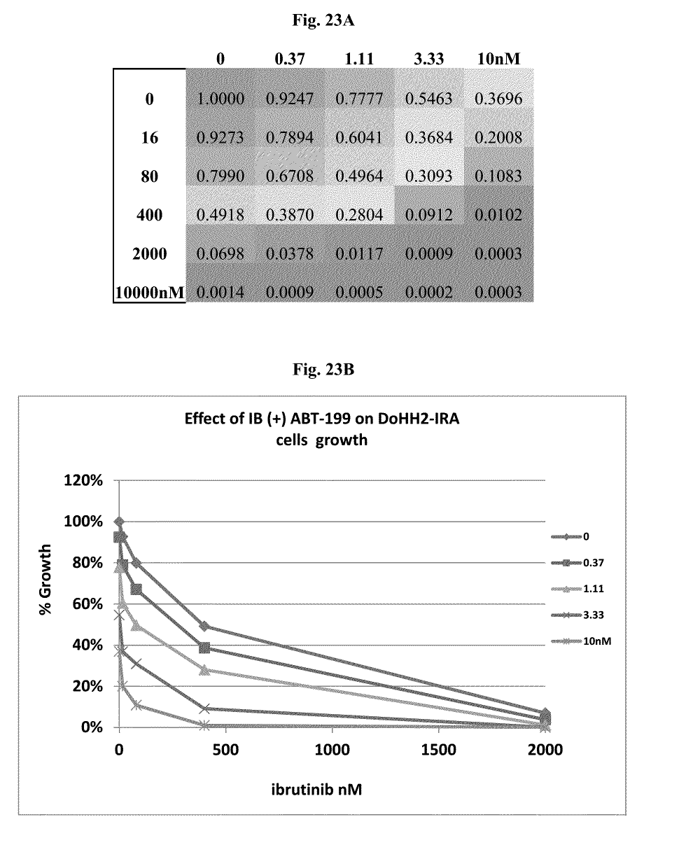

FIG. 22A-FIG. 22D show the effect of the combination of ibrutinib and ABT-199 on wild type DoHH2 proliferation. FIG. 22A illustrates the synergy score heat map of ibrutinib and ABT-199. FIG. 22B shows the percentage of growth of DoHH2 wild-type cells in the presence of ABT-199 and ibruitnib. In some instances, the EC50 is 2.079 nM. FIGS. 22C and 22D show the synergy score of the ibrutinib and ABT-199 combination.

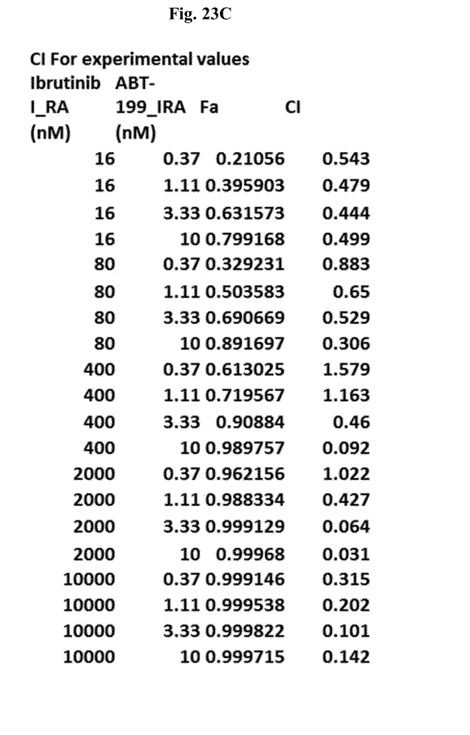

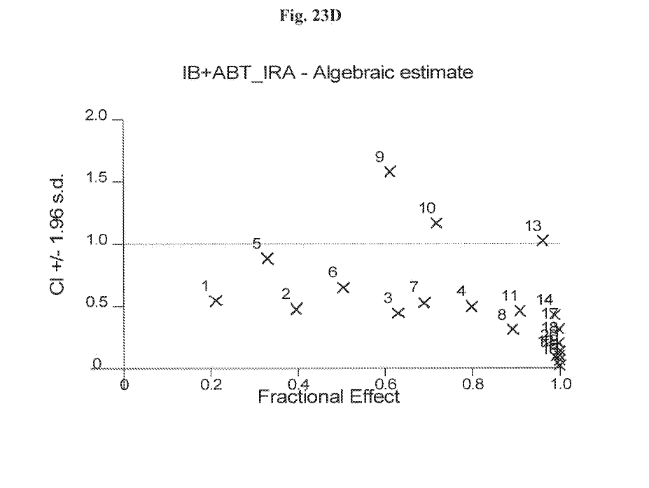

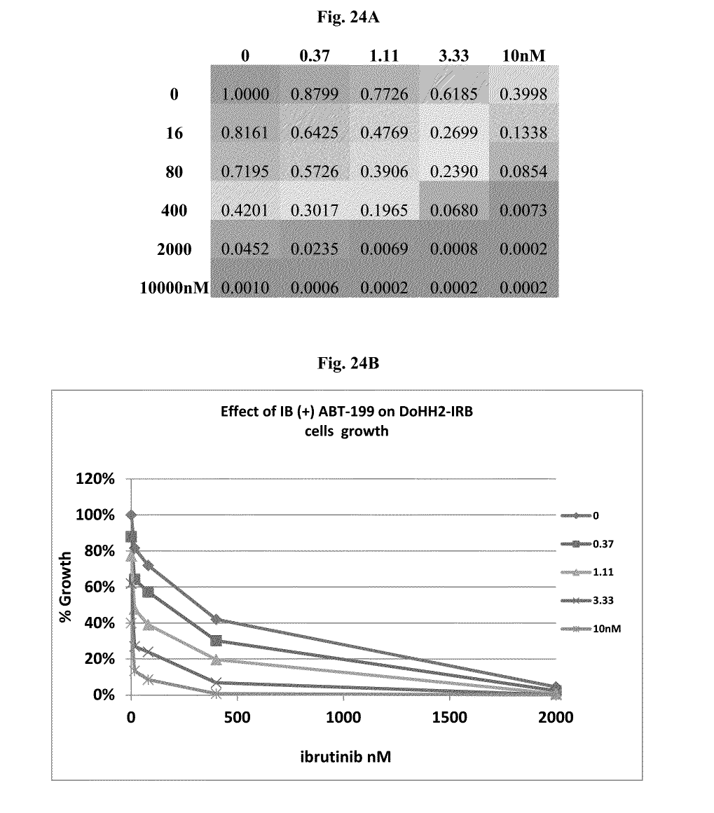

FIG. 23A-FIG. 23D show the effect of the combination of ibrutinib and ABT-199 on ibrutinib resistant DoHH2 proliferation. FIG. 23A illustrates the synergy score heat map of ibrutinib and ABT-199. In some instances, the EC50 is 329.7 nM. FIG. 23B shows the percentage of growth of DoHH2 ibrutinib resistant cells in the presence of ABT-199 and ibruitnib. FIGS. 23C and 23D show the synergy score of the ibrutinib and ABT-199 combination.

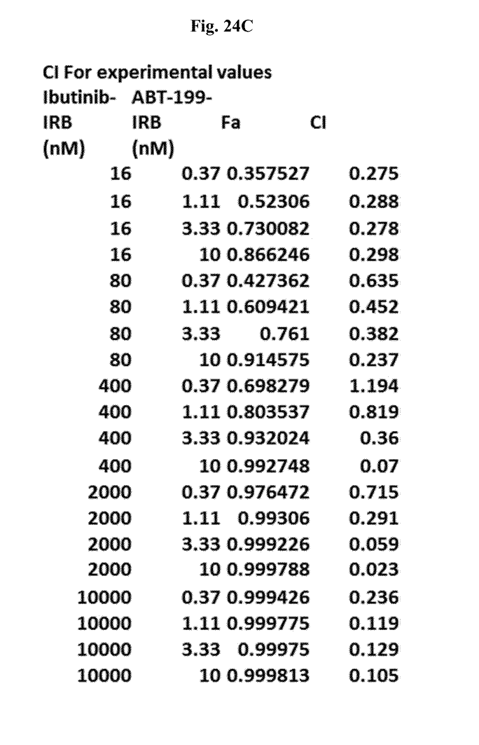

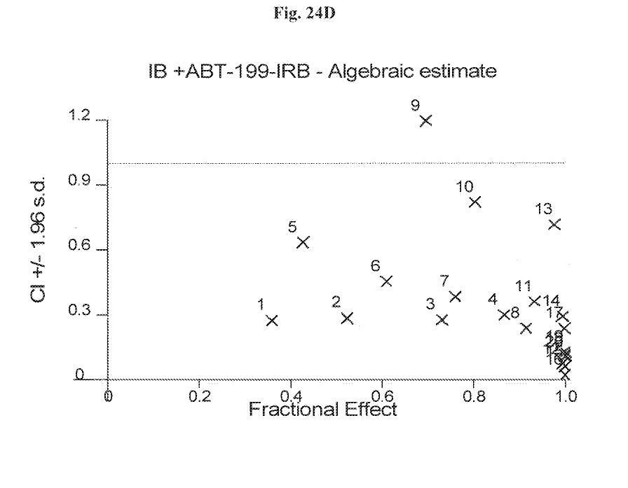

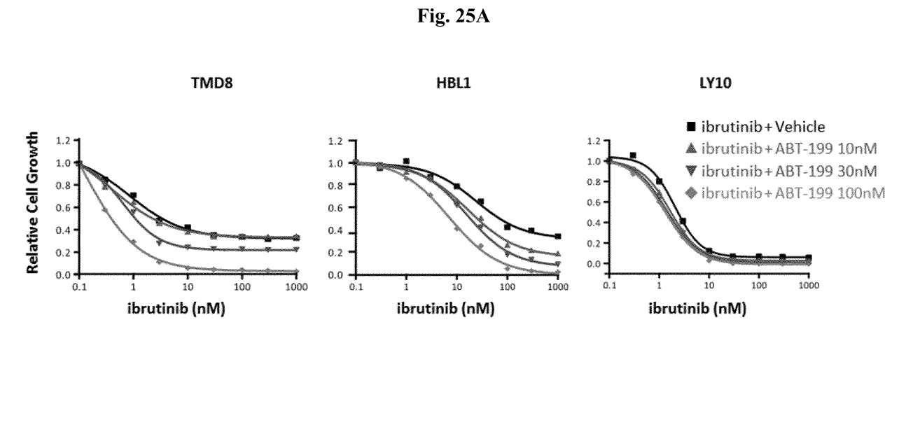

FIG. 24A-FIG. 24D show the effect of the combination of ibrutinib and ABT-199 on ibrutinib resistant DoHH2 proliferation. FIG. 24A illustrates the synergy score heat map of ibrutinib and ABT-199. FIG. 24B shows the percentage of growth of a second population of DoHH2 ibrutinib resistant cells in the presence of ABT-199 and ibruitnib. In some instances, the EC50 is 210.7 nM. FIGS. 24C and 24D show the synergy score of the ibrutinib and ABT-199 combination.

### Example 9

### ABC-DLBCL, GCB-DLBCL, and FL In Vitro and In Vivo Studies

### Methods

### Cell Culture and Drugs

ABC-DLBCL (TMD8, HBL1, and LY10), GCB-DLBCL (DLCL-2, RL, and SU-DHL-4), and FL (DoHH2 and WSU-FSCCL) cell lines were grown to log phase at 37° C. in the presence of 5% CO₂. TMD8 and HBL1 cells were cultured in RPMI 1640 medium (Life Technologies) with 10% FBS (Atlanta Biologicals), 1 mM sodium pyruvate (Life Technologies), and 1% Pen/Strep (Life Technologies). LY10 cells were cultured in IMDM medium (Life Technologies) with 20% heparinized normal human plasma (Equitech-Bio), 55 mM 2-mercaptoethanol (Life Technologies), and 1% Pen/Strep. DLCL-2, RL, SU-DHL-4, DoHH2, and WSU-FSCCL cells were cultured in RPMI 1640 medium (Life Technologies) with 10% FBS (Atlanta Biologicals), and 1% Pen/Strep (Life Technologies). HBL1-, TMD8-, and DoHH2-resistant cells were generated by in vitro culturing the parental cell lines for prolonged periods of time with progressively increasing concentrations of ibrutinib. LY10 (BTK-C481 S) was generated by introducing mutant BTK (C481S) into LY10 cell line.

### Cell Viability Assays

CellTiter-Glo® luminescent cell viability assay was performed according to manufacturer's instructions. Briefly, cells were seeded at 8,000-25,000 cells/well in a 96-well plate in the presence of single drugs or the drug combinations for 3 or 5 days. The number of viable cells in culture was determined by quantification of ATP present, which was proportional to luminescent signal detected. The combination index (C.I.), a drug interactivity measurement, was calculated with CalcuSyn (Biosoft). Synergy scores and isobolograms were calculated by the Chalice Analyzer (Horizon CombinatoRx).

### Adhesion Assays

Adhesion assays were performed in triplicate in 96-well plates coated overnight at 4° C. with PBS containing 10 μg/ml fibronectin or 4% BSA. Cells (5×10⁴) pretreated with indicated drugs overnight were seeded into each well and allowed to adhere in adhesion medium (RPMI-1640 containing 1% BSA) for 30 minutes at 37° C. After 4 times washed with prewarmed adhesion medium, the adherent cells were lysed in 100 μl of CellTiter-Glo reagent by gentle shaking and luminescent signal was measured according to manufacturer's protocol on a luminometer.

### RT-PCR Assays

TaqMan® Fast Cells-to-C_{T}™ kit (Life Technologies) was used to extract total RNA and reverse transcribe RNA to cDNA according to the manufacturer's specifications. 4 μl of cDNA from RT reaction was used to set up Taqman Q-RT-PCR on a QuantStudio™ 7 Flex real-time PCR System (Life Technologies). The TaqMan® gene expression assays used for this study include BCL-2 (Hs00608023_m1), BAX (Hs00180269_m1), MCL-1 (Hs01050896_m1), GAPDH (Hs02758991_g1), and ACTB (Hs01060665_g1).

### Xenograft Study

All animal studies were completed under the Institutional Animal Care and Use Committee (IACUC)-approved protocols for animal welfare. CB17 SCID mice (Charles River Laboratories) were subcutaneously inoculated with 1×10⁷ TMD8 cells in a suspension containing Matrigel (Corning). When tumors reached around 100 mm³ (16 days after tumor inoculation), mice were randomly assigned and treated once daily with ibrutinib (12 mg/kg), ABT-199 (40 mg/kg), or the combination by oral gavage with 10 mice per group. Tumor volume was measured twice a week and calculated as tumor volume=(length×width)×0.4.

### Apoptosis Assays

ApoDETECT™ annexin V-FITC Kit (Life Technologies) was used to detect the apoptotic cell population according to the manufacturer's specifications. Briefly, cells were washed with ice cold PBS and resuspended in 1× binding buffer at a concentration of 5×10⁵ cells/ml Annexin V-FITC (10 μL) was added to 190 μl of cell suspension and incubated at room temperature for 10 min. After being washed with 1× binding buffer, cells were resuspended in 190 μl of binding buffer with 10 μl of 20 μg/ml propidium iodide and analyzed by flow cytometry.

### Colony Formation Assays

HBL1 cells (1000 cells per well) were suspended in 0.9% methylcellulose (Methocult™ H4100, Stem Cell Technology) containing culture medium with vehicle, ibrutinib, ABT-199, or the combination and 0.3 ml of the mixture was plated in each well of 24-well culture plates. The colonies were counted on day 7.

### Microarray Data Analyses and Statistics

GeneChip® human transcriptome array 2.0 (HTA 2.0, Affymetrix) was used to analyze gene expression of TMD8 parental and ibrutinib resistant cell lines. Heatmap of apoptosis-related gene expression was generated using Transcriptome Analysis Console v2.0 (Affymetrix).

Gene expression of FFPE specimens from the phase 2 PCYC-1106 trial (NCT01325701) was analyzed using GeneChip® Human Genome U133 Plus 2.0 Array (Affymetrix) and data were normalized using the Robust Multi-array Average (RMA) algorithm. Subtypes of DLBCL were identified based on the classification algorithm. For the analysis restricted to ABC-DLBCL subtype, only the samples having a gene expression profiling (GEP) call of ABC-DLBCL were used and normalized separately. A test for differential expression of genes between ABC-DLBCL responders (CR+PR) and non-responders (SD+PD) to ibrutinib was performed using the rank product statistic (RankProd R package). For the ABC-DLBCL vs GCB-DLBCL comparison plot and heatmap, all subtypes were normalized together. The data were plotted in linear scale.

### Results

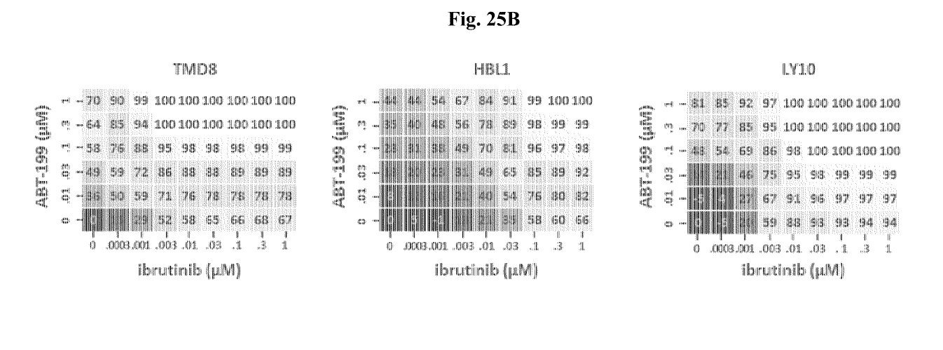

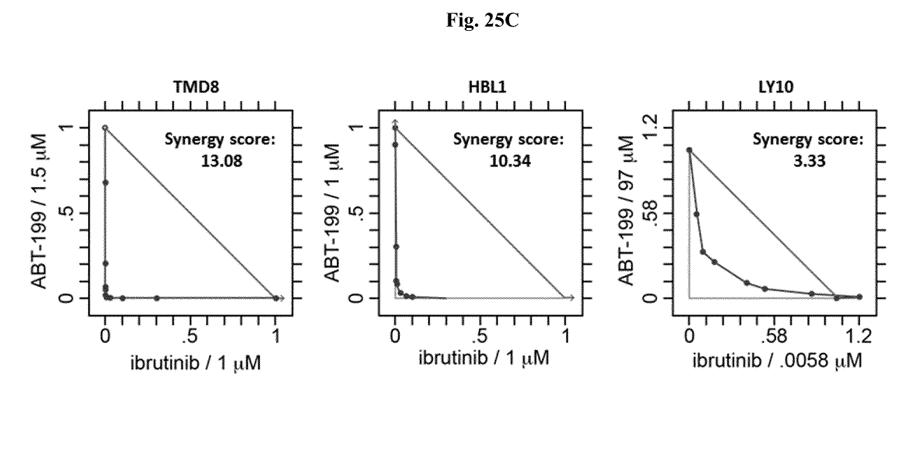

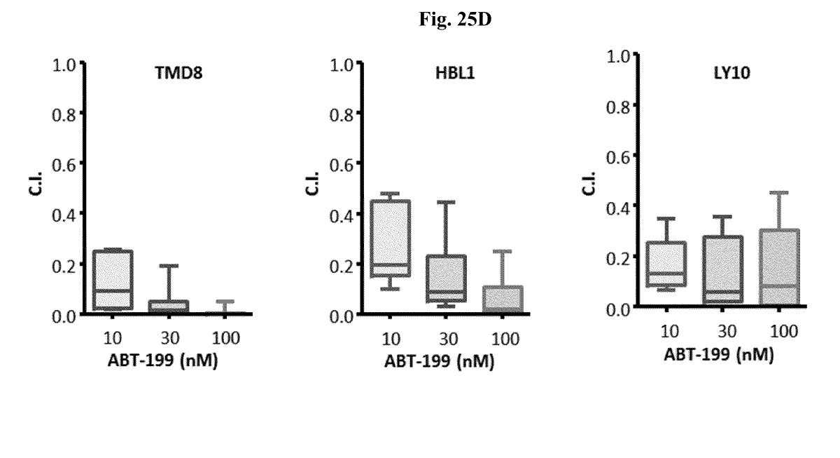

Ibrutinib and ABT-199 synergistically suppressed cell growth in ABC-DLBCL cells (FIGS. 25A-FIG. 25D). (FIG. 25A) TMD8, HBL1, and LY10 cells were treated with the indicated concentrations of ibrutinib combined with ABT-199 (10, 30, 100 nM) or vehicle for 5 days, and the drug effect on cell growth was determined by CellTiter-Glo Luminescent Cell Viability Assay. (FIG. 25B) Drug dose matrix data of TMD8, HBL1, and LY10 cells. The numbers indicated the percentage of growth inhibition of cells treated with the corresponding compound combination relative to vehicle control-treated cells. The data were visualized over matrix using a color scale. (FIG. 25C) Isobologram analysis and synergy scores of the data in FIG. 25B indicated synergy for the combination of ibrutinib and ABT-199. (FIG. 25D) C.I. of ibrutinib and ABT-199 at indicated concentrations in TMD8, HBL1, and LY10 cells.

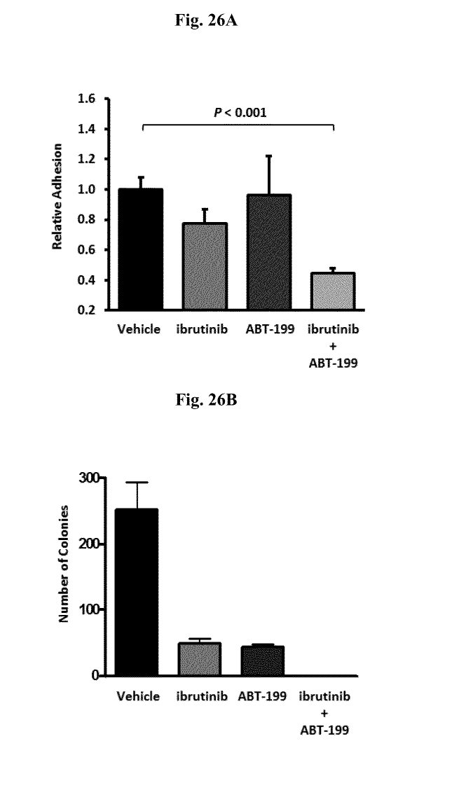

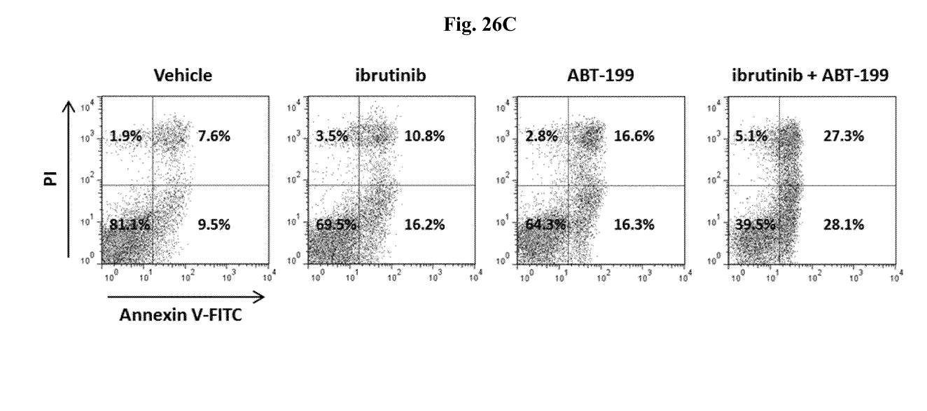

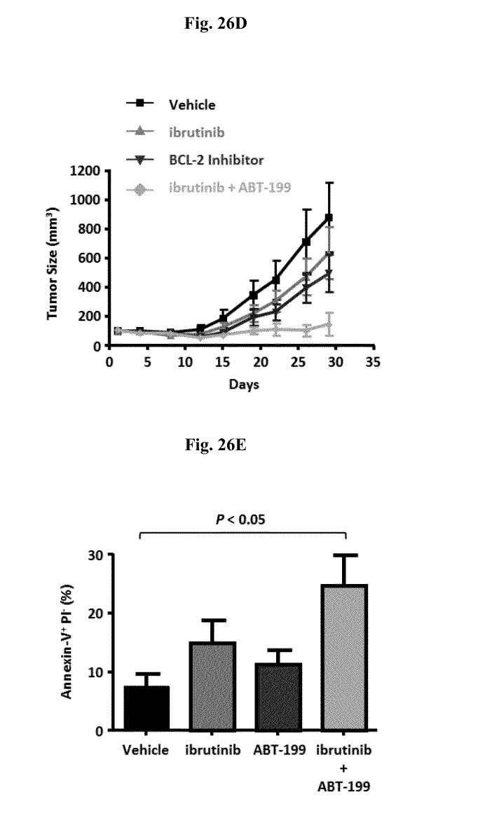

Combinations of ibrutinib and ABT-199 inhibited cell adhesion and colony formation, increased apoptotic cell population, and suppressed tumor growth (FIG. 26A-FIG. 26C). (FIG. 26A) TMD8 cells were pretreated with vehicle, ibrutinib (0.1 μM), ABT-199 (1 μM), or the combination overnight before seeded into plates for adhesion assay. Wells coated with BSA served as negative controls. The luminescent signal obtained in the negative controls was subtracted from that obtained in all treatment groups. All data were presented as luminescent signal fold-change relative to vehicle-treated samples. Graphs represented quantifications of 3 wells, expressed as mean±SD. (FIG. 26B) HBL1 cells were plated in 0.9% MethoCult (1000 cells/well) with vehicle, ibrutinib (10 nM), ABT-199 (50 nM), or the combination and colony formation was scored after 7 days. Graphs represented quantifications of 3 wells, expressed as mean±SD. (FIG. 26C) TMD8 cells were treated for 1 day with ibrutinib (100 nM), ABT-199 (1 μM), or the combination, and analyzed for annexin-V binding as well as for PI uptake. The percentage of cells annexin V positive, PI positive or double positive for both annexin V and PI is indicated. (FIG. 26D) TMD8 tumor cells were implanted into CB17 SCID mice and the indicated drugs were orally administrated daily when the tumors reached 100 mm3. Tumors were measured twice a week. (FIG. 26E) Apoptotic cell population (annexin V positive and PI negative) of TMD8 tumor cells from CB17 SCID mice treated with indicated drugs were analyzed by flow cytometry.

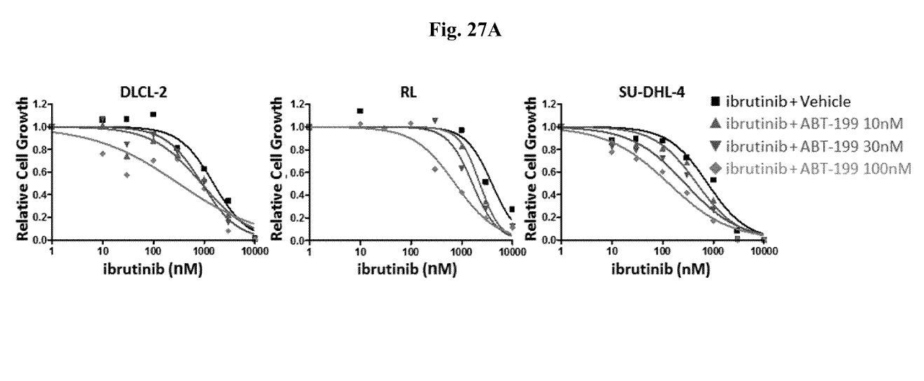

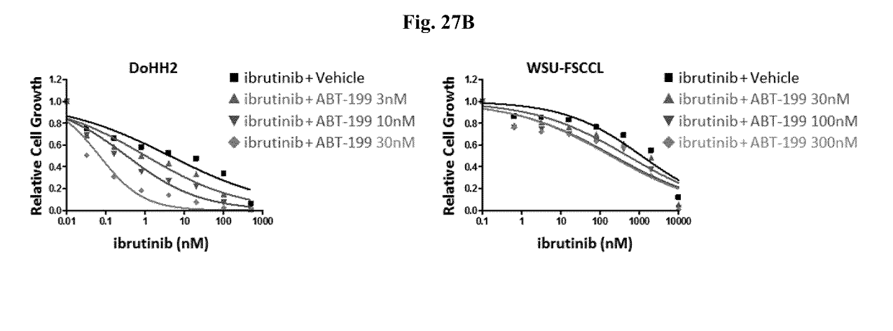

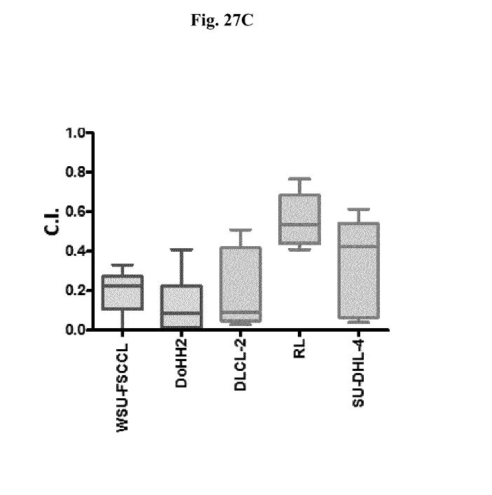

Ibrutinib and ABT-199 synergistically suppressed cell growth in GCB-DLBCL and FL cells (FIGS. 27A-27C). (FIG. 27A) GCB-DLBCL cells (DLCL-2, RL, and SU-DHL-4) were treated with indicated concentrations of ibrutinib combined with ABT-199 (10, 30, 100 nM) or vehicle for 3 days, and the drug effect on cell growth was determined by CellTiter-Glo Luminescent Cell Viability Assay. (FIG. 27B) FL cells (DoHH2 and WSU-FSCCL) were treated with indicated concentrations of ibrutinib combined with ABT-199 or vehicle for 3 days, and the drug effect on cell growth was determined by CellTiter-Glo Luminescent Cell Viability Assay. (FIG. 27C) C.I. of ibrutinib and ABT-199 combination in GCB-DLBCL and FL cells. Shown are C.I.s of different concentrations of ibrutinib combined with ABT-199 at 100 nM (DLCL-2, RL, and SU-DHL-4), 30 nM (DoHH2), and 100 nM (WSU-FSCCL).

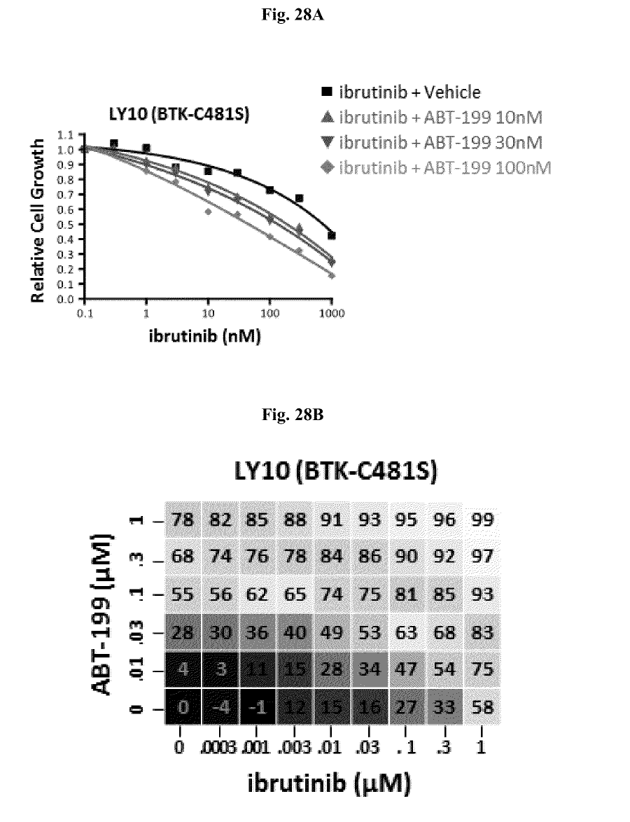

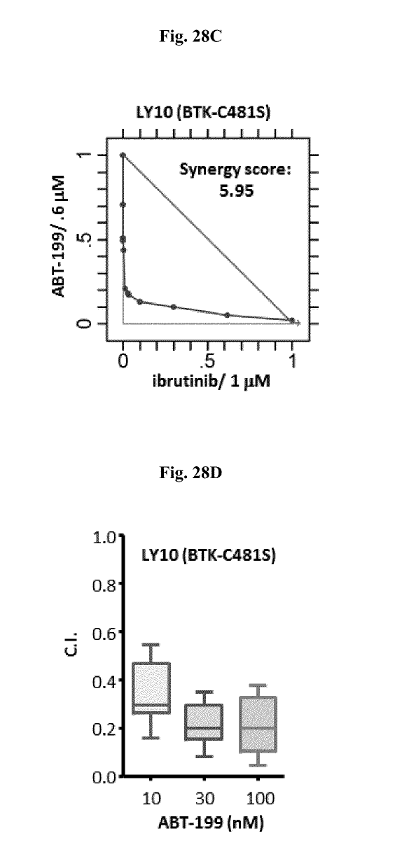

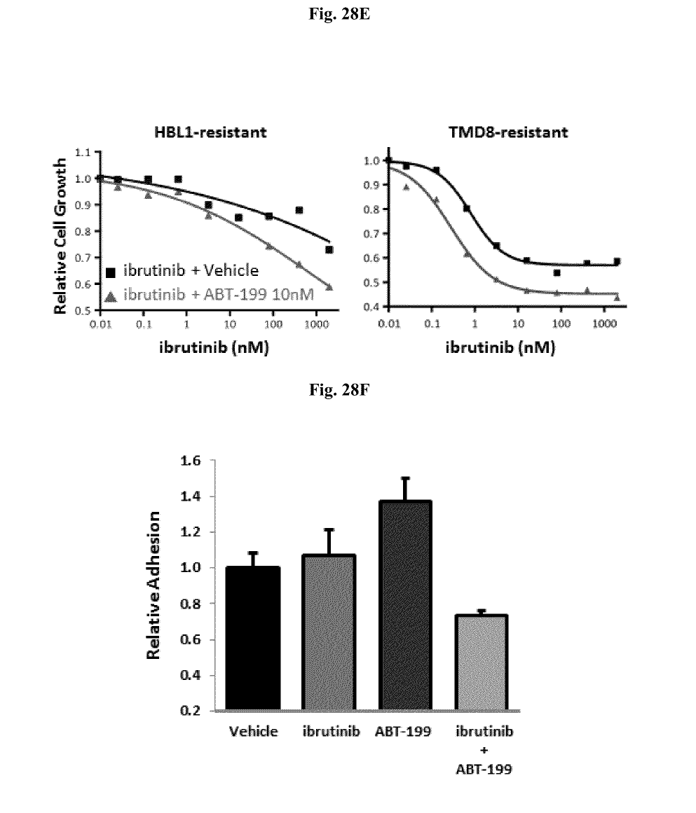

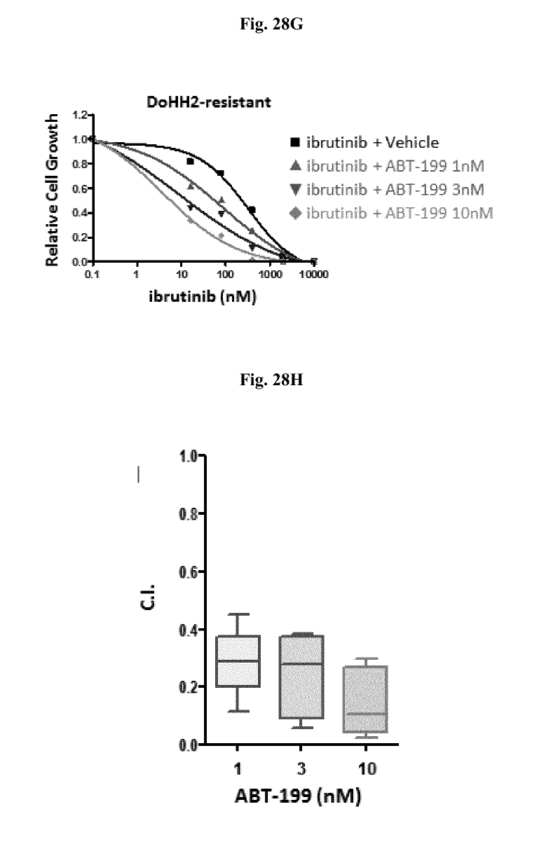

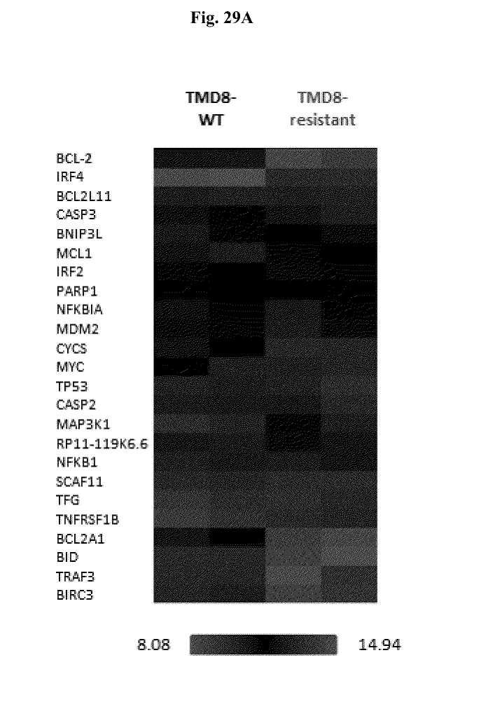

Ibrutinib and ABT-199 synergistically suppressed cell growth in ibrutinib-resistant ABC-DLBCL cells (FIG. 28A-FIG. 28H). (FIG. 28A) LY10 (BTK-C481S) cells were treated with indicated concentrations of ibrutinib combined with ABT-199 (10, 30, 100 nM) or vehicle for 5 days, and the drug effect on cell growth was determined by CellTiter-Glo Luminescent Cell Viability Assay. (FIG. 28B) Drug dose matrix data of LY10 (BTK-C481S) cells. (FIG. 28C) Isobologram analysis and synergy scores of the data in FIG. 28B. (FIG. 28D) C.I. of ibrutinib and ABT-199 at indicated concentrations in LY10 (BTK-C481S) cells. (FIG. 28E) HBL1-resistant and TMD8-resistant cells were treated with indicated concentrations of ibrutinib combined with ABT-199 (10 nM) or vehicle for 3 days, and the drug effect on cell growth was determined by CellTiter-Glo Luminescent Cell Viability Assay. (FIG. 28F) TMD8-resistant cells were pretreated with vehicle, ibrutinib (0.1 μM), ABT-199 (1 μM), or the combination overnight before seeded into plates for adhesion assay. All data were presented as luminescent signal fold-change relative to vehicle-treated samples. Graphs represented quantifications of 3 wells, expressed as mean±SD. (FIG. 28G) DoHH2-resistant cells were treated with indicated concentrations of ibrutinib combined with ABT-199 (1, 3, 10 nM) or vehicle for 3 days, and the drug effect on cell growth was determined by CellTiter-Glo Luminescent Cell Viability Assay. (FIG. 28H) C.I. of ibrutinib and ABT-199 at indicated concentrations in DoHH2-resistant cells.

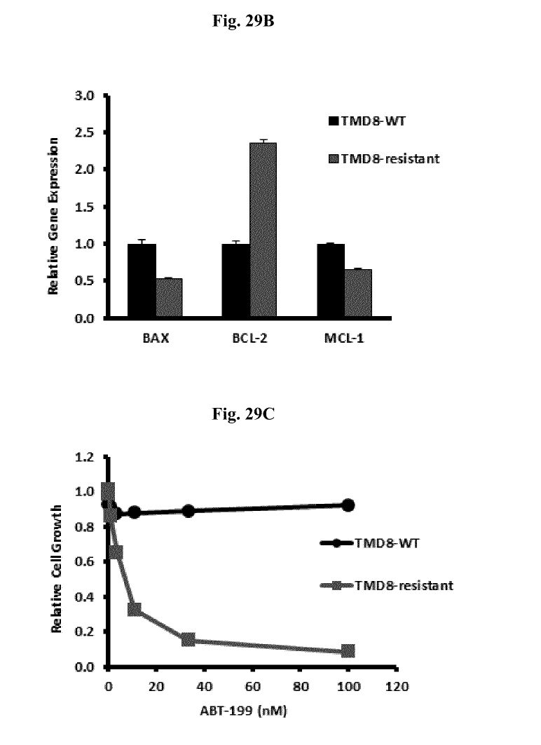

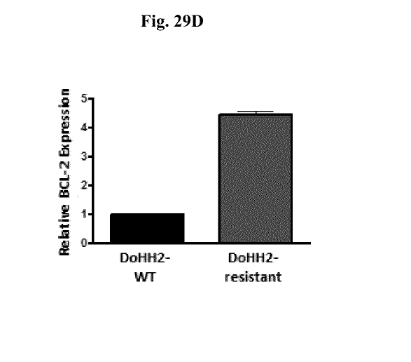

TMD8-resistant cells had higher BCL-2 gene expression and were more sensitive to ABT-199 (FIG. 29A-FIG. 29D). (FIG. 29A) Heat-map presentation of gene-expression profiles of apoptosis-related genes in TMD8-WT versus TMD8-resistant cells. (FIG. 29B) BCL-2 gene expression increased in TMD8-resistant cells. Gene expression levels of BAX, BCL-2, and MCL-1 were determined by RT-QPCR assay and GAPDH and ACTB were used as reference genes. All data were presented as fold change over TMD8-WT samples. (FIG. 29C) TMD8-resistant cells were more sensitive to ABT-199 compared to TMD8-WT cells. Cells were treated with ABT-199 for 3 days and the drug effect on cell growth was determined by CellTiter-Glo luminescent cell viability assay. (FIG. 29D) BCL-2 gene expression increased in DoHH2-resistant cells. Gene expression level of BCL-2 was determined by RT-QPCR assay and GAPDH was used as a reference gene. Data were presented as fold change over DoHH2-WT sample.

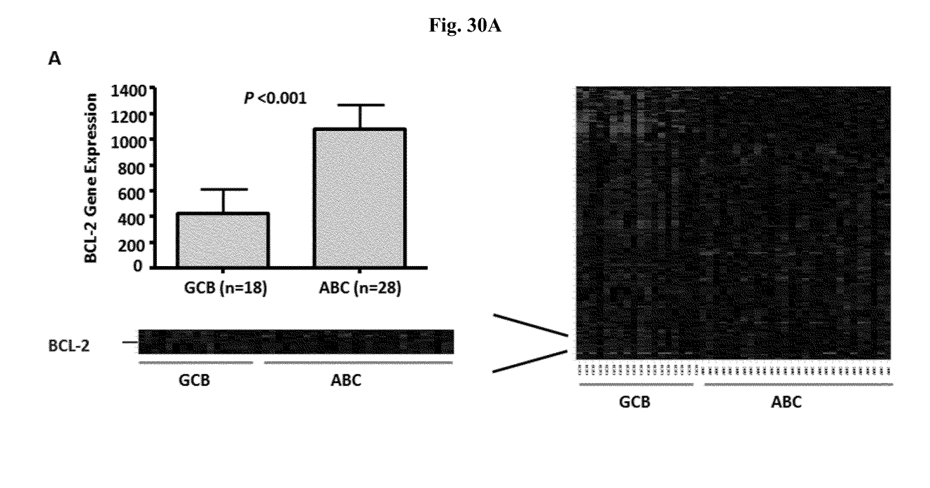

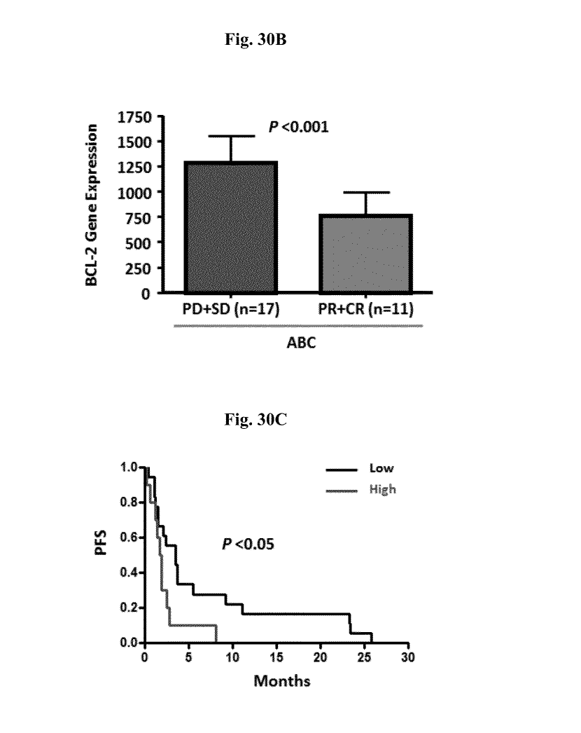

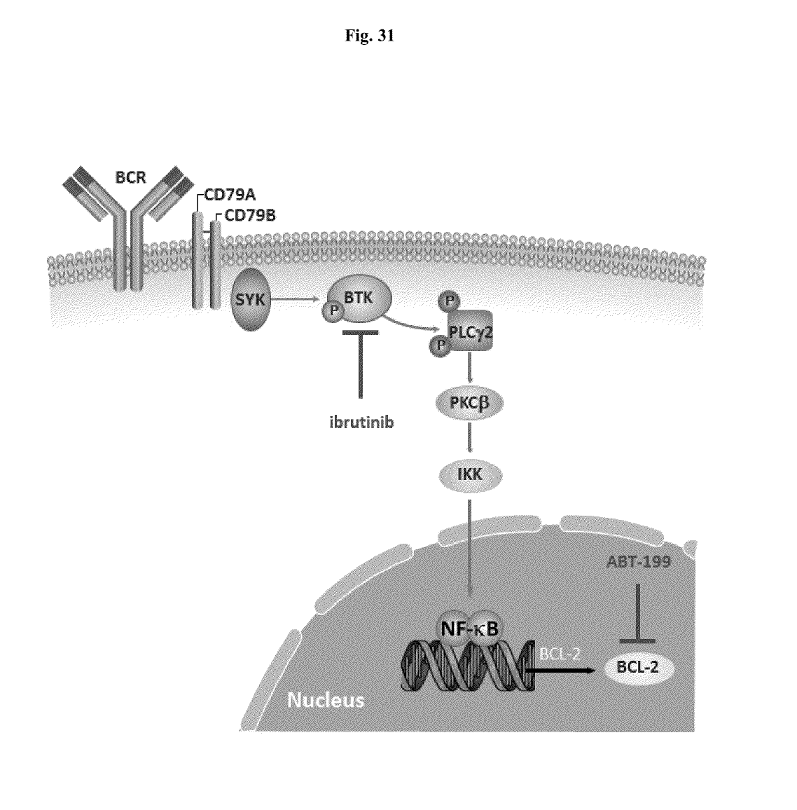

Higher BCL-2 gene expression was observed in the tumors from patients with poorer response to ibrutinib (FIG. 30A-FIG. 30C). (FIG. 30A) Differential BCL-2 gene expression was observed in the tumors from ABC-DLBCL and GCB-DLBCL patients. (FIG. 30B) Higher BCL-2 gene expression was detected in the tumors from ABC-DLBCL patients with poorer response (PD+SD). BCL-2 gene expression levels were analyzed and a rank based statistic (RankProd) was used to determine the significance (p<0.001). (FIG. 30C) Kaplan-Meier survival curves of progression-free survival for patients with low BCL-2 (black) and high BCL-2 (red) gene expression. ABC-DLBCL patients with higher BCL-2 gene expression had significantly worse survival than those with lower BCL-2 gene expression (p<0.05, Logrank test).

### Example 10

### Combination Effect of a Btk Inhibitor, aBcl-2 Inhibitor, and a PI3K Inhibitor

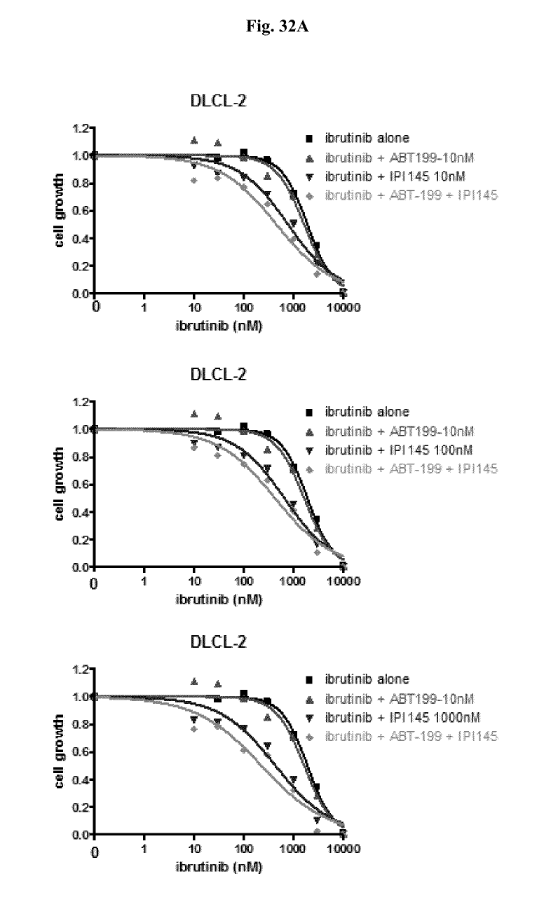

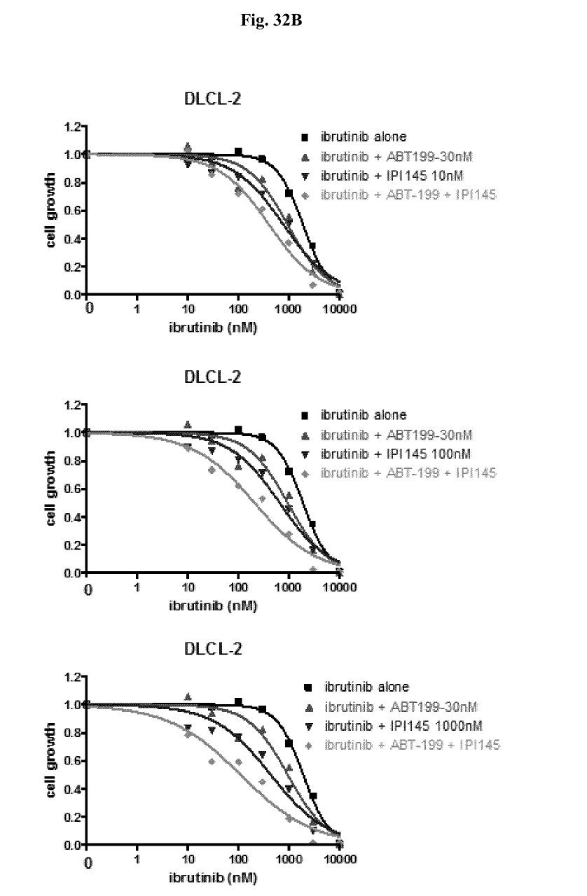

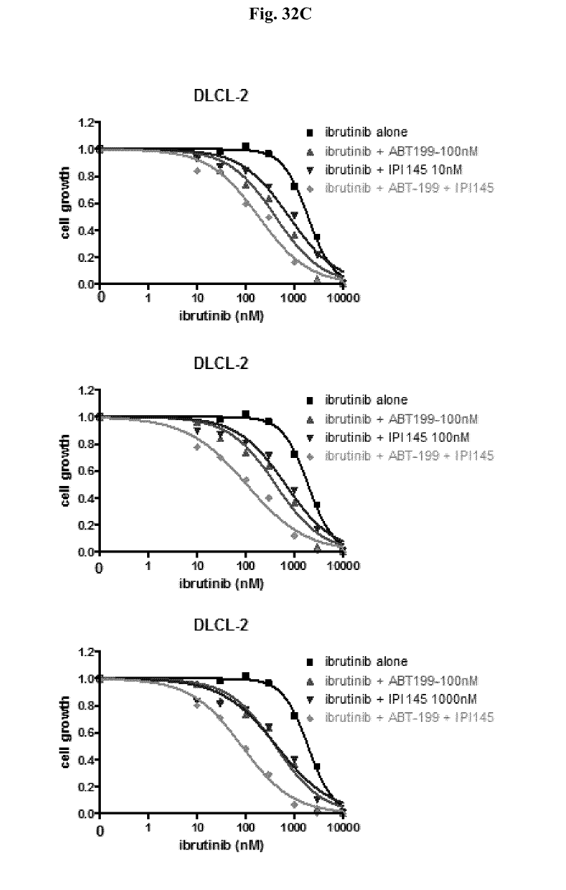

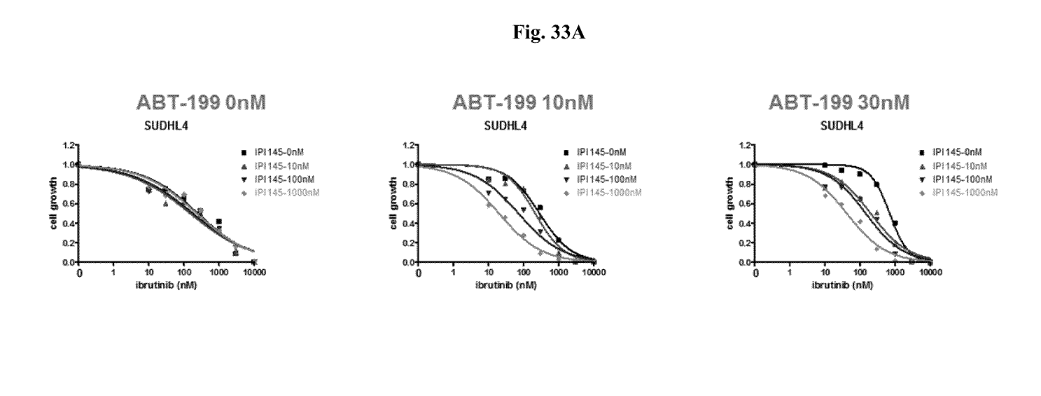

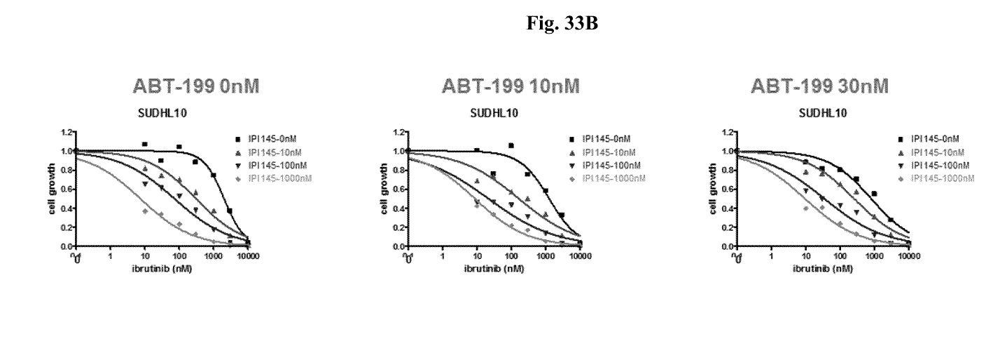

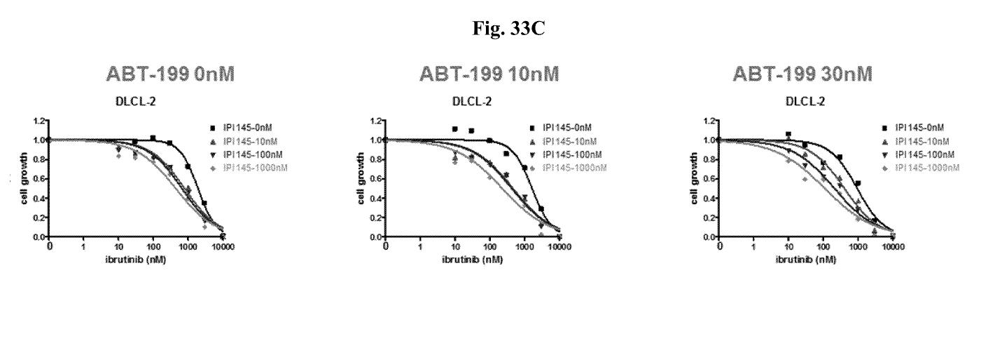

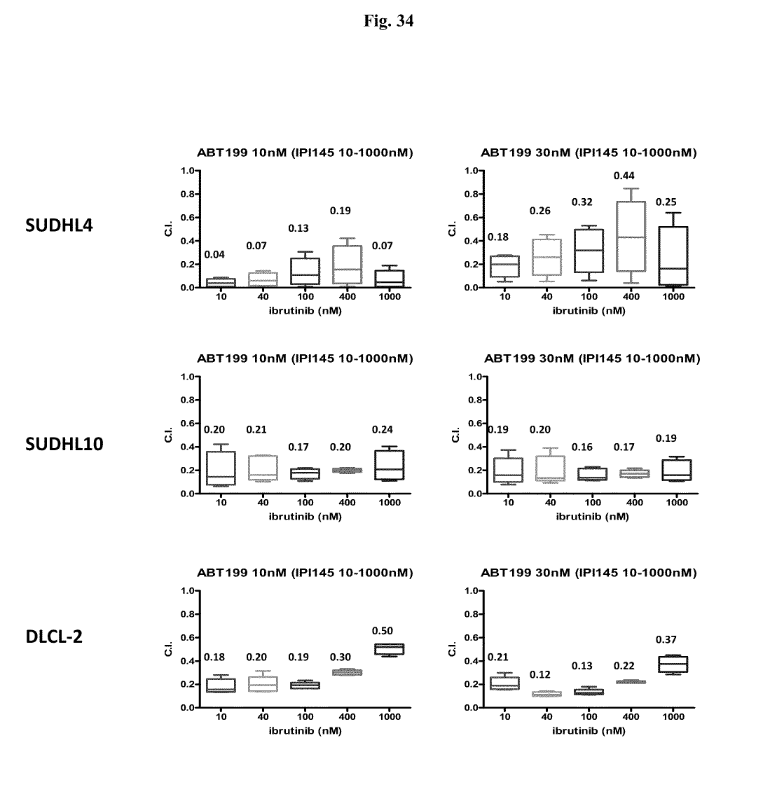

GCB-DLBCL cell lines (SUDHL4, SUDHL5, SUDHL6, SUDHL10, WSU-NHL, DLCL-2, and RL) were cultured in the presence of the Btk inhibitor ibrutinib alone; ibrutinib with the Bcl-2 inhibitor ABT-199; ibrutinib with the PI3K inhibitor IPI-145; or ibrutinib with ABT-199 and IPI-145, and the drug effect on cell growth was determined. Synergy of the ibrutinib/ABT-199/IPI-145 combination was identified in the SUDHL4 cell line, the SUDHL10 cell line, and the DLCL-2 cell line. FIGS. 32A-32C show cell growth plots of DLCL-2 cells that were grown in the presence of ibrutinib alone; ibrutinib and ABT-199; ibrutinib and IPI-145; or ibrutinib with ABT-199 and IPI-145 at the indicated concentrations. FIGS. 33A-33C show cell growth plots of SUDHL4, SUDHL10, and DLCL-2 cells that were grown in the presence of ibrutinib alone; ibrutinib and ABT-199; ibrutinib and IPI-145; or ibrutinib with ABT-199 and IPI-145 at the indicated concentrations. C.I. values of the combination of ibrutinib, ABT-199, and IPI-145 were calculated for the combination in SUDHL4, SUDHL10, and DLCL-2 cells and indicated synergy for these three cell lines (FIG. 34, numbers shown are the average C.I. values).

### Example 11

### Combination Effect of a Btk Inhibitor, aBcl-2 Inhibitor, and a Corticosteroid

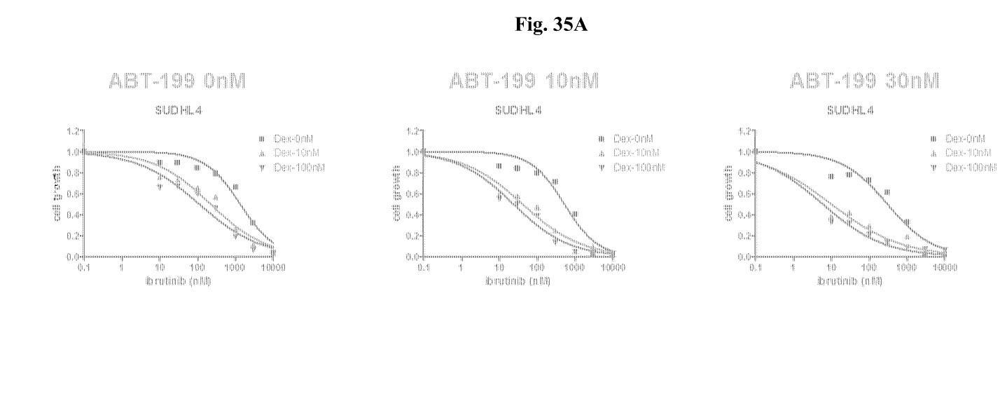

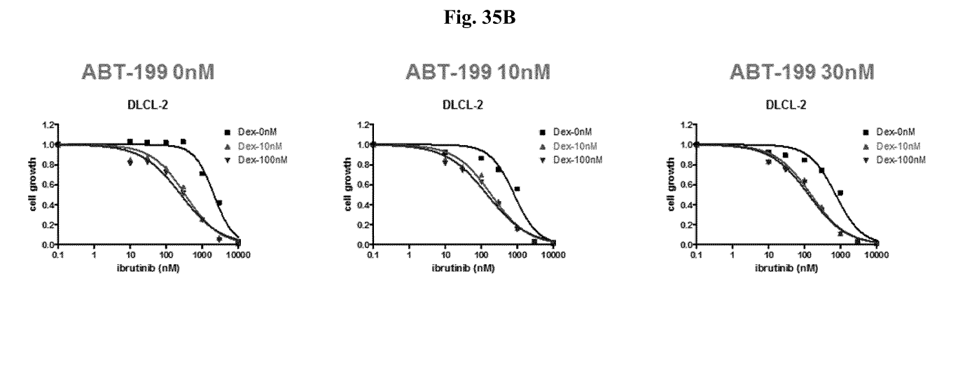

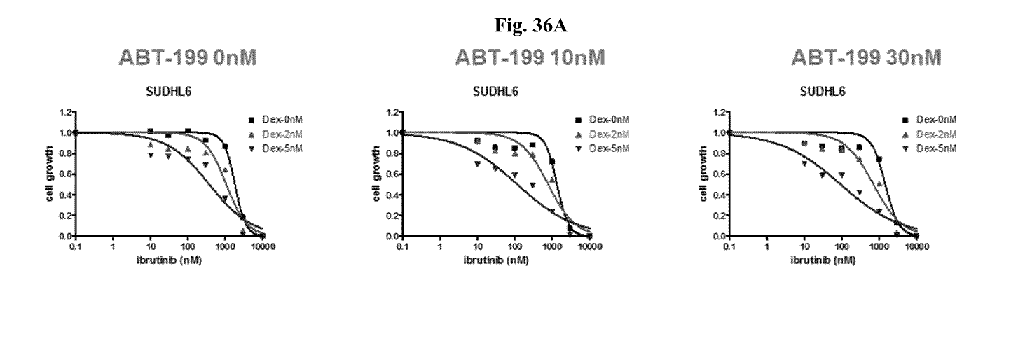

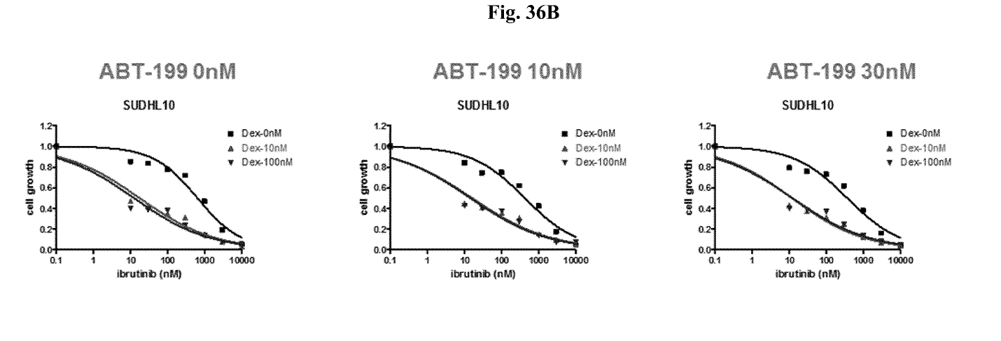

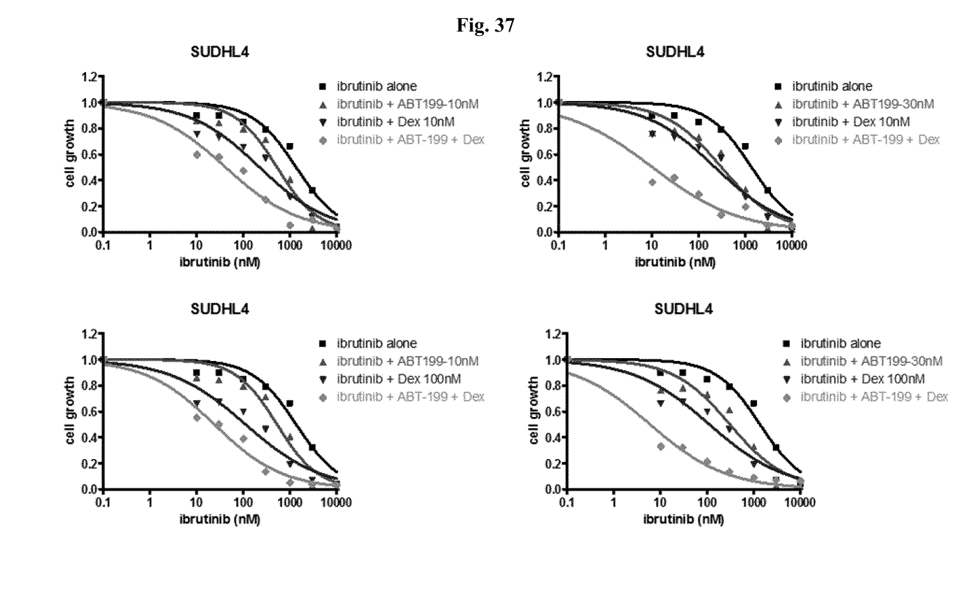

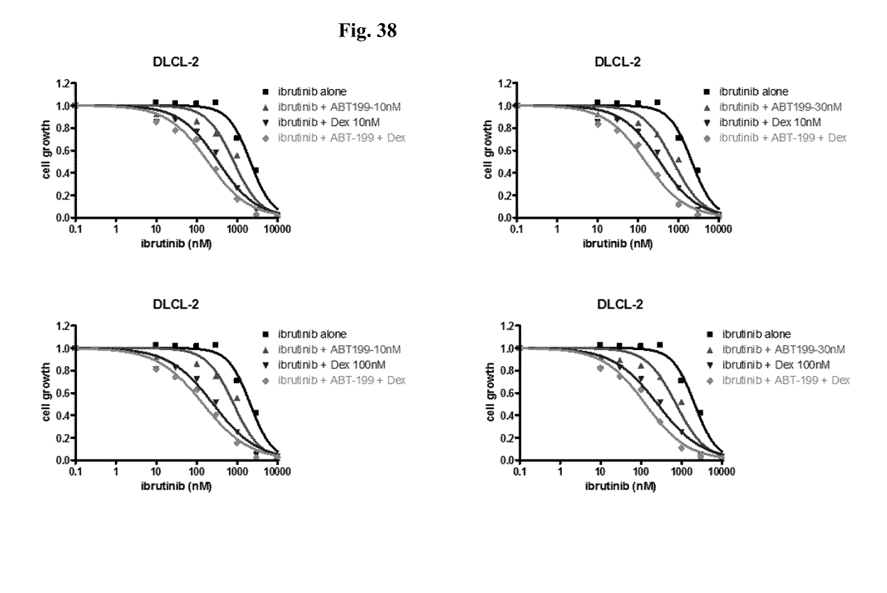

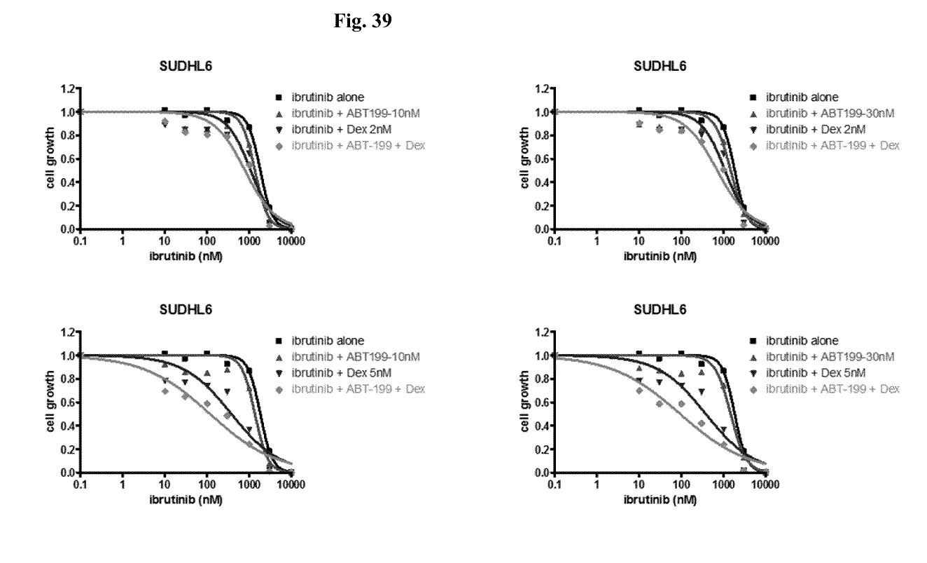

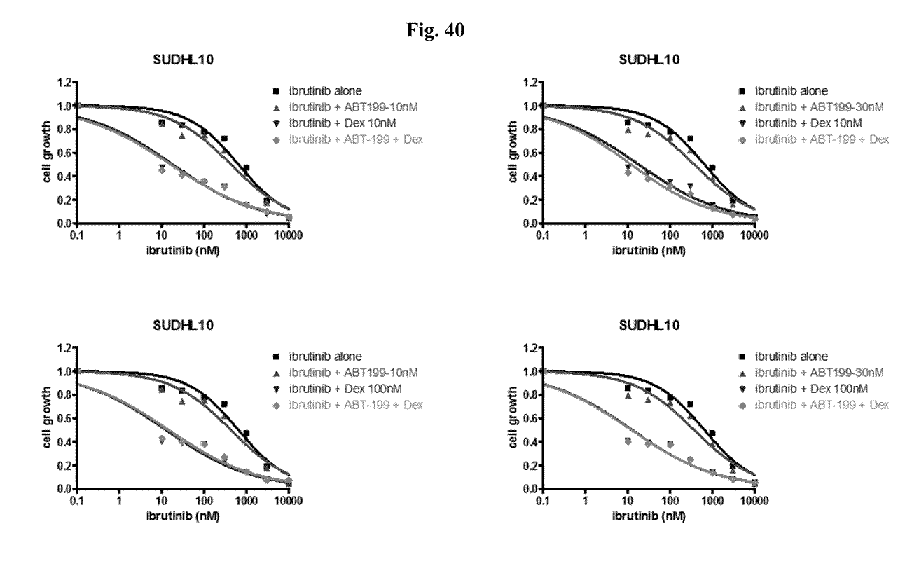

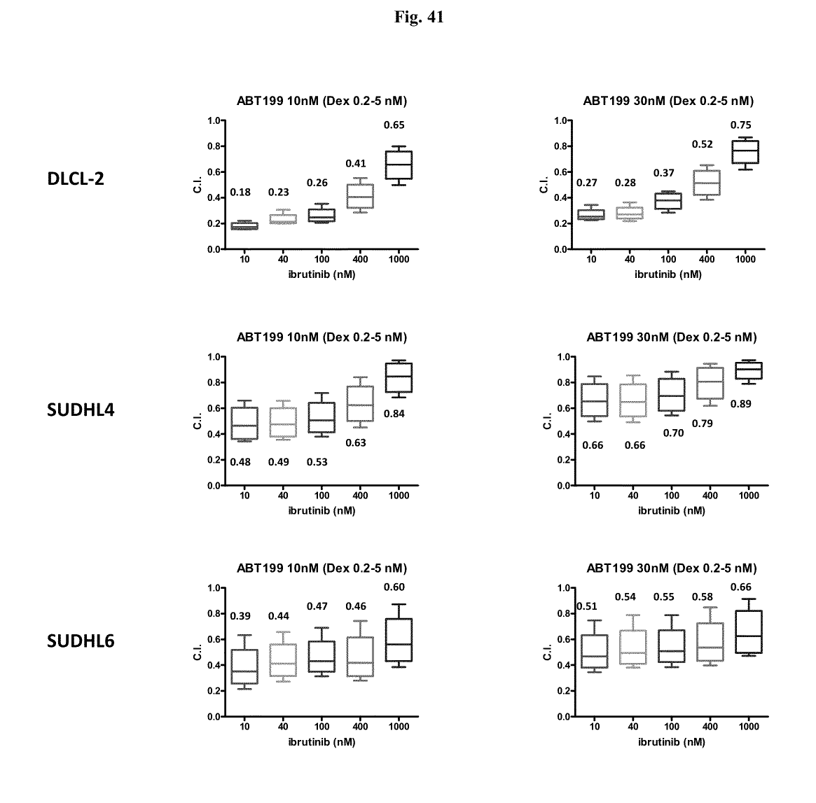

GCB-DLBCL cell lines (SUDHL4, SUDHL6, SUDHL10, and DLCL-2) were cultured in the presence of the Btk inhibitor ibrutinib alone; ibrutinib with the Bcl-2 inhibitor ABT-199; ibrutinib with the corticosteroid dexamethasone; or ibrutinib with ABT-199 and dexamethasone, and the drug effect on cell growth was determined. Synergy of the ibrutinib/ABT-199/dexamethasone combination was identified in the SUDHL4 cell line, the SUDHL6 cell line, and the DLCL-2 cell line. FIGS. 35A and 35B show cell growth plots of SUDHL4 cells and DLCL-2 cells that were grown in the presence of ibrutinib alone; ibrutinib and ABT-199; ibrutinib and dexamethasone; or ibrutinib with ABT-199 and dexamethasone at the indicated concentrations. FIGS. 36A and 36B show cell growth plots of SUDHL6 and SUDHL10 cells that were grown in the presence of ibrutinib alone; ibrutinib and ABT-199; ibrutinib and dexamethasone; or ibrutinib with ABT-199 and dexamethasone at the indicated concentrations. FIGS. 37-40 show cell growth plots of SUDHL4 cells, DLCL-2 cells, SUDHL6, and SUDHL10 cells, respectively that were grown in the presence of ibrutinib alone; ibrutinib and ABT-199; ibrutinib and dexamethasone; or ibrutinib with ABT-199 and dexamethasone at the indicated concentrations. C.I. values of the combination of ibrutinib, ABT-199, and dexamethasone were calculated for the combination in SUDHL4, SUDHL6, and DLCL-2 cells and indicated synergy for these three cell lines (FIG. 41, numbers shown are the average C.I. values).

### Example 12

### Mutation Impact of Targeted Genes in Diffuse Large B-Cell Lymphoma Patients Treated with Ibrutinib

Through targeted deep sequencing, the impact of baseline mutations of 317 targeted genes on clinical response of 51 DLBCL patients treated with ibrutinib was investigated. Based on this mutation impact analysis, potential biomarkers for predicting DLBCL patient response to ibrutinib were identified. In particular, sets of gene mutation patterns indicating poor (or good) clinical response across all subtypes (ABC, non-GCB, GCB) of DLBCL as well as uniquely within a subtype were identified.

Methods: An H&E-stained slide of each DLBCL sample from patients enrolled in either PCYC-04753 (NCT00849654) or PCYC-1106 (NCT01325701) was reviewed to ensure sufficient nucleated cellularity and tumor content. DNA and RNA were extracted from unstained sections of FFPE DLBCL tumor biopsies. Sequencing was performed using the FoundationOne™ Heme panel following the validated NGS-based protocol to interrogate complete coding DNA sequences of 405 genes as well as selected introns of 31 genes involved in rearrangements, and RNA sequence of 265 commonly rearranged genes to better identify gene fusions. A subgroup of samples used earlier versions of FoundationOne™ panels where only DNA was extracted and sequenced. Sequence data were processed and analyzed for base substitutions, insertions, deletions, copy-number alterations, and selected gene fusions. Mutation impact indices of 317 genes were calculated and plotted for overall gene mutation pattern recognition. Chi-square association tests were performed on cases where sufficient sample sizes were available to determine statistical significance of mutation impact. DLBCL subtype classifications by gene expression profiling (GEP) and Hans' IHC were investigated and compared. For GEP, we utilized OmicSoft ArrayStudio's classification module to build linear discriminant analysis (LDA) model/classifier and neural networks with 5-fold cross validation procedure for model selection. The LDA was best performing model and was selected for final GEP classification. Since only 29 (out of 51) patients had central lab Hans' IHC classification information, trends of the mutation impact results based on Hans' classification and GEP classification were compared.

Results: Single or multiple gene mutation impact indices (MII) were generated from baseline tumor biopsies from DLBCL patients treated with ibrutinib monotherapy. The MII were generally consistent between GEP or Hans IHC classification of tumor biopsies. Novel baseline gene mutations identified as associated with poor clinical response (SD or PD) to ibrutinib such as those involved in regulation of transcription (e.g., mutations in EP300 in all DLBCL subtypes combined group [p=0.034], mutations in RB1 in ABC-DLBCL [p=0.031]), epigenetic modification (e.g., mutations in MLL2 in ABC-DLBCL [p=0.053]), programmed cell death (mutations in BCL2 in all DLBCL subtypes [p=0.096]), and PI3K-AKT-mTOR pathway (e.g., mutations in TSC2 in ABC-DLBCL [p=0.031]) were identified. Mutations identified as indicating good clinical response included mutations in CD79B [p=0.072] and MYD88 [p=0.024] in ABC-DLBCL. Co-existence of MYD88 and CD79B mutations (double-mutants) in ABC-DLBCL patients showed a stronger association to good clinical response [p=0.004]. This investigation revealed unique mutation patterns that underlie DLBCL subtypes and highlights the need for personalized medicine approaches to treating these patients.

The examples and embodiments described herein are for illustrative purposes only and various modifications or changes suggested to persons skilled in the art are to be included within the spirit and purview of this application and scope of the appended claims.

What is claimed is:

1. A method for selecting an individual having diffuse large B cell lymphoma (DLBCL) for treatment with ibrutinib, comprising:
a. determining the presence or absence of a modification in one or more biomarker genes selected from EP300, MLL2, BCL-2, RB1, LRP1B, PIM1, TSC2, TNFRSF11A, SMAD4, PAX5, and CARD11; and
b. administering to the individual a therapeutically effective amount of ibrutinib if there is an absence of a modification in the one or more biomarker genes selected from EP300, MLL2, BCL-2, RB1, LRP1B, PIM1, TSC2, TNFRSF11A, SMAD4, PAX5, and CARD11.

2. The method of claim 1, further comprising determining the presence or absence of a modification in two or more biomarker genes selected from EP300, MLL2, BCL-2, RB1, LRP1B, PIM1, TSC2, TNFRSF11A, SMAD4, PAX5, and CARD11.

3. The method of claim 1, wherein the one or more biomarker genes are selected from BCL-2, RB1, LRP1B, PIM1, and TSC2.

4. The method of claim 1, wherein the modification associated with the EP300, MLL2, BCL-2, RB1, LRP1B, PIM1, TSC2, TNFRSF11A, SMAD4, PAX5, and CARD11 genes results in a modification in the EP300, MLL2, BCL2, RB1, LRP1B, PIM1, TSC2, TNFRSF11A, SMAD4, PAX5, and CARD11 proteins.

5. The method of claim 4, wherein the BCL-2 protein comprises one or more modifications at positions corresponding to amino acid residues 4, 9, 33, 47, 48, 49, 60, 68, 74, 113, 114, 120, 122, 129, 131, 165, 197, 198, 200, 201, 203, and 206.

6. The method of claim 5, wherein the modifications include A4S, Y9H, G33R, G47A, I48S, F49L, A60T, R68K, T74N, T74S, A113G, E114A, H120Y, T122S, R129H, A131V, E165D, G197R, G197S, A198V, G200S, D201N, S203N, and 206W.

7. The method of claim 1, wherein DLBCL is activated B-cell DLBCL (ABC-DLBCL), germinal center B-cell like DLBCL (GBC-DLBCL), or unclassified DLBCL.

8. The method of claim 1, wherein the DLBCL is a relapsed or refractory DLBCL.

9. A method for selecting an individual having diffuse large B cell lymphoma (DLBCL) for treatment with ibrutinib, comprising:
a. determining the presence or absence of a modification to an aromatic residue at amino acid position 196 in CD79B and at least one modification at amino acid positions 198 or 265 in MYD88; and
b. administering to the individual a therapeutically effective amount of ibrutinib if there is a presence of the modification to an aromatic residue in CD79B and at least one modification at amino acid positions 198 or 265 in MYD88.

10. The method of claim 9, wherein the modification at amino acid position 196 in CD79B is Y196F.

11. The method of claim 9, wherein the modification at amino acid position 198 in MYD88 is S198N.

12. The method of claim 9, wherein the modification at amino acid position 265 in MYD88 is L265P.

13. The method of claim 9, wherein the combination of the modifications in CD79B and MYD88 is Y196F and S198N or Y196F and L265P.

14. The method of claim 9, wherein the DLBCL is activated B-cell DLBCL (ABC-DLBCL) or unclassified DLBCL.

15. The method of claim 9, wherein the DLBCL is a relapsed or refractory DLBCL.

16. A method for selecting an individual having diffuse large B cell lymphoma (DLBCL) for treatment with ibrutinib, comprising:
a. determining the presence or absence of a modification at amino acid position 15 in ROS1; and
b. administering to the individual a therapeutically effective amount of ibrutinib if there is an absence of the modification at amino acid position 15 in ROS1.

17. The method of claim 16, wherein the modification at amino acid position 15 in ROS1 is A15G.

18. The method of claim 17, wherein the A15G modification in ROS1 further indicates the individual has developed or likely to develop a progressive DLBCL.

19. The method of claim 16, wherein DLBCL is activated B-cell DLBCL (ABC-DLBCL), germinal center B-cell like DLBCL (GBC-DLBCL), or unclassified DLBCL.

20. The method of claim 16, wherein the DLBCL is a relapsed or refractory DLBCL.
